(19) United States
(12) Patent Application Publication
(10) Pub. No.: US 2024/0000767 A1
(43) Pub. Date: Jan. 4, 2024

(54) COMBINATION OF AN ALPHA2-ADRENOCEPTOR SUBTYPE C (ALPHA-2C) ANTAGONIST WITH A TASK1/3 CHANNEL BLOCKER FOR THE TREATMENT OF SLEEP APNEA

(71) Applicant: Bayer Aktiengesellschaft, Leverkusen (DE)

(72) Inventors: Martina DELBECK, Heiligenhaus (DE); Michael HAHN, Langenfeld (DE)

(73) Assignee: Bayer Aktiengesellschaft, Leverkusen (DE)

(21) Appl. No.: 18/014,664

(22) PCT Filed: Jul. 5, 2021
(86) PCT No.: PCT/EP2021/068487

§ 371 Date: Jan. 5, 2023

(30) Foreign Application Priority Data
Jul. 6, 2020 (EP) 20184206.9

(51) Int. Cl. A61K 31/4545; A61P 11/04; A61K 31/55; A61K 31/4439

(52) U.S. Cl. CPC A61K 31/4545; A61K 31/4439; A61K 31/55; A61P 11/04

(57) ABSTRACT

The present invention relates to a combination of selective blockers of TASK-1 and TASK-3 channels, in particular substituted imidazo [1,2-a]pyrimidine and substituted imidazo [1,2-a]pyridine derivatives of formula (II) and α2-Adrenoceptor subtype C (alpha-2C) antagonists, in particular substituted heterocyclic carboxamides of formula (I) for the treatment and/or prophylaxis of sleep-related breathing disorders, preferably obstructive and central sleep apneas and snoring.

The present invention relates to a combination of selective blockers of TASK-1 and TASK-3 channels, in particular substituted imidazo[1,2-a]pyrimidine and substituted imidazo[1,2-a]pyridine derivatives of formula (II) and α2-Adrenoceptor subtype C (alpha-2C) antagonists, in particular substituted heterocyclic carboxamides of formula (I) for the treatment and/or prophylaxis of sleep-related breathing disorders, preferably obstructive and central sleep apneas and snoring.

### BACKGROUND OF THE INVENTION

Obstructive sleep apnoea (OSA) is a sleep-related respiratory disorder which is characterized by repetitive episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper respiratory tract is substantially determined by the coordination and contraction property of the dilating muscles of the upper airways.

Upper airway collapse in OSA is thought to occur at sleep onset because of the reduction of activity of several upper airway dilator muscles, which as a consequence are unable to maintain the anatomically vulnerable airway open. However, some upper airway dilator muscles, including the genioglossus muscle, which is the most important of the dilating muscles of the upper respiratory airway and which is innervated by the hypoglossal nerve, can increase activity during sleep in response to respiratory stimuli, potentially counteracting some of these changes at sleep onset. It was observed that OSA patients have apnea free intervals in which the genioglossus muscle activity is only 25-40% higher compared with sleep phases with frequent obstructive apneas (Jordan A S, White D P, Lo Y L et al., Airway dilator muscle activity and lung volume during stable breathing in obstructive sleep apnea. Sleep 2009, 32(3): 361-8). Noradrenaline is one of the most potent neuromodulators of hypoglossal motoneuron activity (Homer R. L. Neuromodulation of hypoglossal motoneurons during sleep. Respir Physiol Neurobiol 2008, 164 (1-2): 179-196). It is thought, that decreased noradrenergic drive leads to sleep-dependent decline of hypoglossal motoneuron excitability resulting in reduced upper airway dilator muscle activity, especially reduced genioglossus muscle activity.

Alpha2C adrenoceptors regulate the release of noradrenaline from central noradrenergic neurons, they are autoreceptors involved in presynaptic feedback inhibition of noradrenaline (Hein L. et al, Two functionally distinct alpha2-adrenergic receptors regulate sympathetic neurotransmission Nature 1999, 402(6758): 181-184). An increase in the activity of the motoneurons of the hypoglossal nerve through Alpha2c adrenoceptor antagonism can stabilize the upper airways and protect them from collapse and occlusion. Moreover, also snoring can be inhibited through the mechanism of stabilization of the upper respiratory airways.

For simple snoring, there is no obstruction of the upper airways. By the narrowing of the upper airways, the flow velocity of the inhaled and exhaled air increases. This together with the relaxed muscles causes fluttering of the soft tissues of the mouth and throat in the airflow. This slight vibration generated the typical snoring sounds.

Obstructive snoring (upper airway resistance syndrome, heavy snoring, hypopnea syndrome) is caused by a recurrent partial obstruction of the upper airway during sleep. This results in an increase in airway resistance and thus to an increase in work of breathing with significant intrathoracic pressure fluctuations. The negative intrathoracic pressure development during inspiration can thereby reach values as they occur as a result of a complete airway obstruction in OSA. The pathophysiological effects on the heart, circulation and sleep quality are the same as in obstructive sleep apnea. The pathogenesis is likely the same as in OSA. Obstructive snoring often provides the precursor for OSA (Hollandt J H et al., Upper airway resistance syndrome (UARS)-obstructive snoring. HNO 2000, 48(8): 628-634).

Central sleep apnea (CSA) occurs as a result of disturbed brain function or impaired respiratory regulation. CSA is characterized by a lack of drive to breathe during sleep, resulting in repetitive periods of insufficient or absent ventilation and compromised gas exchange. There are several manifestations of CSA. These include high altitude-induced periodic breathing, idiopathic CSA (ICSA), narcotic-induced central apnea, obesity hypoventilation syndrome (OHS), and Cheyne-Stokes breathing (CSB). While the precise precipitating mechanisms involved in the various types of CSA may vary considerably, unstable ventilatory drive during sleep is a principal underlying feature (Eckert D. J. et al., Central sleep apnea: Pathophysiology and treatment. Chest 2007, 131(2): 595-607).

US 2018/0235934 A1 describes methods for treating disorders such as obstructive sleep apnea using agents for promoting hypoglossal motoneuron excitability. As agents for promoting hypoglossal motoneuron excitability a disinhibtor and/or stimulant of central noradrenic neurons is described. In some embodiments the disinhibitor of central noradrenergic neurons is an alpha2-adrenoceptor antagonist such as yohimbine or an alpha2-adrenoceptor subtype A (alpha-2A) antagonists or alpha2-adrenoceptor subtype C (alpha-2C) antagonist. The alpha2-adrenoceptor antagonist are selected from the group consisting of Atipamezole, MK-912, RS-79948, RX 821002, [3H]2-methoxy-idazoxan and JP-1302.

Alpha2C adrenoceptors belong to the family of G-protein coupled receptors. Beside the different Alpha1-adrenoceptors three different Alpha2-adrenoceptor subtypes exist (Alpha2A, Alpha2B and Alpha2C). They are involved in the mediation of several diverse physiologic effects in different tissues upon stimulation by endogeneous catecholamines (epinephrine, norepinephrine), either derived from synapses or via the blood. Alpha2 adrenoceptors plays an important physiological role, mainly in the cardiovascular system and in the central nervous system. Alpha2A- and Alpha2C-adrenoceptors are the main autoreceptors involved in presynaptic feedback inhibition of noradrenaline in the central nervous system. The potency and affinity of noradrenaline at the Alpha2C-adrenoceptor is higher than that for the Alpha2A-adrenoceptor. The Alpha2C-adrenoceptor inhibits noradrenaline release at low endogenous concentrations of noradrenaline, while Alpha2A-adrenoceptors inhibit noradrenaline release at high endogenous noradrenaline concentrations (Uys M. M. et al. Therapeutic Potential of Selectively Targeting the α2C-Adrenoceptor in Cognition, Depression, and Schizophrenia-New Developments and Future Perspective. Frontiers in Psychiatry 2017, Aug. 14; 8:144. doi: 10.3389/fpsyt.2017.00144. eCollection 2017).

A further mechanism to maintain airway patency relies on negative pressure-sensitive nerve endings/mechanoreceptors located in the pharyngeal mucosa. Upon detection of small negative pressures during the respiratory cycle these receptors generate excitatory motor nerve output to the genioglossus muscle via the negative pressure reflex.

The genioglossus muscle plays a decisive role in the pathogenesis of obstructive sleep apnoea. The activity of this muscle increases with decreasing pressure in the pharynx in the sense of a dilative compensation mechanism. Innervated by the Nervus hypoglossus, it drives the tongue forward and downward, thus widening the pharyngeal airway [Verse et al., Somnologie 3, 14-20 (1999)]. Tensioning of the dilating muscles of the upper airways is modulated inter alia via mechanoreceptors/stretch receptors in the nasal cavity/pharynx [Bouillette et al., J. Appl. Physiol. Respir. Environ. Exerc. Physiol. 46, 772-779 (1979)]. In sleeping patients suffering from serious sleep apnoea, under local anaesthesia of the upper airway an additional reduction of the activity of the genioglossus muscle can be observed [Berry et al., Am. J. Respir. Crit. Care Med. 156, 127-132 (1997)].

In a sleep apnoea model in the anaesthetized pig, intranasal administration of a potassium channel blocker which blocks the TASK-1 channel in the nanomolar range led to inhibition of collapsibility of the pharyngeal respiratory musculature and sensibilization of the negative pressure reflex of the upper airways. It is assumed that intranasal administration of the potassium channel blocker depolarizes mechanoreceptors in the upper airways and, via activation of the negative pressure reflex, leads to increased activity of the musculature of the upper airways, thus stabilizing the upper airways and preventing collapse. By virtue of this stabilization of the upper airways, the TASK channel blockade may be of great importance for obstructive sleep apnoea and also for snoring [Wirth et al., Sleep 36, 699-708 (2013); Kiper et al., Pflugers Arch. 467, 1081-1090 (2015)].

Of particular interest are TASK-1 (KCNK3 or K2P3.1) and TASK-3 (KCNK9 or K2P9.1) of the TASK (TWIK-related acid-sensitive K+ channel) subfamily. Functionally, these channels are characterized in that, during maintenance of voltage-independent kinetics, they have “leak” or “background” streams flowing through them, and they respond to numerous physiological and pathological influences by increasing or decreasing their activity. A characteristic feature of TASK channels is the sensitive reaction to a change of the extracellular pH: at acidic pH the channels are inhibited, and at alkaline pH they are activated.

TASK-1 and TASK-3 channels play also a role in respiratory regulation. Both channels are expressed in the respiratory neurons of the respiratory centre in the brain stem, inter alia in neurons which generate the respiratory rhythm (ventral respiratory group with pre-Bötzinger complex), and in the noradrenergic Locus caeruleus, and also in serotonergic neurons of the raphe nuclei. Owing to the pH dependency, here the TASK channels have the function of a sensor which translates changes in extracellular pH into corresponding cellular signals [Bayliss et al., Pflugers Arch. 467, 917-929 (2015)]. TASK-1 and TASK-3 are also expressed in the Glomus caroticum, a peripheral chemoreceptor which measures pH, O2 and CO2 content of the blood and transmits signals to the respiratory centre in the brain stem to regulate respiration. It was shown that TASK-1 knock-out mice have a reduced ventilatory response (increase of respiratory rate and tidal volume) to hypoxia and normoxic hypercapnia [Trapp et al., J. Neurosci. 28, 8844-8850 (2008)]. Furthermore, TASK-1 and TASK-3 channels were demonstrated in motoneurons of the Nervus hypoglossus, the XIIth cranial nerve, which has an important role in keeping the upper airways open [Berg et al., J. Neurosci. 24, 6693-6702 (2004)].

Aryl piperazines as α2-Adrenoceptor subtype C (alpha-2C) antagonists as well as their preparation and the use thereof as a medicament are known from WO 03/082866 A1 where the compounds are disclosed as useful for the treatment for disorders such as disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease or mild cognitive impairment. There is nothing disclosed about the use of these compounds in the treatment of sleep-related breathing disorders, preferably obstructive and central sleep apneas and snoring.

The current gold standard treatment for patients with OSA is continuous positive airway pressure (CPAP). The positive airflow pressure that is generated by an airflow turbine pump splints open the upper airway, reversing all potential causes of pharyngeal collapse, thereby preventing hypopneas, apneas and sleep fragmentation. Unfortunately, up to 50% of all patients with OSA do not tolerate CPAP in the long-term (M. Kohler, D. Smith, V. Tippett et al., Thorax 2010 65(9):829-32: Predictors of long-term compliance with continuous positive airway pressure). Therefore, there is still the need to find effective therapeutic agents for the treatment and/or prophalxis of sleep-related breathing disorders such as obstructive sleep apnea. Therefore the object of the present invention is to provide an effective therapeutic agent for the treatment and/or prophalxis of sleep-related breathing disorders, for example of obstructive sleep apnea, central sleep apnea and snoring.

Surprisingly, it has now been found that the combination of an α2-Adrenoceptor subtype C (alpha-2C) antagonists with a TASK⅓ channel blocker inhibit upper airway collapsibility with improved efficacy compared to each treatment alone and is thus suitable for the production of medicaments for the use in the treatment and/or prophylaxis of sleep-related breathing disorders, preferably obstructive and central sleep apneas and snoring. It was found that a synergism of the combination of an α2-Adrenoceptor subtype C (alpha-2C) antagonists with a TASK⅓ channel blocker allows lower doses of each treatment compared to each treatment alone.

The present invention relates to combinations of compounds of formula (I)

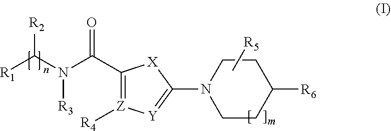

in which
X represents S, N or O;
Y represents N, S or O,
   where
   if X represents S, then Y represents N;
Z represents C, O or N,
   where
   if X represents N and Y represents N, then Z represents O;
R₁ represents 5- or 6-membered heteroaryl or phenyl,
   wherein 5- or 6-membered heteroaryl may be substituted by 1 or 2 substituents independently selected from the group of (C₁-C₄)-alkyl, (C₁-C₄)-alkoxy and halogen;
      in which (C₁-C₄)-alkyl may in turn be substituted up to trisubstituted by halogen,
      in which (C₁-C₄)-alkoxy may in turn be substituted up to trisubstituted by halogen,
   wherein phenyl may be substituted by 1 or 2 substituents independently selected from the group of (C₁-C₄)-alkyl, (C₃-C₅)-cycloalkyl, (C₁-C₄)-alkoxy, cyano, hydroxy and halogen;
      in which (C₁-C₄)-Alkyl may in turn be substituted up to trisubstituted by halogen,
R₂ represents hydrogen or (C₁-C₄)-alkyl;
   wherein (C₁-C₄)-alkyl may in turn be substituted up to trisubstituted by halogen,
   or
   together with the carbon atom to which R₂ is attached, form a (C₃-C₄)-cycloalkyl ring,
R₃ represents hydrogen or (C₁-C₄)-alkyl;
   wherein (C₁-C₄)-alkyl may in turn be substituted up to trisubstituted by halogen,
R₄ represents hydrogen, (C₁-C₄)-alkyl, (C₃-C₄)-cycloalkyl, phenyl or halogen;
   wherein (C₁-C₄)-alkyl may in turn be substituted up to trisubstituted by halogen and phenyl may in turn be substituted by Halogen,
R₅ represents hydrogen, (C₁-C₄)-alkyl, (C₁-C₄)-alkoxy or halogen,
R₆ represents a group of the formula a), b), c), d), e), f) or g)

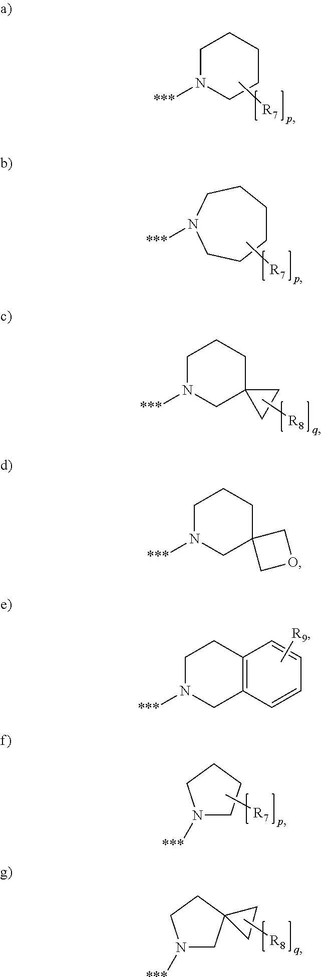

in which *** marks the bond to the adjacent piperidine ring,
wherein R₇ represents hydrogen, (C₁-C₄)-alkyl, (C₃-C₄)-cycloalkyl, (C₁-C₄)-alkoxy, (C₃-C₄)-cycloalkoxy or phenyl,
   in which (C₁-C₄)-alkyl in turn may be substituted by (C₃-C₄)-cycloalkyl, (C₁-C₄)-alkoxy, (C₃-C₄)-cycloalkoxy and may be up to trisubstituted by halogen,
      in which (C₁-C₄)-alkoxy in turn may be substituted by (C₃-C₄)-Cycloalkyl and may be up to trisubstituted by halogen,
      in which (C₃-C₄)-cycloalkyl in turn may be substituted by monofluoromethyl, difluoromethyl or trifluoromethyl und and may be up to disubstituted by halogen,
   in which (C₁-C₄)-alkoxy in turn may be substituted by (C₃-C₄)-cycloalkyl and may be up to trisubstituted by halogen,
      in which (C₃-C₄)-cycloalkyl in turn may be monosubstituted or disubstituted by halogen,
   in which (C₃-C₄)-cycloalkoxy in turn may be up to disubstituted by halogen,
wherein R₈ represents hydrogen or fluoro,
wherein R₉ represents hydrogen, (C₁-C₄)-alkyl, (C₁-C₄)-alkoxy or halogen;
   in which (C₁-C₄)-alkyl in turn may be substituted by (C₁-C₄)-Alkoxy,
n represents 0 or 1,
m represents 0, 1 or 2,
p represents 0, 1 or 2 and
q represents 0, 1 or 2,
with compounds of the formula (II)

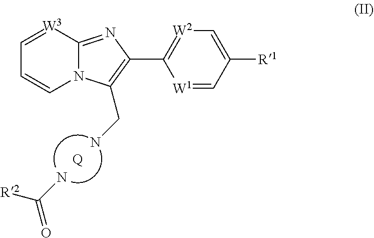

in which
   the ring Q represents a piperazine or a diazaheterobicyclic system of the formula

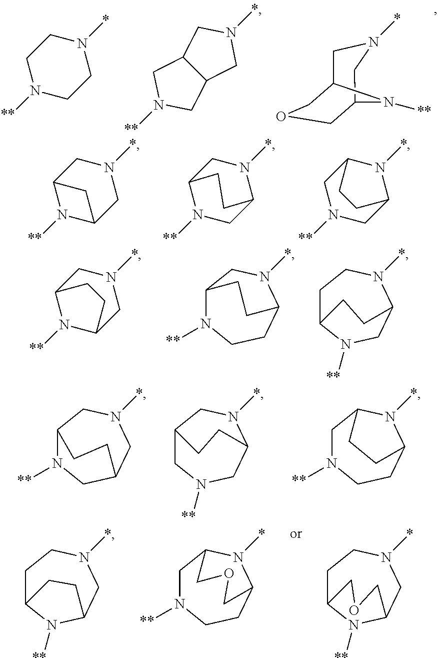

in which * denotes the bond to the adjacent CHR′² group and ** the bond to the carbonyl group,
W¹, W² or W³ represents CH or N,
R′¹ represents halogen, cyano, (C₁-C₄)-alkyl, cyclopropyl or cyclobutyl
   where (C₁-C₄)-alkyl may be up to trisubstituted by fluorine and cyclopropyl and cyclobutyl may be up to disubstituted by fluorine,
and
R′² represents (C₄-C₆)-cycloalkyl in which a ring CH₂ group may be replaced by —O—,
or
R′² represents a phenyl group of the formula (a), a pyridyl group of the formula (b) or (c) or an azole group of the formula (d), (e), (f) or (g),

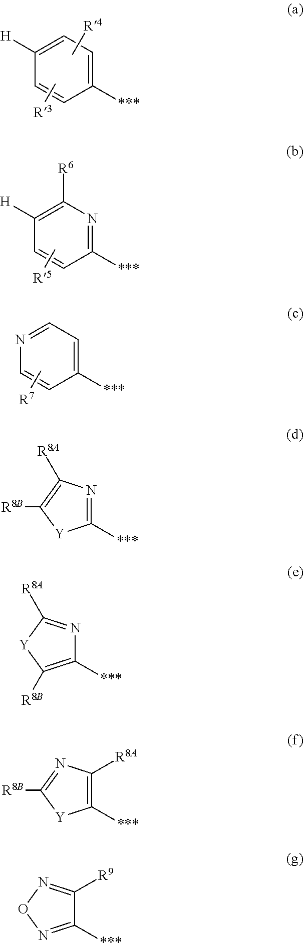

in which *** marks the bond to the adjacent carbonyl group and
R′³ represents hydrogen, fluorine, chlorine, bromine or methyl,
R′⁴ represents hydrogen, fluorine, chlorine, bromine, cyano, (C₁-C₃)-alkyl or (C₁-C₃)-alkoxy,
   where (C₁-C₃)-alkyl and (C₁-C₃)-alkoxy may each be up to trisubstituted by fluorine,
R′⁵ represents hydrogen, fluorine, chlorine, bromine or methyl,
R⁶ represents hydrogen, (C₁-C₃)-alkoxy, cyclobutyloxy, oxetan-3-yloxy, tetrahydrofuran-3-yloxy, tetrahydro-2H-pyran-4-yloxy, mono-(C₁-C₃)-alkylamino, di-(C₁-C₃)-alkylamino or (C₁-C₃)-alkylsulfanyl,
   where (C₁-C₃)-alkoxy may be up to trisubstituted by fluorine,
R⁷ represents hydrogen, fluorine, chlorine, bromine, (C₁-C₃)-alkyl or (C₁-C₃)-alkoxy,
R^{8A} and R^{8B} are identical or different and independently of one another represent hydrogen, fluorine, chlorine, bromine, (C₁-C₃)-alkyl, cyclopropyl or (C₁-C₃)-alkoxy
   where (C₁-C₃)-alkyl and (C₁-C₃)-alkoxy may each be up to trisubstituted by fluorine,
R⁹ represents hydrogen, (C₁-C₃)-alkyl or amino
and
wherein in subformula (d)
   Y represents O, S or N(CH₃),
wherein in subformula (e) and (f)
   Y represents O or S,
or
R′² represents an —OR¹⁰ or —NR¹¹R¹² group in which
   R¹⁰ represents (C₁-C₆)-alkyl, (C₄-C₆)-cycloalkyl or [(C₃-C₆)-cycloalkyl]methyl,
   R¹¹ represents hydrogen or (C₁-C₃)-alkyl
   and
   R¹² represents (C₁-C₆)-alkyl, (C₃-C₆)-cycloalkyl, phenyl or benzyl, 1-phenylethyl or 2-phenylethyl,
      where (C₁-C₆)-alkyl may be up to trisubstituted by fluorine,
      and
      where phenyl and the phenyl group in benzyl, 1-phenylethyl and 2-phenylethyl may be up to trisubstituted by identical or different radicals selected from the group consisting of fluorine, chlorine, methyl, ethyl, trifluoromethyl, methoxy, ethoxy, trifluoromethoxy and (trifluoromethyl)sulfanyl,
   or
   R¹¹ and R¹² are attached to one another and, together with the nitrogen atom to which they are bonded, form a pyrrolidine, piperidine, morpholine or thiomorpholine ring, or
R¹¹ and R¹² are attached to one another and, together with the nitrogen atom to which they are bonded, form a tetrahydroquinoline ring of the formula (c) or a tetrahydroisoquinoline ring of the formula (d),

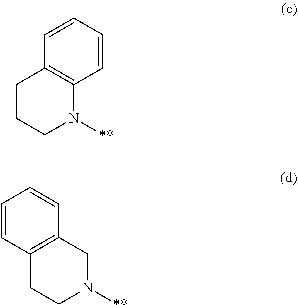

in which ** marks the bond to the carbonyl group,
and the salts, solvates and solvates of the salts thereof.

The present invention relates to combinations of compounds of formula (I)

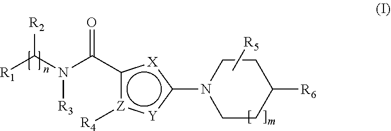

in which
X represents S, N or O;
Y represents N, S or O,
   where
   if X represents S, then Y represents N;
Z represents C, O or N,
   where
   if X represents N and Y represents N, then Z represents 0;
R₁ represents 5- or 6-membered heteroaryl or phenyl,
   wherein 5- or 6-membered heteroaryl may be substituted by 1 or 2 substituents independently selected from the group of (C₁-C₄)-alkyl, (C₁-C₄)-alkoxy and halogen;
      in which (C₁-C₄)-alkyl may in turn be substituted up to trisubstituted by halogen,
      in which (C₁-C₄)-alkoxy may in turn be substituted up to trisubstituted by halogen,
   wherein phenyl may be substituted by 1 or 2 substituents independently selected from the group of (C₁-C₄)-alkyl, (C₃-C₅)-cycloalkyl, (C₁-C₄)-alkoxy, cyano, hydroxy and halogen;
      in which (C₁-C₄)-Alkyl may in turn be substituted up to trisubstituted by halogen,
R₂ represents hydrogen or (C₁-C₄)-alkyl;
   wherein (C₁-C₄)-alkyl may in turn be substituted up to trisubstituted by halogen,
   or
   together with the carbon atom to which R₂ is attached, form a (C₃-C₄)-cycloalkyl ring,
R₃ represents hydrogen or (C₁-C₄)-alkyl;
   wherein (C₁-C₄)-alkyl may in turn be substituted up to trisubstituted by halogen,
R₄ is absent when Z represents N or 0;
   represents hydrogen, (C₁-C₄)-alkyl, (C₃-C₄)-cycloalkyl, phenyl or halogen when Z represents C;
   wherein (C₁-C₄)-alkyl may in turn be substituted up to trisubstituted by halogen and phenyl may in turn be substituted by Halogen,
R₅ represents hydrogen, (C₁-C₄)-alkyl, (C₁-C₄)-alkoxy or halogen,
R₆ represents a group of the formula a), b), c), d), e), f) or g)

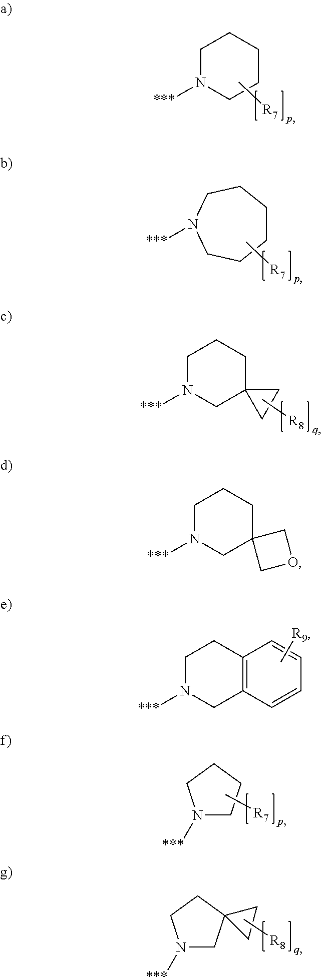

in which *** marks the bond to the adjacent piperidine ring,
wherein R₇ represents hydrogen, (C₁-C₄)-alkyl, (C₃-C₄)-cycloalkyl, (C₁-C₄)-alkoxy, (C₃-C₄)-cycloalkoxy or phenyl,
   in which (C₁-C₄)-alkyl in turn may be substituted by (C₃-C₄)-cycloalkyl, (C₁-C₄)-alkoxy, (C₃-C₄)-cycloalkoxy and may be up to trisubstituted by halogen,
      in which (C₁-C₄)-alkoxy in turn may be substituted by (C₃-C₄)-Cycloalkyl and may be up to trisubstituted by halogen,
      in which (C₃-C₄)-cycloalkyl in turn may be substituted by monofluoromethyl, difluoromethyl or trifluoromethyl und and may be up to disubstituted by halogen,
   in which (C₁-C₄)-alkoxy in turn may be substituted by (C₃-C₄)-cycloalkyl and may be up to trisubstituted by halogen,
      in which (C₃-C₄)-cycloalkyl in turn may be monosubstituted or disubstituted by halogen,
   in which (C₃-C₄)-cycloalkoxy in turn may be up to disubstituted by halogen,
wherein R₈ represents hydrogen or fluoro,
wherein R₉ represents hydrogen, (C₁-C₄)-alkyl, (C₁-C₄)-alkoxy or halogen;
   in which (C₁-C₄)-alkyl in turn may be substituted by (C₁-C₄)-Alkoxy, n represents 0 or 1,
m represents 0, 1 or 2,
p represents 0, 1 or 2 and
q represents 0, 1 or 2,
with compounds of the formula (II)

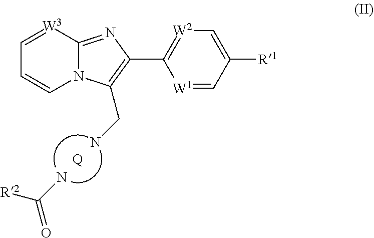

in which
   the ring Q represents a piperazine or a diazaheterobicyclic system of the formula

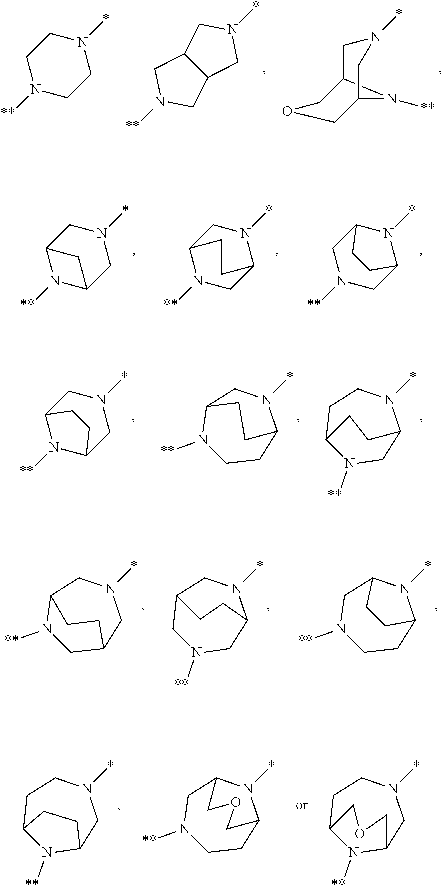

in which * denotes the bond to the adjacent CHR′² group and ** the bond to the carbonyl group,
W¹, W² or W³ represents CH or N,
R′¹ represents halogen, cyano, (C₁-C₄)-alkyl, cyclopropyl or cyclobutyl
   where (C₁-C₄)-alkyl may be up to trisubstituted by fluorine and cyclopropyl and cyclobutyl may be up to disubstituted by fluorine,
and
R′² represents (C₄-C₆)-cycloalkyl in which a ring CH₂ group may be replaced by —O—,
or
R′² represents a phenyl group of the formula (a), a pyridyl group of the formula (b) or (c) or an azole group of the formula (d), (e), (f) or (g),

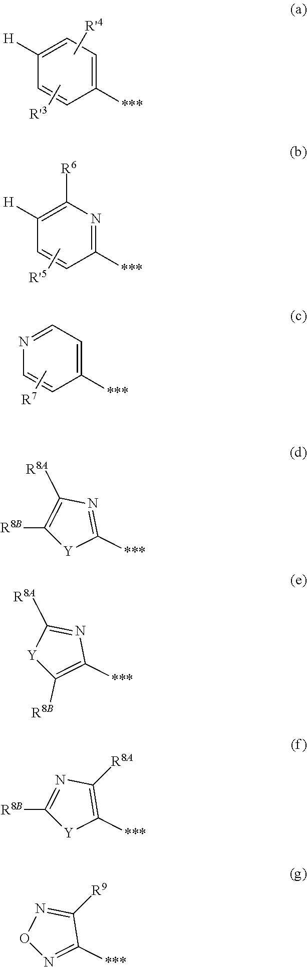

in which *** marks the bond to the adjacent carbonyl group and
R′³ represents hydrogen, fluorine, chlorine, bromine or methyl,
R′⁴ represents hydrogen, fluorine, chlorine, bromine, cyano, (C₁-C₃)-alkyl or (C₁-C₃)-alkoxy,
   where (C₁-C₃)-alkyl and (C₁-C₃)-alkoxy may each be up to trisubstituted by fluorine,
R′⁵ represents hydrogen, fluorine, chlorine, bromine or methyl,
R⁶ represents hydrogen, (C₁-C₃)-alkoxy, cyclobutyloxy, oxetan-3-yloxy, tetrahydrofuran-3-yloxy, tetrahydro-2H-pyran-4-yloxy, mono-(C₁-C₃)-alkylamino, di-(C₁-C₃)-alkylamino or (C₁-C₃)-alkylsulfanyl,
   where (C₁-C₃)-alkoxy may be up to trisubstituted by fluorine,
R⁷ represents hydrogen, fluorine, chlorine, bromine, (C₁-C₃)-alkyl or (C₁-C₃)-alkoxy,
R^{8A} and R^{8B} are identical or different and independently of one another represent hydrogen, fluorine, chlorine, bromine, (C₁-C₃)-alkyl, cyclopropyl or (C₁-C₃)-alkoxy
   where (C₁-C₃)-alkyl and (C₁-C₃)-alkoxy may each be up to trisubstituted by fluorine,
R⁹ represents hydrogen, (C₁-C₃)-alkyl or amino
and
wherein in subformula (d)
   Y represents O, S or N(CH₃),
wherein in subformula (e) and (f)
   Y represents O or S,
or
R′² represents an —OR¹⁰ or —NR¹¹R¹² group in which
   R¹⁰ represents (C₁-C₆)-alkyl, (C₄-C₆)-cycloalkyl or [(C₃-C₆)-cycloalkyl]methyl,
   R¹¹ represents hydrogen or (C₁-C₃)-alkyl
   and
   R¹² represents (C₁-C₆)-alkyl, (C₃-C₆)-cycloalkyl, phenyl or benzyl, 1-phenylethyl or 2-phenylethyl,
      where (C₁-C₆)-alkyl may be up to trisubstituted by fluorine,
      and
      where phenyl and the phenyl group in benzyl, 1-phenylethyl and 2-phenylethyl may be up to trisubstituted by identical or different radicals selected from the group consisting of fluorine, chlorine, methyl, ethyl, trifluoromethyl, methoxy, ethoxy, trifluoromethoxy and (trifluoromethyl)sulfanyl,
   or
   R¹¹ and R¹² are attached to one another and, together with the nitrogen atom to which they are bonded, form a pyrrolidine, piperidine, morpholine or thiomorpholine ring, or
   R¹¹ and R¹² are attached to one another and, together with the nitrogen atom to which they are bonded, form a tetrahydroquinoline ring of the formula (c) or a tetrahydroisoquinoline ring of the formula (d),

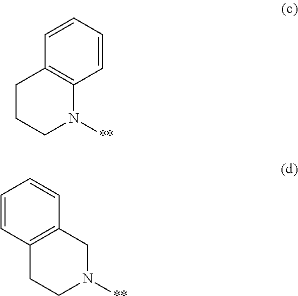

in which ** marks the bond to the carbonyl group,
and the salts, solvates and solvates of the salts thereof.

In a possible subgroup of the compounds of formula I
X represents S,
Y represents N,
and
Z represents C,
where in the resulting group of the formula (h),

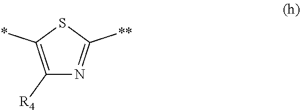

in which * denotes the bond to the carbonyl group and ** the bond to the N-atom of the adjacent piperidine-ring,
R₄ represents hydrogen or chloro,

In another possible subgroup of the compounds of formula I
R₁ represents pyridinyl or phenyl,
   wherein pyridinyl may be substituted by 1 or 2 substituents independently selected from the group of methyl, ethyl, fluoro, chloro, trifluoromethyl and trifluormethoxy;
   wherein phenyl may be substituted by 1 or 2 substituents independently selected from the group of methyl, cyclopropyl, methoxy, cyano, hydroxy, fluoro, chloro and trifluoromethyl;

In another possible subgroup of the compounds of formula I
R₁ represents 3,5-difluoropyridin-2-yl.

In another possible subgroup of the compounds of formula I
R₂ represents hydrogen;
   or
   together with the carbon atom to which R₂ is attached, forms a cyclopropyl ring.

In another possible subgroup of the compounds of formula I
R₆ represents a group of the formula a),

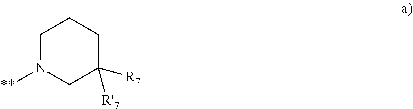

in which *** denotes the bond to the adjacent piperidine-ring,
and
R₇ represents hydrogen,
R′₇ methyl, ethyl, n-propyl, iso-propyl, ethoxy, methoxymethyl, monofluoromethyl, difluoromethyl, trifluoromethyl, 3,3-difluorocyclobutylmethoxy, 2,2,2-trifluoroethoxymethyl, cyclopropylmethyl, 1-fluoromethylcyclopropylmethoxymethyl, 1-difluoromethylcyclopropylmethoxymethyl, 1-trifluoromethylcyclopropylmethoxymethyl, cyclobutylmethoxy, cyclopropylmethoxy, cyclobutyloxymethyl, cyclopropylmethoxymethyl, 3,3-difluorocyclobutylmethoxymethyl, 3-fluorobutyloxymethyl, 2,2-difluorocyclopropylmethoxy, cyclobutyloxy, 3,3-difluorocyclobutyloxy, 2-fluoorethyl, cyclopropyl, cyclobutyl, 2-methoxyethyl or tert.-butyl,
or
R₇ and R′₇ are attached to one another and, together with the carbon atom to which they are bonded, form a cyclopropyl ring.

In another possible subgroup of the compounds of formula I
R₆ represents a group of the formula a),

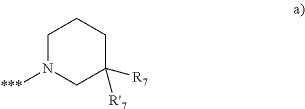

in which *** denotes the bond to the adjacent piperidine-ring,
and
R₇ represents hydrogen,
R′₇ methyl,
or
R₇ and R′₇ are attached to one another and, together with the carbon atom to which they are bonded, form a cyclopropyl ring,

In another possible subgroup of the compounds of formula I n is 1.

In a further possible subgroup of the compounds of formula I m is 1.

In yet another possible subgroup of the compounds of formula I p is 1.

In yet another possible subgroup of the compounds of formula I q is 2.

In a further possible subgroup of the compounds of formula I the compound is N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide or N-[1-(3,5-difluoropyridin-2-yl)cyclopropyl]-2-[(3R)-3-methyl[1,4′-bipiperidin]-1′-yl]-1,3-thiazole-5-carboxamide.

A preferred compound of formula (I) is N-[(3,5-difluoropyridin-2-yl)methyl]-2-[(3R)-3-methyl[1,4′-bipiperidin]-1′-yl]-1,3-thiazole-5-carboxamide.

In a possible subgroup of the compounds of formula II
the ring Q represents a diazaheterobicyclic system of the formula

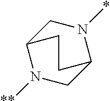

in which * denotes the bond to the adjacent CHR² group and ** the bond to the carbonyl group,

In a possible subgroup of the compounds of formula II
the ring Q represents a diazaheterobicyclic system of the formula

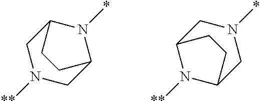

in which * denotes the bond to the adjacent CHR² group and ** the bond to the carbonyl group.

In a further possible subgroup of the compounds of formula II

W¹ represents CH.

In a further possible subgroup of the compounds of formula II

W² represents CH.

In a further possible subgroup of the compounds of formula II

W³ represents N.

In yet a further possible subgroup of the compounds of formula II

R′¹ represents chlorine, bromine, isopropyl or cyclopropyl,

In yet a further possible subgroup of the compounds of formula II

R′² represents a phenyl group of the formula (a)

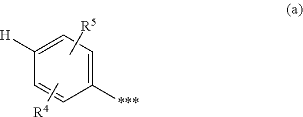

in which *** marks the bond to the adjacent carbonyl group,
R⁴ represents hydrogen, fluorine or chlorine
and
R⁵ represents fluorine, chlorine, (C₁-C₃)-alkyl or (C₁-C₃)-alkoxy,
R′² represents a pyridyl group of the formula (b)

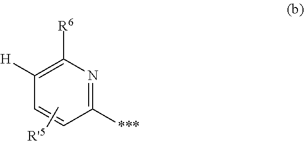

in which *** marks the bond to the adjacent carbonyl group and
R′⁵ represents hydrogen, fluorine or chlorine,

R⁶ represents methoxy, difluoromethoxy or trifluoromethoxy, In a further possible subgroup of the compounds of formula II the compound is (4-{[2-(4-Chlorophenyl)imidazo[1,2-a]pyridin-3-yl]methyl}piperazin-1-yl)(6-methoxypyridin-2-yl)methanone, (5-{[2-(4-Chlorophenyl)imidazo[1,2-a]pyrimidin-3-yl]methyl}-2,5-diazabicyclo[2.2.2]oct-2-yl)(3-fluoro-6-methoxypyridin-2-yl)methanone, (3-Fluoro-6-methoxypyridin-2-yl)(3-{[2-(4-isopropylphenyl)imidazo-[1,2-a]pyrimidin-3-yl]methyl}-3,8-diazabicyclo[3.2.1]oct-8-yl)methanone or (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

In a further possible subgroup of the compounds of formula II the compound is (4-{[2-(4-Chlorophenyl)imidazo[1,2-a]pyridin-3-yl]methyl}piperazin-1-yl)(6-methoxypyridin-2-yl)methanone or (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

A preferred compound of formula (II) is 4-{[2-(4-Chlorophenyl)imidazo[1,2-a]pyridin-3-yl]methyl}piperazin-1-yl)(6-methoxypyridin-2-yl)methanone.

A preferred compound of formula (II) is (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

A further embodiment of the present invention relates to combinations of compounds of formula (I)
in which
X, Y and Z are selected from the group of S, N, O and C to form a group of formulae (h), (i), (j), (k) or (r)

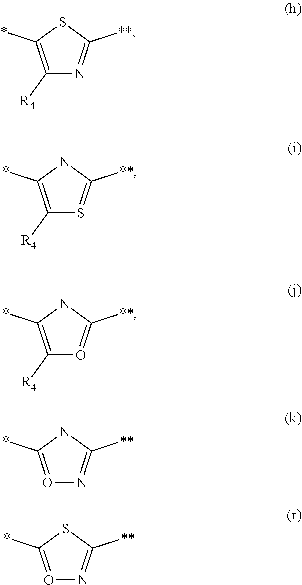

R₁ represents pyridinyl, pyrazolyl, thiazolyl, thienyl or phenyl,
   wherein pyridinyl may be substituted by 1 or 2 substituents independently selected from the group of (C₁-C₂)-alkyl, fluoro, chloro, trifluoromethyl and trifluormethoxy;
   wherein pyrazolyl may be substituted by 1 or 2 substituents independently selected from the group of (C₁-C₂)-alkyl, fluoro, chloro, trifluoromethyl and trifluormethoxy;
   wherein thiazolyl may be substituted by chloro,
   wherein thienyl may be substituted by fluoro,
   wherein phenyl may be substituted by 1 or 2 substituents independently selected from the group of (C₁-C₂)-alkyl, (C₃-C₄)-cycloalkyl, methoxy, cyano, hydroxy, fluoro, chloro and trifluoromethyl;
R₂ represents hydrogen or methyl;
   together with the carbon atom to which R₂ is attached, forms a cyclopropyl ring,
R₃ represents hydrogen or (C₁-C₂)-alkyl;
R₄ represents hydrogen, methyl, ethyl, cyclopropyl, trifluormethyl, bromo, chloro or phenyl;
   wherein phenyl may in turn be substituted by chloro,
R₅ represents hydrogen or fluoro,
R₆ represents a group of the formula a), b′), b″), c′), c″) or e),

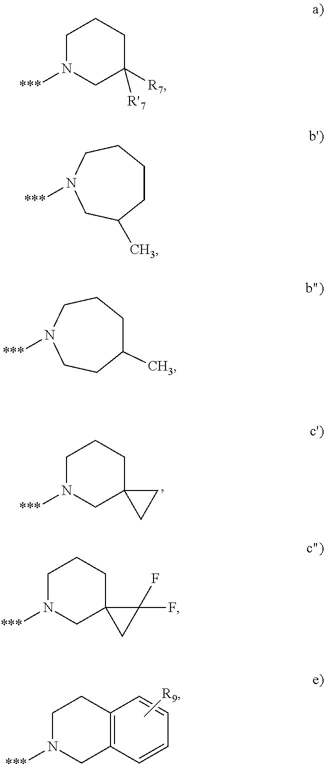

in which *** marks the bond to the adjacent piperidine ring,
wherein R₇ or R′₇ independently from each other represent hydrogen, (C₁-C₄)-alkyl, (C₃-C₄)-cycloalkyl, (C₁-C₂)-alkoxy, (C₃-C₄)-cycloalkoxy, monofluormethyl, difluormethyl, trifluormethyl, difluormethoxy or phenyl,
   in which (C₁-C₄)-alkyl in turn may be substituted by methoxy, n-butoxy, cyclopropyl, cyclobutoxy and may be up to disubstituted by fluoro,
      in which methoxy in turn may be substituted by cyclopropyl, cyclobutyl or trifluoroethyl,
      in which cyclopropyl in turn may be substituted by monofluoromethyl, difluoromethyl or trifluoromethyl,
      in which cyclobutyl in turn may be substituted by monofluoromethyl, difluoromethyl or trifluoromethyl,
   in which (C₁-C₂)-alkoxy in turn may be substituted by (C₃-C₄)-cycloalkyl and may be up to trisubstituted by halogen,
      in which (C₃-C₄)-cycloalkyl in turn may be monosubstituted or disubstituted by fluoro,
   in which (C₃-C₄)-cycloalkoxy in turn may be up to disubstituted by fluoro,
wherein R₉ represents hydrogen, methyl, tert.-butyl, methoxy, methoxymethyl, fluoro or chloro;
n represents 0 or 1,
m represents 1 or 2,
and compounds of formula (II)
wherein
the ring Q represents a piperazine or a diazaheterobicyclic system of the formula

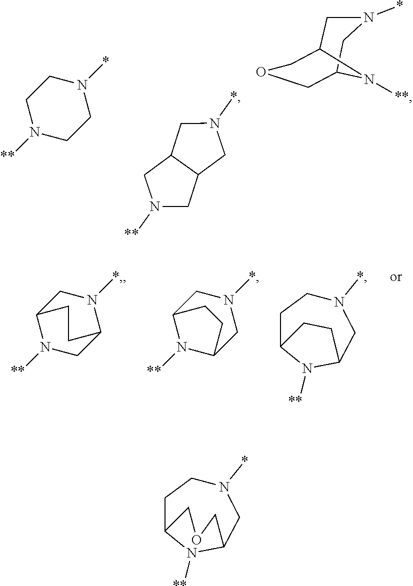

in which * denotes the bond to the adjacent CHR² group and ** the bond to the carbonyl group,
W² represents CH,
W¹, W³ represents CH or N,
R′¹ represents fluorine, chlorine, bromine, methyl, tert.-butyl, isopropyl, cyclopropyl or cyclobutyl,
and
R′² represents cyclobutyl, cyclopentyl or cyclohexyl,
or
R′² represents a phenyl group of the formula (a), a pyridyl group of the formula (b) or an azole group of the formula (d) or formula (g)

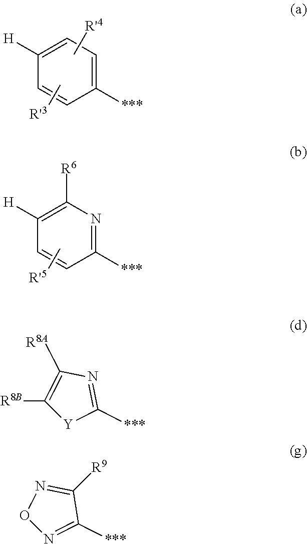

in which *** marks the bond to the adjacent carbonyl group and
R′³ represents hydrogen, fluorine or chlorine,
R′⁴ represents fluorine, chlorine, methyl, isopropyl, methoxy or ethoxy,
R′⁵ represents hydrogen, fluorine, chlorine, bromine or methyl,
R⁶ represents methoxy, difluoromethoxy, trifluoromethoxy, isopropoxy, cyclobutyloxy or methylsulfanyl,
R^{8A} and R^{8B} are identical or different and independently of one another represent hydrogen, methyl, trifluoromethyl, ethyl, isopropyl or cyclopropyl,
and
R⁹ represents methyl or amino
Y represents O or S or N(CH₃)
and the salts, solvates and solvates of the salts thereof.

A further embodiment of the present invention relates to combinations of compounds of formula (I)
in which
X, Y and Z are selected from S, N, O or C to form 1,3-thiazolyl, 1,3-oxazolyl, or 1,2,4-oxadiazolyl;
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen or methyl;
R₄ represents hydrogen, methyl, ethyl or trifluormethyl;
   wherein phenyl may in turn be substituted by chloro,
R₅ represents hydrogen or fluoro,
R₆ represents a group of the formula a), c′) or c″),

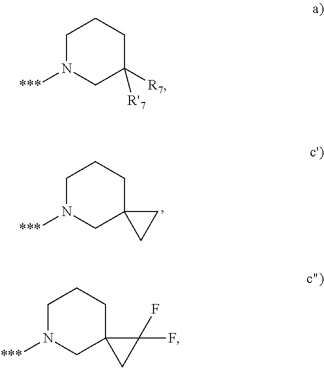

in which *** marks the bond to the adjacent piperidine ring, wherein R₇ or R′₇ independently from each other represent hydrogen, methyl, ethyl, n-propyl, iso-propyl, tert.-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1,
and compounds of formula (II)
wherein
the ring Q represents a piperazine or a diazaheterobicyclic system of the formula

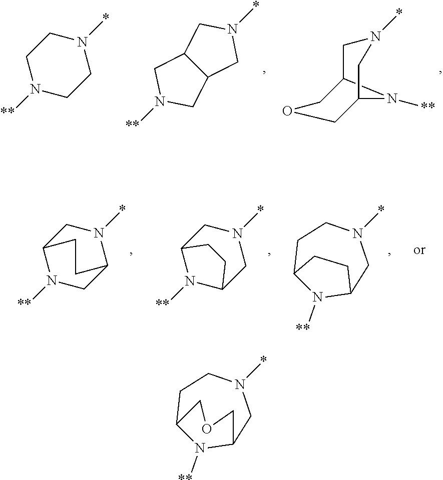

in which * denotes the bond to the adjacent CHR² group and ** the bond to the carbonyl group,
W² represents CH,
W¹, W³ represents CH or N,
R′¹ represents fluorine, chlorine, bromine, methyl, tert.-butyl, isopropyl, cyclopropyl or cyclobutyl,
and
R′² represents cyclobutyl, cyclopentyl or cyclohexyl,
or
R′² represents a phenyl group of the formula (a), a pyridyl group of the formula (b) or an azole group of the formula (d) or formula (g)

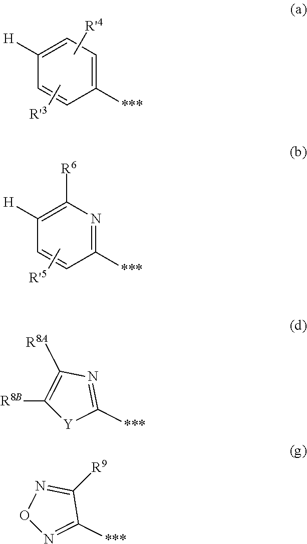

in which *** marks the bond to the adjacent carbonyl group and
R′³ represents hydrogen, fluorine or chlorine,
R′⁴ represents fluorine, chlorine, methyl, isopropyl, methoxy or ethoxy,
R′⁵ represents hydrogen, fluorine, chlorine, bromine or methyl,
R⁶ represents methoxy, difluoromethoxy, trifluoromethoxy, isopropoxy, cyclobutyloxy or methylsulfanyl,
R^{8A} and R^{8B} are identical or different and independently of one another represent hydrogen, methyl, trifluoromethyl, ethyl, isopropyl or cyclopropyl,
and
R⁹ represents methyl or amino,
Y represents O or S or N(CH₃),
and the salts, solvates and solvates of the salts thereof.

A further embodiment of the present invention relates to combinations of compounds of formula (I)
in which
X, Y and Z are selected from the group of S, N, O and C to form 1,3-thiazolyl, 1,3-oxazolyl or 1,2,4-oxadiazolyl,
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R² represents hydrogen or methyl;
R₃ represents hydrogen or methyl;
R⁴ represents hydrogen, methyl, ethyl or trifluormethyl;
   wherein phenyl may in turn be substituted by chloro,
R₅ represents hydrogen or fluoro;
R₆ represents a group of the formula a), c′) or c″),

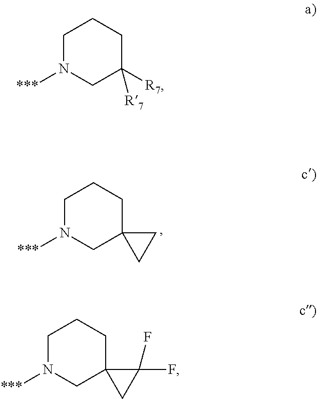

in which *** marks the bond to the adjacent piperidine ring,
wherein R₇ or R′₇ independently from each other represent hydrogen, methyl, ethyl, n-propyl, iso-propyl, tert.-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1,
and compounds of formula (II)
wherein
the ring Q represents a diazaheterobicyclic system of the formula

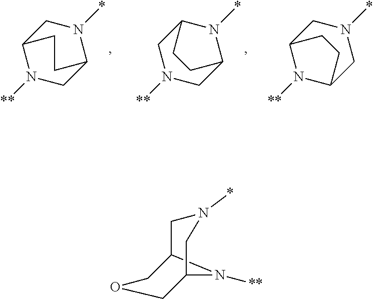

in which * denotes the bond to the adjacent CHR² group and ** the bond to the carbonyl group,
W¹ represents CH,
W² represents CH,
W³ represents N,
R′¹ represents fluorine, chlorine, bromine, methyl, isopropyl, cyclopropyl or cyclobutyl,
R′² represents cyclobutyl, cyclopentyl or cyclohexyl
or
R′² represents a phenyl group of the formula (a), a pyridyl group of the formula (b) or an azole group of the formula (d), (e) or (f)

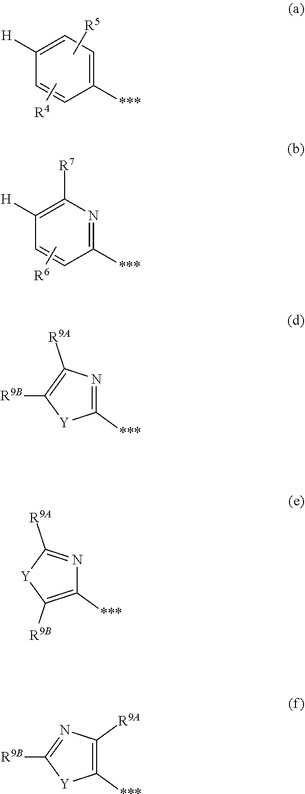

in which *** marks the bond to the adjacent carbonyl group and
R⁴ represents hydrogen, fluorine or chlorine,
R⁵ represents fluorine, chlorine, cyano, (C₁-C₃)-alkyl, (C₁-C₃)-alkoxy or trifluoromethoxy,
R⁶ represents hydrogen, fluorine, chlorine, bromine or methyl,
R⁷ represents (C₁-C₃)-alkoxy, cyclobutyloxy or (C₁-C₃)-alkylsulfanyl,
   where (C₁-C₃)-alkoxy may be up to trisubstituted by fluorine,
R^{9A} and R^{9B} are identical or different and independently of one another represent hydrogen, chlorine, bromine, (C₁-C₃)-alkyl or cyclopropyl,
   where (C₁-C₃)-alkyl may be up to trisubstituted by fluorine,
and
Y represents O or S,
and the salts, solvates and solvates of the salts thereof.

A further embodiment of the present invention relates to combinations of compounds of formula (I)
in which
X, Y and Z are selected from the group of S, N, O and C to form 1,3-thiazolyl, 1,3-oxazolyl, or 1,2,4-oxadiazolyl;
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R³ represents hydrogen or methyl;
R⁴ represents hydrogen, methyl, ethyl or trifluormethyl;
   wherein phenyl may in turn be substituted by chloro,
R₅ represents hydrogen or fluoro,
R₆ represents a group of the formula a), c′) or c″),

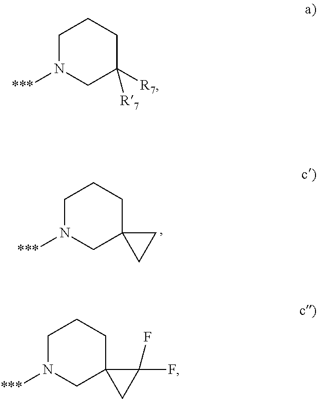

in which *** marks the bond to the adjacent piperidine ring,
wherein R₇ or R′₇ independently from each other represent hydrogen, methyl, ethyl, n-propyl, iso-propyl, tert.-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1,
and compounds of formula (II)
wherein
the ring Q represents a diazaheterobicyclic system of the formula

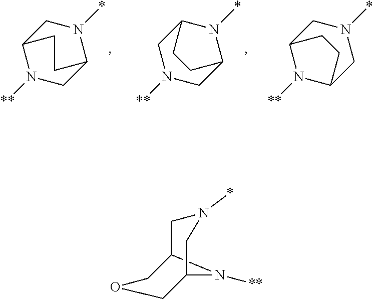

in which * denotes the bond to the adjacent CHR² group and ** the bond to the carbonyl group,
W¹ represents CH,
W² represents CH,
W³ represents N,
R′¹ represents chlorine, bromine, isopropyl or cyclobutyl,
and
R′² represents cyclopentyl or cyclohexyl,
or
R′² represents a phenyl group of the formula (a), a pyridyl group of the formula (b) or an azole group of the formula (d), (e) or (f)

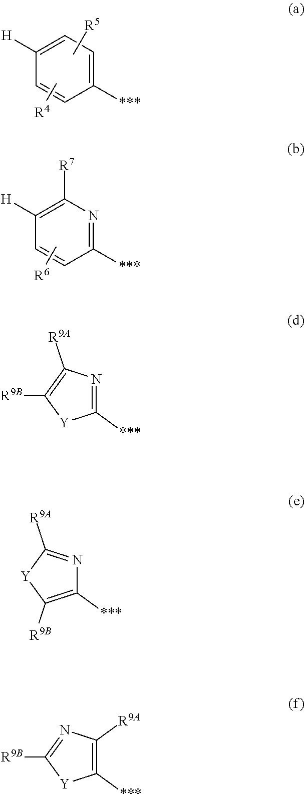

in which *** marks the bond to the adjacent carbonyl group and
R⁴ represents hydrogen, fluorine or chlorine,
R⁵ represents fluorine, chlorine, methyl, isopropyl, methoxy or ethoxy,
R⁶ represents hydrogen, fluorine, chlorine, bromine or methyl,
R⁷ represents methoxy, difluoromethoxy, trifluoromethoxy, isopropoxy, cyclobutyloxy or methylsulfanyl,
R^{9A} and R^{9B} are identical or different and independently of one another represent hydrogen, methyl, trifluoromethyl, ethyl, isopropyl or cyclopropyl,
and
Y represents O or S,
and the salts, solvates and solvates of the salts thereof.

In a preferred embodiment of the present invention is directed to combinations of N-[(3,5-difluoropyridin-2-yl)methyl]-2-[(3R)-3-methyl [1,4′-bipiperidin]-1′-yl]-1,3-thiazole-5-carboxamide, N-[(3,5-difluoropyridin-2-yl)methyl]-2-[4-(3,4-dihydroisoquinolin-2(1H)-yl)piperidin-1-yl]-1,3-thiazole-5-carboxamide, 2-[3-(cyclopropylmethyl)[1,4′-bipiperidin]-1′-yl]-N-[(3,5-difluoropyridin-2-yl)methyl]-1,3-thiazole-5-carboxamide, 2-[3-(difluoromethyl)[1,4′-bipiperidin]-1′-yl]-N-[(3,5-difluoropyridin-2-yl)methyl]-1,3-thiazolo-5-carboxamide, N-[(3,5-difluoropyridin-2-yl)methyl]-2-[3-(trifluoromethyl)[1,4′-bipiperidin]-1′-yl]-1,3-thiazole-5-carboxamide, N-[(3,5-difluoropyridin-2-yl)methyl]-2-[3-(fluoromethyl)-[1,4′-bipiperidin]-1′-yl]-1,3-thiazole-5-carboxamide, 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-4-methyl-2-[(3R)-3-methyl[1,4′-bipiperidin]-1′-yl]-1,3-thiazole-5-carboxamide, N-[(3,5-difluoro-pyridin-2-yl)methyl]-4-methyl-2-[(3R)-3-methyl[1,4′-bipiperidin]-1′-yl]-1,3-thiazole-5-carboxamide, N-[(3,5-difluorpyridin-2-yl)methyl]-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-4-(trifluoromethyl)-1,3-thiazole-5-carboxamide, N-[(3,5-difluoropyridin-2-yl)methyl]-5-ethyl-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-1,3-oxazole-4-carboxamide, N-[(3,5-difluoropyridin-2-yl)methyl]-5-methyl-2-[(3R)-3-methyl[1,4′-bipiperidin]-1′-yl]-1,3-oxazole-4-carboxamide, N-[(3,5-difluoropyridin-2-yl)methyl]-2-[(3R)-3-methoxy[1,4′-bipiperidin]-1′-yl]-1,3-thiazole-5-carboxamide, 2-[3-(difluoromethoxy)[1,4′-bipiperidin]-1′-yl]-N-[(3,5-difluoropyridin-2-yl)methyl]-1,3-thiazoel-5-carboxamide, N-[(3,5-difluoropyridin-2-yl)methyl]-2-(3-ethyl[1,4′-bipiperidin]-1′-yl)-1,3-thiazole-5-carboxamide, 2-[(3R)-3-methyl[1,4′-bipiperidin]-1′-yl]-N-{[4-(trifluoromethyl)pyridin-2-yl]methyl}-1,3-thiazole-5-carboxamide, 2-[(3R)-3-methyl[1,4′-bipiperidin]-1′-yl]-N-[3-(trifluoromethyl)benzyl]-1,3-thiazole-5-carboxamide, N-[(3-fluoropyridin-2-yl)methyl]-2-[(3R)-3-methyl[1,4′-bipiperidin]-1′-yl]-1,3-thiazole-5-carboxamide, N-(5-chloro-2-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[4-(trifluoromethyl)benzyl]-1,3-thiazole-5-carboxamide, N-[(5-chloro-3-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(3-methylpyridin-2-yl)methyl]-1,3-thiazole-5-carboxamide, 2-[(3R)-3-methyl[1,4′-bipiperidin]-1′-yl]-N-[(4-methylpyridin-2-yl)methyl]-1,3-thiazole-5-carboxamide, N-[(3-chloropyridin-2-yl)methyl]-2-[(3R)-3-methyl[1,4′-bipiperidin]-1′-yl]-1,3-thiazole-5-carboxamide, N-[(3-fluoropyridin-2-yl)methyl]-N-methyl-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethyl)pyridin-2-yl]methyl}-1,3-thiazole-5-carboxamide, N-[(5-chloropyridin-2-yl)methyl]-2-[(3R)-3-methyl[1,4′-bipiperidin]-1′-yl]-1,3-thiazole-5-carboxamide, N-[1-(2,5-difluorophenyl)ethyl]-2-[(3R)-3-methyl[1,4′-bipiperidin]-1′-yl]-1,3-thiazole-5-carboxamide, N-[(3-chloro-5-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethoxy)pyridin-2-yl]methyl}-1,3-thiazole-5-carboxamide, N-(4-chlorobenzyl)-2-[(3R)-3-methyl[1,4′-bipiperidin]-1′-yl]-1,3-thiazole-5-carboxamide, N-(2-chloro-5-fluorobenzyl)-2-[(3R)-3-methyl[1,4′-bipiperidin]-1′-yl]-1,3-thiazole-5-carboxamide, N-(4-methylbenzyl)-2-[(3R)-3-methyl[1,4′-bipiperidin]-1′-yl]-1,3-thiazole-5-carboxamide, N-(3-methylbenzyl)-2-[(3R)-3-methyl[1,4′-bipiperidin]-1′-yl]-1,3-thiazole-5-carboxamide, N-(2-methylbenzyl)-2-[(3R)-3-methyl[1,4′-bipiperidin]-1′-yl]-1,3-thiazole-5-carboxamide, 2-[(3S)-(difluoromethyl)[1,4′-bipiperidin]-1′-yl]-N-[(3,5-difluoropyridin-2-yl)methyl]-1,3-thiazole-5-carboxamide, 2-[(3R)-3-(difluoromethyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[(3S)-3-(fluoromethyl)[1,4′-bipiperidin]-1′-yl]-1,3-thiazole-5-carboxamide, N-[(3,5-difluoropyridin-2-yl)methyl]-2-[(3R)-3-(fluoromethyl)[1,4′-bipiperidin]-1′-yl]-1,3-thiazole-5-carboxamide, N-[(3,5-difluoropyridin-2-yl)methyl]-2-[(3S)-3-(trifluoromethyl)[1,4′-bipiperidin]-1′-yl]-1,3-thiazole-5-carboxamide, N-[(3,5-difluoropyridin-2-yl)methyl]-2-[(3R)-3-(trifluoromethyl)[1,4′-bipiperidin]-1′-yl]-1,3-thiazole-5-carboxamide, 2-{(3S)-3-[(3,3-difluorocyclobutyl)methoxy][1,4′-bipiperidin]-1′-yl}-N-[(3,5-difluoropyridin-2-yl)methyl]-1,3-thiazole-5-carboxamide, 2-{(3R)-3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(1S)-1-(2,5-difluorophenyl)ethyl]-2-[(3R)-3-methyl[1,4′-bipiperidin]-1′-yl]-1,3-thiazole-5-carboxamide, N-[(1R)-1-(2,5-difluorophenyl)ethyl]-2-[(3R)-3-methyl[1,4′-bipiperidin]-1′-yl]-1,3-thiazole-5-carboxamide, N-[(3,5-difluoropyridin-2-yl)methyl]-2-[3-(methoxymethyl)[1,4′-bipiperidin]-1′-yl]-1,3-thiazole-5-carboxamide, N-[(3,5-difluoropyridin-2-yl)methyl]-3-[(3R)-3-methyl[1,4′-bipiperidin]-1′-yl]-1,2,4-oxadiazole-5-carboxamide, N-[(3,5-difluoropyridin-2-yl)methyl]-2-[(3R)-3′-fluor-3-methyl[1,4′-bipiperidin]-1′-yl]-1,3-thiazole-5-carboxamide, ent-N-[(3,5-difluoropyridin-2-yl)methyl]-2-[(3R), (3′R)-3′-fluoro-3-methyl[1,4′-bipiperidin]-1′-yl]-1,3-thiazole-5-carboxamide, ent-N-[(3,5-difluoropyridin-2-yl)methyl]-2-[(3R), (3'S)-3′-fluoro-3-methyl[1,4′-bipiperidin]-1′-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[(5-fluoro-2-thienyl)methyl]-2-[(3R)-3-methyl[1,4′-bipiperidin]-1′-yl]-1,3-thiazole-5-carboxamide, 2-[(3R)-3-methyl[1,4′-bipiperidin]-1-yl]-N-(pyridin-4-ylmethyl)-1,3-thiazole-5-carboxamide, N-[(3,5-difluoropyridin-2-yl)methyl]-2-{3-[(2,2,2-trifluoroethoxy)methyl][1,4′-bipiperidin]-1′-yl}-1,3-thiazole-5-carboxamide, N-[(3,5-difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide, 2-[3-({[1-(difluoromethyl)cyclopropyl]methoxy}methyl) [1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[3-({[1-(trifluoromethyl)cyclopropyl]methoxy}methyl)[1,4′-bipiperidin]-1′-yl]-1,3-thiazoel-5-carboxamide, N-[(3,5-difluoropyridin-2-yl)methyl]-2-(3,3-dimethyl[1,4′-bipiperidin]-1′-yl)-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, 2-[4-(1,1-difluoro-5-azaspiro[2.5]octan-5-yl)piperidin-1-yl]-N-[(3,5-difluoropyridin-2-yl)methyl]-1,3-thiazoel-5-carboxamide, 2-[3-(cyclobutylmethoxy)[1,4′-bipiperidin]-1′-yl]-N-[(3,5-difluoropyridin-2-yl)methyl]-1,3-thiazole-5-carboxamide, 2-[3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclobutyloxy)methyl]-[1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[3-ethoxy[1,4′-bipiperidin]-1′-yl]-1,3-thiazole-5-carboxamide, N-[(3,5-difluoropyridin-2-yl)methyl]-2-{4-[(3R)-3-methylpiperidin-1-yl]azepan-1-yl}-1,3-thiazole-5-carboxamide, 2-[(3R)-3-methyl[1,4′-bipiperidin]-1′-yl]-N-[(6-methylpyridin-3-yl)methyl]-1,3-thiazole-5-carboxamide, N-benzyl-2-[(3R)-3-methyl[1,4′-bipiperidin]-1′-yl]-1,3-thiazole-5-carboxamide, N-[(3,5-difluoropyridin-2-yl)methyl]-2-[3-({[3-fluorobutyl]oxy}methyl)[1,4′-bipiperidin]-1′-yl]-1,3-thiazole-5-carboxamide, 2-(3-{[3.3-difluorocyclobutyl)methoxy]methyl}[1,4′-bipiperidin]-1′-yl)-N-[(3,5-difluoropyridin-2-yl)methyl]-1,3-thiazole-5-carboxamide, N-[(3-fluoropyridin-4-yl)methyl]-2-[(3R)-3-methyl[1,4′-bipiperidin]-1′-yl]-1,3-thiazole-5-carboxamide, N-[(3,5-difluoropyridin-2-yl)methyl]-2-[3-(2,2,2-trifluoroethoxy)[1,4′-bipiperidin]-1′-yl]-1,3-thiazole-5-carboxamide, N-[(4,6-dimethylpyridin-3-yl)methyl]-2-[(3R)-3-methyl[1,4′-bipiperidin]-1′-yl]-1,3-thiazole-5-carboxamide, N-[(4-chloro-1-methyl-1H-pyrazol-5-yl)methyl]-2-[(3R)-3-methyl[1,4′-bipiperidin]-1′-yl]-1,3-thiazole-5-carboxamide, N-(3-methoxybenzyl)-2-[(3R)-3-methyl[1,4′-bipiperidin]-1′-yl]-1,3-thiazole-5-carboxamide, N-(2,5-difluorobenzyl)-2-[(3R)-3-methyl[1,4′-bipiperidin]-1′-yl]-1,3-thiazole-5-carboxamide, N-(3-hydroxybenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(2R)-2-phenylpropyl]-1,3-thiazole-5-carboxamide, N-(4-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-3-ylmethyl)-1,3-thiazole-5-carboxamide, N-(3-fluorobenzyl)-2-[(3R)-3-methyl[1,4′-bipiperidin]-1′-yl]-1,3-thiazole-5-carboxamide, N-(2-fluorobenzyl)-2-[(3R)-3-methyl[1,4′-bipiperidin]-1′-yl]-1,3-thiazole-5-carboxamide, N-(2-chloro-4-fluorophenyl)-2-[(3R)-3-methyl[1,4′-bipiperidin]-1′-yl]-1,3-thiazole-5-carboxamide, N-(3-cyano-4-fluorophenyl)-2-[(3R)-3-methyl[1,4′-bipiperidin]-1′-yl]-1,3-thiazole-5-carboxamide, N-methyl-2-[(3R)-3-methyl[1,4′-bipiperidin]-1′-yl]-N-(pyridin-3-ylmethyl)-1,3-thiazole-5-carboxamide, N-methyl-2-[(3R)-3-methyl[1,4′-bipiperidin]-1′-yl]-N-(pyridin-4-ylmethyl)-1,3-thiazole-5-carboxamide, N-benzyl-N-methyl-2-[(3R)-3-methyl[1,4′-bipiperidin]-1′-yl]-1,3-thiazole-5-carboxamide, N-(2-cyclopropylphenyl)-2-[(3R)-3-methyl[1,4′-bipiperidin]-1′-yl]-1,3-thiazole-5-carboxamide, N-(3-chlorobenzyl)-2-[(3R)-3-methyl[1,4′-bipiperidin]-1′-yl]-1,3-thiazol-5-carboxamide, 2-[(3R)-3-methyl[1,4′-bipiperidin]-1′-yl]-N-[(1R)-1-(4-methylphenyl)ethyl]-1,3-thiazole-5-carboxamide, N-(2-ethylpyridin-4-yl)-2-[(3R)-3-methyl[1,4′-bipiperidin]-1′-yl]-1,3-thiazole-5-carboxamide, N-[(3,5-difluoropyridin-2-yl)methyl]-2-[(3S)-3-(methoxymethyl)[1,4′-bipiperidin]-1′-yl]-1,3-thiazole-5-carboxamide, N-[(3,5-difluoropyridin-2-yl)methyl]-2-[(3R)-3-(methoxymethyl)[1,4′-bipiperidin]-1′-yl]-1,3-thiazole-5-carboxamide, 2-{(3S)-3-[(cyclobutyloxy)methyl][1,4′-bipiperidin]-1′-yl}-N-[(3,5-difluorpyridin-2-yl)methyl]-1,3-thiazole-5-carboxamide, 2-{(3R)-3-[(cyclobutyloxy)methyl][1,4′-bipiperidin]-1′-yl}-N-[(3,5-difluorpyridin-2-yl)methyl]-1,3-thiazole-5-carboxamide, N-[(3,5-difluoropyridin-2-yl)methyl]-2-(3-isopropyl[1,4′-bipiperidin]-1′-yl)-1,3-thiazole-5-carboxamide, N-[(3,5-difluoropyridin-2-yl)methyl]-2-[4-((4S)-4-methylazepan-1-yl)piperidin-1-yl]-1,3-thiazole-5-carboxamide, N-[(3,5-difluoropyridin-2-yl)methyl]-2-[4-((4R)-4-methylazepan-1-yl)piperidin-1-yl]-1,3-thiazole-5-carboxamide, N-[(3,5-difluoropyridin-2-yl)methyl]-2-{(3S)-3-[(2,2,2-trifluoroethoxy)methyl][1,4′-bipiperidin]-1′-yl}-1,3-thiazole-5-carboxamide, N-[(3,5-difluoropyridin-2-yl)methyl]-2-{(3R)-3-[(2,2,2-trifluoroethoxy)methyl][1,4′-bipiperidin]-1′-yl}-1,3-thiazole-5-carboxamide, 2-{3-[(2,2-difluorocyclopropyl)methoxy][1,4′-bipiperidin]-1′-yl}-N-[(3,5-difluoropyridin-2-yl)methyl]-1,3-thiazole-5-carboxamide, 2-[3-(cyclobutyloxy)[1,4′-bipiperidin]-1′-yl]-N-[(3,5-difluoropyridin-2-yl)methyl]-1,3-thiazole-5-carboxamide, 2-{3-[(3,3-difluorocyclobutyl)oxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[(3R)-3′-fluoro-3-methyl[1,4′-bipiperidin]-1′-yl]-1,3-thiazole-4-carboxamide, N-[(3,5-difluoropyridin-2-yl)methyl]-2-[(3R)-3′-fluoro-3-methyl[1,4′-bipiperidin]-1′-yl]-1,3-oxazole-4-carboxamide, N-(5-chloro-2-fluorobenzyl)-2-[(3R)-3′-fluoro-3-methyl[1,4′-bipiperidin]-1′-yl]-1,3-thiazole-5-carboxamide, 2-[(3R)-3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[1-(2,5-difluorophenyl)ethyl]-2-[(3R)-3′-fluoro-3-methyl[1,4′-bipiperidin]-1′-yl]-1,3-thiazole-5-carboxamide, 4-(2-chlorophenyl)-N-[(3,5-difluoropyridin-2-yl)methyl]-2-[(3R)-3-methyl[1,4′-bipiperidin]-1′-yl]-1,3-thiazole-5-carboxamide, 4-bromo-N-[(3,5-difluoropyridin-2-yl)methyl]-2-[(3R)-3-methyl[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-(3-propyl[1,4′-bipiperidin]-1′-yl)-1,3-thiazole-5-carboxamide, 4-cyclopropyl-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-((3S)-3-ethoxy[1,4′-bipiperidin]-1′-yl)-1,3-thiazole-5-carboxamide, N-[(3,5-difluoropyridin-2-yl)methyl]-2-((3R)-3-ethoxy[1,4′-bipiperidin]-1′-yl)-1,3-thiazole-5-carboxamide, 2-[(3S)-3-(cyclobutylmethoxy)[1,4′-bipiperidin]-1′-yl]-N-[(3,5-difluoropyridin-2-yl)methyl]-1,3-thiazole-5-carboxamide, 2-[(3R)-3-(cyclobutylmethoxy)[1,4′-bipiperidin]-1′-yl]-N-[(3,5-difluoropyridin-2-yl)methyl]-1,3-thiazole-5-carboxamide, formic acid-N-[(3,5-difluoropyridin-2-yl)methyl]-2-[3-(2-fluoroethyl)[1,4′-bipiperidin]-1′-yl]-1,3-thiazole-5-carboxamide, 2-([1,4′-bipiperidin]-1′-yl)-N-[(3,5-difluoropyridin-2-yl)methyl]-1,3-thiazole-5-carboxamide, N-[1-(3,5-difluoropyridin-2-yl)cyclopropyl]-2-[(3R)-3-methyl[1,4′-bipiperidin]-1′-yl]-1,3-thiazole-5-carboxamide, N-[(3,5-difluoropyridin-2-yl)methyl]-4-ethyl-2-[(3R)-3-methyl[1,4′-bipiperidin]-1′-yl]-1,3-thiazole-5-carboxamide, 2-[4-(3S)-(1,1-difluoro-5-azaspiro[2.5]octan-5-yl)piperidin-1-yl]-N-[(3,5-difluoropyridin-2-yl)methyl]-1,3-thiazole-5-carboxamide, 2-[4-(3R)-(1,1-difluoro-5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-(3-phenyl[1,4′-bipiperidin]-1′-yl)-1,3-thiazole-5-carboxamide, 2-[4-(1,1-difluoro-5-azaspiro[2.5]octan-5-yl)-3-fluoropiperidin-1-yl]-N-[(3,5-difluoropyridin-2-yl)methyl]-1,3-thiazole-5-carboxamide, 2-[4-(5-azaspiro[2.5]octan-5-yl)-3-fluoropiperidin-1-yl]-N-[(3,5-difluoropyridin-2-yl)methyl]-1,3-thiazole-5-carboxamide
as compound of formula (I)
and compounds of formula (II) which are selected from the group consisting of:
(4-{[2-(4-Bromophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(6-methoxypyridin-2-yl)methanone, (4-{[2-(4-Bromophenyl)imidazo[1,2-a]pyridin-3-yl]methyl}piperazin-1-yl)(2-fluorophenyl)methanone, (4-{[2-(4-Bromophenyl)imidazo[1,2-a]pyridin-3-yl]methyl}piperazin-1-yl)(3-methoxyphenyl)methanone, (4-{[2-(4-Bromophenyl)imidazo[1,2-a]pyridin-3-yl]methyl}piperazin-1-yl)(2-chloro-5-fluorophenyl)methanone, (4-{[2-(4-Chlorophenyl)imidazo[1,2-a]pyridin-3-yl]methyl}piperazin-1-yl)(2-fluorophenyl)methanone, (4-{[2-(4-Fluorophenyl)imidazo[1,2-a]pyridin-3-yl]methyl}piperazin-1-yl)(cyclohexyl)methanone, (4-{[2-(4-bromophenyl)imidazo[1,2-a]pyridin-3-yl]methyl}piperazin-1-yl)(cyclohexyl)methanone, (4-{[2-(4-bromophenyl)imidazo[1,2-a]pyridin-3-yl]methyl}piperazin-1-yl)(tetrahydrofuran-3-yl)methanone, (4-{[2-(4-bromophenyl)imidazo[1,2-a]pyridin-3-yl]methyl}piperazin-1-yl)(cyclobutyl)methanone, (4-{[2-(4-bromophenyl)imidazo[1,2-a]pyridin-3-yl]methyl}piperazin-1-yl)(2-methoxyphenyl)methanone, (4-{[2-(4-bromophenyl)imidazo[1,2-a]pyridin-3-yl]methyl}piperazin-1-yl)(5-fluoro-2-methoxyphenyl)methanone, (4-{[2-(4-bromophenyl)imidazo[1,2-a]pyridin-3-yl]methyl}piperazin-1-yl)(2-methylphenyl)methanone, (4-{[2-(4-bromophenyl)imidazo[1,2-a]pyridin-3-yl]methyl}piperazin-1-yl)(5-fluoro-2-methylphenyl)methanone, (2-chloro-5-fluorophenyl)(4-{[2-(4-chlorophenyl)imidazo[1,2-a]pyridin-3-yl]methyl}piperazin-1-yl)methanone, (4-{[2-(4-chlorophenyl)imidazo[1,2-a]pyridin-3-yl]methyl}piperazin-1-yl)(cyclohexyl)methanone, ((4-{[2-(4-chlorophenyl)imidazo[1,2-a]pyridin-3-yl]methyl}piperazin-1-yl)(cyclobutyl)methanone, (4-{[2-(4-chlorophenyl)imidazo[1,2-a]pyridin-3-yl]methyl}piperazin-1-yl)(3-methoxyphenyl)methanone, (4-{[2-(4-chlorophenyl)imidazo[1,2-a]pyridin-3-yl]methyl}piperazin-1-yl)(2-methoxyphenyl)methanone, (4-{[2-(4-chlorophenyl)imidazo[1,2-a]pyridin-3-yl]methyl}piperazin-1-yl)(5-fluoro-2-methoxyphenyl)methanone, (4-{[2-(4-chlorophenyl)imidazo[1,2-a]pyridin-3-yl]methyl}piperazin-1-yl)(2-methylphenyl)methanone, (4-{[2-(4-chlorophenyl)imidazo[1,2-a]pyridin-3-yl]methyl}piperazin-1-yl)(5-fluoro-2-methylphenyl)methanone, (4-{[2-(4-chlorophenyl)imidazo[1,2-a]pyridin-3-yl]methyl}piperazin-1-yl)[3-(trifluoromethoxy)phenyl]methanone, (4-{[2-(4-chlorophenyl)imidazo[1,2-a]pyridin-3-yl]methyl}piperazin-1-yl)[3-(trifluoromethyl)phenyl]methanone, ((4-{[2-(4-chlorophenyl)imidazo[1,2-a]pyridin-3-yl]methyl}piperazin-1-yl)(pyridin-2-yl)methanone, (4-{[2-(4-chlorophenyl)imidazo[1,2-a]pyridin-3-yl]methyl}piperazin-1-yl)(2-fluoro-5-methoxyphenyl)methanone, (4-{[2-(4-chlorophenyl)imidazo[1,2-a]pyridin-3-yl]methyl}piperazin-1-yl)(2-ethoxyphenyl)methanone, (2-chloro-5-methoxyphenyl)(4-{[2-(4-chlorophenyl)imidazo[1,2-a]pyridin-3-yl]methyl}piperazin-1-yl)methanone, (4-{[2-(4-chlorophenyl)imidazo[1,2-a]pyridin-3-yl]methyl}piperazin-1-yl)(tetrahydro-2H-pyran-2-yl)methanone, (4-{[2-(4-chlorophenyl)imidazo[1,2-a]pyridin-3-yl]methyl}piperazin-1-yl)(3-isopropoxyphenyl)methanone, 2-[(4-{[2-(4-chlorophenyl)imidazo[1,2-a]pyridin-3-yl]methyl}piperazin-1-yl)carbonyl]benzonitrile, (4-{[2-(4-chlorophenyl)imidazo[1,2-a]pyridin-3-yl]methyl}piperazin-1-yl)(3-isopropylphenyl)methanone, (4-{[2-(4-chlorophenyl)imidazo[1,2-a]pyridin-3-yl]methyl}piperazin-1-yl)(2-isopropylphenyl)methanone, (4-{[2-(4-chlorophenyl)imidazo[1,2-a]pyridin-3-yl]methyl}piperazin-1-yl)(tetrahydrofuran-2-yl)methanone, (3-chlorophenyl)(4-{[2-(4-chlorophenyl)imidazo[1,2-a]pyridin-3-yl]methyl}piperazin-1-yl)methanone, (2-chlorophenyl)(4-{[2-(4-chlorophenyl)imidazo[1,2-a]pyridin-3-yl]methyl}piperazin-1-yl)methanone, (4-{[2-(4-chlorophenyl)imidazo[1,2-a]pyridin-3-yl]methyl}piperazin-1-yl)[6-(2,2,2-trifluoroethoxy)pyridin-2-yl]methanone, (4-{[2-(4-chlorophenyl)imidazo[1,2-a]pyridin-3-yl]methyl}piperazin-1-yl)(6-isopropoxypyridin-2-yl)methanone, (4-{[2-(4-chlorophenyl)imidazo[1,2-a]pyridin-3-yl]methyl}piperazin-1-yl)(6-methoxy-4-methylpyridin-2-yl)methanone, (4-{[2-(4-chlorophenyl)imidazo[1,2-a]pyridin-3-yl]methyl}piperazin-1-yl)[6-(cyclobutyloxy)pyridin-2-yl]methanone, (3-bromo-6-methoxypyridin-2-yl)(4-{[2-(4-chlorophenyl)imidazo[1,2-a]pyridin-3-yl]methyl}piperazin-1-yl)methanone, (3-chloro-6-methoxypyridin-2-yl)(4-{[2-(4-chlorophenyl)imidazo[1,2-a]pyridin-3-yl]methyl}piperazin-1-yl)methanone, (4-{[2-(4-chlorophenyl)imidazo[1,2-a]pyridin-3-yl]methyl}piperazin-1-yl)[6-(difluoromethoxy)pyridin-2-yl]methanone, (4-{[2-(4-chlorophenyl)imidazo[1,2-a]pyridin-3-yl]methyl}piperazin-1-yl)(6-ethoxypyridin-2-yl)methanone, (4-{[2-(4-chlorophenyl)imidazo[1,2-a]pyridin-3-yl]methyl}piperazin-1-yl)[6-(tetrahydro-2H-pyran-4-yloxy)pyridin-2-yl]methanone, (4-{[2-(4-bromophenyl)imidazo[1,2-a]pyridin-3-yl]methyl}piperazin-1-yl)(6-methoxypyridin-2-yl)methanone, (4-{[2-(4-fluorophenyl)imidazo[1,2-a]pyridin-3-yl]methyl}piperazin-1-yl)(cyclopentyl)methanone, (4-{[2-(4-fluorophenyl)imidazo[1,2-a]pyridin-3-yl]methyl}piperazin-1-yl)(cyclobutyl)methanone, (5-fluoro-2-methoxyphenyl)(4-{[2-(4-fluorophenyl)imidazo[1,2-a]pyridin-3-yl]methyl}piperazin-1-yl)methanone, (2-chloro-5-fluorophenyl)(4-{[2-(4-fluorophenyl)imidazo[1,2-a]pyridin-3-yl]methyl}piperazin-1-yl)methanone, (4-{[2-(4-fluorophenyl)imidazo[1,2-a]pyridin-3-yl]methyl}piperazin-1-yl)(2-methoxyphenyl)methanone, (2-fluorophenyl)(4-{[2-(4-isopropylphenyl)imidazo[1,2-a]pyridin-3-yl]methyl}piperazin-1-yl)methanone, cyclopentyl(4-{[2-(4-isopropylphenyl)imidazo[1,2-a]pyridin-3-yl]methyl}piperazin-1-yl)methanone, (4-{[2-(4-isopropylphenyl)imidazo[1,2-a]pyridin-3-yl]methyl}piperazin-1-yl)(6-methoxypyridin-2-yl)methanone, cyclopentyl(4-{[2-(4-methylphenyl)imidazo[1,2-a]pyridin-3-yl]methyl}piperazin-1-yl)methanone, cyclohexyl(4-{[2-(4-methylphenyl)imidazo[1,2-a]pyridin-3-yl]methyl}piperazin-1-yl)methanone, (2-methoxyphenyl)(4-{[2-(4-methylphenyl)imidazo[1,2-a]pyridin-3-yl]methyl}piperazin-1-yl)methanone, (6-methoxypyridin-2-yl)(4-{[2-(4-methylphenyl)imidazo[1,2-a]pyridin-3-yl]methyl}piperazin-1-yl)methanone, (4-(3-{[4-(2-fluorobenzoyl)piperazin-1-yl]methyl}imidazo[1,2-a]pyridin-2-yl)benzonitrile, 4-[3-({4-[(6-methoxypyridin-2-yl)carbonyl]piperazin-1-yl}methyl)imidazo[1,2-a]pyridin-2-yl]benzonitrile, 4-(3-{[4-(cyclopentylcarbonyl)piperazin-1-yl]methyl}imidazo[1,2-a]pyridin-2-yl)benzonitrile, 4-(3-{[4-(cyclohexylcarbonyl)piperazin-1-yl]methyl}imidazo[1,2-a]pyridin-2-yl)benzonitrile, (4-{[2-(4-tert-butylphenyl)imidazo[1,2-a]pyridin-3-yl]methyl}piperazin-1-yl)(6-methoxypyridin-2-yl)methanone, (4-{[2-(4-tert-butylphenyl)imidazo[1,2-a]pyridin-3-yl]methyl}piperazin-1-yl)(2-fluorophenyl)methanone, (4-{[2-(4-tert-butylphenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)[6-(trifluoromethoxy)pyridin-2-yl]methanone, (4-{[2-(4-Chlorophenyl)imidazo[1,2-a]pyridin-3-yl]methyl}piperazin-1-yl)(3-fluoro-6-methoxypyridin-2-yl)methanone, (4-{[2-(4-Cyclopropylphenyl)imidazo[1,2-a]pyridin-3-yl]methyl}piperazin-1-yl)(2-fluorophenyl)methanone, 4-(3-{[4-(2-fluoro-5-methoxybenzoyl)piperazin-1-yl]methyl}imidazo[1-2-a]pyridin-2-yl)benzo-nitrile, 4-[3-({4-[(6-methoxy-3-methylpyridin-2-yl)carbonyl]piperazin-1-yl}methyl)imidazo[1,2-a]pyridin-2-yl)benzonitrile, (4-{[2-(4-chlorophenyl)imidazo[1,2-a]pyridin-3 yl],methyl}piperazin-1-yl)(6-methoxy-3-methylpyridin-2-yl)methanone, (4-{[2-(4-tert.-butylphenyl)imidazo[1,2-a]-pyridin-3-yl]methyl}piperazin-1-yl)(6-methoxy-3-methylpyridin-2-yl)methanone, (4-{[2-(4-bromophenyl)imidazo[1,2-a]pyridin-3-yl],methyl}piperazin-1-yl)(6-methoxy-3-methyl-pyridin-2-yl)methanone; tert-butyl 5-{[2-(4-chlorophenyl)imidazo[1,2-a]pyridin-3-yl]methyl}hexahydropyrrolo[3,4-c]pyrrole-2(1H)-carboxylate, tert-butyl 5-{[2-(4-bromophenyl)imidazo[1,2-a]pyridin-3-yl]methyl}hexahydropyrrolo[3,4-c]pyrrole-2(1H)-carboxylate, tert-butyl 5-{[2-(4-isopropylphenyl)imidazo[1,2-a]pyridin-3-yl]methyl}hexahydropyrrolo[3,4-c]pyrrole-2(1H)-carboxylate, [5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Bromophenyl)imidazo[1,2-a]pyridin-3-yl]methyl}hexahydropyrrolo[3,4-c]pyrrol-2(1H)-yl](cyclopentyl)methanone, [5-{[2-(4-Bromophenyl)imidazo[1,2-a]pyridin-3-yl]methyl}hexahydropyrrolo[3,4-c]pyrrol-2(1H)-yl](2-fluorophenyl)methanone, [5-{[2-(4-Bromophenyl)imidazo[1,2-a]pyridin-3-yl]methyl}hexahydropyrrolo[3,4-c]pyrrol-2(1H)-yl](2-chloro-5-fluorophenyl)methanone, [5-{[2-(4-Bromophenyl)imidazo[1,2-a]pyridin-3-yl]methyl}hexahydropyrrolo[3,4-c]pyrrol-2(1H)-yl](cyclohexyl)methanone, [5-{[2-(4-Bromophenyl)imidazo[1,2-a]pyridin-3-yl]methyl}hexahydropyrrolo[3,4-c]pyrrol-2(1H)-yl](cyclobutyl)methanone, [5-{[2-(4-Bromophenyl)imidazo[1,2-a]pyridin-3-yl]methyl}hexahydropyrrolo[3,4-c]pyrrol-2(1H)-yl](3-methoxyphenyl)methanone, [5-{[2-(4-Bromophenyl)imidazo[1,2-a]pyridin-3-yl]methyl}hexahydropyrrolo[3,4-c]pyrrol-2(1H)-yl](2-methoxyphenyl)methanone, [5-{[2-(4-Bromophenyl)imidazo[1,2-a]pyridin-3-yl]methyl}hexahydropyrrolo[3,4-c]pyrrol-2(1H)-yl](5-fluoro-2-methoxyphenyl)methanone, [5-{[2-(4-Bromophenyl)imidazo[1,2-a]pyridin-3-yl]methyl}hexahydropyrrolo[3,4-c]pyrrol-2(1H)-yl](2-methylphenyl)methanone, [5-{[2-(4-Chlorophenyl)imidazo[1,2-a]pyridin-3-yl]methyl}hexahydropyrrolo[3,4-c]pyrrol-2(1H)-yl](2-fluorophenyl)methanone, (2-Chloro-5-fluorophenyl)[5-{[2-(4-chlorophenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](cyclohexyl)methanone, [5-{[2-(4-Chlorophenyl)imidazo[1,2-a]pyridin-3-yl]methyl}hexahydropyrrolo[3,4-c]pyrrol-2(1H)-yl](3-methoxyphenyl)methanone, [5-{[2-(4-Chlorophenyl)imidazo[1,2-a]pyridin-3-yl]methyl}hexahydropyrrolo[3,4-c]pyrrol-2(1H)-yl](2-methoxyphenyl)methanone, [5-{[2-(4-Chlorophenyl)imidazo[1,2-a]pyridin-3-yl]methyl}hexahydropyrrolo[3,4-c]pyrrol-2(1H)-yl](5-fluoro-2-methoxyphenyl)methanone, [5-{[2-(4-Chlorophenyl)imidazo[1,2-a]pyridin-3-yl]methyl}hexahydropyrrolo[3,4-c]pyrrol-2(1H)-yl](2-methylphenyl)methanone, [5-{[2-(4-Chlorophenyl)imidazo[1,2-a]pyridin-3-yl]methyl}hexahydropyrrolo[3,4-c]pyrrol-2(1H)-yl](5-fluoro-2-methylphenyl)methanone, [5-{[2-(4-Chlorophenyl)imidazo[1,2-a]pyridin-3-yl]methyl}hexahydropyrrolo[3,4-c]pyrrol-2(1H)-yl][3-(trifluoromethoxy)phenyl]methanone, [5-{[2-(4-Chlorophenyl)imidazo[1,2-a]pyridin-3-yl]methyl}hexahydropyrrolo[3,4-c]pyrrol-2(1H)-yl][3-(trifluoromethyl)phenyl]methanone, [5-{[2-(4-Bromophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Isopropylphenyl)imidazo[1,2-a]pyridin-3-yl]methyl}hexahydropyrrolo[3,4-c]pyrrol-2(1H)-yl](6-methoxypyridin-2-yl)methanone, (2-Fluorophenyl)[5-{[2-(4-isopropylphenyl)imidazo[1,2-a]pyridin-3-yl]methyl}hexahydropyrrolo[3,4-c]pyrrol-2(1H)-yl]methanone, [5-{[2-(4-Isopropylphenyl)imidazo[1,2-a]pyridin-3-yl]methyl}hexahydropyrrolo[3,4-c]pyrrol-2(1H)-yl](3-methoxyphenyl)methanone, Cyclopentyl[5-{[2-(4-isopropylphenyl)imidazo[1,2-a]pyridin-3-yl]methyl}hexahydropyrrolo[3,4-c]pyrrol-2(1H)-yl]methanone, 5-{[2-(4-Bromophenyl)imidazo[1,2-a]pyridin-3-yl]methyl}-N-methyl-N-phenylhexahydropyrrolo[3,4-c]pyrrole-2(1H)-carboxamide, [5-{[2-(4-Bromophenyl)imidazo[1,2-a]pyridin-3-yl]methyl}hexahydropyrrolo[3,4-c]pyrrol-2(1H)-yl](3,4-dihydroquinoline-1(2H)-yl)methanone, [5-{[2-(4-Bromophenyl)imidazo[1,2-a]pyridin-3-yl]methyl}hexahydropyrrolo[3,4-c]pyrrol-2(1H)-yl](3,4-dihydroisoquinoline-2(1H)-yl)methanone, Isobutyl 5-{[2-(4-bromophenyl)imidazo[1,2-a]pyridin-3-yl]methyl}hexahydropyrrolo[3,4-c]pyrrole-2(1H)-carboxylate, Benzyl 5-{[2-(4-bromophenyl)imidazo[1,2-a]pyridin-3-yl]methyl}hexahydropyrrolo[3,4-c]pyrrole-2(1H)-carboxylate, Cyclopentyl 5-{[2-(4-bromophenyl)imidazo[1,2-a]pyridin-3-yl]methyl}hexahydropyrrolo[3,4-c]pyrrole-2(1H)-carboxylate, Isopropyl 5-{[2-(4-bromophenyl)imidazo[1,2-a]pyridin-3-yl]methyl}hexahydropyrrolo[3,4-c]pyrrole-2(1H)-carboxylate, 3-(Trifluoromethyl)phenyl 5-{[2-(4-bromophenyl)imidazo[1,2-a]pyridin-3-yl]methyl}hexahydropyrrolo[3,4-c]pyrrole-2(1H)-carboxylate, Fluoroethyl 5-{[2-(4-bromophenyl)imidazo[1,2-a]pyridin-3-yl]methyl}hexahydropyrrolo[3,4-c]pyrrole-2(1H)-carboxylate, 5-{[2-(4-Bromophenyl)imidazo[1,2-a]pyridin-3-yl]methyl}-N-(2,4-difluorophenyl)hexahydropyrrolo[3,4-c]pyrrole-2(1H)-carboxamide, 5-{[2-(4-Bromophenyl)imidazo[1,2-a]pyridin-3-yl]methyl}-N-(2,6-difluorobenzyl)hexahydropyrrolo[3,4-c]pyrrole-2(1H)-carboxamide, 5-{[2-(4-Bromophenyl)imidazo[1,2-a]pyridin-3-yl]methyl}-N-(2,6-dimethylphenyl)hexahydropyrrolo[3,4-c]pyrrole-2(1H)-carboxamide, 5-{[2-(4-Bromophenyl)imidazo[1,2-a]pyridin-3-yl]methyl}-N-(2-fluorophenyl)hexahydropyrrolo[3,4-c]pyrrole-2(1H)-carboxamide, 5-{[2-(4-Bromophenyl)imidazo[1,2-a]pyridin-3-yl]methyl}-N-(2-ethoxyphenyl)hexahydropyrrolo[3,4-c]pyrrole-2(1H)-carboxamide, 5-{[2-(4-Bromophenyl)imidazo[1,2-a]pyridin-3-yl]methyl}-N-(4-chloro-3-(trifluoromethyl)phenyl]hexahydropyrrolo[3,4-c]pyrrole-2(1H)-carboxamide, 5-{[2-(4-Bromophenyl)imidazo[1,2-a]pyridin-3-yl]methyl}-N-[2-chloro-5-(trifluoromethyl)phenyl]hexahydropyrrolo[3,4-c]pyrrole-2(1H)-carboxamide, 5-{[2-(4-Bromophenyl)imidazo[1,2-a]pyridin-3-yl]methyl}-N-(cyclohexyl)hexahydropyrrolo[3,4-c]pyrrole-2(1H)-carboxamide, rac-5-{[2-(4-Bromophenyl)imidazo[1,2-a]pyridin-3-yl]methyl}-N-(1-phenylethyl)hexahydropyrrolo[3,4-c]pyrrole-2(1H)-carboxamide, 5-{[2-(4-Bromophenyl)imidazo[1,2-a]pyridin-3-yl]methyl}-N-(4-fluorophenyl)hexahydropyrrolo[3,4-c]pyrrole-2(1H)-carboxamide, (3-Fluoro-6-methoxypyridin-2-yl)[5-{[2-(4-isopropylphenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone, [5-{[2-(4-Chlorophenyl)imidazo[1,2-a]pyridin-3-yl]methyl}hexahydropyrrolo[3,4-c]pyrrol-2(1H)-yl](3-fluoro-6-methoxypyridin-2-yl)methanone, 3-Chloro-6-methoxypyridin-2-yl)[5-{[2-(4-chlorophenyl)imidazo[1,2-a]pyridin-3-yl]methyl}hexahydropyrrolo[3,4-c]pyrrol-2(1H)-yl]methanone, tert-Butyl 5-{[2-(4-chlorophenyl)imidazo[1,2-a]pyridin-3-yl]methyl}-2,5-diazabicyclo[2.2.2]octane-2-carboxylate, tert-Butyl 5-{[2-(5-chloropyridin-2-yl)imidazo[1,2-a]pyridin-3-yl]methyl}-2,5-diazabicyclo[2.2.2]octane-2-carboxylate, tert-Butyl 7-{[2-(4-chlorophenyl)imidazo[1,2-a]pyridin-3-yl]methyl}-3-oxa-7,9-diazabicyclo[3.3.1]nonane-9-carboxylate, tert-Butyl 8-{[2-(4-bromophenyl)imidazo[1,2-a]pyridin-3-yl]methyl}-3,8-diazabicyclo[3.2.1]octane-3-carboxylate, tert-Butyl 8-{[2-(4-chlorophenyl)imidazo[1,2-a]pyridin-3-yl]methyl}-3,8-diazabicyclo[3.2.1]octane-3-carboxylate, tert-Butyl 8-{[2-(4-isopropylphenyl)imidazo[1,2-a]pyridin-3-yl]methyl}-3,8-diazabicyclo[3.2.1]octane-3-carboxylate, tert-Butyl 3-{[2-(4-chlorophenyl)imidazo[1,2-a]pyridin-3-yl]methyl}-3,6-diazabicyclo[3.1.1]heptane-6-carboxylate, tert-Butyl 3-{[2-(4-isopropylphenyl)imidazo[1,2-a]pyridin-3-yl]methyl}-3,6-diazabicyclo[3.1.1]heptane-6-carboxylate, tert-Butyl 3-{[2-(4-chlorophenyl)imidazo[1,2-a]pyridin-3-yl]methyl}-3,8-diazabicyclo[3.2.1]octane-8-carboxylate, tert-Butyl 3-{[2-(4-bromophenyl)imidazo[1,2-a]pyridine-3-yl]methyl}-3,8-diazabicyclo[3.2.1]octane-8-carboxylate, tert-Butyl 3-{[2-(4-isopropylphenyl)imidazo[1,2-a]pyridin-3-yl]methyl}-3,8-diazabicyclo[3.2.1]octane-8-carboxylate, tert-Butyl 5-{[2-(4-isopropylphenyl)imidazo[1,2-a]pyridin-3-yl]methyl}-2,5-diazabicyclo[2.2.2]octane-2-carboxylate, tert-Butyl 3-{[2-(5-chloropyridin-2-yl)imidazo[1,2-a]pyridin-3-yl]methyl}-3,8-diazabicyclo[3.2.1]octane-8-carboxylate, tert-Butyl 5-{[2-(4-chlorophenyl)imidazo[1,2-a]pyridin-3-yl]methyl}-2,5-diazabicyclo[2.2.2]octane-2-carboxylate, tert-Butyl 5-{[2-(4-isopropylphenyl)imidazo[1,2-a]pyridin-3-yl]methyl}-2,5-diazabicyclo[2.2.2]octane-2-carboxylate, tert-Butyl 5-{[2-(5-chloropyridin-2-yl)imidazo[1,2-a]pyridin-3-yl]methyl}-2,5-diazabicyclo[2.2.2]octane-2-carboxylate, (−)-[(1S,4S)-5-{[2-(4-Chlorophenyl)imidazo[1,2-a]pyridin-3-yl]methyl}-2,5-diazabicyclo[2.2.2]oct-2-yl](6-methoxypyridin-2-yl)methanone, (−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone, (−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(6-Isopropylpyridin-3-yl)imidazo[1,2-a]pyridin-3-yl]methyl}-2,5-diazabicyclo[2.2.2]oct-2-yl)(6-methoxypyridin-2-yl)methanone, (3-Fluoro-6-methoxypyridin-2-yl)(5-{[2-(6-isopropylpyridin-3-yl)imidazo[1,2-a]pyridin-3-yl]methyl}-2,5-diazabicyclo[2.2.2]oct-2-yl)methanone, (7-{[2-(4-Chlorophenyl)imidazo[1,2-a]pyridin-3-yl]methyl}-3-oxa-7,9-diazabicyclo[3.3.1]non-9-yl)(6-methoxypyridin-2-yl)methanone, (7-{[2-(4-Chlorophenyl)imidazo[1,2-a]pyridin-3-yl]methyl}-3-oxa-7,9-diazabicyclo[3.3.1]non-9-yl)(3-fluoro-6-methoxypyridin-2-yl)methanone, (7-{[2-(4-Chlorophenyl)imidazo[1,2-a]pyridin-3-yl]methyl}-3-oxa-7,9-diazabicyclo[3.3.1]non-9-yl)[6-(cyclobutyloxy)pyridin-2-yl]methanone, (3-Chloro-6-methoxypyridin-2-yl)(7-{[2-(4-chlorophenyl)imidazo[1,2-a]pyridin-3-yl]methyl}-3-oxa-7,9-diazabicyclo[3.3.1]non-9-yl)methanone, (3-{[2-(5-Chloropyridin-2-yl)imidazo[1,2-a]pyridin-3-yl]methyl}-3,8-diazabicyclo[3.2.1]oct-8-yl)(6-methoxypyridin-2-yl)methanone, (+)-[(1R,4R)-5-{[2-(4-Chlorophenyl)imidazo[1,2-a]pyridin-3-yl]methyl}-2,5-diazabicyclo[2.2.2]oct-2-yl](6-methoxypyridin-2-yl)methanone, (−)-(5-{[2-(4-Chlorophenyl)imidazo[1,2-a]pyridin-3-yl]methyl}-2,5-diazabicyclo[2.2.2]oct-2-yl)(2-fluorophenyl)methanone, (+)-(5-{[2-(4-Chlorophenyl)imidazo[1,2-a]pyridin-3-yl]methyl}-2,5-diazabicyclo[2.2.2]oct-2-yl)(2-fluorophenyl)methanone, 5-{[2-(4-Chlorophenyl)imidazo[1,2-a]pyridin-3-yl]methyl}-2,5-diazabicyclo[2.2.2]oct-2-yl)(cyclopentyl)methanone, (5-{[2-(4-Chlorophenyl)imidazo[1,2-a]pyridin-3-yl]methyl}-2,5-diazabicyclo[2.2.2]oct-2-yl)(cyclopentyl)methanone, (−)-(5-{[2-(4-Chlorophenyl)imidazo[1,2-a]pyridin-3-yl]methyl}-2,5-diazabicyclo[2.2.2]oct-2-yl)(3-methoxyphenyl)methanone, (+)-(5-{[2-(4-Chlorophenyl)imidazo[1,2-a]pyridin-3-yl]methyl}-2,5-diazabicyclo[2.2.2]oct-2-yl)(3-methoxyphenyl)methanone, (2-Chloro-5-fluorophenyl)(5-{[2-(4-chlorophenyl)imidazo[1,2-a]pyridin-3-yl]methyl}-2,5-diazabicyclo[2.2.2]oct-2-yl)methanone, (5-{[2-(4-Chlorophenyl)imidazo[1,2-a]pyridin-3-yl]methyl}-2,5-diazabicyclo[2.2.2]oct-2-yl)(cyclohexyl)methanone, (5-{[2-(4-Chlorophenyl)imidazo[1,2-a]pyridin-3-yl]methyl}-2,5-diazabicyclo[2.2.2]oct-2-yl)(cyclobutyl)methanone, (5-{[2-(4-Chlorophenyl)imidazo[1,2-a]pyridin-3-yl]methyl}-2,5-diazabicyclo[2.2.2]oct-2-yl)(2-methoxyphenyl)methanone, (5-{[2-(4-Chlorophenyl)imidazo[1,2-a]pyridin-3-yl]methyl}-2,5-diazabicyclo[2.2.2]oct-2-yl)(5-fluoro-2-methoxyphenyl)methanone, (5-{[2-(4-Chlorophenyl)imidazo[1,2-a]pyridin-3-yl]methyl}-2,5-diazabicyclo[2.2.2]oct-2-yl)(2-methylphenyl)methanone, (5-{[2-(4-Chlorophenyl)imidazo[1,2-a]pyridin-3-yl]methyl}-2,5-diazabicyclo[2.2.2]oct-2-yl)(5-fluoro-2-methylphenyl)methanone, (5-{[2-(4-Chlorophenyl)imidazo[1,2-a]pyridin-3-yl]methyl}-2,5-diazabicyclo[2.2.2]oct-2-yl)[3-(trifluoromethoxy)phenyl]methanone, (3-Chlorophenyl)(5-{[2-(4-chlorophenyl)imidazo[1,2-a]pyridin-3-yl]methyl}-2,5-diazabicyclo[2.2.2]oct-2-yl)methanone, (5-{[2-(4-Chlorophenyl)imidazo[1,2-a]pyridin-3-yl]methyl}-2,5-diazabicyclo[2.2.2]oct-2-yl)[3-(trifluoromethyl)phenyl]methanone, (5-{[2-(4-Chlorophenyl)imidazo[1,2-a]pyridin-3-yl]methyl}-2,5-diazabicyclo[2.2.2]oct-2-yl)(pyridin-2-yl)methanone, (5-{[2-(4-Chlorophenyl)imidazo[1,2-a]pyridin-3-yl]methyl}-2,5-diazabicyclo[2.2.2]oct-2-yl)(1-methyl-1H-imidazol-2-yl)methanone, (5-{[2-(4-Chlorophenyl)imidazo[1,2-a]pyridin-3-yl]methyl}-2,5-diazabicyclo[2.2.2]oct-2-yl)(3-methylphenyl)methanone, (5-{[2-(4-Chlorophenyl)imidazo[1,2-a]pyridin-3-yl]methyl}-2,5-diazabicyclo[2.2.2]oct-2-yl)(3-ethoxyphenyl)methanone, (5-{[2-(4-Chlorophenyl)imidazo[1,2-a]pyridin-3-yl]methyl}-2,5-diazabicyclo[2.2.2]oct-2-yl)(pyridin-4-yl)methanone, (−)-(2-Fluorophenyl)(5-{[2-(4-isopropylphenyl)imidazo[1,2-a]pyridin-3-yl]methyl}-2,5-diazabicyclo[2.2.2]oct-2-yl)methanone, (+)-(2-Fluorophenyl)(5-{[2-(4-isopropylphenyl)imidazo[1,2-a]pyridin-3-yl]methyl}-2,5-diazabicyclo[2.2.2]oct-2-yl)methanone, (−)-(5-{[2-(4-Isopropylphenyl)imidazo[1,2-a]pyridin-3-yl]methyl}-2,5-diazabicyclo[2.2.2]oct-2-yl)(3-methoxyphenyl)methanone, (+)-(5-{[2-(4-Isopropylphenyl)imidazo[1,2-a]pyridin-3-yl]methyl}-2,5-diazabicyclo[2.2.2]oct-2-yl)(3-methoxyphenyl)methanone, (−)-(5-{[2-(4-Isopropylphenyl)imidazo[1,2-a]pyridin-3-yl]methyl}-2,5-diazabicyclo[2.2.2]oct-2-yl)(6-methoxypyridin-2-yl)methanone, (+)-(5-{[2-(4-Isopropylphenyl)imidazo[1,2-a]pyridin-3-yl]methyl}-2,5-diazabicyclo[2.2.2]oct-2-yl)(6-methoxypyridin-2-yl)methanone, Cyclopentyl(5-{[2-(4-isopropylphenyl)imidazo[1,2-a]pyridin-3-yl]methyl}-2,5-diazabicyclo[2.2.2]oct-2-yl)methanone, Cyclopentyl(5-{[2-(4-isopropylphenyl)imidazo[1,2-a]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5-diazabicyclo[2.2.2]oct-2-yl)(2-fluorophenyl)methanone, (+)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone, (2-Fluorophenyl)(5-{[2-(6-isopropylpyridin-3-yl)imidazo[1,2-a]pyridin-3-yl]methyl}-2,5-diazabicyclo[2.2.2]oct-2-yl)methanone, (7-{[2-(4-Chlorophenyl)imidazo[1,2-a]pyridin-3-yl]methyl}-3-oxa-7,9-diazabicyclo[3.3.1]non-9-yl)(2-fluorophenyl)methanone, (7-{[2-(4-Chlorophenyl)imidazo[1,2-a]pyridin-3-yl]methyl}-3-oxa-7,9-diazabicyclo[3.3.1]non-9-yl)(3-methoxyphenyl)methanone, (7-{[2-(4-Chlorophenyl)imidazo[1,2-a]pyridin-3-yl]methyl}-3-oxa-7,9-diazabicyclo[3.3.1]non-9-yl)(cyclopentyl)methanone, (7-{[2-(4-Chlorophenyl)imidazo[1,2-a]pyridin-3-yl]methyl}-3-oxa-7,9-diazabicyclo[3.3.1]non-9-yl)[3-(trifluoromethoxy)phenyl]methanone, (7-{[2-(4-Chlorophenyl)imidazo[1,2-a]pyridin-3-yl]methyl}-3-oxa-7,9-diazabicyclo[3.3.1]non-9-yl)(2-isopropylphenyl)methanone, (2-Chloro-5-methoxyphenyl)(7-{[2-(4-chlorophenyl)imidazo[1,2-a]pyridin-3-yl]methyl}-3-oxa-7,9-diazabicyclo[3.3.1]non-9-yl)methanone, (7-{[2-(4-Chlorophenyl)imidazo[1,2-a]pyridin-3-yl]methyl}-3-oxa-7,9-diazabicyclo[3.3.1]non-9-yl)(5-fluoro-2-methoxyphenyl)methanone, (7-{[2-(4-Chlorophenyl)imidazo[1,2-a]pyridin-3-yl]methyl}-3-oxa-7,9-diazabicyclo[3.3.1]non-9-yl)(3-isopropylphenyl)methanone, (7-{[2-(4-Chlorophenyl)imidazo[1,2-a]pyridin-3-yl]methyl}-3-oxa-7,9-diazabicyclo[3.3.1]non-9-yl)[6-(2,2,2-trifluoroethoxy)pyridin-2-yl]methanone, (7-{[2-(4-Chlorophenyl)imidazo[1,2-a]pyridin-3-yl]methyl}-3-oxa-7,9-diazabicyclo[3.3.1]non-9-yl)(tetrahydrofuran-3-yl)methanone, (3-Chlorophenyl)(7-{[2-(4-chlorophenyl)imidazo[1,2-a]pyridin-3-yl]methyl}-3-oxa-7,9-diazabicyclo[3.3.1]non-9-yl)methanone, (7-{[2-(4-Chlorophenyl)imidazo[1,2-a]pyridin-3-yl]methyl}-3-oxa-7,9-diazabicyclo[3.3.1]non-9-yl)[6-(trifluoromethoxy)pyridin-2-yl]methanone, (7-{[2-(4-Chlorophenyl)imidazo[1,2-a]pyridin-3-yl]methyl}-3-oxa-7,9-diazabicyclo[3.3.1]non-9-yl)(6-methoxy-3-methylpyridin-2-yl)methanone, (8-{[2-(4-Bromophenyl)imidazo[1,2-a]pyridin-3-yl]methyl}-3,8-diazabicyclo[3.2.1]oct-3-yl)(2-fluorophenyl)methanone, (8-{[2-(4-Bromophenyl)imidazo[1,2-a]pyridin-3-yl]methyl}-3,8-diazabicyclo[3.2.1]oct-3-yl)(6-methoxypyridin-2-yl)methanone, (8-{[2-(4-Bromophenyl)imidazo[1,2-a]pyridin-3-yl]methyl}-3,8-diazabicyclo[3.2.1]oct-3-yl)(3-methoxyphenyl)methanone, (8-{[2-(4-Bromophenyl)imidazo[1,2-a]pyridin-3-yl]methyl}-3,8-diazabicyclo[3.2.1]oct-3-yl)(cyclopentyl)methanone, (8-{[2-(4-Chlorophenyl)imidazo[1,2-a]pyridin-3-yl]methyl}-3,8-diazabicyclo[3.2.1]oct-3-yl)(cyclopentyl)methanone, (8-{[2-(4-Chlorophenyl)imidazo[1,2-a]pyridin-3-yl]methyl}-3,8-diazabicyclo[3.2.1]oct-3-yl)(2-fluorophenyl)methanone, (8-{[2-(4-Chlorophenyl)imidazo[1,2-a]pyridin-3-yl]methyl}-3,8-diazabicyclo[3.2.1]oct-3-yl)(5-fluoro-2-methylphenyl)methanone, (8-{[2-(4-Chlorophenyl)imidazo[1,2-a]pyridin-3-yl]methyl}-3,8-diazabicyclo[3.2.1]oct-3-yl)(5-fluoro-2-methoxyphenyl)methanone, (8-{[2-(4-Chlorophenyl)imidazo[1,2-a]pyridin-3-yl]methyl}-3,8-diazabicyclo[3.2.1]oct-3-yl)(2-methylphenyl)methanone, (8-{[2-(4-Chlorophenyl)imidazo[1,2-a]pyridin-3-yl]methyl}-3,8-diazabicyclo[3.2.1]oct-3-yl)(2-methoxyphenyl)methanone, (8-{[2-(4-Chlorophenyl)imidazo[1,2-a]pyridin-3-yl]methyl}-3,8-diazabicyclo[3.2.1]oct-3-yl)(6-methoxypyridin-2-yl)methanone, (8-{[2-(4-Chlorophenyl)imidazo[1,2-a]pyridin-3-yl]methyl}-3,8-diazabicyclo[3.2.1]oct-3-yl)(cyclohexyl)methanone, (2-Fluorophenyl)(8-{[2-(4-isopropylphenyl)imidazo[1,2-a]pyridin-3-yl]methyl}-3,8-diazabicyclo[3.2.1]oct-3-yl)methanone, (8-{[2-(4-Isopropylphenyl)imidazo[1,2-a]pyridin-3-yl]methyl}-3,8-diazabicyclo[3.2.1]oct-3-yl)(6-methoxypyridin-2-yl)methanone, (3-{[2-(4-Bromophenyl)imidazo[1,2-a]pyridin-3-yl]methyl}-3,8-diazabicyclo[3.2.1]oct-8-yl)(cyclopentyl)methanone, (3-{[2-(4-Bromophenyl)imidazo[1,2-a]pyridin-3-yl]methyl}-3,8-diazabicyclo[3.2.1]oct-8-yl)(2-fluorophenyl)methanone, (3-{[2-(4-Bromophenyl)imidazo[1,2-a]pyridin-3-yl]methyl}-3,8-diazabicyclo[3.2.1]oct-8-yl)(6-methoxypyridin-2-yl)methanone, (3-{[2-(4-Bromophenyl)imidazo[1,2-a]pyridin-3-yl]methyl}-3,8-diazabicyclo[3.2.1]oct-8-yl)(3-methoxyphenyl)methanone, (3-{[2-(4-Chlorophenyl)imidazo[1,2-a]pyridin-3-yl]methyl}-3,8-diazabicyclo[3.2.1]oct-8-yl)(2-methylphenyl)methanone, (3-{[2-(4-Chlorophenyl)imidazo[1,2-a]pyridin-3-yl]methyl}-3,8-diazabicyclo[3.2.1]oct-8-yl)(cyclobutyl)methanone, (3-{[2-(4-Chlorophenyl)imidazo[1,2-a]pyridin-3-yl]methyl}-3,8-diazabicyclo[3.2.1]oct-8-yl)(2-fluorophenyl)methanone, (3-{[2-(4-Chlorophenyl)imidazo[1,2-a]pyridin-3-yl]methyl}-3,8-diazabicyclo[3.2.1]oct-8-yl)(5-fluoro-2-methoxyphenyl)methanone, (3-{[2-(4-Chlorophenyl)imidazo[1,2-a]pyridin-3-yl]methyl}-3,8-diazabicyclo[3.2.1]oct-8-yl)(6-methoxypyridin-2-yl)methanone, (3-{[2-(4-Chlorophenyl)imidazo[1,2-a]pyridin-3-yl]methyl}-3,8-diazabicyclo[3.2.1]oct-8-yl)(cyclohexyl)methanone, (2-Chloro-5-fluorophenyl)(3-{[2-(4-chlorophenyl)imidazo[1,2-a]pyridin-3-yl]methyl}-3,8-diazabicyclo[3.2.1]oct-8-yl)methanone, (3-{[2-(4-Chlorophenyl)imidazo[1,2-a]pyridin-3-yl]methyl}-3,8-diazabicyclo[3.2.1]oct-8-yl)(5-fluoro-2-methylphenyl)methanone, (3-{[2-(4-Chlorophenyl)imidazo[1,2-a]pyridin-3-yl]methyl}-3,8-diazabicyclo[3.2.1]oct-8-yl)(3-methoxyphenyl)methanone, (3-{[2-(4-Chlorophenyl)imidazo[1,2-a]pyridin-3-yl]methyl}-3,8-diazabicyclo[3.2.1]oct-8-yl)(2-methoxyphenyl)methanone, (2-Fluorophenyl)(3-{[2-(4-isopropylphenyl)imidazo[1,2-a]pyridin-3-yl]methyl}-3,8-diazabicyclo[3.2.1]oct-8-yl)methanone, (3-{[2-(5-Chloropyridin-2-yl)imidazo[1,2-a]pyridin-3-yl]methyl}-3,8-diazabicyclo[3.2.1]oct-8-yl)(2-fluorophenyl)methanone, (3-{[2-(5-Chloropyridin-2-yl)imidazo[1,2-a]pyridin-3-yl]methyl}-3,8-diazabicyclo[3.2.1]oct-8-yl)(3-methoxyphenyl)methanone, (3-{[2-(5-Chloropyridin-2-yl)imidazo[1,2-a]pyridin-3-yl]methyl}-3,8-diazabicyclo[3.2.1]oct-8-yl)(cyclopentyl)methanone, (3-Chlorophenyl)(3-{[2-(4-chlorophenyl)imidazo[1,2-a]pyridin-3-yl]methyl}-3,6-diazabicyclo[3.1.1]hept-6-yl)methanone, (3-{[2-(4-Chlorophenyl)imidazo[1,2-a]pyridin-3-yl]methyl}-3,6-diazabicyclo[3.1.1]hept-6-yl)(tetrahydrofuran-2-yl)methanone, (3-{[2-(4-Chlorophenyl)imidazo[1,2-a]pyridin-3-yl]methyl}-3,6-diazabicyclo[3.1.1]hept-6-yl)(cyclopentyl)methanone, (3-{[2-(4-Chlorophenyl)imidazo[1,2-a]pyridin-3-yl]methyl}-3,6-diazabicyclo[3.1.1]hept-6-yl)(2-fluorophenyl)methanone, (3-{[2-(4-Chlorophenyl)imidazo[1,2-a]pyridin-3-yl]methyl}-3,6-diazabicyclo[3.1.1]hept-6-yl)(cyclohexyl)methanone, (2-Chloro-5-fluorophenyl)(3-{[2-(4-chlorophenyl)imidazo[1,2-a]pyridin-3-yl]methyl}-3,6-diazabicyclo[3.1.1]hept-6-yl)methanone, (3-{[2-(4-Chlorophenyl)imidazo[1,2-a]pyridin-3-yl]methyl}-3,6-diazabicyclo[3.1.1]hept-6-yl)[3-(trifluoromethoxy)phenyl]methanone, (3-{[2-(4-Chlorophenyl)imidazo[1,2-a]pyridin-3-yl]methyl}-3,6-diazabicyclo[3.1.1]hept-6-yl)(cyclobutyl)methanone, (3-{[2-(4-Chlorophenyl)imidazo[1,2-a]pyridin-3-yl]methyl}-3,6-diazabicyclo[3.1.1]hept-6-yl)(3-ethoxyphenyl)methanone, Cyclopentyl(3-{[2-(4-isopropylphenyl)imidazo[1,2-a]pyridin-3-yl]methyl}-3,6-diazabicyclo[3.1.1]hept-6-yl)methanone, (5-{[2-(6-Isopropylpyridin-3-yl)imidazo[1,2-a]pyridin-3-yl]methyl}-2,5-diazabicyclo[2.2.2]oct-2-yl)(6-methoxypyridin-2-yl)methanone, (3-Fluoro-6-methoxypyridin-2-yl)(5-{[2-(6-isopropylpyridin-3-yl)imidazo[1,2-a]pyridin-3-yl]methyl}-2,5-diazabicyclo[2.2.2]oct-2-yl)methanone, (2-Fluorophenyl)(5-{[2-(6-isopropylpyridin-3-yl)imidazo[1,2-a]pyridin-3-yl]methyl}-2,5-diazabicyclo[2.2.2]oct-2-yl)methanone, tert-Butyl 7-{[2-(5-chloropyridin-2-yl)imidazo[1,2-a]pyridine-3-yl]methyl}-3-oxa-7,9-diazabicyclo[3.3.1]nonane-9-carboxylate, tert-Butyl 3-{[2-(6-isopropylpyridin-3-yl)imidazo[1,2-a]pyridine-3-yl]methyl}-3,8-diazabicyclo[3.2.1]octane-8-carboxylate, tert-Butyl 5-{[2-(4-bromophenyl)imidazo[1,2-a]pyridin-3-yl]methyl}-2,5-diazabicyclo[2.2.2]octane-2-carboxylate, tert-Butyl 5-{[2-(6-isopropylpyridin-3-yl)imidazo[1,2-a]pyridin-3-yl]methyl}-2,5-diazabicyclo[2.2.2]octane-2-carboxylate, (7-{[2-(5-Chloropyridin-2-yl)imidazo[1,2-a]pyridin-3-yl]methyl}-3-oxa-7,9-diazabicyclo[3.3.1]non-9-yl)[6-(trifluoromethoxy)pyridin-2-yl]methanone, (3-Chloro-6-methoxypyridin-2-yl)(7-{[2-(5-chloropyridin-2-yl)imidazo[1,2-a]pyridin-3-yl]methyl}-3-oxa-7,9-diazabicyclo[3.3.1]non-9-yl)methanone, 5-{[2-(4-Isopropylphenyl)imidazo[1,2-a]pyridin-3-yl]methyl}-2,5-diazabicyclo[2.2.2]oct-2-yl](6-methoxy-3-methylpyridin-2-yl)methanone, 5-{[2-(4-Chlorophenyl)imidazo[1,2-a]pyridin-3-yl]methyl}-2,5-diazabicyclo[2.2.2]oct-2-yl](6-methoxy-3-methylpyridin-2-yl)methanone, (3-{[2-(4-Bromophenyl)imidazo[1,2-a]pyridin-3-yl]methyl}-3,8-diazabicyclo[3.2.1]oct-8-yl)(3-chloro-6-methoxypyridin-2-yl)methanone, (3-{[2-(4-Bromophenyl)imidazo[1,2-a]pyridin-3-yl]methyl}-3,8-diazabicyclo[3.2.1]oct-8-yl)(3-fluoro-6-methoxypyridin-2-yl)methanone, (3-{[2-(4-Bromophenyl)imidazo[1,2-a]pyridin-3-yl]methyl}-3,8-diazabicyclo[3.2.1]oct-8-yl)(2-isopropylphenyl)methanone, (3-{[2-(4-Chlorophenyl)imidazo[1,2-a]pyridin-3-yl]methyl}-3,6-diazabicyclo[3.1.1]hept-6-yl)(6-methoxy-3-methylpyridin-2-yl)methanone, (8-{[2-(4-Chlorophenyl)imidazo[1,2-a]pyridin-3-yl]methyl}-3,8-diazabicyclo[3.2.1]oct-3-yl)(6-methoxy-3-methylpyridin-2-yl)methanone, (8-{[2-(4-Isopropylphenyl)imidazo[1,2-a]pyridin-3-yl]methyl}-3,8-diazabicyclo[3.2.1]oct-3-yl)(6-methoxy-3-methylpyridin-2-yl)methanone, (3-{[2-(4-Isopropylphenyl)imidazo[1,2-a]pyridin-3-yl]methyl}-3,8-diazabicyclo[3.2.1]oct-8-yl)(6-methoxy-3-methylpyridin-2-yl)methanone, (3-{[2-(4-Chlorophenyl)imidazo[1,2-a]pyridin-3-yl]methyl}-3,8-diazabicyclo[3.2.1]oct-8-yl)(4-isopropyl-1,3-thiazol-2-yl)methanone, (3-{[2-(4-Chlorophenyl)imidazo[1,2-a]pyridin-3-yl]methyl}-3,8-diazabicyclo[3.2.1]oct-8-yl)(1,3-thiazol-2-yl)methanone, (3-{[2-(4-Chlorophenyl)imidazo[1,2-a]pyridin-3-yl]methyl}-3,8-diazabicyclo[3.2.1]oct-8-yl)(4-methyl-1,3-thiazol-2-yl)methanone, (3-{[2-(4-Chlorophenyl)imidazo[1,2-a]pyridin-3-yl]methyl}-3,8-diazabicyclo[3.2.1]oct-8-yl)(5-methyl-1,3-thiazol-2-yl)methanone, (3-{[2-(4-Chlorophenyl)imidazo[1,2-a]pyridin-3-yl]methyl}-3,8-diazabicyclo[3.2.1]oct-8-yl)(4,5-dimethyl-1,3-thiazol-2-yl)methanone, (5-{[2-(4-Bromophenyl)imidazo[1,2-a]pyridin-3-yl]methyl}-2,5-diazabicyclo[2.2.2]oct-2-yl)(6-methoxypyridin-2-yl)methanone, (5-{[2-(4-Bromophenyl)imidazo[1,2-a]pyridin-3-yl]methyl}-2,5-diazabicyclo[2.2.2]oct-2-yl)(2-fluorophenyl)methanone, (5-{[2-(4-Bromophenyl)imidazo[1,2-a]pyridin-3-yl]methyl}-2,5-diazabicyclo[2.2.2]oct-2-yl)(3-fluoro-6-methoxypyridin-2-yl)methanone, 3-{[2-(4-Chlorophenyl)imidazo[1,2-a]pyridin-3-yl]methyl}-N-isopropyl-3,8-diazabicyclo[3.2.1]octane-8-carboxamide, 3-{[2-(4-Chlorophenyl)imidazo[1,2-a]pyridin-3-yl]methyl}-N-(2-fluorophenyl)-3,8-diazabicyclo[3.2.1]octane-8-carboxamide, 3-{[2-(4-Chlorophenyl)imidazo[1,2-a]pyridin-3-yl]methyl}-N-(2,6-dichlorophenyl)-3,8-diazabicyclo[3.2.1]octane-8-carboxamide, 3-{[2-(4-Chlorophenyl)imidazo[1,2-a]pyridin-3-yl]methyl}-N-(2,6-dimethylphenyl)-3,8-diazabicyclo[3.2.1]octane-8-carboxamide, 3-{[2-(4-Chlorophenyl)imidazo[1,2-a]pyridin-3-yl]methyl}-N-pentyl-3,8-diazabicyclo[3.2.1]octane-8-carboxamide, 3-{[2-(4-Chlorophenyl)imidazo[1,2-a]pyridin-3-yl]methyl}-N-(2-methylphenyl)-3,8-diazabicyclo[3.2.1]octane-8-carboxamide, 3-{[2-(4-Chlorophenyl)imidazo[1,2-a]pyridin-3-yl]methyl}-N-[2-chloro-5-(trifluoromethyl)phenyl]-3,8-diazabicyclo[3.2.1]octane-8-carboxamide, N-(4-Chlorophenyl)-3-{[2-(4-chlorophenyl)imidazo[1,2-a]pyridin-3-yl]methyl}-3,8-diazabicyclo[3.2.1]octane-8-carboxamide, 3-{[2-(4-Chlorophenyl)imidazo[1,2-a]pyridin-3-yl]methyl}-N-(2-ethyl-6-methylphenyl)-3,8-diazabicyclo[3.2.1]octane-8-carboxamide, 3-{[2-(4-Chlorophenyl)imidazo[1,2-a]pyridin-3-yl]methyl}-N-(2,5-dimethylphenyl)-3,8-diazabicyclo[3.2.1]octane-8-carboxamide, 3-{[2-(4-Chlorophenyl)imidazo[1,2-a]pyridin-3-yl]methyl}-N-cyclohexyl-3,8-diazabicyclo[3.2.1]octane-8-carboxamide, N-(2-Chloro-6-methylphenyl)-3-{[2-(4-chlorophenyl)imidazo[1,2-a]pyridin-3-yl]methyl}-3,8-diazabicyclo[3.2.1]octane-8-carboxamide, 3-{[2-(4-Chlorophenyl)imidazo[1,2-a]pyridin-3-yl]methyl}-N-(2,6-difluorophenyl)-3,8-diazabicyclo[3.2.1]octane-8-carboxamide, 3-{[2-(4-Chlorophenyl)imidazo[1,2-a]pyridin-3-yl]methyl}-N-(2,4-dimethylphenyl)-3,8-diazabicyclo[3.2.1]octane-8-carboxamide, 7-{[2-(4-Chlorophenyl)imidazo[1,2-a]pyridin-3-yl]methyl}-N-isopropyl-3-oxa-7,9-diazabicyclo[3.3.1]nonane-9-carboxamide, N-(2-Chloro-6-methylphenyl)-7-{[2-(4-chlorophenyl)imidazo[1,2-a]pyridin-3-yl]methyl}-3-oxa-7,9-diazabicyclo[3.3.1]nonane-9-carboxamide, 3-{[2-(4-Chlorophenyl)imidazo[1,2-a]pyridin-3-yl]methyl}-N-cyclopropyl-3,8-diazabicyclo[3.2.1]octane-8-carboxamide, N-(2-Chlorophenyl)-3-{[2-(4-chlorophenyl)imidazo[1,2-a]pyridin-3-yl]methyl}-3,8-diazabicyclo[3.2.1]octane-8-carboxamide, 3-{[2-(4-Chlorophenyl)imidazo[1,2-a]pyridin-3-yl]methyl}-N-methyl-N-phenyl-3,8-diazabicyclo[3.2.1]octane-8-carboxamide, (3-{[2-(4-Chlorophenyl)imidazo[1,2-a]pyridin-3-yl]methyl}-3,8-diazabicyclo[3.2.1]oct-8-yl)(morpholin-4-yl)methanone, 3-{[2-(4-Chlorophenyl)imidazo[1,2-a]pyridin-3-yl]methyl}-N,N-diisopropyl-3,8-diazabicyclo[3.2.1]octane-8-carboxamide, 3-{[2-(4-Chlorophenyl)imidazo[1,2-a]pyridin-3-yl]methyl}-N-cyclohexyl-N-ethyl-3,8-diazabicyclo[3.2.1]octane-8-carboxamide, (3-{[2-(4-Chlorophenyl)imidazo[1,2-a]pyridin-3-yl]methyl}-3,8-diazabicyclo[3.2.1]oct-8-yl)(pyrrolidin-1-yl)methanone, 3-{[2-(4-Chlorophenyl)imidazo[1,2-a]pyridin-3-yl]methyl}-N-ethyl-N-phenyl-3,8-diazabicyclo[3.2.1]octane-8-carboxamide, 3-{[2-(4-Chlorophenyl)imidazo[1,2-a]pyridin-3-yl]methyl}-N-isopropyl-N-methyl-3,8-diazabicyclo[3.2.1]octane-8-carboxamide, (3-{[2-(4-Chlorophenyl)imidazo[1,2-a]pyridin-3-yl]methyl}-3,8-diazabicyclo[3.2.1]oct-8-yl)(piperidin-1-yl)methanone, 3-{[2-(4-Chlorophenyl)imidazo[1,2-a]pyridin-3-yl]methyl}-N,N-dimethyl-3,8-diazabicyclo[3.2.1]octane-8-carboxamide, 3-{[2-(4-Chlorophenyl)imidazo[1,2-a]pyridin-3-yl]methyl}-N-ethyl-N-(4-methylphenyl)-3,8-diazabicyclo[3.2.1]octane-8-carboxamide, N-(4-Chlorophenyl)-3-{[2-(4-chlorophenyl)imidazo[1,2-a]pyridin-3-yl]methyl}-N-isopropyl-3,8-diazabicyclo[3.2.1]octane-8-carboxamide, (3-{[2-(4-Chlorophenyl)imidazo[1,2-a]pyridin-3-yl]methyl}-3,8-diazabicyclo[3.2.1]oct-8-yl)(thiomorpholin-4-yl)methanone, Methyl 3-{[2-(4-chlorophenyl)imidazo[1,2-a]pyridin-3-yl]methyl}-3,8-diazabicyclo[3.2.1]octane-8-carboxylate, Ethyl 3-{[2-(4-chlorophenyl)imidazo[1,2-a]pyridin-3-yl]methyl}-3,8-diazabicyclo[3.2.1]octane-8-carboxylate, Cyclopentyl 3-{[2-(4-chlorophenyl)imidazo[1,2-a]pyridin-3-yl]methyl}-3,8-diazabicyclo[3.2.1]octane-8-carboxylate, Cyclohexyl 3-{[2-(4-chlorophenyl)imidazo[1,2-a]pyridin-3-yl]methyl}-3,8-diazabicyclo[3.2.1]octane-8-carboxylate, 7-{[2-(4-Chlorophenyl)imidazo[1,2-a]pyridin-3-yl]methyl}-N,N-diethyl-3-oxa-7,9-diazabicyclo[3.3.1]nonane-9-carboxamide, (7-{[2-(4-Chlorophenyl)imidazo[1,2-a]pyridin-3-yl]methyl}-3-oxa-7,9-diazabicyclo[3.3.1]non-9-yl)(morpholin-4-yl)methanone, 7-{[2-(4-Chlorophenyl)imidazo[1,2-a]pyridin-3-yl]methyl}-N,N-diisopropyl-3-oxa-7,9-diazabicyclo[3.3.1]nonane-9-carboxamide, 7-{[2-(4-Chlorophenyl)imidazo[1,2-a]pyridin-3-yl]methyl}-N-cyclohexyl-N-ethyl-3-oxa-7,9-diazabicyclo[3.3.1]nonane-9-carboxamide, (7-{[2-(4-Chlorophenyl)imidazo[1,2-a]pyridin-3-yl]methyl}-3-oxa-7,9-diazabicyclo[3.3.1]non-9-yl)(pyrrolidin-1-yl)methanone, 7-{[2-(4-Chlorophenyl)imidazo[1,2-a]pyridin-3-yl]methyl}-N-ethyl-N-phenyl-3-oxa-7,9-diazabicyclo[3.3.1]nonane-9-carboxamide, 7-{[2-(4-Chlorophenyl)imidazo[1,2-a]pyridin-3-yl]methyl}-N-isopropyl-N-methyl-3-oxa-7,9-diazabicyclo[3.3.1]nonane-9-carboxamide, Ethyl 7-{[2-(4-chlorophenyl)imidazo[1,2-a]pyridin-3-yl]methyl}-3-oxa-7,9-diazabicyclo[3.3.1]nonane-9-carboxylate, Cyclopentyl 7-{[2-(4-chlorophenyl)imidazo[1,2-a]pyridin-3-yl]methyl}-3-oxa-7,9-diazabicyclo[3.3.1]nonane-9-carboxylate, Propyl 7-{[2-(4-chlorophenyl)imidazo[1,2-a]pyridin-3-yl]methyl}-3-oxa-7,9-diazabicyclo[3.3.1]nonane-9-carboxylate, (7-{[2-(4-Chlorophenyl)imidazo[1,2-a]pyridin-3-yl]methyl}-3-oxa-7,9-diazabicyclo[3.3.1]non-9-yl)(piperidin-1-yl)methanone, (5-{[2-(4-Bromophenyl)imidazo[1,2-a]pyridin-3-yl]methyl}-2,5-diazabicyclo[2.2.2]oct-2-yl)(3-chlor-6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)[6-(difluoromethoxy)pyridin-2-yl]methanone, tert-Butyl 7-{[2-(4-chlorophenyl)imidazo[1,2-a]pyrimidin-3-yl]methyl}-3-oxa-7,9-diazabicyclo[3.3.1]nonane-9-carboxylate, tert-Butyl 7-{[2-(4-isopropylphenyl)imidazo[1,2-a]pyrimidin-3-yl]methyl}-3-oxa-7,9-diazabicyclo[3.3.1]nonane-9-carboxylate, tert-Butyl 5-{[2-(4-chlorophenyl)imidazo[1,2-a]pyrimidin-3-yl]methyl}-2,5-diazabicyclo[2.2.2]octane-2-carboxylate, tert-Butyl 5-{[2-(4-chlorophenyl)imidazo[1,2-a]pyrimidin-3-yl]methyl}-2,5-diazabicyclo[2.2.2]octane-2-carboxylate, tert-Butyl 3-{[2-(4-chlorophenyl)imidazo[1,2-a]pyrimidin-3-yl]methyl}-3,8-diazabicyclo[3.2.1]octane-8-carboxylate, tert-Butyl 5-{[2-(4-isopropylphenyl)imidazo[1,2-a]pyrimidine-3-yl]methyl}-2,5-diazabicyclo[2.2.2]octane-2-carboxylate, tert-Butyl 5-{[2-(4-isopropylphenyl)imidazo[1,2-a]pyrimidin-3-yl]methyl}-2,5-diazabicyclo[2.2.2]octane-2-carboxylate, tert-Butyl 3-{1-[2-(4-chlorophenyl)imidazo[1,2-a]pyrimidin-3-yl]ethyl}-3,8-diazabicyclo[3.2.1]octane-8-carboxylate, tert-butyl 5-{[2-(4-bromophenyl)imidazo[1,2-a]pyrimidin-3-yl]methyl}-2,5-diazabicyclo[2.2.2]octane-2-carboxylate, tert-butyl 3-{[2-(4-bromophenyl)imidazo[1,2-a]pyrimidin-3-yl]methyl}-3,8-diazabicyclo[3.2.1]octane-8-carboxylate, (7-{[2-(4-Chlorophenyl)imidazo[1,2-a]pyrimidin-3-yl]methyl}-3-oxa-7,9-diazabicyclo[3.3.1]non-9-yl)(6-methoxypyridin-2-yl)methanone, (3-Chloro-6-methoxypyridin-2-yl)(7-{[2-(4-chlorophenyl)imidazo[1,2-a]pyrimidin-3-yl]methyl}-3-oxa-7,9-diazabicyclo[3.3.1]non-9-yl)methanone, (7-{[2-(4-Chlorophenyl)imidazo[1,2-a]pyrimidin-3-yl]methyl}-3-oxa-7,9-diazabicyclo[3.3.1]non-9-yl)(3-fluoro-6-methoxypyridin-2-yl)methanone, (7-{[2-(4-Chlorophenyl)imidazo[1,2-a]pyrimidin-3-yl]methyl}-3-oxa-7,9-diazabicyclo[3.3.1]non-9-yl)[6-(methylsulfanyl)pyridin-2-yl]methanone, (7-{[2-(4-Chlorophenyl)imidazo[1,2-a]pyrimidin-3-yl]methyl}-3-oxa-7,9-diazabicyclo[3.3.1]non-9-yl)(cyclopentyl)methanone, (3-Fluoro-6-methoxypyridin-2-yl)(7-{[2-(4-isopropylphenyl)imidazo[1,2-a]pyrimidin-3-yl]methyl}-3-oxa-7,9-diazabicyclo[3.3.1]non-9-yl)methanone, [6-(Difluoromethoxy)pyridin-2-yl](7-{[2-(4-isopropylphenyl)imidazo[1,2-a]pyrimidin-3-yl]methyl}-3-oxa-7,9-diazabicyclo[3.3.1]non-9-yl)methanone, (3-{[2-(4-Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone, (3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone, (3-Chloro-6-methoxypyridin-2-yl)(5-{[2-(4-chlorophenyl)imidazo[1,2-a]pyrimidin-3-yl]methyl}-2,5-diazabicyclo[2.2.2]oct-2-yl)methanone, (5-{[2-(4-Chlorophenyl)imidazo[1,2-a]pyrimidin-3-yl]methyl}-2,5-diazabicyclo[2.2.2]oct-2-yl)(3-fluoro-6-methoxypyridin-2-yl)methanone, (5-{[2-(4-Chlorophenyl)imidazo[1,2-a]pyrimidin-3-yl]methyl}-2,5-diazabicyclo[2.2.2]oct-2-yl)(6-methoxy-3-methylpyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (5-Cyclopropyl-1,3-oxazol-4-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (7-{[2-(4-chlorophenyl)imidazo[1,2-a]pyrimidin-3-yl]methyl}-3-oxa-7,9-diazabicyclo[3.3.1]non-9-yl)(2-fluorophenyl)methanone, (7-{[2-(4-chlorophenyl)imidazo[1,2-a]pyrimidin-3-yl]methyl}-3-oxa-7,9-diazabicyclo[3.3.1]non-9-yl)(3-methoxyphenyl)methanone, (3-{[2-(4-chlorophenyl)imidazo[1,2-a]pyrimidin-3-yl]methyl}-3,8-diazabicyclo[3.2.1]oct-8-yl)(2-fluorophenyl)methanone, (3-{[2-(4-chlorophenyl)imidazo[1,2-a]pyrimidin-3-yl]methyl}-3,8-diazabicyclo[3.2.1]oct-8-yl)[6-(methylsulfanyl)pyridin-2-yl]methanone, (3-{[2-(4-chlorophenyl)imidazo[1,2-a]pyrimidin-3-yl]methyl}-3,8-diazabicyclo[3.2.1]oct-8-yl)(cyclopentyl)methanone, (3-{[2-(4-chlorophenyl)imidazo[1,2-a]pyrimidin-3-yl]methyl}-3,8-diazabicyclo[3.2.1]oct-8-yl)[6-(methylamino)pyridin-2-yl]methanone, (3-{[2-(4-chlorophenyl)imidazo[1,2-a]pyrimidin-3-yl]methyl}-3,8-diazabicyclo[3.2.1]oct-8-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(cyclopentyl)methanone, (5-{[2-(4-chlorophenyl)imidazo[1,2-a]pyrimidin-3-yl]methyl}-2,5-diazabicyclo[2.2.2]oct-2-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(2-fluorophenyl)methanone, (5-{[2-(4-chlorophenyl)imidazo[1,2-a]pyrimidin-3-yl]methyl}-2,5-diazabicyclo[2.2.2]oct-2-yl)(6-methoxypyridin-2-yl)methanone, (5-{[2-(4-chlorophenyl)imidazo[1,2-a]pyrimidin-3-yl]methyl}-2,5-diazabicyclo[2.2.2]oct-2-yl)(3-fluoro-6-methoxypyridin-2-yl)methanone, (5-{[2-(4-chlorophenyl)imidazo[1,2-a]pyrimidin-3-yl]methyl}-2,5-diazabicyclo[2.2.2]oct-2-yl)(2-fluorophenyl)methanone, (5-{[2-(4-chlorophenyl)imidazo[1,2-a]pyrimidin-3-yl]methyl}-2,5-diazabicyclo[2.2.2]oct-2-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(6-methoxypyridin-2-yl)methanone, (3-chloro-6-methoxypyridin-2-yl)(5-{[2-(4-chlorophenyl)imidazo[1,2-a]pyrimidin-3-yl]methyl}-2,5-diazabicyclo[2.2.2]oct-2-yl)methanone, (7-{[2-(4-chlorophenyl)imidazo[1,2-a]pyrimidin-3-yl]methyl}-3-oxa-7,9-diazabicyclo[3.3.1]non-9-yl)(5-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(5-methyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(5-isopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2,4-dimethyl-1,3-oxazol-5-yl)methanone, (3-{[2-(4-chlorophenyl)imidazo[1,2-a]pyrimidin-3-yl]methyl}-3,8-diazabicyclo[3.2.1]oct-8-yl)(5-ethyl-1,3-oxazol-4-yl)methanone, (4-bromo-5-methyl-1,3-thiazol-2-yl)(3-{[2-(4-chlorphenyl)imidazo[1,2-a]pyrimidin-3-yl]methyl}-3,8-diazabicyclo[3.2.1]oct-8-yl)methanone, (3-{[2-(4-chlorophenyl)imidazo[1,2-a]pyrimidin-3-yl]methyl}-3,8-diazabicyclo[3.2.1]oct-8-yl)(5-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2-isopropyl-1,3-thiazol-4-yl)methanone, (3-{[2-(4-chlorophenyl)imidazo[1,2-a]pyrimidin-3-yl]methyl}-3,8-diazabicyclo[3.2.1]oct-8-yl)(1,3-thiazol-5-yl)methanone, (3-{[2-(4-chlorophenyl)imidazo[1,2-a]pyrimidin-3-yl]methyl}-3,8-diazabicyclo[3.2.1]oct-8-yl)(2,5-dimethyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)[2-methoxy-4-(trifluoromethyl)-1,3-thiazol-5-yl]methanone, (3-{[2-(4-chlorophenyl)imidazo[1,2-a]pyrimidin-3-yl]methyl}-3,8-diazabicyclo[3.2.1]oct-8-yl)[2-(trifluoromethyl)-1,3-thiazol-4-yl]methanone, (3-{[2-(4-chlorophenyl)imidazo[1,2-a]pyrimidin-3-yl]methyl}-3,8-diazabicyclo[3.2.1]oct-8-yl)(5-methyl-1,3-thiazol-4-yl)methanone, (3-{[2-(4-chlorophenyl)imidazo[1,2-a]pyrimidin-3-yl]methyl}-3,8-diazabicyclo[3.2.1]oct-8-yl)[4-(trifluoromethyl)-1,3-thiazol-2-yl]methanone, (3-{[2-(4-chlorophenyl)imidazo[1,2-a]pyrimidin-3-yl]methyl}-3,8-diazabicyclo[3.2.1]oct-8-yl)(1,3-thiazol-4-yl)methanone, (3-{[2-(4-isopropylphenyl)imidazo[1,2-a]pyrimidin-3-yl]methyl}-3,8-diazabicyclo[3.2.1]oct-8-yl)[6-(methylamino)pyridin-2-yl]methanone, (3-{[2-(4-isopropylphenyl)imidazo[1,2-a]pyrimidin-3-yl]methyl}-3,8-diazabicyclo[3.2.1]oct-8-yl)(6-methoxypyridin-2-yl)methanone, (2-fluorophenyl)(3-{[2-(4-isopropylphenyl)imidazo[1,2-a]pyrimidin-3-yl]methyl}-3,8-diazabicyclo[3.2.1]oct-8-yl)methanone, (3-{1-[2-(4-chlorophenyl)imidazo[1,2-a]pyrimidin-3-yl]ethyl}-3,8-diazabicyclo[3.2.1]oct-8-yl)(3-fluoro-6-methoxypyridin-2-yl)methanone, (7-{[2-(4-isopropylphenyl)imidazo[1,2-a]pyrimidin-3-yl]methyl}-3-oxa-7,9-diazabicyclo[3.3.1]non-9-yl)(6-methoxypyridin-2-yl)methanone, (3-chloro-6-methoxypyridin-2-yl)(7-{[2-(4-isopropylphenyl)imidazo[1,2-a]pyrimidin-3-yl]methyl}-3-oxa-7,9-diazabicyclo[3.3.1]non-9-yl)methanone, (2-fluorophenyl)(7-{[2-(4-isopropylphenyl)imidazo[1,2-a]pyrimidin-3-yl]methyl}-3-oxa-7,9-diazabicyclo[3.3.1]non-9-yl)methanone, (3-chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (5-cyclopropyl-1,3-oxazol-4-yl)(5-{[2-(4-isopropylphenyl)imidazo[1,2-a]pyrimidin-3-yl]methyl}-2,5-diazabicyclo[2.2.2]oct-2-yl)methanone, (3-fluoro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-fluoro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (5-{[2-(4-isopropylphenyl)imidazo[1,2-a]pyrimidin-3-yl]methyl}-2,5-diazabicyclo[2.2.2]oct-2-yl)(6-methoxypyridin-2-yl)methanone, (5-{[2-(4-isopropylphenyl)imidazo[1,2-a]pyrimidin-3-yl]methyl}-2,5-diazabicyclo[2.2.2]oct-2-yl)(6-methoxypyridin-2-yl)methanone, [6-(difluoromethoxy)pyridin-2-yl](5-{[2-(4-isopropylphenyl)imidazo[1,2-a]pyrimidin-3-yl]methyl}-2,5-diazabicyclo[2.2.2]oct-2-yl)methanone, (5-{[2-(4-isopropylphenyl)imidazo[1,2-a]pyrimidin-3-yl]methyl}-2,5-diazabicyclo[2.2.2]oct-2-yl)(6-methoxy-3-methylpyridin-2-yl)methanone, (5-{[2-(4-bromophenyl)imidazo[1,2-a]pyrimidin-3-yl]methyl}-2,5-diazabicyclo[2.2.2]oct-2-yl)(2-fluorophenyl)methanone, (5-{[2-(4-bromophenyl)imidazo[1,2-a]pyrimidin-3-yl]methyl}-2,5-diazabicyclo[2.2.2]oct-2-yl)(cyclopentyl)methanone, (5-{[2-(4-bromophenyl)imidazo[1,2-a]pyrimidin-3-yl]methyl}-2,5-diazabicyclo[2.2.2]oct-2-yl)(3-fluoro-6-methoxypyridin-2-yl)methanone, (3-{[2-(4-bromophenyl)imidazo[1,2-a]pyrimidin-3-yl]methyl}-3,8-diazabicyclo[3.2.1]oct-8-yl)(3-fluoro-6-methoxypyridin-2-yl)methanone, (3-{[2-(4-bromophenyl)imidazo[1,2-a]pyrimidin-3-yl]methyl}-3,8-diazabicyclo[3.2.1]oct-8-yl)(2-fluorophenyl)methanone, (3-{[2-(4-bromophenyl)imidazo[1,2-a]pyrimidin-3-yl]methyl}-3,8-diazabicyclo[3.2.1]oct-8-yl)(cyclopentyl)methanone, 3-{[2-(4-Chlorophenyl)imidazo[1,2-a]pyrimidin-3-yl]methyl}-N-(2,4-difluorophenyl)-3,8-diazabicyclo[3.2.1]octane-8-carboxamide, 3-{[2-(4-chlorophenyl)imidazo[1,2-a]pyrimidin-3-yl]methyl}-N-isopropyl-3,8-diazabicyclo[3.2.1]octane-8-carboxamide, 3-{[2-(4-chlorophenyl)imidazo[1,2-a]pyrimidin-3-yl]methyl}-N-cyclopropyl-3,8-diazabicyclo[3.2.1]octane-8-carboxamide, 3-{[2-(4-chlorophenyl)imidazo[1,2-a]pyrimidin-3-yl]methyl}-N-(2,5-dichloro-4-methoxyphenyl)-3,8-diazabicyclo[3.2.1]octane-8-carboxamide, N-(3-chlorophenyl)-3-{[2-(4-chlorophenyl)imidazo[1,2-a]pyrimidin-3-yl]methyl}-3,8-diazabicyclo[3.2.1]octane-8-carboxamide, 3-{[2-(4-chlorophenyl)imidazo[1,2-a]pyrimidin-3-yl]methyl}-N-(2,6-difluorobenzyl)-3,8-diazabicyclo[3.2.1]octane-8-carboxamide, 3-{[2-(4-chlorophenyl)imidazo[1,2-a]pyrimidin-3-yl]methyl}-N-(2,6-dichlorophenyl)-3,8-diazabicyclo[3.2.1]octane-8-carboxamide, 3-{[2-(4-chlorophenyl)imidazo[1,2-a]pyrimidin-3-yl]methyl}-N-(2,6-dimethylphenyl)-3,8-diazabicyclo[3.2.1]octane-8-carboxamide, 3-{[2-(4-chlorophenyl)imidazo[1,2-a]pyrimidin-3-yl]methyl}-N-(2-fluorophenyl)-3,8-diazabicyclo[3.2.1]octane-8-carboxamide, 3-{[2-(4-chlorophenyl)imidazo[1,2-a]pyrimidin-3-yl]methyl}-N-(2,3-dichlorophenyl)-3,8-diazabicyclo[3.2.1]octane-8-carboxamide, 3-{[2-(4-chlorophenyl)imidazo[1,2-a]pyrimidin-3-yl]methyl}-N-(2-ethylphenyl)-3,8-diazabicyclo[3.2.1]octane-8-carboxamide, N-(2-chlorophenyl)-3-{[2-(4-chlorophenyl)imidazo[1,2-a]pyrimidin-3-yl]methyl}-3,8-diazabicyclo[3.2.1]octane-8-carboxamide, 3-{[2-(4-chlorophenyl)imidazo[1,2-a]pyrimidin-3-yl]methyl}-N-[2-chloro-5-(trifluoromethyl)phenyl]-3,8-diazabicyclo[3.2.1]octane-8-carboxamide, 3-{[2-(4-chlorophenyl)imidazo[1,2-a]pyrimidin-3-yl]methyl}-N-(2-ethyl-6-methylphenyl)-3,8-diazabicyclo[3.2.1]octane-8-carboxamide, 3-{[2-(4-chlorophenyl)imidazo[1,2-a]pyrimidin-3-yl]methyl}-N-(2,5-dimethylphenyl)-3,8-diazabicyclo[3.2.1]octane-8-carboxamide, 3-{[2-(4-Chlorophenyl)imidazo[1,2-a]pyrimidin-3-yl]methyl}-N-cyclohexyl-3,8-diazabicyclo[3.2.1]octane-8-carboxamide, 3-{[2-(4-Chlorophenyl)imidazo[1,2-a]pyrimidin-3-yl]methyl}-N-isobutyl-3,8-diazabicyclo[3.2.1]octane-8-carboxamide, 3-{[2-(4-chlorophenyl)imidazo[1,2-a]pyrimidin-3-yl]methyl}-N-(3,4-dimethoxyphenyl)-3,8-diazabicyclo[3.2.1]octane-8-carboxamide, 3-{[2-(4-chlorophenyl)imidazo[1,2-a]pyrimidin-3-yl]methyl}-N-{4-[(trifluoromethyl)sulfanyl]phenyl}-3,8-diazabicyclo[3.2.1]octane-8-carboxamide, 3-{[2-(4-chlorophenyl)imidazo[1,2-a]pyrimidin-3-yl]methyl}-N-(3-fluorophenyl)-3,8-diazabicyclo[3.2.1]octane-8-carboxamide, 3-{[2-(4-chlorophenyl)imidazo[1,2-a]pyrimidin-3-yl]methyl}-N-(2,6-difluorophenyl)-3,8-diazabicyclo[3.2.1]octane-8-carboxamide, 3-{[2-(4-chlorophenyl)imidazo[1,2-a]pyrimidin-3-yl]methyl}-N-[4-chloro-2-(trifluoromethyl)phenyl]-3,8-diazabicyclo[3.2.1]octane-8-carboxamide, 3-{[2-(4-chlorophenyl)imidazo[1,2-a]pyrimidin-3-yl]methyl}-N-(2-methylbenzyl)-3,8-diazabicyclo[3.2.1]octane-8-carboxamide, 3-{[2-(4-chlorophenyl)imidazo[1,2-a]pyrimidin-3-yl]methyl}-N-methyl-N-phenyl-3,8-diazabicyclo[3.2.1]octane-8-carboxamide, 3-{[2-(4-chlorophenyl)imidazo[1,2-a]pyrimidin-3-yl]methyl}-N,N-diethyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(morpholin-4-yl)methanone, 3-{[2-(4-chlorophenyl)imidazo[1,2-a]pyrimidin-3-yl]methyl}-N,N-diisopropyl-3,8-diazabicyclo[3.2.1]octane-8-carboxamide, 3-{[2-(4-chlorophenyl)imidazo[1,2-a]pyrimidin-3-yl]methyl}-N-cyclohexyl-N-ethyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(pyrrolidin-1-yl)methanone, 3-{[2-(4-chlorophenyl)imidazo[1,2-a]pyrimidin-3-yl]methyl}-N-ethyl-N-phenyl-3,8-diazabicyclo[3.2.1]octane-8-carboxamide, 3-{[2-(4-chlorophenyl)imidazo[1,2-a]pyrimidin-3-yl]methyl}-N-isopropyl-N-methyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(piperidin-1-yl)methanone, 3-{[2-(4-chlorophenyl)imidazo[1,2-a]pyrimidin-3-yl]methyl}-N-ethyl-N-(4-methylphenyl)-3,8-diazabicyclo[3.2.1]octane-8-carboxamide, N-(4-chlorophenyl)-3-{[2-(4-chlorophenyl)imidazo[1,2-a]pyrimidin-3-yl]methyl}-N-isopropyl-3,8-diazabicyclo[3.2.1]octane-8-carboxamide, 3-{[2-(4-chlorophenyl)imidazo[1,2-a]pyrimidin-3-yl]methyl}-N,N-dimethyl-3,8-diazabicyclo[3.2.1]octane-8-carboxamide, 3-{[2-(4-chlorophenyl)imidazo[1,2-a]pyrimidin-3-yl]methyl}-N-(4-ethoxyphenyl)-N-methyl-3,8-diazabicyclo[3.2.1]octane-8-carboxamide, 3-{[2-(4-chlorophenyl)imidazo[1,2-a]pyrimidin-3-yl]methyl}-N-(3-methoxybenzyl)-N-methyl-3,8-diazabicyclo[3.2.1]octane-8-carboxamide, (3-{[2-(4-Chlorophenyl)imidazo[1,2-a]pyrimidin-3-yl]methyl}-3,8-diazabicyclo[3.2.1]oct-8-yl)(thiomorpholin-4-yl)methanone, methyl 3-{[2-(4-chlorophenyl)imidazo[1,2-a]pyrimidin-3-yl]methyl}-3,8-diazabicyclo[3.2.1]octane-8-carboxylate, ethyl 3-{[2-(4-chlorophenyl)imidazo[1,2-a]pyrimidin-3-yl]methyl}-3,8-diazabicyclo[3.2.1]octane-8-carboxylate, cyclopentyl 3-{[2-(4-chlorophenyl)imidazo[1,2-a]pyrimidin-3-yl]methyl}-3,8-diazabicyclo[3.2.1]octane-8-carboxylate, propyl 3-{[2-(4-chlorophenyl)imidazo[1,2-a]pyrimidin-3-yl]methyl}-3,8-diazabicyclo[3.2.1]octane-8-carboxylate, cyclohexylmethyl 3-{[2-(4-chlorophenyl)imidazo[1,2-a]pyrimidin-3-yl]methyl}-3,8-diazabicyclo[3.2.1]octane-8-carboxylate, cyclohexyl 3-{[2-(4-chlorophenyl)imidazo[1,2-a]pyrimidin-3-yl]methyl}-3,8-diazabicyclo[3.2.1]octane-8-carboxylate, 2,2-dimethylpropyl 3-{[2-(4-chlorophenyl)imidazo[1,2-a]pyrimidin-3-yl]methyl}-3,8-diazabicyclo[3.2.1]octane-8-carboxylate, tert-Butyl 3-{[2-(4-isopropylphenyl)imidazo[1,2-a]pyrimidin-3-yl]methyl}-3,8-diazabicyclo[3.2.1]octane-8-carboxylate, (5-Cyclopropyl-1,3-oxazol-4-yl)(3-{[2-(4-isopropylphenyl)imidazo[1,2-a]pyrimidin-3-yl]methyl}-3,8-diazabicyclo[3.2.1]oct-8-yl)methanone, tert-Butyl 3-{[2-(4-cyclopropylphenyl)imidazo[1,2-a]pyrimidin-3-yl]methyl}-3,8-diazabicyclo[3.2.1]octane-8-carboxylate, (3-{[2-(4-cyclopropylphenyl)imidazo[1,2-a]pyrimidin-3-yl]methyl}-3,8-diazabicyclo[3.2.1]octan-8-yl)(2-fluorophenyl)methanone, cyclopentyl(3-{[2-(4-cyclopropylphenyl)imidazo[1,2-a]pyrimidin-3-yl]methyl}-3,8-diazabicyclo[3.2.1]octan-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, (3-{[2-(4-cyclopropylphenyl)imidazo[1,2-a]pyrimidin-3-yl]methyl}-3,8-diazabicyclo[3.2.1]octan-8-yl)(6-methoxypyridin-2-yl)methanone, (3-{[2-(4-cyclopropylphenyl)imidazo[1,2-a]pyrimidin-3-yl]methyl}-3,8-diazabicyclo[3.2.1]octan-8-yl)[6-(difluoromethoxy)pyridin-2-yl]methanone, (5-cyclopropyl-1,3-oxazol-4-yl)(3-{[2-(4-cyclopropylphenyl)imidazo[1,2-a]pyrimidin-3-yl]methyl}-3,8-diazabicyclo[3.2.1]octan-8-yl)methanone, tert-Butyl 6-{[2-(4-chlorophenyl)imidazo[1,2-a]pyrimidin-3-yl]methyl}-2,6-diazabicyclo[3.2.2]nonane-2-carboxylate, tert-butyl 6-{[2-(5-chloropyridin-2-yl)imidazo[1,2-a]pyridin-3-yl]methyl}-2,6-diazabicyclo[3.2.2]nonane-2-carboxylate, tert-butyl 6-{[2-(4-chlorophenyl)imidazo[1,2-a]pyridin-3-yl]methyl}-2,6-diazabicyclo[3.2.2]nonane-2-carboxylate, (−)-tert-butyl 6-{[2-(4-isopropylphenyl)imidazo[1,2-a]pyrimidin-3-yl]methyl}-2,6-diazabicyclo[3.2.2]nonane-2-carboxylate, tert-Butyl 9-{[2-(4-isopropylphenyl)imidazo[1,2-a]pyrimidin-3-yl]methyl}-3,9-diazabicyclo[4.2.1]nonane-3-carboxylate, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [3-{[2-(5-Chloropyridin-2-yl)imidazo[1,2-a]pyridin-3-yl]methyl}-3,9-diazabicyclo[4.2.1]non-9-yl](3-fluoro-6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](3-fluoro-6-methoxypyridin-2-yl)methanone, [6-{[2-(5-chloropyridin-2-yl)imidazo[1,2-a]pyridin-3-yl]methyl}-2,6-diazabicyclo[3.2.2]non-2-yl](6-methoxypyridin-2-yl)methanone, [6-{[2-(5-Chloropyridin-2-yl)imidazo[1,2-a]pyridin-3-yl]methyl}-2,6-diazabicyclo[3.2.2]non-2-yl](3-fluoro-6-methoxypyridin-2-yl)methanone, (3-Chloro-6-methoxypyridin-2-yl)[6-{[2-(5-chloropyridin-2-yl)imidazo[1,2-a]pyridin-3-yl]methyl}-2,6-diazabicyclo[3.2.2]non-2-yl]methanone, [6-{[2-(4-Chlorophenyl)imidazo[1,2-a]pyrimidin-3-yl]methyl}-2,6-diazabicyclo[3.2.2]non-2-yl](6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](2-fluorophenyl)methanone, (3-chloro-6-methoxypyridin-2-yl)[6-{[2-(4-chlorophenyl)imidazo[1,2-a]pyrimidin-3-yl]methyl}-2,6-diazabicyclo[3.2.2]non-2-yl]methanone, (4-amino-1,2-oxadiazol-3-yl)[6-{[2-(4-chlorophenyl)imidazo[1,2-a]pyrimidin-3-yl]methyl}-2,6-diazabicyclo[3.2.2]non-2-yl]methanone, [6-{[2-(4-Chlorophenyl)imidazo[1,2-a]pyridin-3-yl]methyl}-2,6-diazabicyclo[3.2.2]non-2-yl](2-fluorophenyl)methanone, [6-{[2-(4-chlorophenyl)imidazo[1,2-a]pyridin-3-yl]methyl}-2,6-diazabicyclo[3.2.2]non-2-yl](6-methoxypyridin-2-yl)methanone, [6-{[2-(4-chlorophenyl)imidazo[1,2-a]pyridin-3-yl]methyl}-2,6-diazabicyclo[3.2.2]non-2-yl](3-fluoro-6-methoxypyridin-2-yl)methanone, (3-chloro-6-methoxypyridin-2-yl)[6-{[2-(4-chlorophenyl)imidazo[1,2-a]pyridin-3-yl]methyl}-2,6-diazabicyclo[3.2.2]non-2-yl]methanone, (4-amino-1,2,5-oxadiazol-3-yl)[6-{[2-(4-chlorophenyl)imidazo[1,2-a]pyridin-3-yl]methyl}-2,6-diazabicyclo[3.2.2]non-2-yl]methanone, [6-{[2-(5-Chloropyridin-2-yl)imidazo[1,2-a]pyridin-3-yl]methyl}-2,6-diazabicyclo[3.2.2]non-2-yl](2-fluorophenyl)methanone, [6-{[2-(4-Chlorophenyl)imidazo[1,2-a]pyrimidin-3-yl]methyl}-2,6-diazabicyclo[3.2.2]non-2-yl](2-fluorophenyl)methanone, [6-{[2-(4-Chlorophenyl)imidazo[1,2-a]pyrimidin-3-yl]methyl}-2,6-diazabicyclo[3.2.2]non-2-yl](6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](3-fluoro-6-methoxypyridin-2-yl)methanone, (3-chloro-6-methoxypyridin-2-yl)[6-{[2-(4-chlorophenyl)imidazo[1,2-a]pyrimidin-3-yl]methyl}-2,6-diazabicyclo[3.2.2]non-2-yl]methanone, (4-amino-1,2,5-oxadiazol-3-yl)[6-{[2-(4-chlorophenyl)imidazo[1,2-a]pyrimidin-3-yl]methyl}-2,6-diazabicyclo[3.2.2]non-2-yl]methanone, [6-{[2-(4-Chlorophenyl)imidazo[1,2-a]pyridin-3-yl]methyl}-2,6-diazabicyclo[3.2.2]non-2-yl](2-fluorophenyl)methanone, [6-{[2-(4-chlorophenyl)imidazo[1,2-a]pyridin-3-yl]methyl}-2,6-diazabicyclo[3.2.2]non-2-yl](6-methoxypyridin-2-yl)methanone, [6-{[2-(4-chlorophenyl)imidazo[1,2-a]pyridin-3-yl]methyl}-2,6-diazabicyclo[3.2.2]non-2-yl](3-fluoro-6-methoxypyridin-2-yl)methanone, (3-chloro-6-methoxypyridin-2-yl)[6-{[2-(4-chlorophenyl)imidazo[1,2-a]pyridin-3-yl]methyl}-2,6-diazabicyclo[3.2.2]non-2-yl]methanone, (4-amino-1,2,5-oxadiazol-3-yl)[6-{[2-(4-chlorophenyl)imidazo[1,2-a]pyridin-3-yl]methyl}-2,6-diazabicyclo[3.2.2]non-2-yl]methanone, [6-{[2-(4-isopropylphenyl)imidazo[1,2-a]pyrimidin-3-yl]methyl}-2,6-diazabicyclo[3.2.2]non-2-yl](6-methoxypyridin-2-yl)methanone, (3-fluoro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, (3-chloro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, cyclopentyl[6-{[2-(4-isopropylphenyl)imidazo[1,2-a]pyrimidin-3-yl]methyl}-2,6-diazabicyclo[3.2.2]non-2-yl]methanone, [6-(difluoromethoxy)pyridin-2-yl][6-{[2-(4-isopropylphenyl)imidazo[1,2-a]pyrimidin-3-yl]methyl}-2,6-diazabicyclo[3.2.2]non-2-yl]methanone, (2-fluorophenyl)[6-{[2-(4-isopropylphenyl)imidazo[1,2-a]pyrimidin-3-yl]methyl}-2,6-diazabicyclo[3.2.2]non-2-yl]methanone, (2-fluorophenyl)[6-{[2-(4-isopropylphenyl)imidazo[1,2-a]pyrimidin-3-yl]methyl}-2,6-diazabicyclo[3.2.2]non-2-yl]methanone, [6-{[2-(4-isopropylphenyl)imidazo[1,2-a]pyrimidin-3-yl]methyl}-2,6-diazabicyclo[3.2.2]non-2-yl](6-methoxypyridin-2-yl)methanone, (3-fluoro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, (3-chloro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, [6-(difluoromethoxy)pyridin-2-yl][6-{[2-(4-isopropylphenyl)imidazo[1,2-a]pyrimidin-3-yl]methyl}-2,6-diazabicyclo[3.2.2]non-2-yl]methanone, cyclopentyl[6-{[2-(4-isopropylphenyl)imidazo[1,2-a]pyrimidin-3-yl]methyl}-2,6-diazabicyclo[3.2.2]non-2-yl]methanone, (3-fluoro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, (3-chloro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, [9-{[2-(4-isopropylphenyl)imidazo[1,2-a]pyrimidin-3-yl]methyl}-3,9-diazabicyclo[4.2.1]non-3-yl](6-methoxypyridin-2-yl)methanone, [6-(difluoromethoxy)pyridin-2-yl][9-{[2-(4-isopropylphenyl)imidazo[1,2-a]pyrimidin-3-yl]methyl}-3,9-diazabicyclo[4.2.1]non-3-yl]methanone, cyclopentyl[9-{[2-(4-isopropylphenyl)imidazo[1,2-a]pyrimidin-3-yl]methyl}-3,9-diazabicyclo[4.2.1]non-3-yl]methanone, (3-fluoro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, (3-chloro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, [9-{[2-(4-isopropylphenyl)imidazo[1,2-a]pyrimidin-3-yl]methyl}-3,9-diazabicyclo[4.2.1]non-3-yl](6-methoxypyridin-2-yl)methanone, [6-(difluoromethoxy)pyridin-2-yl][9-{[2-(4-isopropylphenyl)imidazo[1,2-a]pyrimidin-3-yl]methyl}-3,9-diazabicyclo[4.2.1]non-3-yl]methanone, cyclopentyl[9-{[2-(4-isopropylphenyl)imidazo[1,2-a]pyrimidin-3-yl]methyl}-3,9-diazabicyclo[4.2.1]non-3-yl]methanone, (−)-(2-fluorophenyl)[9-{[2-(4-isopropylphenyl)imidazo[1,2-a]pyrimidin-3-yl]methyl}-3,9-diazabicyclo[4.2.1]non-3-yl]methanone, [6-{[2-(4-chlorophenyl)imidazo[1,2-a]pyrimidin-3-yl]methyl}-2,6-diazabicyclo[3.2.2]non-2-yl][6-(trifluoromethoxy)pyridin-2-yl]methanone, [6-{[2-(4-chlorophenyl)imidazo[1,2-a]pyrimidin-3-yl]methyl}-2,6-diazabicyclo[3.2.2]non-2-yl][6-(difluoromethoxy)pyridin-2-yl]methanone, [3-{[2-(4-chlorophenyl)imidazo[1,2-a]pyridin-3-yl]methyl}-3,9-diazabicyclo[4.2.1]non-9-yl](2-fluorophenyl)methanone, [3-{[2-(4-chlorophenyl)imidazo[1,2-a]pyridin-3-yl]methyl}-3,9-diazabicyclo[4.2.1]non-9-yl](6-methoxypyridin-2-yl)methanone, (3-{[2-(4-chlorophenyl)imidazo[1,2-a]pyridin-3-yl]methyl}-8-oxa-3,10-diazabicyclo[4.3.1]dec-10-yl](2-fluorophenyl)methanone, [3-{[2-(4-chlorophenyl)imidazo[1,2-a]pyridin-3-yl]methyl}-8-oxa-3,10-diazabicyclo[4.3.1]dec-10-yl](6-methoxypyridin-2-yl)methanone, [3-{[2-(4-chlorophenyl)imidazo[1,2-a]pyridin-3-yl]methyl}-8-oxa-3,10-diazabicyclo[4.3.1]dec-10-yl](4-methyl-1,2,5-oxadiazol-3-yl)methanone, [3-{[2-(4-chlorophenyl)imidazo[1,2-a]pyrimidin-3-yl]methyl}-8-oxa-3,10-diazabicyclo[4.3.1]dec-10-yl](2-fluorophenyl)methanone, [3-{[2-(4-chlorophenyl)imidazo[1,2-a]pyrimidin-3-yl]methyl}-8-oxa-3,10-diazabicyclo[4.3.1]dec-10-yl](6-methoxypyridin-2-yl)methanone, [3-{[2-(4-chlorophenyl)imidazo[1,2-a]pyrimidin-3-yl]methyl}-8-oxa-3,10-diazabicyclo[4.3.1]dec-10-yl](4-methyl-1,2,5-oxadiazol-3-yl)methanone, (4-amino-1,2,5-oxadiazol-3-yl)[3-{[2-(4-chlorophenyl)imidazo[1,2-a]pyrimidin-3-yl]methyl}-8-oxa-3,10-diazabicyclo[4.3.1]dec-10-yl]methanone, [3-{[2-(4-chlorophenyl)imidazo[1,2-a]pyridin-3-yl]methyl}-3,9-diazabicyclo[4.2.1]non-9-yl](3-fluoro-6-methoxypyridin-2-yl)methanone, [3-{[2-(4-chlorophenyl)imidazo[1,2-a]pyrimidin-3-yl]methyl}-3,9-diazabicyclo[4.2.1]non-9-yl](2-fluorophenyl)methanone, [3-{[2-(4-chlorophenyl)imidazo[1,2-a]pyrimidin-3-yl]methyl}-3,9-diazabicyclo[4.2.1]non-9-yl](3-fluoro-6-methoxypyridin-2-yl)methanone, [3-{[2-(5-chloropyridin-2-yl)imidazo[1,2-a]pyridin-3-yl]methyl}-3,9-diazabicyclo[4.2.1]non-9-yl](6-methoxypyridin-2-yl)methanone, [3-{[2-(4-chlorophenyl)imidazo[1,2-a]pyrimidin-3-yl]methyl}-3,9-diazabicyclo[4.2.1]non-9-yl](6-methoxypyridin-2-yl)methanone, [3-{[2-(4-Chlorophenyl)imidazo[1,2-a]pyridin-3-yl]methyl}-3,9-diazabicyclo[4.2.1]non-9-yl](2-fluorophenyl)methanone, [3-{[2-(4-Chlorophenyl)imidazo[1,2-a]pyridin-3-yl]methyl}-3,9-diazabicyclo[4.2.1]non-9-yl](6-methoxypyridin-2-yl)methanone, [3-{[2-(4-chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, [3-{[2-(4-chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [3-{[2-(4-chlorophenyl)imidazo[1,2-a]pyridin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyridin-3-yl]methyl}-3,6-diazabicyclo[3.2.2]non-6-yl](6-methoxypyridin-2-yl)methanone, [3-{[2-(5-chloropyridin-2-yl)imidazo[1,2-a]pyridin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyrimidin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyrimidin-3-yl]methyl}-3,6-diazabicyclo[3.2.2]non-6-yl](6-methoxypyridin-2-yl)methanone, (3-chloro-6-methoxypyridin-2-yl)(3-{[2-(4-chlorophenyl)imidazo[1,2-a]pyrimidin-3-yl]methyl}-3,9-diazabicyclo[4.2.1]non-9-yl)methanone, (3-chloro-6-methoxypyridin-2-yl)(3-{[2-(5-chloropyridin-2-yl)imidazo[1,2-a]pyridin-3-yl]methyl}-3,9-diazabicyclo[4.2.1]non-9-yl)methanone, (3-Chloro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone, [3-{[2-(4-Isopropylphenyl)imidazo[1,2-a]pyrimidin-3-yl]methyl}-3,9-diazabicyclo[4.2.1]nonan-9-yl](6-methoxypyridin-2-yl)methanone, (3-Chloro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

In a preferred embodiment of the present invention is directed to combinations of N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-[4-(3,4-dihydroisoquinolin-2(1H)-yl)piperidin-1-yl]-1,3-thiazole-5-carboxamide, 2-[3-(cyclopropylmethyl)[1,4′-bipiperidin]-1′-yl]-N-[(3,5-difluoropyridin-2-yl)methyl]-1,3-thiazole-5-carboxamide, 2-[3-(difluoromethyl)[1,4′-bipiperidin]-1′-yl]-N-[(3,5-difluoropyridin-2-yl)methyl]-1,3-thiazolo-5-carboxamide, N-[(3,5-difluoropyridin-2-yl)methyl]-2-[3-(trifluoromethyl)[1,4′-bipiperidin]-1′-yl]-1,3-thiazole-5-carboxamide, N-[(3,5-difluoropyridin-2-yl)methyl]-2-[3-(fluoromethyl)-[1,4′-bipiperidin]-1′-yl]-1,3-thiazole-5-carboxamide, 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-4-methyl-2-[(3R)-3-methyl[1,4′-bipiperidin]-1′-yl]-1,3-thiazole-5-carboxamide, N-[(3,5-difluoro-pyridin-2-yl)methyl]-4-methyl-2-[(3R)-3-methyl[1,4′-bipiperidin]-1′-yl]-1,3-thiazole-5-carboxamide, N-[(3,5-difluorpyridin-2-yl)methyl]-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-4-(trifluoromethyl)-1,3-thiazole-5-carboxamide, N-[(3,5-difluoropyridin-2-yl)methyl]-5-ethyl-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-1,3-oxazole-4-carboxamide, N-[(3,5-difluoropyridin-2-yl)methyl]-5-methyl-2-[(3R)-3-methyl[1,4′-bipiperidin]-1′-yl]-1,3-oxazole-4-carboxamide, N-[(3,5-difluoropyridin-2-yl)methyl]-2-[(3R)-3-methoxy[1,4′-bipiperidin]-1′-yl]-1,3-thiazole-5-carboxamide, 2-[3-(difluoromethoxy)[1,4′-bipiperidin]-1′-yl]-N-[(3,5-difluoropyridin-2-yl)methyl]-1,3-thiazoel-5-carboxamide, N-[(3,5-difluoropyridin-2-yl)methyl]-2-(3-ethyl[1,4′-bipiperidin]-1′-yl)-1,3-thiazole-5-carboxamide, 2-[(3R)-3-methyl[1,4′-bipiperidin]-1′-yl]-N-{[4-(trifluoromethyl)pyridin-2-yl]methyl}-1,3-thiazole-5-carboxamide, 2-[(3R)-3-methyl[1,4′-bipiperidin]-1′-yl]-N-[3-(trifluoromethyl)benzyl]-1,3-thiazole-5-carboxamide, N-[(3-fluoropyridin-2-yl)methyl]-2-[(3R)-3-methyl[1,4′-bipiperidin]-1′-yl]-1,3-thiazole-5-carboxamide, N-(5-chloro-2-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[4-(trifluoromethyl)benzyl]-1,3-thiazole-5-carboxamide, N-[(5-chloro-3-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(3-methylpyridin-2-yl)methyl]-1,3-thiazole-5-carboxamide, 2-[(3R)-3-methyl[1,4′-bipiperidin]-1′-yl]-N-[(4-methylpyridin-2-yl)methyl]-1,3-thiazole-5-carboxamide, N-[(3-chloropyridin-2-yl)methyl]-2-[(3R)-3-methyl[1,4′-bipiperidin]-1′-yl]-1,3-thiazole-5-carboxamide, N-[(3-fluoropyridin-2-yl)methyl]-N-methyl-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethyl)pyridin-2-yl]methyl}-1,3-thiazole-5-carboxamide, N-[(5-chloropyridin-2-yl)methyl]-2-[(3R)-3-methyl[1,4′-bipiperidin]-1′-yl]-1,3-thiazole-5-carboxamide, N-[1-(2,5-difluorophenyl)ethyl]-2-[(3R)-3-methyl[1,4′-bipiperidin]-1′-yl]-1,3-thiazole-5-carboxamide, N-[(3-chloro-5-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethoxy)pyridin-2-yl]methyl}-1,3-thiazole-5-carboxamide, N-(4-chlorobenzyl)-2-[(3R)-3-methyl[1,4′-bipiperidin]-1′-yl]-1,3-thiazole-5-carboxamide, N-(2-chloro-5-fluorobenzyl)-2-[(3R)-3-methyl[1,4′-bipiperidin]-1′-yl]-1,3-thiazole-5-carboxamide, N-(4-methylbenzyl)-2-[(3R)-3-methyl[1,4′-bipiperidin]-1′-yl]-1,3-thiazole-5-carboxamide, N-(3-methylbenzyl)-2-[(3R)-3-methyl[1,4′-bipiperidin]-1′-yl]-1,3-thiazole-5-carboxamide, N-(2-methylbenzyl)-2-[(3R)-3-methyl[1,4′-bipiperidin]-1′-yl]-1,3-thiazole-5-carboxamide, 2-[(3S)-(difluoromethyl)[1,4′-bipiperidin]-1′-yl]-N-[(3,5-difluoropyridin-2-yl)methyl]-1,3-thiazole-5-carboxamide, 2-[(3R)-3-(difluoromethyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[(3S)-3-(fluoromethyl)[1,4′-bipiperidin]-1′-yl]-1,3-thiazole-5-carboxamide, N-[(3,5-difluoropyridin-2-yl)methyl]-2-[(3R)-3-(fluoromethyl)[1,4′-bipiperidin]-1′-yl]-1,3-thiazole-5-carboxamide, N-[(3,5-difluoropyridin-2-yl)methyl]-2-[(3S)-3-(trifluoromethyl)[1,4′-bipiperidin]-1′-yl]-1,3-thiazole-5-carboxamide, N-[(3,5-difluoropyridin-2-yl)methyl]-2-[(3R)-3-(trifluoromethyl)[1,4′-bipiperidin]-1′-yl]-1,3-thiazole-5-carboxamide, 2-{(3S)-3-[(3,3-difluorocyclobutyl)methoxy][1,4′-bipiperidin]-1′-yl}-N-[(3,5-difluoropyridin-2-yl)methyl]-1,3-thiazole-5-carboxamide, 2-{(3R)-3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(1S)-1-(2,5-difluorophenyl)ethyl]-2-[(3R)-3-methyl[1,4′-bipiperidin]-1′-yl]-1,3-thiazole-5-carboxamide, N-[(1R)-1-(2,5-difluorophenyl)ethyl]-2-[(3R)-3-methyl[1,4′-bipiperidin]-1′-yl]-1,3-thiazole-5-carboxamide, N-[(3,5-difluoropyridin-2-yl)methyl]-2-[3-(methoxymethyl)[1,4′-bipiperidin]-1′-yl]-1,3-thiazole-5-carboxamide, N-[(3,5-difluoropyridin-2-yl)methyl]-3-[(3R)-3-methyl[1,4′-bipiperidin]-1′-yl]-1,2,4-oxadiazole-5-carboxamide, N-[(3,5-difluoropyridin-2-yl)methyl]-2-[(3R)-3′-fluor-3-methyl[1,4′-bipiperidin]-1′-yl]-1,3-thiazole-5-carboxamide, ent-N-[(3,5-difluoropyridin-2-yl)methyl]-2-[(3R), (3′R)-3′-fluoro-3-methyl[1,4′-bipiperidin]-1′-yl]-1,3-thiazole-5-carboxamide, ent-N-[(3,5-difluoropyridin-2-yl)methyl]-2-[(3R), (3'S)-3′-fluoro-3-methyl[1,4′-bipiperidin]-1′-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[(5-fluoro-2-thienyl)methyl]-2-[(3R)-3-methyl[1,4′-bipiperidin]-1′-yl]-1,3-thiazole-5-carboxamide, 2-[(3R)-3-methyl[1,4′-bipiperidin]-1-yl]-N-(pyridin-4-ylmethyl)-1,3-thiazole-5-carboxamide, N-[(3,5-difluoropyridin-2-yl)methyl]-2-{3-[(2,2,2-trifluoroethoxy)methyl][1,4′-bipiperidin]-1′-yl}-1,3-thiazole-5-carboxamide, N-[(3,5-difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide, 2-[3-({[1-(difluoromethyl)cyclopropyl]methoxy}methyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[3-({[1-(trifluoromethyl)cyclopropyl]methoxy}methyl)[1,4′-bipiperidin]-1′-yl]-1,3-thiazoel-5-carboxamide, N-[(3,5-difluoropyridin-2-yl)methyl]-2-(3,3-dimethyl[1,4′-bipiperidin]-1′-yl)-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, 2-[4-(1,1-difluoro-5-azaspiro[2.5]octan-5-yl)piperidin-1-yl]-N-[(3,5-difluoropyridin-2-yl)methyl]-1,3-thiazoel-5-carboxamide, 2-[3-(cyclobutylmethoxy)[1,4′-bipiperidin]-1′-yl]-N-[(3,5-difluoropyridin-2-yl)methyl]-1,3-thiazole-5-carboxamide, 2-[3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclobutyloxy)methyl]-[1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[3-ethoxy[1,4′-bipiperidin]-1′-yl]-1,3-thiazole-5-carboxamide, N-[(3,5-difluoropyridin-2-yl)methyl]-2-{4-[(3R)-3-methylpiperidin-1-yl]azepan-1-yl}-1,3-thiazole-5-carboxamide, 2-[(3R)-3-methyl[1,4′-bipiperidin]-1′-yl]-N-[(6-methylpyridin-3-yl)methyl]-1,3-thiazole-5-carboxamide, N-benzyl-2-[(3R)-3-methyl[1,4′-bipiperidin]-1′-yl]-1,3-thiazole-5-carboxamide, N-[(3,5-difluoropyridin-2-yl)methyl]-2-[3-({[3-fluorobutyl]oxy}methyl)[1,4′-bipiperidin]-1′-yl]-1,3-thiazole-5-carboxamide, 2-(3-{[(3,3-difluorocyclobutyl)methoxy]methyl}[1,4′-bipiperidin]-1′-yl)-N-[(3,5-difluoropyridin-2-yl)methyl]-1,3-thiazole-5-carboxamide, N-[(3-fluoropyridin-4-yl)methyl]-2-[(3R)-3-methyl[1,4′-bipiperidin]-1′-yl]-1,3-thiazole-5-carboxamide, N-[(3,5-difluoropyridin-2-yl)methyl]-2-[3-(2,2,2-trifluoroethoxy)[1,4′-bipiperidin]-1′-yl]-1,3-thiazole-5-carboxamide, N-[(4,6-dimethylpyridin-3-yl)methyl]-2-[(3R)-3-methyl[1,4′-bipiperidin]-1′-yl]-1,3-thiazole-5-carboxamide, N-[(4-chloro-1-methyl-1H-pyrazol-5-yl)methyl]-2-[(3R)-3-methyl[1,4′-bipiperidin]-1′-yl]-1,3-thiazole-5-carboxamide, N-(3-methoxybenzyl)-2-[(3R)-3-methyl[1,4′-bipiperidin]-1′-yl]-1,3-thiazole-5-carboxamide, N-(2,5-difluorobenzyl)-2-[(3R)-3-methyl[1,4′-bipiperidin]-1′-yl]-1,3-thiazole-5-carboxamide, N-(3-hydroxybenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(2R)-2-phenylpropyl]-1,3-thiazole-5-carboxamide, N-(4-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-3-ylmethyl)-1,3-thiazole-5-carboxamide, N-(3-fluorobenzyl)-2-[(3R)-3-methyl[1,4′-bipiperidin]-1′-yl]-1,3-thiazole-5-carboxamide, N-(2-fluorobenzyl)-2-[(3R)-3-methyl[1,4′-bipiperidin]-1′-yl]-1,3-thiazole-5-carboxamide, N-(2-chloro-4-fluorophenyl)-2-[(3R)-3-methyl[1,4′-bipiperidin]-1′-yl]-1,3-thiazole-5-carboxamide, N-(3-cyano-4-fluorophenyl)-2-[(3R)-3-methyl[1,4′-bipiperidin]-1′-yl]-1,3-thiazole-5-carboxamide, N-methyl-2-[(3R)-3-methyl[1,4′-bipiperidin]-1′-yl]-N-(pyridin-3-ylmethyl)-1,3-thiazole-5-carboxamide, N-methyl-2-[(3R)-3-methyl[1,4′-bipiperidin]-1′-yl]-N-(pyridin-4-ylmethyl)-1,3-thiazole-5-carboxamide, N-benzyl-N-methyl-2-[(3R)-3-methyl[1,4′-bipiperidin]-1′-yl]-1,3-thiazole-5-carboxamide, N-(2-cyclopropylphenyl)-2-[(3R)-3-methyl[1,4′-bipiperidin]-1′-yl]-1,3-thiazole-5-carboxamide, N-(3-chlorobenzyl)-2-[(3R)-3-methyl[1,4′-bipiperidin]-1′-yl]-1,3-thiazol-5-carboxamide, 2-[(3R)-3-methyl[1,4′-bipiperidin]-1′-yl]-N-[(1R)-1-(4-methylphenyl)ethyl]-1,3-thiazole-5-carboxamide, N-(2-ethylpyridin-4-yl)-2-[(3R)-3-methyl[1,4′-bipiperidin]-1′-yl]-1,3-thiazole-5-carboxamide, N-[(3,5-difluoropyridin-2-yl)methyl]-2-[(3S)-3-(methoxymethyl)[1,4′-bipiperidin]-1′-yl]-1,3-thiazole-5-carboxamide, N-[(3,5-difluoropyridin-2-yl)methyl]-2-[(3R)-3-(methoxymethyl)[1,4′-bipiperidin]-1′-yl]-1,3-thiazole-5-carboxamide, 2-{(3S)-3-[(cyclobutyloxy)methyl][1,4′-bipiperidin]-1′-yl}-N-[(3,5-difluorpyridin-2-yl)methyl]-1,3-thiazole-5-carboxamide, 2-{(3R)-3-[(cyclobutyloxy)methyl][1,4′-bipiperidin]-1′-yl}-N-[(3,5-difluorpyridin-2-yl)methyl]-1,3-thiazole-5-carboxamide, N-[(3,5-difluoropyridin-2-yl)methyl]-2-(3-isopropyl[1,4′-bipiperidin]-1′-yl)-1,3-thiazole-5-carboxamide, N-[(3,5-difluoropyridin-2-yl)methyl]-2-[4-((4S)-4-methylazepan-1-yl)piperidin-1-yl]-1,3-thiazole-5-carboxamide, N-[(3,5-difluoropyridin-2-yl)methyl]-2-[4-((4R)-4-methylazepan-1-yl)piperidin-1-yl]-1,3-thiazole-5-carboxamide, N-[(3,5-difluoropyridin-2-yl)methyl]-2-{(3S)-3-[(2,2,2-trifluoroethoxy)methyl][1,4′-bipiperidin]-1′-yl}-1,3-thiazole-5-carboxamide, N-[(3,5-difluoropyridin-2-yl)methyl]-2-{(3R)-3-[(2,2,2-trifluoroethoxy)methyl][1,4′-bipiperidin]-1′-yl}-1,3-thiazole-5-carboxamide, 2-{3-[(2,2-difluorocyclopropyl)methoxy][1,4′-bipiperidin]-1′-yl}-N-[(3,5-difluoropyridin-2-yl)methyl]-1,3-thiazole-5-carboxamide, 2-[3-(cyclobutyloxy)[1,4′-bipiperidin]-1′-yl]-N-[(3,5-difluoropyridin-2-yl)methyl]-1,3-thiazole-5-carboxamide, 2-{3-[(3,3-difluorocyclobutyl)oxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[(3R)-3′-fluoro-3-methyl[1,4′-bipiperidin]-1′-yl]-1,3-thiazole-4-carboxamide, N-[(3,5-difluoropyridin-2-yl)methyl]-2-[(3R)-3′-fluoro-3-methyl[1,4′-bipiperidin]-1′-yl]-1,3-oxazole-4-carboxamide, N-(5-chloro-2-fluorobenzyl)-2-[(3R)-3′-fluoro-3-methyl[1,4′-bipiperidin]-1′-yl]-1,3-thiazole-5-carboxamide, 2-[(3R)-3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[1-(2,5-difluorophenyl)ethyl]-2-[(3R)-3′-fluoro-3-methyl[1,4′-bipiperidin]-1′-yl]-1,3-thiazole-5-carboxamide, 4-(2-chlorophenyl)-N-[(3,5-difluoropyridin-2-yl)methyl]-2-[(3R)-3-methyl[1,4′-bipiperidin]-1′-yl]-1,3-thiazole-5-carboxamide, 4-bromo-N-[(3,5-difluoropyridin-2-yl)methyl]-2-[(3R)-3-methyl[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-(3-propyl[1,4′-bipiperidin]-1′-yl)-1,3-thiazole-5-carboxamide, 4-cyclopropyl-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-((3S)-3-ethoxy[1,4′-bipiperidin]-1′-yl)-1,3-thiazole-5-carboxamide, N-[(3,5-difluoropyridin-2-yl)methyl]-2-((3R)-3-ethoxy[1,4′-bipiperidin]-1′-yl)-1,3-thiazole-5-carboxamide, 2-[(3S)-3-(cyclobutylmethoxy)[1,4′-bipiperidin]-1′-yl]-N-[(3,5-difluoropyridin-2-yl)methyl]-1,3-thiazole-5-carboxamide, 2-[(3R)-3-(cyclobutylmethoxy)[1,4′-bipiperidin]-1′-yl]-N-[(3,5-difluoropyridin-2-yl)methyl]-1,3-thiazole-5-carboxamide, formic acid-N-[(3,5-difluoropyridin-2-yl)methyl]-2-[3-(2-fluoroethyl)[1,4′-bipiperidin]-1′-yl]-1,3-thiazole-5-carboxamide, 2-([1,4′-bipiperidin]-1′-yl)-N-[(3,5-difluoropyridin-2-yl)methyl]-1,3-thiazole-5-carboxamide, N-[1-(3,5-difluoropyridin-2-yl)cyclopropyl]-2-[(3R)-3-methyl[1,4′-bipiperidin]-1′-yl]-1,3-thiazole-5-carboxamide, N-[(3,5-difluoropyridin-2-yl)methyl]-4-ethyl-2-[(3R)-3-methyl[1,4′-bipiperidin]-1′-yl]-1,3-thiazole-5-carboxamide, 2-[4-(3S)-(1,1-difluoro-5-azaspiro[2.5]octan-5-yl)piperidin-1-yl]-N-[(3,5-difluoropyridin-2-yl)methyl]-1,3-thiazole-5-carboxamide, 2-[4-(3R)-(1,1-difluoro-5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-(3-phenyl[1,4′-bipiperidin]-1′-yl)-1,3-thiazole-5-carboxamide, 2-[4-(1,1-difluoro-5-azaspiro[2.5]octan-5-yl)-3-fluoropiperidin-1-yl]-N-[(3,5-difluoropyridin-2-yl)methyl]-1,3-thiazole-5-carboxamide, 2-[4-(5-azaspiro[2.5]octan-5-yl)-3-fluoropiperidin-1-yl]-N-[(3,5-difluoropyridin-2-yl)methyl]-1,3-thiazole-5-carboxamide
as compound of formula (I)
and compounds of formula (II) which are selected from the group consisting of:
tert-Butyl 7-{[2-(4-chlorophenyl)imidazo[1,2-a]pyrimidin-3-yl]methyl}-3-oxa-7,9-diazabicyclo[3.3.1]nonane-9-carboxylate, tert-Butyl 7-{[2-(4-isopropylphenyl)imidazo[1,2-a]pyrimidin-3-yl]methyl}-3-oxa-7,9-diazabicyclo[3.3.1]nonane-9-carboxylate, tert-Butyl 5-{[2-(4-chlorophenyl)imidazo[1,2-a]pyrimidin-3-yl]methyl}-2,5-diazabicyclo[2.2.2]octane-2-carboxylate, tert-Butyl 5-{[2-(4-chlorophenyl)imidazo[1,2-a]pyrimidin-3-yl]methyl}-2,5-diazabicyclo[2.2.2]octane-2-carboxylate, tert-Butyl 3-{[2-(4-chlorophenyl)imidazo[1,2-a]pyrimidin-3-yl]methyl}-3,8-diazabicyclo[3.2.1]octane-8-carboxylate, tert-Butyl 5-{[2-(4-isopropylphenyl)imidazo[1,2-a]pyrimidine-3-yl]methyl}-2,5-diazabicyclo[2.2.2]octane-2-carboxylate, tert-Butyl 5-{[2-(4-isopropylphenyl)imidazo[1,2-a]pyrimidin-3-yl]methyl}-2,5-diazabicyclo[2.2.2]octane-2-carboxylate, tert-Butyl 3-{1-[2-(4-chlorophenyl)imidazo[1,2-a]pyrimidin-3-yl]ethyl}-3,8-diazabicyclo[3.2.1]octane-8-carboxylate, tert-butyl 5-{[2-(4-bromophenyl)imidazo[1,2-a]pyrimidin-3-yl]methyl}-2,5-diazabicyclo[2.2.2]octane-2-carboxylate, tert-butyl 3-{[2-(4-bromophenyl)imidazo[1,2-a]pyrimidin-3-yl]methyl}-3,8-diazabicyclo[3.2.1]octane-8-carboxylate, (7-{[2-(4-Chlorophenyl)imidazo[1,2-a]pyrimidin-3-yl]methyl}-3-oxa-7,9-diazabicyclo[3.3.1]non-9-yl)(6-methoxypyridin-2-yl)methanone, (3-Chloro-6-methoxypyridin-2-yl)(7-{[2-(4-chlorophenyl)imidazo[1,2-a]pyrimidin-3-yl]methyl}-3-oxa-7,9-diazabicyclo[3.3.1]non-9-yl)methanone, (7-{[2-(4-Chlorophenyl)imidazo[1,2-a]pyrimidin-3-yl]methyl}-3-oxa-7,9-diazabicyclo[3.3.1]non-9-yl)(3-fluoro-6-methoxypyridin-2-yl)methanone, (7-{[2-(4-Chlorophenyl)imidazo[1,2-a]pyrimidin-3-yl]methyl}-3-oxa-7,9-diazabicyclo[3.3.1]non-9-yl)[6-(methylsulfanyl)pyridin-2-yl]methanone, (7-{[2-(4-Chlorophenyl)imidazo[1,2-a]pyrimidin-3-yl]methyl}-3-oxa-7,9-diazabicyclo[3.3.1]non-9-yl)(cyclopentyl)methanone, (3-Fluoro-6-methoxypyridin-2-yl)(7-{[2-(4-isopropylphenyl)imidazo[1,2-a]pyrimidin-3-yl]methyl}-3-oxa-7,9-diazabicyclo[3.3.1]non-9-yl)methanone, [6-(Difluoromethoxy)pyridin-2-yl](7-{[2-(4-isopropylphenyl)imidazo[1,2-a]pyrimidin-3-yl]methyl}-3-oxa-7,9-diazabicyclo[3.3.1]non-9-yl)methanone, (3-{[2-(4-Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone, (3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone, (3-Chloro-6-methoxypyridin-2-yl)(5-{[2-(4-chlorophenyl)imidazo[1,2-a]pyrimidin-3-yl]methyl}-2,5-diazabicyclo[2.2.2]oct-2-yl)methanone, (5-{[2-(4-Chlorophenyl)imidazo[1,2-a]pyrimidin-3-yl]methyl}-2,5-diazabicyclo[2.2.2]oct-2-yl)(3-fluoro-6-methoxypyridin-2-yl)methanone, (5-{[2-(4-Chlorophenyl)imidazo[1,2-a]pyrimidin-3-yl]methyl}-2,5-diazabicyclo[2.2.2]oct-2-yl)(6-methoxy-3-methylpyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (5-Cyclopropyl-1,3-oxazol-4-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (7-{[2-(4-chlorophenyl)imidazo[1,2-a]pyrimidin-3-yl]methyl}-3-oxa-7,9-diazabicyclo[3.3.1]non-9-yl)(2-fluorophenyl)methanone, (7-{[2-(4-chlorophenyl)imidazo[1,2-a]pyrimidin-3-yl]methyl}-3-oxa-7,9-diazabicyclo[3.3.1]non-9-yl)(3-methoxyphenyl)methanone, (3-{[2-(4-chlorophenyl)imidazo[1,2-a]pyrimidin-3-yl]methyl}-3,8-diazabicyclo[3.2.1]oct-8-yl)(2-fluorophenyl)methanone, (3-{[2-(4-chlorophenyl)imidazo[1,2-a]pyrimidin-3-yl]methyl}-3,8-diazabicyclo[3.2.1]oct-8-yl)[6-(methylsulfanyl)pyridin-2-yl]methanone, (3-{[2-(4-chlorophenyl)imidazo[1,2-a]pyrimidin-3-yl]methyl}-3,8-diazabicyclo[3.2.1]oct-8-yl)(cyclopentyl)methanone, (3-{[2-(4-chlorophenyl)imidazo[1,2-a]pyrimidin-3-yl]methyl}-3,8-diazabicyclo[3.2.1]oct-8-yl) [6-(methylamino)pyridin-2-yl]methanone, (3-{[2-(4-chlorophenyl)imidazo[1,2-a]pyrimidin-3-yl]methyl}-3,8-diazabicyclo[3.2.1]oct-8-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(cyclopentyl)methanone, (5-{[2-(4-chlorophenyl)imidazo[1,2-a]pyrimidin-3-yl]methyl}-2,5-diazabicyclo[2.2.2]oct-2-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(2-fluorophenyl)methanone, (5-{[2-(4-chlorophenyl)imidazo[1,2-a]pyrimidin-3-yl]methyl}-2,5-diazabicyclo[2.2.2]oct-2-yl)(6-methoxypyridin-2-yl)methanone, (5-{[2-(4-chlorophenyl)imidazo[1,2-a]pyrimidin-3-yl]methyl}-2,5-diazabicyclo[2.2.2]oct-2-yl)(3-fluoro-6-methoxypyridin-2-yl)methanone, (5-{[2-(4-chlorophenyl)imidazo[1,2-a]pyrimidin-3-yl]methyl}-2,5-diazabicyclo[2.2.2]oct-2-yl)(2-fluorophenyl)methanone, (5-{[2-(4-chlorophenyl)imidazo[1,2-a]pyrimidin-3-yl]methyl}-2,5-diazabicyclo[2.2.2]oct-2-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(6-methoxypyridin-2-yl)methanone, (3-chloro-6-methoxypyridin-2-yl)(5-{[2-(4-chlorophenyl)imidazo[1,2-a]pyrimidin-3-yl]methyl}-2,5-diazabicyclo[2.2.2]oct-2-yl)methanone, (7-{[2-(4-chlorophenyl)imidazo[1,2-a]pyrimidin-3-yl]methyl}-3-oxa-7,9-diazabicyclo[3.3.1]non-9-yl)(5-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(5-methyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(5-isopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2,4-dimethyl-1,3-oxazol-5-yl)methanone, (3-{[2-(4-chlorophenyl)imidazo[1,2-a]pyrimidin-3-yl]methyl}-3,8-diazabicyclo[3.2.1]oct-8-yl)(5-ethyl-1,3-oxazol-4-yl)methanone, (4-bromo-5-methyl-1,3-thiazol-2-yl)(3-{[2-(4-chlorphenyl)imidazo[1,2-a]pyrimidin-3-yl]methyl}-3,8-diazabicyclo[3.2.1]oct-8-yl)methanone, (3-{[2-(4-chlorophenyl)imidazo[1,2-a]pyrimidin-3-yl]methyl}-3,8-diazabicyclo[3.2.1]oct-8-yl)(5-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2-isopropyl-1,3-thiazol-4-yl)methanone, (3-{[2-(4-chlorophenyl)imidazo[1,2-a]pyrimidin-3-yl]methyl}-3,8-diazabicyclo[3.2.1]oct-8-yl)(1,3-thiazol-5-yl)methanone, (3-{[2-(4-chlorophenyl)imidazo[1,2-a]pyrimidin-3-yl]methyl}-3,8-diazabicyclo[3.2.1]oct-8-yl)(2,5-dimethyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)[2-methoxy-4-(trifluoromethyl)-1,3-thiazol-5-yl]methanone, (3-{[2-(4-chlorophenyl)imidazo[1,2-a]pyrimidin-3-yl]methyl}-3,8-diazabicyclo[3.2.1]oct-8-yl)[2-(trifluoromethyl)-1,3-thiazol-4-yl]methanone, (3-{[2-(4-chlorophenyl)imidazo[1,2-a]pyrimidin-3-yl]methyl}-3,8-diazabicyclo[3.2.1]oct-8-yl)(5-methyl-1,3-thiazol-4-yl)methanone, (3-{[2-(4-chlorophenyl)imidazo[1,2-a]pyrimidin-3-yl]methyl}-3,8-diazabicyclo[3.2.1]oct-8-yl)[4-(trifluoromethyl)-1,3-thiazol-2-yl]methanone, (3-{[2-(4-chlorophenyl)imidazo[1,2-a]pyrimidin-3-yl]methyl}-3,8-diazabicyclo[3.2.1]oct-8-yl)(1,3-thiazol-4-yl)methanone, (3-{[2-(4-isopropylphenyl)imidazo[1,2-a]pyrimidin-3-yl]methyl}-3,8-diazabicyclo[3.2.1]oct-8-yl)[6-(methylamino)pyridin-2-yl]methanone, (3-{[2-(4-isopropylphenyl)imidazo[1,2-a]pyrimidin-3-yl]methyl}-3,8-diazabicyclo[3.2.1]oct-8-yl)(6-methoxypyridin-2-yl)methanone, (2-fluorophenyl)(3-{[2-(4-isopropylphenyl)imidazo[1,2-a]pyrimidin-3-yl]methyl}-3,8-diazabicyclo[3.2.1]oct-8-yl)methanone, (3-{1-[2-(4-chlorophenyl)imidazo[1,2-a]pyrimidin-3-yl]ethyl}-3,8-diazabicyclo[3.2.1]oct-8-yl)(3-fluoro-6-methoxypyridin-2-yl)methanone, (7-{[2-(4-isopropylphenyl)imidazo[1,2-a]pyrimidin-3-yl]methyl}-3-oxa-7,9-diazabicyclo[3.3.1]non-9-yl)(6-methoxypyridin-2-yl)methanone, (3-chloro-6-methoxypyridin-2-yl)(7-{[2-(4-isopropylphenyl)imidazo[1,2-a]pyrimidin-3-yl]methyl}-3-oxa-7,9-diazabicyclo[3.3.1]non-9-yl)methanone, (2-fluorophenyl)(7-{[2-(4-isopropylphenyl)imidazo[1,2-a]pyrimidin-3-yl]methyl}-3-oxa-7,9-diazabicyclo[3.3.1]non-9-yl)methanone, (3-chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (5-cyclopropyl-1,3-oxazol-4-yl)(5-{[2-(4-isopropylphenyl)imidazo[1,2-a]pyrimidin-3-yl]methyl}-2,5-diazabicyclo[2.2.2]oct-2-yl)methanone, (3-fluoro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-fluoro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (5-{[2-(4-isopropylphenyl)imidazo[1,2-a]pyrimidin-3-yl]methyl}-2,5-diazabicyclo[2.2.2]oct-2-yl)(6-methoxypyridin-2-yl)methanone, (5-{[2-(4-isopropylphenyl)imidazo[1,2-a]pyrimidin-3-yl]methyl}-2,5-diazabicyclo[2.2.2]oct-2-yl)(6-methoxypyridin-2-yl)methanone, [6-(difluoromethoxy)pyridin-2-yl](5-{[2-(4-isopropylphenyl)imidazo[1,2-a]pyrimidin-3-yl]methyl}-2,5-diazabicyclo[2.2.2]oct-2-yl)methanone, (5-{[2-(4-isopropylphenyl)imidazo[1,2-a]pyrimidin-3-yl]methyl}-2,5-diazabicyclo[2.2.2]oct-2-yl)(6-methoxy-3-methylpyridin-2-yl)methanone, (5-{[2-(4-bromophenyl)imidazo[1,2-a]pyrimidin-3-yl]methyl}-2,5-diazabicyclo[2.2.2]oct-2-yl)(2-fluorophenyl)methanone, (5-{[2-(4-bromophenyl)imidazo[1,2-a]pyrimidin-3-yl]methyl}-2,5-diazabicyclo[2.2.2]oct-2-yl)(cyclopentyl)methanone, (5-{[2-(4-bromophenyl)imidazo[1,2-a]pyrimidin-3-yl]methyl}-2,5-diazabicyclo[2.2.2]oct-2-yl)(3-fluoro-6-methoxypyridin-2-yl)methanone, (3-{[2-(4-bromophenyl)imidazo[1,2-a]pyrimidin-3-yl]methyl}-3,8-diazabicyclo[3.2.1]oct-8-yl)(3-fluoro-6-methoxypyridin-2-yl)methanone, (3-{[2-(4-bromophenyl)imidazo[1,2-a]pyrimidin-3-yl]methyl}-3,8-diazabicyclo[3.2.1]oct-8-yl)(2-fluorophenyl)methanone, (3-{[2-(4-bromophenyl)imidazo[1,2-a]pyrimidin-3-yl]methyl}-3,8-diazabicyclo[3.2.1]oct-8-yl)(cyclopentyl)methanone, 3-{[2-(4-Chlorophenyl)imidazo[1,2-a]pyrimidin-3-yl]methyl}-N-(2,4-difluorophenyl)-3,8-diazabicyclo[3.2.1]octane-8-carboxamide, 3-{[2-(4-chlorophenyl)imidazo[1,2-a]pyrimidin-3-yl]methyl}-N-isopropyl-3,8-diazabicyclo[3.2.1]octane-8-carboxamide, 3-{[2-(4-chlorophenyl)imidazo[1,2-a]pyrimidin-3-yl]methyl}-N-cyclopropyl-3,8-diazabicyclo[3.2.1]octane-8-carboxamide, 3-{[2-(4-chlorophenyl)imidazo[1,2-a]pyrimidin-3-yl]methyl}-N-(2,5-dichloro-4-methoxyphenyl)-3,8-diazabicyclo[3.2.1]octane-8-carboxamide, N-(3-chlorophenyl)-3-{[2-(4-chlorophenyl)imidazo[1,2-a]pyrimidin-3-yl]methyl}-3,8-diazabicyclo[3.2.1]octane-8-carboxamide, 3-{[2-(4-chlorophenyl)imidazo[1,2-a]pyrimidin-3-yl]methyl}-N-(2,6-difluorobenzyl)-3,8-diazabicyclo[3.2.1]octane-8-carboxamide, 3-{[2-(4-chlorophenyl)imidazo[1,2-a]pyrimidin-3-yl]methyl}-N-(2,6-dichlorophenyl)-3,8-diazabicyclo[3.2.1]octane-8-carboxamide, 3-{[2-(4-chlorophenyl)imidazo[1,2-a]pyrimidin-3-yl]methyl}-N-(2,6-dimethylphenyl)-3,8-diazabicyclo[3.2.1]octane-8-carboxamide, 3-{[2-(4-chlorophenyl)imidazo[1,2-a]pyrimidin-3-yl]methyl}-N-(2-fluorophenyl)-3,8-diazabicyclo[3.2.1]octane-8-carboxamide, 3-{[2-(4-chlorophenyl)imidazo[1,2-a]pyrimidin-3-yl]methyl}-N-(2,3-dichlorophenyl)-3,8-diazabicyclo[3.2.1]octane-8-carboxamide, 3-{[2-(4-chlorophenyl)imidazo[1,2-a]pyrimidin-3-yl]methyl}-N-(2-ethylphenyl)-3,8-diazabicyclo[3.2.1]octane-8-carboxamide, N-(2-chlorophenyl)-3-{[2-(4-chlorophenyl)imidazo[1,2-a]pyrimidin-3-yl]methyl}-3,8-diazabicyclo[3.2.1]octane-8-carboxamide, 3-{[2-(4-chlorophenyl)imidazo[1,2-a]pyrimidin-3-yl]methyl}-N-[2-chloro-5-(trifluoromethyl)phenyl]-3,8-diazabicyclo[3.2.1]octane-8-carboxamide, 3-{[2-(4-chlorophenyl)imidazo[1,2-a]pyrimidin-3-yl]methyl}-N-(2-ethyl-6-methylphenyl)-3,8-diazabicyclo[3.2.1]octane-8-carboxamide, 3-{[2-(4-chlorophenyl)imidazo[1,2-a]pyrimidin-3-yl]methyl}-N-(2,5-dimethylphenyl)-3,8-diazabicyclo[3.2.1]octane-8-carboxamide, 3-{[2-(4-Chlorophenyl)imidazo[1,2-a]pyrimidin-3-yl]methyl}-N-cyclohexyl-3,8-diazabicyclo[3.2.1]octane-8-carboxamide, 3-{[2-(4-Chlorophenyl)imidazo[1,2-a]pyrimidin-3-yl]methyl}-N-isobutyl-3,8-diazabicyclo[3.2.1]octane-8-carboxamide, 3-{[2-(4-chlorophenyl)imidazo[1,2-a]pyrimidin-3-yl]methyl}-N-(3,4-dimethoxyphenyl)-3,8-diazabicyclo[3.2.1]octane-8-carboxamide, 3-{[2-(4-chlorophenyl)imidazo[1,2-a]pyrimidin-3-yl]methyl}-N-{4-[(trifluoromethyl)sulfanyl]phenyl}-3,8-diazabicyclo[3.2.1]octane-8-carboxamide, 3-{[2-(4-chlorophenyl)imidazo[1,2-a]pyrimidin-3-yl]methyl}-N-(3-fluorophenyl)-3,8-diazabicyclo[3.2.1]octane-8-carboxamide, 3-{[2-(4-chlorophenyl)imidazo[1,2-a]pyrimidin-3-yl]methyl}-N-(2,6-difluorophenyl)-3,8-diazabicyclo[3.2.1]octane-8-carboxamide, 3-{[2-(4-chlorophenyl)imidazo[1,2-a]pyrimidin-3-yl]methyl}-N-[4-chloro-2-(trifluoromethyl)phenyl]-3,8-diazabicyclo[3.2.1]octane-8-carboxamide, 3-{[2-(4-chlorophenyl)imidazo[1,2-a]pyrimidin-3-yl]methyl}-N-(2-methylbenzyl)-3,8-diazabicyclo[3.2.1]octane-8-carboxamide, 3-{[2-(4-chlorophenyl)imidazo[1,2-a]pyrimidin-3-yl]methyl}-N-methyl-N-phenyl-3,8-diazabicyclo[3.2.1]octane-8-carboxamide, 3-{[2-(4-chlorophenyl)imidazo[1,2-a]pyrimidin-3-yl]methyl}-N,N-diethyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(morpholin-4-yl)methanone, 3-{[2-(4-chlorophenyl)imidazo[1,2-a]pyrimidin-3-yl]methyl}-N,N-diisopropyl-3,8-diazabicyclo[3.2.1]octane-8-carboxamide, 3-{[2-(4-chlorophenyl)imidazo[1,2-a]pyrimidin-3-yl]methyl}-N-cyclohexyl-N-ethyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(pyrrolidin-1-yl)methanone, 3-{[2-(4-chlorophenyl)imidazo[1,2-a]pyrimidin-3-yl]methyl}-N-ethyl-N-phenyl-3,8-diazabicyclo[3.2.1]octane-8-carboxamide, 3-{[2-(4-chlorophenyl)imidazo[1,2-a]pyrimidin-3-yl]methyl}-N-isopropyl-N-methyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(piperidin-1-yl)methanone, 3-{[2-(4-chlorophenyl)imidazo[1,2-a]pyrimidin-3-yl]methyl}-N-ethyl-N-(4-methylphenyl)-3,8-diazabicyclo[3.2.1]octane-8-carboxamide, N-(4-chlorophenyl)-3-{[2-(4-chlorophenyl)imidazo[1,2-a]pyrimidin-3-yl]methyl}-N-isopropyl-3,8-diazabicyclo[3.2.1]octane-8-carboxamide, 3-{[2-(4-chlorophenyl)imidazo[1,2-a]pyrimidin-3-yl]methyl}-N,N-dimethyl-3,8-diazabicyclo[3.2.1]octane-8-carboxamide, 3-{[2-(4-chlorophenyl)imidazo[1,2-a]pyrimidin-3-yl]methyl}-N-(4-ethoxyphenyl)-N-methyl-3,8-diazabicyclo[3.2.1]octane-8-carboxamide, 3-{[2-(4-chlorophenyl)imidazo[1,2-a]pyrimidin-3-yl]methyl}-N-(3-methoxybenzyl)-N-methyl-3,8-diazabicyclo[3.2.1]octane-8-carboxamide, (3-{[2-(4-Chlorophenyl)imidazo[1,2-a]pyrimidin-3-yl]methyl}-3,8-diazabicyclo[3.2.1]oct-8-yl)(thiomorpholin-4-yl)methanone, methyl 3-{[2-(4-chlorophenyl)imidazo[1,2-a]pyrimidin-3-yl]methyl}-3,8-diazabicyclo[3.2.1]octane-8-carboxylate, ethyl 3-{[2-(4-chlorophenyl)imidazo[1,2-a]pyrimidin-3-yl]methyl}-3,8-diazabicyclo[3.2.1]octane-8-carboxylate, cyclopentyl 3-{[2-(4-chlorophenyl)imidazo[1,2-a]pyrimidin-3-yl]methyl}-3,8-diazabicyclo[3.2.1]octane-8-carboxylate, propyl 3-{[2-(4-chlorophenyl)imidazo[1,2-a]pyrimidin-3-yl]methyl}-3,8-diazabicyclo[3.2.1]octane-8-carboxylate, cyclohexylmethyl 3-{[2-(4-chlorophenyl)imidazo[1,2-a]pyrimidin-3-yl]methyl}-3,8-diazabicyclo[3.2.1]octane-8-carboxylate, cyclohexyl 3-{[2-(4-chlorophenyl)imidazo[1,2-a]pyrimidin-3-yl]methyl}-3,8-diazabicyclo[3.2.1]octane-8-carboxylate, 2,2-dimethylpropyl 3-{[2-(4-chlorophenyl)imidazo[1,2-a]pyrimidin-3-yl]methyl}-3,8-diazabicyclo[3.2.1]octane-8-carboxylate, tert-Butyl 3-{[2-(4-isopropylphenyl)imidazo[1,2-a]pyrimidin-3-yl]methyl}-3,8-diazabicyclo[3.2.1]octane-8-carboxylate, (5-Cyclopropyl-1,3-oxazol-4-yl)(3-{[2-(4-isopropylphenyl)imidazo[1,2-a]pyrimidin-3-yl]methyl}-3,8-diazabicyclo[3.2.1]oct-8-yl)methanone, tert-Butyl 3-{[2-(4-cyclopropylphenyl)imidazo[1,2-a]pyrimidin-3-yl]methyl}-3,8-diazabicyclo[3.2.1]octane-8-carboxylate, (3-{[2-(4-cyclopropylphenyl)imidazo[1,2-a]pyrimidin-3-yl]methyl}-3,8-diazabicyclo[3.2.1]octan-8-yl)(2-fluorophenyl)methanone, cyclopentyl(3-{[2-(4-cyclopropylphenyl)imidazo[1,2-a]pyrimidin-3-yl]methyl}-3,8-diazabicyclo[3.2.1]octan-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, (3-{[2-(4-cyclopropylphenyl)imidazo[1,2-a]pyrimidin-3-yl]methyl}-3,8-diazabicyclo[3.2.1]octan-8-yl)(6-methoxypyridin-2-yl)methanone, (3-{[2-(4-cyclopropylphenyl)imidazo[1,2-a]pyrimidin-3-yl]methyl}-3,8-diazabicyclo[3.2.1]octan-8-yl)[6-(difluoromethoxy)pyridin-2-yl]methanone, (5-cyclopropyl-1,3-oxazol-4-yl)(3-{[2-(4-cyclopropylphenyl)imidazo[1,2-a]pyrimidin-3-yl]methyl}-3,8-diazabicyclo[3.2.1]octan-8-yl)methanone, tert-Butyl 6-{[2-(4-chlorophenyl)imidazo[1,2-a]pyrimidin-3-yl]methyl}-2,6-diazabicyclo[3.2.2]nonane-2-carboxylate, (−)-tert-butyl 6-{[2-(4-isopropylphenyl)imidazo[1,2-a]pyrimidin-3-yl]methyl}-2,6-diazabicyclo[3.2.2]nonane-2-carboxylate, tert-Butyl 9-{[2-(4-isopropylphenyl)imidazo[1,2-a]pyrimidin-3-yl]methyl}-3,9-diazabicyclo[4.2.1]nonane-3-carboxylate, 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](3-fluoro-6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](2-fluorophenyl)methanone, (3-chloro-6-methoxypyridin-2-yl)[6-{[2-(4-chlorophenyl)imidazo[1,2-a]pyrimidin-3-yl]methyl}-2,6-diazabicyclo[3.2.2]non-2-yl]methanone, (4-amino-1,2-oxadiazol-3-yl)[6-{[2-(4-chlorophenyl)imidazo[1,2-a]pyrimidin-3-yl]methyl}-2,6-diazabicyclo[3.2.2]non-2-yl]methanone, [6-{[2-(4-Chlorophenyl)imidazo[1,2-a]pyrimidin-3-yl]methyl}-2,6-diazabicyclo[3.2.2]non-2-yl](2-fluorophenyl)methanone, [6-{[2-(4-Chlorophenyl)imidazo[1,2-a]pyrimidin-3-yl]methyl}-2,6-diazabicyclo[3.2.2]non-2-yl](6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](3-fluoro-6-methoxypyridin-2-yl)methanone, (3-chloro-6-methoxypyridin-2-yl)[6-{[2-(4-chlorophenyl)imidazo[1,2-a]pyrimidin-3-yl]methyl}-2,6-diazabicyclo[3.2.2]non-2-yl]methanone, (4-amino-1,2,5-oxadiazol-3-yl)[6-{[2-(4-chlorophenyl)imidazo[1,2-a]pyrimidin-3-yl]methyl}-2,6-diazabicyclo[3.2.2]non-2-yl]methanone, [6-{[2-(4-isopropylphenyl)imidazo[1,2-a]pyrimidin-3-yl]methyl}-2,6-diazabicyclo[3.2.2]non-2-yl](6-methoxypyridin-2-yl)methanone, (3-fluoro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, (3-chloro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, cyclopentyl[6-{[2-(4-isopropylphenyl)imidazo[1,2-a]pyrimidin-3-yl]methyl}-2,6-diazabicyclo[3.2.2]non-2-yl]methanone, [6-(difluoromethoxy)pyridin-2-yl][6-{[2-(4-isopropylphenyl)imidazo[1,2-a]pyrimidin-3-yl]methyl}-2,6-diazabicyclo[3.2.2]non-2-yl]methanone, (2-fluorophenyl)[6-{[2-(4-isopropylphenyl)imidazo[1,2-a]pyrimidin-3-yl]methyl}-2,6-diazabicyclo[3.2.2]non-2-yl]methanone, (2-fluorophenyl)[6-{[2-(4-isopropylphenyl)imidazo[1,2-a]pyrimidin-3-yl]methyl}-2,6-diazabicyclo[3.2.2]non-2-yl]methanone, [6-{[2-(4-isopropylphenyl)imidazo[1,2-a]pyrimidin-3-yl]methyl}-2,6-diazabicyclo[3.2.2]non-2-yl](6-methoxypyridin-2-yl)methanone, (3-fluoro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, (3-chloro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, [6-(difluoromethoxy)pyridin-2-yl][6-{[2-(4-isopropylphenyl)imidazo[1,2-a]pyrimidin-3-yl]methyl}-2,6-diazabicyclo[3.2.2]non-2-yl]methanone, cyclopentyl[6-{[2-(4-isopropylphenyl)imidazo[1,2-a]pyrimidin-3-yl]methyl}-2,6-diazabicyclo[3.2.2]non-2-yl]methanone, (3-fluoro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, (3-chloro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, [9-{[2-(4-isopropylphenyl)imidazo[1,2-a]pyrimidin-3-yl]methyl}-3,9-diazabicyclo[4.2.1]non-3-yl](6-methoxypyridin-2-yl)methanone, [6-(difluoromethoxy)pyridin-2-yl][9-{[2-(4-isopropylphenyl)imidazo[1,2-a]pyrimidin-3-yl]methyl}-3,9-diazabicyclo[4.2.1]non-3-yl]methanone, cyclopentyl[9-{[2-(4-isopropylphenyl)imidazo[1,2-a]pyrimidin-3-yl]methyl}-3,9-diazabicyclo[4.2.1]non-3-yl]methanone, (3-fluoro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, (3-chloro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone, [9-{[2-(4-isopropylphenyl)imidazo[1,2-a]pyrimidin-3-yl]methyl}-3,9-diazabicyclo[4.2.1]non-3-yl](6-methoxypyridin-2-yl)methanone, [6-(difluoromethoxy)pyridin-2-yl][9-{[2-(4-isopropylphenyl)imidazo[1,2-a]pyrimidin-3-yl]methyl}-3,9-diazabicyclo[4.2.1]non-3-yl]methanone, cyclopentyl[9-{[2-(4-isopropylphenyl)imidazo[1,2-a]pyrimidin-3-yl]methyl}-3,9-diazabicyclo[4.2.1]non-3-yl]methanone, (−)-(2-fluorophenyl)[9-{[2-(4-isopropylphenyl)imidazo[1,2-a]pyrimidin-3-yl]methyl}-3,9-diazabicyclo[4.2.1]non-3-yl]methanone, [6-{[2-(4-chlorophenyl)imidazo[1,2-a]pyrimidin-3-yl]methyl}-2,6-diazabicyclo[3.2.2]non-2-yl][6-(trifluoromethoxy)pyridin-2-yl]methanone, [6-{[2-(4-chlorophenyl)imidazo[1,2-a]pyrimidin-3-yl]methyl}-2,6-diazabicyclo[3.2.2]non-2-yl][6-(difluoromethoxy)pyridin-2-yl]methanone, [3-{[2-(4-chlorophenyl)imidazo[1,2-a]pyrimidin-3-yl]methyl}-8-oxa-3,10-diazabicyclo[4.3.1]dec-10-yl](2-fluorophenyl)methanone, [3-{[2-(4-chlorophenyl)imidazo[1,2-a]pyrimidin-3-yl]methyl}-8-oxa-3,10-diazabicyclo[4.3.1]dec-10-yl](6-methoxypyridin-2-yl)methanone, [3-{[2-(4-chlorophenyl)imidazo[1,2-a]pyrimidin-3-yl]methyl}-8-oxa-3,10-diazabicyclo[4.3.1]dec-10-yl](4-methyl-1,2,5-oxadiazol-3-yl)methanone, (4-amino-1,2,5-oxadiazol-3-yl)[3-{[2-(4-chlorophenyl)imidazo[1,2-a]pyrimidin-3-yl]methyl}-8-oxa-3,10-diazabicyclo[4.3.1]dec-10-yl]methanone, [3-{[2-(4-chlorophenyl)imidazo[1,2-a]pyrimidin-3-yl]methyl}-3,9-diazabicyclo[4.2.1]non-9-yl](2-fluorophenyl)methanone, [3-{[2-(4-chlorophenyl)imidazo[1,2-a]pyrimidin-3-yl]methyl}-3,9-diazabicyclo[4.2.1]non-9-yl](3-fluoro-6-methoxypyridin-2-yl)methanone, [3-{[2-(4-chlorophenyl)imidazo[1,2-a]pyrimidin-3-yl]methyl}-3,9-diazabicyclo[4.2.1]non-9-yl](6-methoxypyridin-2-yl)methanone, {[3-{[2-(4-chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [3-{[2-(4-chlorophenyl)imidazo[1,2-a]pyridin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyrimidin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyrimidin-3-yl]methyl}-3,6-diazabicyclo[3.2.2]non-6-yl](6-methoxypyridin-2-yl)methanone, (3-chloro-6-methoxypyridin-2-yl) (3-{[2-(4-chlorophenyl)imidazo[1,2-a]pyrimidin-3-yl]methyl}-3,9-diazabicyclo[4.2.1]non-9-yl)methanone, (3-Chloro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone, [3-{[2-(4-Isopropylphenyl)imidazo[1,2-a]pyrimidin-3-yl]methyl}-3,9-diazabicyclo[4.2.1]nonan-9-yl](6-methoxypyridin-2-yl)methanone, (3-Chloro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

In a more preferred embodiment of the present invention is directed to combinations the present invention is directed to combinations of compounds of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide
   and
   of compounds of formula (II) which are selected from the group consisting of:
(4-{[2-(4-Bromophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(6-methoxypyridin-2-yl)methanone, (4-{[2-(4-Bromophenyl)imidazo[1,2-a]pyridin-3-yl]methyl}piperazin-1-yl)(2-fluorophenyl)methanone, (4-{[2-(4-chlorophenyl)imidazo[1,2-a]pyridin-3-yl]methyl}piperazin-1-yl)(6-isopropoxypyridin-2-yl)methanone, (4-{[2-(4-bromophenyl)imidazo[1,2-a]pyridin-3-yl]methyl}piperazin-1-yl)(6-methoxypyridin-2-yl)methanone, (4-{[2-(4-Chlorophenyl)imidazo[1,2-a]pyridin-3-yl]methyl}piperazin-1-yl)[6-(trifluoromethoxy)pyridin-2-yl]methanone, (4-{[2-(4-Chlorophenyl)imidazo[1,2-a]pyridin-3-yl]methyl}piperazin-1-yl)(3-fluoro-6-methoxypyridin-2-yl)methanone, [5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Isopropylphenyl)imidazo[1,2-a]pyridin-3-yl]methyl}hexahydropyrrolo[3,4-c]pyrrol-2(1H)-yl](6-methoxypyridin-2-yl)methanone, (3-Fluoro-6-methoxypyridin-2-yl)[5-{[2-(4-isopropylphenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone, (−)-[(1S,4S)-5-{[2-(4-Chlorophenyl)imidazo[1,2-a]pyridin-3-yl]methyl}-2,5-diazabicyclo[2.2.2]oct-2-yl](6-methoxypyridin-2-yl)methanone, (−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone, (−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)[6-(difluoromethoxy)pyridin-2-yl]methanone, (3-{[2-(4-Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone, (3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [3-{[2-(5-Chloropyridin-2-yl)imidazo[1,2-a]pyridin-3-yl]methyl}-3,9-diazabicyclo[4.2.1]non-9-yl](3-fluoro-6-methoxypyridin-2-yl)methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

In a most preferred embodiment the present invention is directed to combinations of compounds of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide
   and
   of compounds of formula (II) which are selected from the group consisting of.
(3-{[2-(4-Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone, (3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

An another preferred embodiment the present invention is directed to combinations of compounds of of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide
   and
   of compounds of formula (II) which are selected from the group consisting of:
(4-{[2-(4-Chlorophenyl)imidazo[1,2-a]pyridin-3-yl]methyl}piperazin-1-yl)(6-methoxypyridin-2-yl)methanone, (5-{[2-(4-Chlorophenyl)imidazo[1,2-a]pyrimidin-3-yl]methyl}-2,5-diazabicyclo[2.2.2]oct-2-yl)(3-fluoro-6-methoxypyridin-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone and (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

An another preferred embodiment the present invention is directed to combinations of compounds of of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide
   and
   of compounds of formula (II) which are selected from the group consisting of:
(4-{[2-(4-Chlorophenyl)imidazo[1,2-a]pyridin-3-yl]methyl}piperazin-1-yl)(6-methoxypyridin-2-yl)methanone, and (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

An another preferred embodiment the present invention is directed to combinations of compounds of
N-[(3,5-difluoropyridin-2-yl)methyl]-2-[(3R)-3-methyl[1,4′-bipiperidin]-1′-yl]-1,3-thiazole-5-carboxamide
   and
(4-{[2-(4-Chlorophenyl)imidazo[1,2-a]pyridin-3-yl]methyl}piperazin-1-yl)(6-methoxypyridin-2-yl)methanone.

An another preferred embodiment the present invention is directed to combinations of compounds of
N-[(3,5-difluoropyridin-2-yl)methyl]-2-[(3R)-3-methyl[1,4′-bipiperidin]-1′-yl]-1,3-thiazole-5-carboxamide
   and
(3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

The terms employed herein have the meanings indicated below. The term “at least one” employed in the meanings below refers to one or several, such as one.

The term “hydroxy”, as employed herein as such or as part of another group, refers to a —OH group.

In the context of the invention, (C₁-C₆)-alkyl is a straight-chain or branched alkyl radical having 1 to 6 carbon atoms. Examples include: methyl, ethyl, n-propyl, isopropyl, n-butyl, isobutyl, sec-butyl, tert-butyl, n-pentyl, 2-pentyl, 3-pentyl, neopentyl, n-hexyl, 2-hexyl and 3-hexyl.

In the context of the invention, (C₁-C₄)-alkyl is a straight-chain or branched alkyl radical having 1 to 4 carbon atoms. Examples include: methyl, ethyl, n-propyl, isopropyl, n-butyl, isobutyl, sec-butyl and tert-butyl.

In the context of the invention, (C₁-C₃)-alkyl is a straight-chain or branched alkyl radical having 1 to 3 carbon atoms. Examples include: methyl, ethyl, n-propyl and isopropyl.

The term (C₁-C₆)alkoxy, as employed herein as such or as part of another group, refers to an (C₁-C₆)alkyl group, as defined herein, appended to the parent molecular moiety through an oxygen atom. Representative examples of (C₁-C₆)alkoxy include, but are not limited to, methoxy, ethoxy, n-propoxy, n-butoxy, iso-butoxy, sec-butoxy, tert-butoxy, 2,2-dimethylpropoxy, 3-methylbutoxy, and n-hexoxy.

The term “halo” or “halogen”, as employed herein as such or as part of another group, refers to fluorine, chlorine, bromine or iodine.

Mono-(C₁-C₃)-alkylamino in the context of the invention is an amino group having a straight-chain or branched alkyl substituent having 1 to 3 carbon atoms. Examples include: methylamino, ethylamino, n-propylamino and isopropylamino.

Di-(C₁-C₃)-alkylamino in the context of the invention is an amino group having two identical or different straight-chain or branched alkyl substituents each having 1 to 3 carbon atoms. Examples include: N,N-dimethylamino, N,N-diethylamino, N-ethyl-N-methylamino, N-methyl-N-n-propylamino, N-isopropyl-N-methylamino, N,N-di-n-propylamino, N-isopropyl-N-n-propylamino and N,N-diisopropylamino.

(C₁-C₃)-Alkylsulfanyl[also referred to as (C₁-C₃)-alkylthio] in the context of the invention is a straight-chain or branched alkyl radical having 1 to 3 carbon atoms which is attached to the remainder of the molecule via a sulfur atom. Examples include: methylsulfanyl, ethylsulfanyl, n-propylsulfanyl and isopropylsulfanyl.

(C₃-C₆)-Cycloalkyl in the context of the invention is a monocyclic saturated cycloalkyl group having 3 to 6 ring carbon atoms. Examples include: cyclopropyl, cyclobutyl, cyclopentyl and cyclohexyl.

(C₄-C₆)-Cycloalkyl in the context of the invention is a monocyclic saturated cycloalkyl group having 4 to 6 carbon atoms. Examples include: cyclobutyl, cyclopentyl and cyclohexyl.

The term hydroxy(C₁-C₆)alkyl, as employed herein as such or as part of another group, refers to at least one hydroxy group, as defined herein, appended to the parent molecular moiety through an (C₁-C₆)alkyl group, as defined herein. Representative examples of hydroxy(C₁-C₆)alkyl include, but are not limited to, hydroxymethyl, 1-hydroxyethyl, 2-hydroxyethyl, 2,2-dihydroxyethyl, 1-hydroxypropyl, 3-hydroxypropyl, 1-hydroxy-1-methylethyl, and 1-hydroxy-1-methylpropyl.

The term (C₁-C₆)alkoxy(C₁-C₆)alkyl, as employed herein as such or as part of another group, refers to at least one (C₁-C₆)alkoxy group, as defined herein, appended to the parent molecular moiety through an (C₁-C₆)alkyl group, as defined herein. When there are several (C₁-C₆)alkoxy groups, the (C₁-C₆)alkoxy groups can be identical or different.

Representative examples of (C₁-C₆)alkoxy(C₁-C₆)alkyl include, but are not limited to, methoxymethyl, ethoxymethyl, propoxymethyl, 2-methoxyethyl, 2-ethoxyethyl, 2,2-dimethoxyethyl, 1-methyl-2-propoxyethyl, 1-methoxy-1-methylethyl, and 4-methoxybutyl.

The term hydroxy(C₁-C₆)alkoxy, as employed herein as such or as part of another group, refers to at least one hydroxy group, as defined herein, appended to the parent molecular moiety through an (C₁-C₆)alkoxy group, as defined herein. Representative examples of hydroxy(C₁-C₆)alkoxy include, but are not limited to, hydroxymethoxy, dihydroxymethoxy, 2-hydroxyethoxy, 2-hydroxypropoxy, 3-hydroxypropoxy, 2-hydroxybutoxy, and 2-hydroxy-1-methylethoxy.

The term (C₁-C₆)alkoxy(C₁-C₆)alkoxy, as employed herein as such or as part of another group, refers to at least one (C₁-C₆)alkoxy group, as defined herein, appended to the parent molecular moiety through an (C₁-C₆)alkoxy group, as defined herein. The (C₁-C₆)alkoxy groups can be identical or different. Representative examples of (C₁-C₆)alkoxy(C₁-C₆)alkoxy include, but are not limited to, methoxymethoxy, propoxymethoxy, 2-methoxyethoxy, 2-ethoxyethoxy, 2-butoxyethoxy, 2,2-dimethoxyethoxy, 1-methyl-2-propoxyethoxy, 2-methoxypropoxy and 4-methoxybutoxy.

The term halo(C₁-C₆)alkoxy, as employed herein as such or as part of another group, refers to at least one halogen, as defined herein, appended to the parent molecular moiety through an (C₁-C₆)alkoxy group, as defined herein. When there are several halogens, the halogens can be identical or different. Representative examples of halo(C₁-C₆)alkoxy include, but are not limited to, fluoromethoxy, chloromethoxy, difluoromethoxy, trifluoromethoxy, 2-bromoethoxy, 2,2,2-trichloroethoxy, 3-bromopropoxy, 2-chloropropoxy, and 4-chlorobutoxy.

The expression “compounds of the invention” as employed herein refers to the compounds of formula I.

Pharmaceutically acceptable salts, e.g. acid addition salts, with both organic and inorganic acids, are known in the field of pharmaceuticals. Representative examples of pharmaceutically acceptable acid addition salts include, but are not limited to, chlorides, bromides, sulfates, nitrates, phosphates, sulfonates, methane sulfonates, formates, tartrates, maleates, citrates, benzoates, salicylates, ascorbates, acetates and oxalates.

Hydrates or solvates are designated according to the invention as those forms of the compounds of the formula (I) which in the solid or liquid state form a molecular compound or a complex by hydration with water or coordination with solvent molecules. Examples of hydrates are sesqui-hydrates, monohydrates, dihydrates or trihydrates. Equally, the hydrates or solvates of salts of the compounds according to the invention are also suitable.

Pharmaceutically acceptable esters, when applicable, may be prepared by known methods using pharmaceutically acceptable acids that are conventional in the field of pharmaceuticals and that retain the pharmacological properties of the free form. Nonlimiting examples of these esters include esters of aliphatic or aromatic alcohols. Representative examples of pharmaceutically acceptable esters include, but are not limited to, methyl, ethyl, n-propyl, iso-propyl, n-butyl, iso-butyl, sec-butyl, tert-butyl, and benzylesters.

The invention includes within its scope all the possible geometric isomers, e.g. Z and E isomers (cis and trans isomers), of the compounds as well as all the possible optical isomers, e.g. diastereomers and enantiomers, of the compounds. Furthermore, the invention includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof, conventional resolution methods, e.g. fractional crystallization, may be used.

The compounds of formula (II), their production and their action as selective blockers of TASK-1 and TASK-3 channels or the treatment of of respiratory disorders, sleep-related respiratory disorders, obstructive sleep apnoea, central sleep apnoea, snoring, cardiac arrhythmias, neurodegenerative disorders, neuroinflammatory disorders and neuroimmunological disorders are disclosed in WO 2017/097792 A1, WO 2017/097671 A1, WO 2018/015196 A1, WO 2018/228907 A1 and WO 2018/228909 A1 in general and especially the compounds specifically are an explicit part of the description of the present invention and are hereby incorporated by reference.

The term effective amount as used herein refers to an amount of a compound of formula (I) that is effective for treatment and/or prophylaxis of sleep-related breathing disorders, preferably obstructive and central sleep apneas and snoring.

The present invention relates to combinations of compounds of formula (I) and compounds formula (II) according to the invention for use in a method of treatment and/or prevention of respiratory disorders, sleep-related respiratory disorders, obstructive sleep apnoea, central sleep apnoea, snoring, cardiac arrhythmias, neurodegenerative disorders, neuroinflammatory disorders and neuroimmunological disorders.

The present invention relates also to the use of combinations of compounds of formula (I) and compounds of formula (II) according to the invention for production of a medicament for treatment and/or prevention of respiratory disorders, sleep-related respiratory disorders, obstructive sleep apnoea, central sleep apnoea, snoring, cardiac arrhythmias, neurodegenerative disorders, neuroinflammatory disorders and neuroimmunological disorders, preferably obstructive and central sleep apneas and snoring.

Moreover, the present invention relates to the use of one or more selective blockers of TASK-1 and TASK-3 channels in combination with one or more α2-Adrenoceptor subtype C (alpha-2C) antagonists for preparing a pharmaceutical composition for the treatment sleep-related breathing disorders.

A further subject of the present invention is the use of a combination of compounds of formula (I) and compounds of formula (II) according to the invention with one or more other active compounds in a method for the treatment and/or prophylaxis sleep-related breathing disorders, preferably obstructive and central sleep apneas and snoring.

A further subject of the present invention is a medicament comprising at least one a combination of compounds of formula (I) and compounds of formula (II) according to the invention in combination with one or more inert non-toxic pharmaceutically suitable excipients for use in a method for the treatment and/or prophylaxis sleep-related breathing disorders, preferably obstructive and central sleep apneas and snoring.

The present invention further relates to a medicament comprising at least one a combination of compounds of formula (I) and compounds of formula (II) according to the invention with one or more other active compounds in combination with one or more inert non-toxic pharmaceutically suitable excipients for use in a method for the treatment and/or prophylaxis sleep-related breathing disorders, preferably obstructive and central sleep apneas and snoring.

The present invention is also directed to a method for the treatment and/or prophylaxis of sleep-related breathing disorders, by administering systemically and/or locally a therapeutically effective amount of at least one combination of compounds of formula (I) and compounds of formula (II) or a medicament comprising at least one combination of compounds of formula (I) and compounds of formula (II) according to the invention in combination with a inert, non-toxic, pharmaceutically acceptable additive.

Combination of compounds of formula (I) and compounds of formula (II) according to the invention can be used alone or, if required, in combination with one or more other pharmacologically active substances, provided that this combination does not lead to undesirable and unacceptable side effects. Preferred examples of combination suitable for the purpose to treat sleep-related breathing disorders, preferably obstructive and central sleep apneas and snoring, include:
respiratory stimulants such as, by way of example and with preference, theophylline, doxapram, nikethamide or caffeine;
psychostimulants such as, by way of example and with preference, modafinil or armodafinil;
amphetamines and amphetamine derivatives such as, by way of example and with preference, amphetamine, metamphetamine or methylphenidate;
serotonin reuptake inhibitors such as, by way of example and with preference, fluoxetine, paroxetine, citalopram, escitalopram, sertraline, fluvoxamine or trazodone;
serotonin precursors such as, by way of example and with preference, L-tryptophan;
selective serotonin noradrenaline reuptake inhibitors such as, by way of example and with preference, venlafaxine or duloxetine;
noradrenergic and specific serotonergic antidepressants such as, by way of example and with preference, mirtazapine;
selective noradrenaline reuptake inhibitors such as, by way of example and with preference, reboxetine or atomoxetine;
tricyclic antidepressants such as, by way of example and with preference, amitriptyline, protriptyline, doxepine, trimipramine, imipramine, clomipramine or desipramine;
muscarinic receptor antagonists, by way of example and with preference oxybutynin;
GABA agonists such as, by way of example and with preference, baclofen;
glucocorticoids such as, by way of example and with preference, fluticasone, budesonide, beclometasone, mometasone, tixocortol or triamcinolone;
cannabinoid receptor agonists;
carboanhydrase inhibitors such as, by way of example and with preference, acetazolamide, methazolamide or diclofenamide;
opioid and benzodiazepine receptor antagonists such as, by way of example and with preference, flumazenil, naloxone or naltrexone;
cholinesterase inhibitors such as, by way of example and with preference, neostigmine, pyridostigmine, physostigmine donepezil, galantamine or rivastigmine;
appetite suppressants such as, by way of example and with preference, sibutramin, opiramate, phentermine, lipase inhibitors or cannabinoid receptor antagonists;
mineralocorticoid receptor antagonists.

Medicament comprising combinations as defined in any of Claims 1 to 5 in combination with one or more further active ingredients selected from the group consisting of muscarinic receptor antagonists, mineralocorticoid receptor antagonists, diuretics, corticosteroids.

A preferred subject of the present invention is a combination comprising combinations of compounds of formula (I) and compounds of formula (II) according to the invention and one or more other active compounds selected from the groups consisting of muscarinic receptor antagonists, mineralocorticoid receptor antagonists, diuretics, corticosteroids for use in a method for the treatment and/or prophylaxis sleep-related breathing disorders, preferably obstructive and central sleep apneas and snoring.

Another preferred subject of the present invention is a medicament comprising combinations of compounds of formula (I) and compounds of formula (II) according to the invention in combination with one or more other active compounds selected from the groups consisting of muscarinic receptor antagonists In a preferred embodiment of the invention, the combinations of the invention are administered in combination with a muscarinic receptor antagonist, by way of example and with preference oxybutynin.

In a preferred embodiment of the invention, the combinations of the invention are administered in combination with a mineralocorticoid receptor antagonist, by way of example and with preference spironolactone, eplerenone or finerenone.

In a preferred embodiment of the invention, the combinations of the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

In a preferred embodiment of the invention, the compounds of the invention are administered in combination with a corticosteroid, by way of example and with preference prednisone, prednisolone, methylprednisolone, triamcinolone, dexamethasone, betamethasone, beclomethasone, flunisolide, budesonide or fluticasone.

If required, aryl piperazines of formula (I) according to the invention can also be employed in conjunction with the use of one or more medical technical devices or auxiliaries, provided this does not lead to unwanted and unacceptable side-effects. Medical devices and auxiliaries suitable for such a combined application are, by way of example and with preference:
devices for positive airway pressure ventilation such as, by way of example and with preference, CPAP (continuous positive airway pressure) devices, BiPAP (bilevel positive airway pressure) devices and IPPV (intermittent positive pressure ventilation) devices;
neurostimulators of the Nervus hypoglossus;
intraoral auxiliaries such as, by way of example and with preference, protrusion braces;
nasal disposable valves;
nasal stents.

Substituted heterocyclic carboxamides of formula (I) and compounds of formula (II) according to the invention can act systemically and/or locally. For this purpose, they can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent.

A further subject of the present invention is a pharmaceutical composition comprising a combination of a compound of the formula (I) and a compound of formula (II) according to the invention for the systemic and/or local administration by the oral, parenteral, pulmonal, intrapulmonal (inhalative), nasal, intranasal, pharyngeal, lingual, sublingual, buccal, rectal, dermal, transdermal, conjunctival or otic route, or as an implant or stent. The preferred administrations are the oral, nasal and pharyngeal routes.

For these administration routes, the compounds according to the invention can be administered in suitable administration forms.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

Parenteral administration can be effected omitting an absorption step (e.g. intravenous, intra-arterial, intracardial, intraspinal or intralumbar administration) or involving absorption (e.g. intra-muscular, subcutaneous, intracutaneous, percutaneous or intraperitoneal administration). Suitable administration forms for parenteral administration include injection and infusion preparations in the form of solutions, suspensions, emulsions, lyophilisates or sterile powders.

For the other administration routes, for example inhalation formulations (including powder inhalers and nebulisers), nasal drops, solutions or sprays, pharyngeal sprays, tablets for lingual, sublingual or buccal administration, tablets, films/wafers or capsules, suppositories, oral or ophthalmic preparations, vaginal capsules, aqueous suspensions (lotions, shakable mixtures), lipophilic suspensions, ointments, creams, transdermal therapeutic systems (e.g. plasters), milk, pastes, foams, dusting powders, implants or stents are suitable.

Oral or nasal and pharyngeal administrationare preferred.

The compounds according to the invention can be converted into the stated administration forms. This can be effected in a manner known per se by mixing with inert, non-toxic, pharmaceutically suitable additives. These additives include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as for example ascorbic acid), colourants (e.g. inorganic pigments such as for example iron oxides) and flavour or odour correctors.

In general, to achieve effective results in oral administration it has been found advantageous to administer quantities of about 0.01 to 100 mg/kg, preferably about 0.01 to 10 mg/kg body weight. In nasal or pharyngeal administration, the dosage is about 0.01 μg/kg to 1000 μg/kg, preferably about 0.1 to 10 μg/kg body weight. Nonetheless it can sometimes be necessary to deviate from the said quantities, namely depending on body weight, administration route, individual response to the active substance, nature of the preparation and time or interval at which administration takes place. Thus in some cases it can be sufficient to manage with less than the aforesaid minimum quantity, while in other cases the stated upper limit must be exceeded. In the event of administration of larger quantities, it may be advisable to divide these into several individual administrations through the day.

A further subject of the present invention is the combination of the systemic administration of a compound of formula (I) with the local administration of a compound of formula (II).

For this purpose, compound of formula (I) can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent and compounds of formula (II) can be administered for example by the nasal, intranasal, pharyngeal, lingual, sublingual, and buccal route.

The preferred administration is the oral route for a compound of of formula (I) and the nasal and pharyngeal route for a compound of formula (II).

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

For the nasal and pharyngeal administration routes, for example nasal drops, solutions or sprays, pharyngeal sprays, tablets for lingual, sublingual or buccal administration, tablets, films/wafers or capsules, suppositories or oral preparations are suitable.

The following practical examples illustrate the invention. The invention is not limited to the examples.

### EXAMPLES

The synthesis of compounds of formula (I) are described in this section.

Abbreviations and Acronyms:
abs. absolute
Ac acetyl
aq. aqueous, aqueous solution
Boc tert-butoxycarbonyl
br. broad (in NMR signal)
Ex. Example
Bu butyl
c concentration
cat. catalytic
CI chemical ionization (in MS)
d doublet (in NMR)
d day(s)
DCI direct chemical ionization (in MS)
dd doublet of doublets (in NMR)
diamix diastereomer mixture
DMF N,N-dimethylformamide
DMSO dimethyl sulfoxide
dq doublet of quartets (in NMR)
dt doublet of triplet (in NMR)
o. t. of theory (in chemical yield)
EI electron impact ionization (in MS)
eq. equivalent(s)
ESI electrospray ionization (in MS)
Et ethyl
h hour(s)
HATU O-(7-azabenzotriazol-1-yl)-N,N,N′,N′-tetramethyluronium hexafluorophosphate
HOBt 1-hydroxy-1H-benzotriazole hydrate
HPLC high-pressure, high-performance liquid chromatography
iPr isopropyl
conc. concentrated (in the case of a solution)
LC liquid chromatography
LC-MS liquid chromatography-coupled mass spectrometry
lit. literature (reference)
m multiplet (in NMR)
Me methyl
min minute(s)
MS mass spectrometry
NMR nuclear magnetic resonance spectrometry
Ph phenyl
Pr propyl
q quartet (in NMR)
quant. quantitative (in chemical yield)
RP reverse phase (in HPLC)
RT room temperature
Rₜ retention time (in HPLC, LC-MS)
s singlet (in NMR)
t triplet (in NMR)
tBu tert-butyl
TFA trifluoroacetic acid
THF tetrahydrofuran
UV ultraviolet spectrometry
v/v volume to volume ratio (of a solution)
tog. together

LC-MS, GC-MS and HPLC Methods

Method 1 (LC-MS):

MS instrument type: Thermo Scientific FT-MS; instrument type UHPLC+: Thermo Scientific UltiMate 3000; column: Waters, HSST3, 2.1×75 mm, C18 1.8 μm; mobile phase A: 1 1 of water+0.01% formic acid; mobile phase B: 1 1 of acetonitrile+0.01% formic acid; gradient: 0.0 min 10% B→2.5 min 95% B→3.5 min 95% B; oven: 50° C.; flow rate: 0.90 ml/min; UV detection: 210 nm/optimum integration path 210-300 nm.

Method 2 (LC-MS):

MS instrument type: Waters TOF instrument; UPLC instrument type: Waters Acquity I-CLASS; column: Waters Acquity UPLC HSS T3 1.8 μm 50×1 mm; mobile phase A: 1 1 of water+0.100 ml of 99% strength formic acid; mobile phase B: 1 1 of acetonitrile+0.100 ml of 99% strength formic acid; gradient: 0.0 min 90% A→1.2 min 5% A→2.0 min 5% A; oven: 50° C.; flow rate: 0.40 ml/min; UV detection: 210 nm.

Method 3 (GC-MS):

Instrument: Thermo Scientific DSQII, Thermo Scientific Trace GC Ultra; column: Restek RTX-35MS, 15 m×200 μm×0.33 μm; constant flow rate with helium: 1.20 ml/min; oven: 60° C.; inlet: 220° C.; gradient: 60° C., 30° C./min→300° C. (maintain for 3.33 min).

Method 4 (LC-MS):

Instrument: Waters ACQUITY SQD UPLC System; column: Waters Acquity UPLC HSS T3 1.8 μm 50×1 mm; mobile phase A: 1 1 of water+0.25 ml of 99% strength formic acid, mobile phase B: 1 1 of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210 nm.

Method 5 (LC-MS):

Instrument: Waters Single Quad MS System; instrument Waters UPLC Acquity; column: Waters BEH C18 1.7 μ50×2.1 mm; mobile phase A: 1 1 of water+1.0 ml of (25% strength ammonia)/1, mobile phase B: 1 1 of acetonitrile; gradient: 0.0 min 92% A→0.1 min 92% A→1.8 min 5% A→3.5 min 5% A; oven: 50° C.; flow rate: 0.45 ml/min; UV detection: 210 nm.

Method 6 (LC-MS):

MS instrument: Waters SQD2 HPLC instrument: Waters UPLC; column: Zorbax SB-Aq (Agilent), 50 mm×2.1 mm, 1.8 μm; mobile phase A: water+0.025% formic acid, mobile phase B: acetonitrile (ULC)+0.025% formic acid; gradient: 0.0 min 98% A-0.9 min 25% A-1.0 min 5% A→1.4 min 5% A-1.41 min 98% A-1.5 min 98% A; oven: 40° C.; flow rate: 0.600 ml/min; UV detection: DAD; 210 nm.

Method 7 (Preparative HPLC):

Instrument: Waters Prep LC/MS System, column: XBridge C18 5 μm 100×30 mm.

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection).

Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time.

Method 8 (Preparative HPLC):

Instrument: Waters Prep LC/MS System, column: XBridge C18 5 μm 100×30 mm.

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection).

Gradient profile: mobile phase A 0 to 2 min 63 ml, mobile phase B 0 to 2 min 7 ml, mobile phase A 2 to 10 min from 63 ml to 39 ml and mobile phase B from 7 ml to 31 ml, 10 to 12 min 0 ml of mobile phase A and 70 ml of mobile phase B. Mobile phase C and mobile phase D constant flow rate of 5 ml/min each over the entire running time.

Method 9 (Preparative HPLC):

Instrument: Waters Prep LC/MS System, column: XBridge C18 5 μm 100×30 mm.

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection).

Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time.

Method 10 (Preparative HPLC):

Instrument: Waters Prep LC/MS System, column: XBridge C18 5 μm 100×30 mm.

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection).

Gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time.

Method 11 (Preparative HPLC):

Instrument: Abimed Gilson 305; column: Reprosil C18 10 μm, 250 mm×30 mm; mobile phase A: water, mobile phase B: acetonitrile; gradient: 0-3 min 10% B, 3-27 min 10% B→95% B, 27-34.5 min 95% B, 34.5-35.5 min 95% B→10% B, 35.5-36.5 min 10% B; flow rate: 50 ml/min; room temperature; UV detection: 210 nm.

Method 12 (LC-MS):

Instrument: Waters ACQUITY SQD UPLC System; column: Waters Acquity UPLC HSS T3 1.8 μm 50×1 mm; mobile phase A: 1 1 of water+0.25 ml of 99% strength formic acid, mobile phase B: 1 1 of acetonitrile+0.25 ml of 99% strength formic acid; gradient: 0.0 min 95% A→6.0 min 5% A→7.5 min 5% A; oven: 50° C.; flow rate: 0.35 ml/min; UV detection: 210 nm.

Further Details:

The descriptions of the coupling patterns of ¹H NMR signals which follow are guided by the visual appearance of the signals in question and do not necessarily correspond to a strict, physically correct interpretation. In general, the stated chemical shift refers to the center of the signal in question; in the case of broad multiplets, an interval is generally given.

Melting points and melting ranges, if stated, are uncorrected.

In cases where the reaction products were obtained by trituration, stirring or recrystallization, it was frequently possible to isolate further amounts of product from the respective mother liquor by chromatography. However, a description of this chromatography is dispensed with hereinbelow unless a large part of the total yield could only be isolated in this step.

All reactants or reagents whose preparation is not described explicitly hereinafter were purchased commercially from generally accessible sources. For all other reactants or reagents whose preparation is likewise not described hereinafter and which were not commercially obtainable or were obtained from sources which are not generally accessible, a reference is given to the published literature in which their preparation is described.

Starting Materials and Intermediates:

### Example 1A

### 2-Bromo-N-[(3,5-difluoropyridin-2-yl)methyl]-1,3-thiazole-5-carboxamide

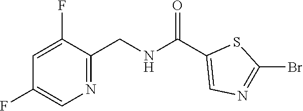 [SMILES: O=C(NCc1ncc(F)cc1F)c1cnc(Br)s1]

50.24 ml (288.41 mmol) of N,N-diisopropylethylamine were added to a solution of 20 g (96.14 mmol) of 2-bromo-1,3-thiazole-5-carboxylic acid and 29.21 g (134.59 mmol) of 1-(3,5-difluoropyridin-2-yl)methanamine dihydrochloride in 450 ml of acetonitrile, the mixture was cooled to 0° C. using an ice bath and 74.4 ml (124.98 mmol) of a 50% strength solution of T3P (2,4,6-tripropyl-1,3,5,2,4,6-trioxatriphosphorinane 2,4,6-trioxide) in ethyl acetate were then added dropwise to the reaction solution. After the addition had ended, the reaction solution was warmed to room temperature and stirred at this temperature for 4 h. About 250 ml of water were then added to the solution. The resulting aqueous phase was then extracted 3× with ethyl acetate. The combined organic phases were subsequently filtered through a hydrophobic filter (pleated filter MN 616 WA ¼, D=12.5 cm), dried and concentrated to dryness under reduced pressure. The residue obtained was triturated with diethyl ether and then air-dried. This gave 27.3 g (81.7 mmol, 85% of theory) of the target product as a light-beige solid. The recovered mother liquor was evaporated to dryness under reduced pressure and the resulting residue was purified further by column chromatography on silica gel (Isolera Biotage SNAP-Ultra 100 g column; mobile phase: cyclohexane/ethyl acetate 9:1→gradient over 15 CV (CV=column volumes)→cyclohexane/ethyl acetate 1:1). This gave a further 2.1 g (6.28 mmol, 6.5% of theory) of the target compound as a white solid.

¹H-NMR (600 MHz, DMSO-d₆, δ/ppm): 4.59 (d, 2H), 7.90-7.95 (m, 1H), 8.27 (s, 1H), 8.48 (d, 1H), 9.32 (br. t, 1H).

LC-MS (Methode 1): Rₜ=1.38 min; m/z=333/335 (M+H)⁺.

Analogously to Example 1A, the following compounds Example 2A to 8A were prepared from the starting materials stated in each case:

| Example | Name/Structure/Starting materials | Analytical data |
|---|---|---|
| 2A | 2-bromo-N-[(3,5-difluoropyridin-2-yl)methyl]-4- methyl-1,3-thiazole-5-carboxamide 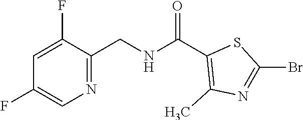 [SMILES: Cc1nc(Br)sc1C(=O)NCc1ncc(F)cc1F] | ¹H-NMR (600 MHz, DMSO-d₆, δ/ppm): 2.48-2.56 (s, 3H, partially obscured by DMSO), 4.56 (d, 2H), 7.91-7.97 (m, 1H), 8.48 (d, 1H), 8.83 (br. t, 1H). LC-MS (Methode 1): Rₜ = 1.55 min; m/z = 349/347 (M + H)⁺. |
| | from 2-bromo-4-methyl-1,3-thiazole-5- | |
| | carboxylic acid and 1-(3,5-difluoropyridin-2- | |
| | yl)methanamine dihydrochloride | |
| 3A | 2-bromo-N-[(3,5-difluoropyridin-2-yl)methyl]-5- methyl-1,3-thiazole-4-carboxamide 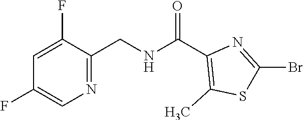 [SMILES: Cc1sc(Br)nc1C(=O)NCc1ncc(F)cc1F] | ¹H-NMR (600 MHz, DMSO-d₆, δ/ppm): 2.48-2.56 (s, 3H, partially obscured by DMSO), 4.56 (d, 2H), 7.90-7.97 (m, 1H), 8.48 (d, 1H), 8.83 (br. t, 1H). LC-MS (Methode 1): Rₜ = 1.51 min; m/z = 349/347 (M + H)⁺. |
| | from 2-bromo-5-methyl-1,3-thiazole-4- | |
| | carboxylic acid and 1-(3,5-difluoropyridin-2- | |
| | yl)methanamine dihydrochloride | |
| 4A | 2-bromo-N-[(3,5-difluoropyridin-2-yl)methyl]- 1,3-thiazole-4-carboxamide 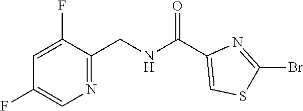 [SMILES: O=C(NCc1ncc(F)cc1F)c1csc(Br)n1] | ¹H-NMR (600 MHz, DMSO-d₆, δ/ppm): 4.60 (d, 2H), 7.89-7.96 (m, 1H), 8.31 (s, 1H), 8.47 (d, 1H), 8.89 (br. t, 1H). LC-MS (Methode 1): Rₜ = 1.56 min; m/z = 333/335 (M + H)⁺. |
| | from 2-bromo-1,3-thiazole-4-carboxylic acid and | |
| | 1-(3,5-difluoropyridin-2-yl)methanamine | |
| | dihydrochloride | |
| 5A | 2-bromo-N-[(3,5-difluoropyridin-2-yl)methyl]-4- (trifluoromethyl)-1,3-thiazole-5-carboxamide 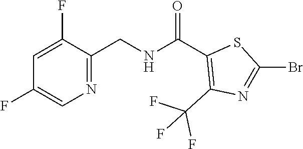 [SMILES: O=C(NCc1ncc(F)cc1F)c1sc(Br)nc1C(F)(F)F] | LC-MS (Methode 1): Rₜ = 1.71 min; m/z = 401/403 (M + H)⁺ |
| | from 2-bromo-4-(trifluoromethyl)-1,3-thiazole-5- | |
| | carboxylic acid and 1-(3,5-difluoropyridin-2- | |
| | yl)methanamine dihydrochloride | |
| 6A | 2-bromo-N-[(3,5-difluoropyridin-2-yl)methyl]-5- ethyl-1,3-thiazole-4-carboxamide 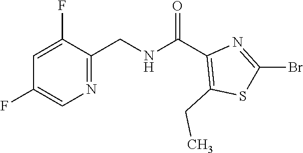 [SMILES: CCc1sc(Br)nc1C(=O)NCc1ncc(F)cc1F] | ¹H-NMR (600 MHz, DMSO-d₆, δ/ppm): 1.20 (t, 3H), 3.23 (q, 2H), 4.58 (d, 2H), 7.89-7.96 (m, 1H), 8.47 (d, 1H), 8.73 (br. t, 1H). LC-MS (Methode 1): Rₜ = 2.06 min; m/z = 361/363 (M + H)⁺. |
| | from 2-bromo-5-ethyl-1,3-thiazole-4-carboxylic | |
| | acid and 1-(3,5-difluoropyridin-2- | |
| | yl)methanamine dihydrochloride | |
| 7A | 2-bromo-N-[(3,5-difluoropyridin-2-yl)methyl]- 1,3-oxazole-4-carboxamide 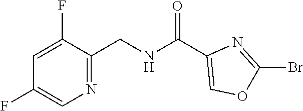 [SMILES: O=C(NCc1ncc(F)cc1F)c1coc(Br)n1] | ¹H-NMR (600 MHz, DMSO-d₆, δ/ppm): 4.57 (d, 2H), 7.89-7.95 (m, 1H), 8.46 (d, 1H), 8.77 (s, 1H), 8.81 (br. t, 1H). LC-MS (Methode 1): Rₜ = 1.37 min; m/z = 317/319 (M + H)⁺. |
| | from 2-bromo-1,3-oxazole-4-carboxylic acid and | |
| | 1-(3,5-difluoropyridin-2-yl)methanamine | |
| | dihydrochloride | |
| 8A | 2-bromo-N-[(3,5-difluoropyridin-2-yl)methyl]-5- methyl-1,3-oxazole-4-carboxamide 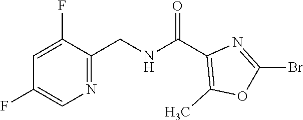 [SMILES: Cc1oc(Br)nc1C(=O)NCc1ncc(F)cc1F] | ¹H-NMR (600 MHz, DMSO-d₆, δ/ppm): 2.56 (s, 3H), 4.54 (d, 2H), 7.88-7.94 (m, 1H), 8.46 (d, 1H), 8.85 (br. t, 1H). LC-MS (Methode 1): Rₜ = 1.64 min; m/z = 331/333 (M + H)⁺. |
| | from 2-bromo-5-methyl-1,3-oxazole-4- | |
| | carboxylic acid and 1-(3,5-difluoropyridin-2- | |
| | yl)methanamine dihydrochloride | |

### Example 9A

### N-[(3,5-Difluoropyridin-2-yl)methyl]-2-(1,4-dioxa-8-azaspiro[4.5]decan-8-yl)-1,3-thiazole-5-carboxamide

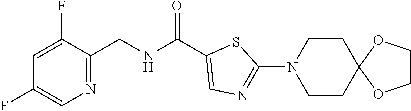 [SMILES: O=C(NCc1ncc(F)cc1F)c1cnc(N2CCC3(CC2)OCCO3)s1]

2 g (5.99 mmol) of 2-bromo-N-[(3,5-difluoropyridin-2-yl)methyl]-1,3-thiazole-5-carboxamide were dissolved in 30 ml of THF, and 4.88 g (14.96 mmol) of caesium carbonate were added. 1.29 g (8.98 mmol) of 1,4-dioxa-8-azaspiro[4.5]decane were then metered into the reaction solution which was subsequently stirred at reflux temperature overnight. After cooling, the reaction mixture was applied directly to silica gel and purified by column chromatography on silica gel (Isolera Biotage SNAP-Ultra 50 g column; mobile phase: cyclohexane/ethyl acetate 85:15→gradient over 15 CV (CV=column volumes)→ethyl acetate). The product fractions obtained were then combined, concentrated on a rotary evaporator and dried under reduced pressure. This gave 1.40 g (3.53 mmol, 99% of theory) of the target compound as a light-beige solid.

¹H-NMR (600 MHz, DMSO-d₆, δ/ppm): 1.71 (t, 4H), 3.56 (t, 4H), 3.92 (s, 4H), 4.53 (br. d, 2H), 7.84 (s, 1H), 7.89-7.94 (m, 1H), 8.47 (d, 1H), 8.74 (t, 1H).

LC-MS (Methode 2): Rₜ=0.73 min; m/z=397 (M+H)⁺.

### Example 10A

### N-[(3,5-Difluoropyridin-2-yl)methyl]-2-(4-oxopiperidin-1-yl)-1,3-thiazole-5-carboxamide

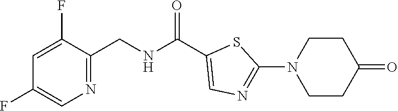 [SMILES: O=C1CCN(c2ncc(C(=O)NCc3ncc(F)cc3F)s2)CC1]

2.3 g (5.80 mmol) of N-[(3,5-difluoropyridin-2-yl)methyl]-2-(1,4-dioxa-8-azaspiro[4.5]decan-8-yl)-1,3-thiazole-5-carboxamide were dissolved in 15 ml of acetone, and 15 ml of semiconcentrated aqueous hydrochloric acid were added. The reaction solution was then stirred at room temperature overnight. The reaction mixture was then concentrated on a rotary evaporator and subsequently taken up in water. The aqueous solution was then adjusted to pH 7 using a saturated sodium bicarbonate solution. The resulting precipitate was filtered off with suction, repeatedly washed with water and dried under reduced pressure. This gave 1.96 g (5.49 mmol, 95% of theory) of the target compound as a white solid.

¹H-NMR (600 MHz, DMSO-d₆, δ/ppm): 2.48-2.56 (t, 4H, partially obscured by DMSO), 3.82 (t, 4H), 4.54 (br. d, 2H), 7.89 (s, 1H), 7.90-7.94 (m, 1H), 8.48 (d, 1H), 8.78 (t, 1H).

LC-MS (Methode 1): Rₜ=1.09 min; m/z=353 (M+H)⁺.

### Example 11A

### 3-[(3,3-Difluorocyclobutyl)methoxy]pyridine

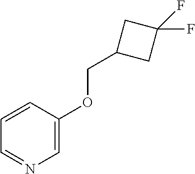 [SMILES: FC1(F)CC(COc2cccnc2)C1]

2 g (21.03 mmol) of pyridin-3-ol were dissoved in 40 ml of THF, and 7.17 g (27.34 mmol) of triphenylphoshine were added. The clear solution was then cooled to 0° C. A further 30 ml of THF were added to the resulting suspension. 5.53 g (27.34 mmol) of diisopropyl azodicarboxylate were added to this suspension and the mixture was stirred at this temperature for 5 min. 3.34 g (27.34 mmol) of (difluorocyclobutyl)methanol, dissolved in 10 ml of THF, were then added dropwise and after the end of the addition the ice bath was removed. After about one hour of stirring at room temperature a clear yellow solution had formed, which was stirred at this temperature overnight.

Water was then added, and the reaction solution was extracted three times with ethyl acetate. The combined organic phases were washed with saturated sodium chloride solution, separated off and filtered through a hydrophobic filter (pleated filter MN 616 WA ¼, D=12.5 cm), dried and concentrated to dryness under reduced pressure. The residue obtained was stirred with about 150 ml of cyclohexane. The precipitated triphenylphosphine oxide was then filtered off with suction and washed repeatedly with cyclohexane. The filtrates obtained were combined and concentrated to dryness under reduced pressure. This gave 3.69 g (18.52 mmol, 88% of theory) of the target compound as a yellow oil. The target compound obtained was reacted further without further purification.

¹H-NMR (600 MHz, DMSO-d₆, δ/ppm): 2.42-2.55 (m, 2H, partially obscured by DMSO), 2.55-2.64 (m, 1H), 2.68-2.78 (m, 2H). 4.11 (d, 2H), 7.30-7.36 (m, 1H), 7.37-7.43 (m, 1H), 8.18 (dd, 1H), 8.30 (d, 1H).

LC-MS (Methode 1): Rₜ=1.12 min; m/z=200 (M+H)⁺.

### Example 12A

### 3-[(3,3-Difluorocyclobutyl)methoxy]piperidine acetate (1:1) (racemate)

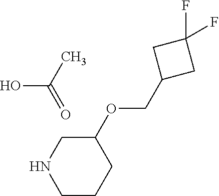

2.5 g (12.55 mmol) of 3-[(3,3-difluorocyclobutyl)methoxy]pyridine were dissolved in 20 ml of glacial acetic acid and hydrogenated using an H-Cube (ThalesNano H-Cube Pro™-1.7).

Reaction Conditions:

catalyst: Pd/C 10%; solvent: glacial acetic acid; cartridge pressure: 80 bar of hydrogen; flow rate: 1 ml/min; temperature: 80° C.

After the reaction had gone to completion, the reaction mixture was concentrated to dryness. The residue obtained was dried under reduced pressure at room temperature overnight. This gave 4.2 g of the target compound as a yellow oil. The target compound was reacted further without further purification.

GC-MS (Methode 3): Rₜ=3.87 min; m/z=205 (M-C₂H₄O₂)⁺.

### Example 13A

### Benzyl 3-(difluoromethyl)[1,4′-bipiperidine]-1′-carboxylate (racemate)

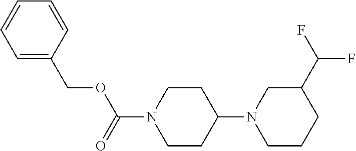 [SMILES: O=C(OCc1ccccc1)N1CCC(N2CCCC(C(F)F)C2)CC1]

1 g (4.29 mmol) of benzyl 4-oxopiperidine-1-carboxylate, 883 mg (5.14 mmol) of 3-(difluoromethyl)piperidine hydrochloride (1:1) and 0.9 ml (5.14 mmol) of N,N-diisopropylethylamine in 15 ml of dichloromethane (a small amount of 4 Å molecular sieve was additionally added to the reaction solution) was stirred at room temperature for 1 h. 1.363 g (6.43 mmol) of sodium acetoxyborohydride were then added and stirring of the reaction mixture was then continued at room temperature overnight. The molecular sieve was then filtered off and washed with dichloromethane and the resulting filtrate was washed twice with sodium bicarbonate solution and once with saturated sodium chloride solution. The organic phase was finally separated off and the organic solution obtained was then filtered through hydrophobic filters (pleated filter MN 616 WA ¼, D=12.5 cm), dried and concentrated to dryness under reduced pressure. This gave 1.39 g (3.54 mmol, purity 89%, 83% of theory) of the target compound as a clear colourless oil. The target compound was reacted further without further purification.

LC-MS (Methode 1): Rₜ=1.04 min; m/z=353 (M+H)⁺.

Analogously to Example 13A, the following compounds of Examples 14A to 17A were prepared from the starting materials stated in each case:

| Example | Name/Structure/Starting materials | Analytical data |
|---|---|---|
| 14A | Benzyl 3-(trifluoromethyl)[1,4′-bipiperidine]-1′- carboxylate (racemate) 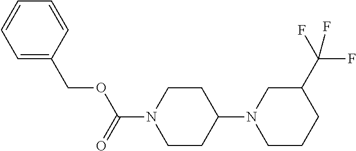 [SMILES: O=C(OCc1ccccc1)N1CCC(N2CCCC(C(F)(F)F)C2)CC1] | ¹H-NMR (600 MHz, DMSO-d₆, δ/ppm): 1.15-1.24 (m, 1H), 1.24- 1.38 (m, 2H), 1.38-1.48 (m, 1H), 1.69 (br. d, 3H), 1.84 (br. d, 1H), 2.06-2.17 (m, 2H), 2.31-2.42 (m, 1H), 2.48-2.58 (m, 1H, partially obscured by DMSO), 2.68-2.88 (m, 3H), 2.92 (br. d, 1H), 4.16 (br. d, 2H), 5.06 (s, 2H), 7.29-7.41 (m, 5H). LC-MS (Methode 4): Rₜ = 0.62 min; m/z = 371 (M + H)⁺. |
| | from benzyl 4-oxopiperidine-1-carboxylate and | |
| | 3-(trifluoromethyl)piperidine | |
| 15A | benzyl 3-(fluoromethyl)[1,4′-bipiperidine]-1′- carboxylate (racemate) 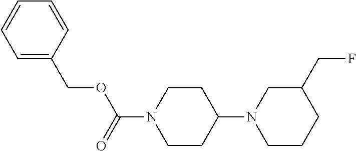 [SMILES: O=C(OCc1ccccc1)N1CCC(N2CCCC(CF)C2)CC1] | ¹H-NMR (600 MHz, DMSO-d₆, δ/ppm): 0.96-1.04 (m, 1H), 1.25- 1.35 (m, 2H), 1.37-1.46 (m, 1H), 1.60 (br. d, 2H), 1.68 (br. d, 2H), 1.77-1.89 (m, 1H), 2.00 (t, 1H), 2.13 (t, 1H), 2.39-2.47 (m, 1H), 2.64-2.88 (m, 4H), 4.02 (br. d, 2H), 4.22-4.29 (m, 1H), 4.30-4.37 (m, 1H), 5.05 (s, 2H), 7.29-7.40 (m, 5H). LC-MS (Methode 1): Rₜ = 1.02 min; m/z = 335 (M + H)⁺. |
| | from benzyl 4-oxopiperidine-1-carboxylate and | |
| | 3-(fluoromethyl)piperidine hydrochloride (1:1) | |
| 16A | benzyl 3-[(3,3-difluorocyclobutyl)methoxy][1,4′- bipiperidine]-1′-carboxylate (racemate) 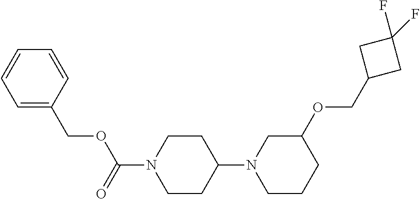 [SMILES: O=C(OCc1ccccc1)N1CCC(N2CCCC(OCC3CC(F)(F)C3)C2)CC1] | ¹H-NMR (600 MHz, DMSO-d₆, δ/ppm): 1.00-1.12 (m, 1H), 1.25- 1.40 (m, 3H), 1.57-1.73 (m, 3H), 1.85-1.92 (m, 1H), 1.96 (t, 1H), 2.08 (t, 1H), 2.22-2.36 (m, 3H), 2.46 (t, 1H), 2.48-2.66 (m, 3H, partially obscured by DMSO), 2.66-2.87 (m, 2H), 2.92 (br. d, 1H), 3.21-3.29 (m, 1H), 3.40-3.50 (m, 2H), 4.03 (br. d, 2H), 5.06 (s, 2H), 7.28-7.40 (m, 5H). LC-MS (Methode 4): Rₜ = 0.69 min; m/z = 423 (M + H)⁺. |
| | from benzyl 4-oxopiperidine-1-carboxylate and | |
| | 3-[(3,3-difluorocyclobutyl)methoxy]piperidine | |
| | acetate (1:1) (racemate) | |
| 17A | benzyl 3-(cyclopropylmethyl)[1,4′-bipiperidine]- 1′-carboxylate (racemate) 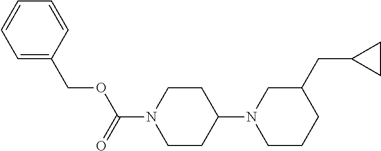 [SMILES: O=C(OCc1ccccc1)N1CCC(N2CCCC(CC3CC3)C2)CC1] | LC-MS (Methode 4): Rₜ = 0.68 min; m/z = 357 (M + H)⁺. |
| | from benzyl 4-oxopiperidine-1-carboxylate and | |
| | 3-(cyclopropylmethyl)piperidine (racemate) | |

### Example 18A

### rac-Benzyl 3-(hydroxymethyl)[1,4′-bipiperidine]-1′-carboxylate

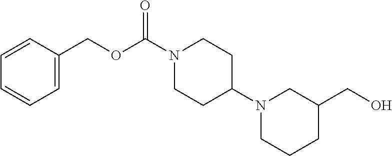 [SMILES: O=C(OCc1ccccc1)N1CCC(N2CCCC(CO)C2)CC1]

Acetic acid (1.8 ml, 32 mmol) was added to a solution of rac-benzyl 4-oxopiperidine-1-carboxylate (5.00 g, 21.4 mmol) and piperidin-3-ylmethanol (4.94 g, 42.9 mmol) in 50 ml of dichloromethane, and the mixture was stirred at room temperature overnight. Sodium triacetoxyborohydride (5.45 g, 25.7 mmol) was then added to the reaction and stirring was continued at room temperature. After 2 h, sat. NaHCO₃ solution was added and the reaction mixture was extracted with dichloromethane. The organic phase was washed with water and dried over Na₂SO₄. The drying agent was filtered off with suction, the filtrate was concentrated and the residue was applied to Isolute®. The mixture was then purified by column chromatography (Biotage® Isolera One; column: Snap Ultra 100 g; DCM/MeOH gradient: 2% MeOH-20% MeOH; flow rate 100 ml/min). The product-containing fractions were combined and concentrated and the residue was dried under high vacuum. This gave 4.37 g (purity 100%, 61% of theory) of the target compound.

LC-MS (Methode 1): Rₜ=0.92 min; MS (ESIpos): m/z=333 [M+H]⁺.

### Example 19A

### rac-Benzyl 3-{[(methylsulfonyl)oxy]methyl}[1,4′-bipiperidine]-1′-carboxylate

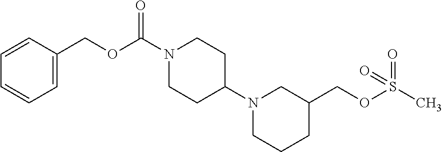 [SMILES: CS(=O)(=O)OCC1CCCN(C2CCN(C(=O)OCc3ccccc3)CC2)C1]

Under argon, rac-benzyl 3-(hydroxymethyl)[1,4′-bipiperidine]-1′-carboxylate (5.42 g, 16.3 mmol) was initially charged in 65 ml of dichloromethane, triethylamine (3.0 ml, 21 mmol) was added and the mixture was cooled to 0° C. At this temperature, methanesulfonyl chloride (1.5 ml, 20 mmol) was added dropwise. The mixture was then stirred at 0° C. for 15 min, after which the ice bath was removed and stirring was continued at room temperature. After 15 min, the reaction mixture was diluted with dichloromethane and washed successively with 1 N hydrochloric acid, sat. NaHCO₃ solution and sat. NaCl solution. The organic phase was dried over Na₂SO₄, filtered and concentrated. The residue was dried under high vacuum and reacted further without further purification. This gave 6.16 g (purity 100%, 92% of theory) of the target compound.

LC-MS (Methode 12): Rₜ=1.39 min; MS (ESIpos): m/z=411 [M+H]⁺.

### Example 20A

### rac-Benzyl 3-(methoxymethyl)[1,4′-bipiperidine]-1′-carboxylate

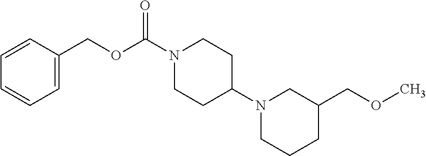 [SMILES: COCC1CCCN(C2CCN(C(=O)OCc3ccccc3)CC2)C1]

Sodium methoxide solution (840 μl, 25% in methanol, 3.7 mmol) was added to a solution of rac-benzyl 3-{[(methylsulfonyl)oxy]methyl}[1,4′-bipiperidine]-1′-carboxylate (500 mg, 1.22 mmol) in 10 ml of DMF, and the mixture was stirred at 50° C. overnight. The solvent was removed on a rotary evaporator and the residue was taken up in ethyl acetate and washed successively with water and sat. NaCl solution. The organic phase was dried over Na₂SO₄, filtered and concentrated. The residue was applied to Isolute® and the mixture was purified by column chromatography (Biotage® Isolera One; column: Snap Ultra 25 g; DCM/MeOH gradient: 2% MeOH-20% MeOH; flow rate 75 ml/min). The product-containing fractions were combined and concentrated and the residue was dried under high vacuum. This gave 146 mg (purity 100%, 35% of theory) of the target compound.

LC-MS (Methode 4): Rₜ=0.59 min; MS (ESIpos): m/z=347 [M+H]⁺.

### Example 21A

### diamix-Benzyl (3R)-3′-fluoro-3-methyl[1,4′-bipiperidine]-1′-carboxylate

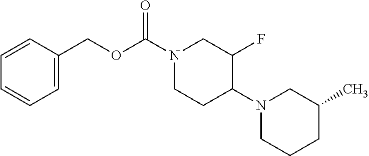 [SMILES: C[C@@H]1CCCN(C2CCN(C(=O)OCc3ccccc3)CC2F)C1]

Acetic acid (1.71 ml, 29.85 mmol) was added to a solution of rac-benzyl 3-fluoro-4-oxopiperidine-1-carboxylate (5 g, 19.9 mmol) and (3R)-3-methylpiperidine (5.4 g, 39.8 mmol) in 200 ml of dichloromethane, and the mixture was stirred at room temperature for 4 h. Subsequently, sodium triacetoxyborohydride (5.06 g, 23.88 mmol) was added and the mixture was stirred at room temperature overnight. The reaction mixture was diluted with dichloromethane and washed successively with sat. NaHCO₃ solution, water and sat. NaCl solution. The organic phase was dried over Na₂SO₄, filtered and concentrated on a rotary evaporator. The residue was applied to Isolute® and purified by column chromatography (Biotage® Isolera One; column: Snap Ultra 100 g; DCM/MeOH gradient: 2% MeOH-20% MeOH; flow rate 100 ml/min). The product-containing fractions were combined and concentrated and the residue was dried under high vacuum. This gave 5.13 g (purity 55%, 42% of theory) of the target compound.

LC-MS (Methode 1): Rₜ=1.05 min; MS (ESIpos): m/z=335 [M+H]⁺.

### Example 22A

### diamix-tert-Butyl (3R)-3′-fluoro-3-methyl[1,4′-bipiperidine]-1-carboxylate

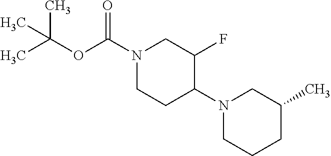 [SMILES: C[C@@H]1CCCN(C2CCN(C(=O)OC(C)(C)C)CC2F)C1]

(3R)-3-Methylpiperidine hydrochloride (6.24 g, 46.0 mmol) was initially charged in 250 ml of 1,2-dichloroethane. N,N-Diisopropylethylamine (8.0 ml, 46 mmol) was added and the mixture was stirred at room temperature for 5 min. rac-tert-Butyl 3-fluoro-4-oxopiperidine-1-carboxylate (5.00 g, 23.0 mmol) and acetic acid (2.0 ml, 35 mmol) were added and the mixture was stirred at room temperature for 4 h. Subsequently, sodium triacetoxyborohydride (5.85 g, 27.6 mmol) was added and the reaction mixture was then stirred at room temperature overnight. The reaction mixture was diluted with dichloromethane and washed successively with sat. NaHCO₃ solution, water and sat.

NaCl solution. The organic phase was dried over Na₂SO₄, filtered and concentrated. The residue was purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and concentrated on a rotary evaporator, and the residue was dried under high vacuum. This gave 5.30 g (purity 100%, 77% of theory) of the target compound.

LC-MS (Methode 4): Rₜ=0.52 min; MS (ESIpos): m/z=301 [M+H]⁺.

### Example 23A

### rac-Benzyl 3-[(2,2,2-trifluoroethoxy)methyl][1,4′-bipiperidine]-1′-carboxylate

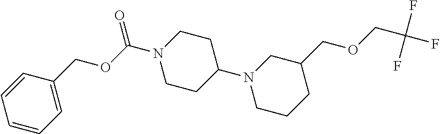 [SMILES: O=C(OCc1ccccc1)N1CCC(N2CCCC(COCC(F)(F)F)C2)CC1]

Under argon, 2,2,2-trifluoroethanol (66 μl, 910 μmol) was initially charged in 5 ml of DMF, and the mixture was cooled in an ice bath to 0° C. At this temperature, sodium hydride (36.5 mg, purity 60%, 913 μmol) was added and the mixture was stirred at room temperature for 30 min.

Subsequently, rac-benzyl 3-{[(methylsulfonyl)oxy]methyl}[1,4′-bipiperidine]-1-carboxylate (250 mg, 609 μmol) was added and the reaction mixture was stirred at 60° C. After 6 h, water was added and the reaction mixture was extracted with ethyl acetate. The organic phase was washed with water and sat. NaCl solution, dried over Na₂SO₄, filtered and concentrated. The residue was dried under high vacuum. This gave 218 mg (purity 81%, 70% of theory) of the target compound.

LC-MS (Methode 1): Rₜ=1.33 min; MS (ESIpos): m/z=415 [M+H]⁺.

### Example 24A

### rac-Benzyl 3-({[1-(fluoromethyl)cyclopropyl]methoxy}methyl)[1,4′-bipiperidine]-1′-carboxylate

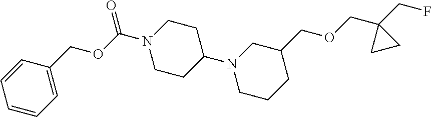 [SMILES: O=C(OCc1ccccc1)N1CCC(N2CCCC(COCC3(CF)CC3)C2)CC1]

Under argon, [1-(fluoromethyl)cyclopropyl]methanol (95.1 mg, 913 μmol) was initially charged in 5 ml of DMF, and the mixture was cooled with an ice bath to 0° C. At this temperature, sodium hydride (36.5 mg, purity 60%, 913 μmol) was added and the mixture was stirred at room temperature for 30 min. Subsequently, rac-benzyl 3-{[(methylsulfonyl)oxy]methyl}[1,4′-bipiperidine]-1-carboxylate (250 mg, 609 μmol) was added and the reaction mixture was stirred at 60° C. overnight. Water was then added, and the reaction mixture was extracted with ethyl acetate. The organic phase was washed with water and sat. NaCl solution, dried over Na₂SO₄, filtered and concentrated. The residue was dried under high vacuum. This gave 204 mg (purity 40%, 32% of theory) of the target compound.

LC-MS (Methode 1): Rₜ=1.36 min; MS (ESIpos): m/z=419 [M+H]⁺.

### Example 25A

### rac-Benzyl 3-({[1-(difluoromethyl)cyclopropyl]methoxy}methyl)[1,4′-bipiperidine]-1′-carboxylate

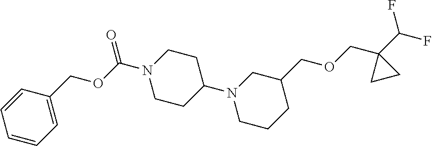 [SMILES: O=C(OCc1ccccc1)N1CCC(N2CCCC(COCC3(C(F)F)CC3)C2)CC1]

Under argon, [1-(difluoromethyl)cyclopropyl]methanol (112 mg, 913 μmol) was initially charged in 5 ml of DMF, and the mixture was cooled in an ice bath to 0° C. At this temperature, sodium hydride (36.5 mg, purity 60%, 913 μmol) was added and the mixture was stirred at room temperature for 30 min. Subsequently, rac-benzyl 3-{[(methylsulfonyl)oxy]methyl}[1,4′-bipiperidine]-1-carboxylate (250 mg, 609 μmol) was added and the reaction mixture was stirred at 60° C. After 6 h, water was added and the reaction mixture was extracted with ethyl acetate. The organic phase was washed with water and sat. NaCl solution, dried over Na₂SO₄, filtered and concentrated. The residue was dried under high vacuum. This gave 197 mg (purity 51%, 37% of theory) of the target compound.

LC-MS (Methode 1): Rₜ=1.41 min; MS (ESIpos): m/z=437 [M+H]⁺.

### Example 26A

### rac-Benzyl 3-({[1-(trifluoromethyl)cyclopropyl]methoxy}methyl)[1,4′-bipiperidine]-1′-carboxylate

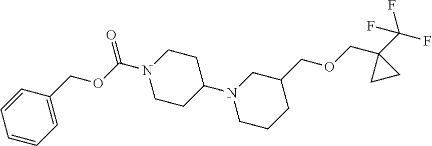 [SMILES: O=C(OCc1ccccc1)N1CCC(N2CCCC(COCC3(C(F)(F)F)CC3)C2)CC1]

Under argon, [1-(trifluoromethyl)cyclopropyl]methanol (128 mg, 913 μmol) was initially charged in 5 ml of DMF, and the mixture was cooled with an ice bath to 0° C. At this temperature, sodium hydride (36.5 mg, purity 60%, 913 μmol) was added and the mixture was stirred at room temperature for 30 min. Subsequently, rac-benzyl 3-{[(methylsulfonyl)oxy]methyl}[1,4′-bipiperidine]-1-carboxylate (250 mg, 609 μmol) was added and the reaction mixture was stirred at 60° C. After 6 h, water was added and the reaction mixture was extracted with ethyl acetate. The organic phase was washed with water and sat. NaCl solution, dried over Na₂SO₄, filtered and concentrated. The residue was dried under high vacuum. This gave 212 mg (purity 58%, 44% of theory) of the target compound.

LC-MS (Methode 1): Rₜ=1.48 min; MS (ESIpos): m/z=455 [M+H]⁺.

### Example 27A

### Benzyl 3,3-dimethyl[1,4′-bipiperidine]-1′-carboxylate

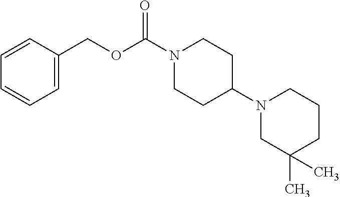 [SMILES: CC1(C)CCCN(C2CCN(C(=O)OCc3ccccc3)CC2)C1]

Acetic acid (74 μl, 1.3 mmol) was added to a solution of benzyl 4-oxopiperidine-1-carboxylate (200 mg, purity 58%, 857 μmol) and 3,3-dimethylpiperidine (240 μl, 1.7 mmol) in 7 ml of dichloromethane, and the mixture was stirred at room temperature for 5 h. Subsequently, sodium triacetoxyborohydride (218 mg, 1.03 mmol) was added to the reaction and the mixture was stirred at room temperature overnight. Sat. NaHCO₃ solution was added and the reaction mixture was extracted with dichloromethane. The organic phase was washed with water and sat. NaCl solution and dried over Na₂SO₄. The drying agent was filtered off, the filtrate was concentrated and the residue was dried under high vacuum. This gave 280 mg (purity 81%, 80% of theory) of the target compound.

LC-MS (Methode 1): Rₜ=1.18 min; MS (ESIpos): m/z=331 [M+H]⁺.

### Example 28A

### Benzyl 4-(5-azaspiro[2.5]octan-5-yl)piperidine-1-carboxylate

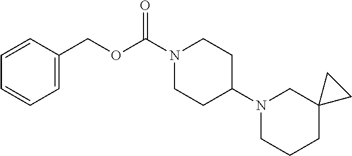 [SMILES: O=C(OCc1ccccc1)N1CCC(N2CCCC3(CC3)C2)CC1]

Acetic acid (110 μl, 1.9 mmol) was added to a solution of benzyl 4-oxopiperidine-1-carboxylate (300 mg, 1.29 mmol) and 5-azaspiro[2.5]octane (286 mg, 2.57 mmol) in 10 ml of dichloromethane, and the mixture was stirred at room temperature for 5 h. Subsequently, sodium triacetoxyborohydride (327 mg, 1.54 mmol) was added to the reaction and the mixture was stirred at room temperature overnight. Sat. NaHCO₃ solution was added and the reaction mixture was extracted with dichloromethane. The organic phase was washed with water and dried over Na₂SO₄. The drying agent was filtered off, the filtrate was concentrated and the residue was dried under high vacuum. This gave 368 mg (purity 40%, 35% of theory) of the target compound.

LC-MS (Methode 1): Rₜ=1.12 min; MS (ESIpos): m/z=329 [M+H]⁺.

### Example 29A

### rac-Benzyl 4-(1,1-difluoro-5-azaspiro[2.5]octan-5-yl)piperidine-1-carboxylate

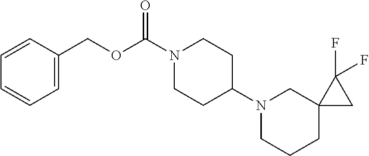 [SMILES: O=C(OCc1ccccc1)N1CCC(N2CCCC3(C2)CC3(F)F)CC1]

Acetic acid (110 μl, 1.9 mmol) was added to a solution of benzyl 4-oxopiperidine-1-carboxylate (300 mg, 1.29 mmol) and rac-1,1-difluoro-5-azaspiro[2.5]octane hydrochloride (354 mg, 1.93 mmol) in 10 ml of dichloromethane, and the mixture was stirred at room temperature for 4 h. Subsequently, sodium triacetoxyborohydride (327 mg, 1.54 mmol) was added to the reaction and the mixture was stirred at room temperature overnight. Sat. NaHCO₃ solution was added and the reaction mixture was extracted with dichloromethane. The organic phase was washed with water and dried over Na₂SO₄. The drying agent was filtered off, the filtrate was concentrated on a rotary evaporator and the residue was dried under high vacuum. This gave 405 mg (purity 61%, 53% of theory) of the target compound.

LC-MS (Methode 1): Rₜ=1.14 min; MS (ESIpos): m/z=365 [M+H]⁺.

### Example 30A

### rac-Benzyl 3-hydroxy[1,4′-bipiperidine]-1′-carboxylate

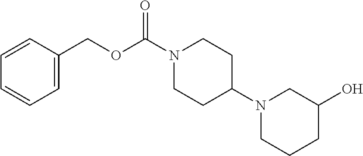 [SMILES: O=C(OCc1ccccc1)N1CCC(N2CCCC(O)C2)CC1]

Triethylamine (1.8 ml, 13 mmol) and acetic acid (740 μl, 13 mmol) were added to a solution of benzyl 4-oxopiperidine-1-carboxylate (2.00 g, 8.57 mmol) and piperidin-3-ol (1.73 g, 17.1 mmol) in 100 ml of dichloromethane, and the mixture was stirred at room temperature for 4 h. Subsequently, sodium triacetoxyborohydride (2.18 g, 10.3 mmol) was added to the reaction and the mixture was stirred at room temperature for 48 h. Sat. NaHCO₃ solution was added and the reaction mixture was extracted with dichloromethane. The organic phase was washed with water and dried over Na₂SO₄. The drying agent was filtered off and the filtrate was concentrated. The residue was applied to Isolute® and the mixture was purified by column chromatography (Biotage® Isolera One; column: Snap Ultra 50 g; DCM/MeOH gradient: 2% MeOH-20% MeOH; flow rate 100 ml/min). The product-containing fractions were combined and concentrated and the residue was dried under high vacuum. This gave 1.87 g (purity 100%, 68% of theory) of the target compound.

LC-MS (Methode 1): Rₜ=0.88 min; MS (ESIpos): m/z=319 [M+H]⁺.

### Example 31A

### rac-Benzyl 3-(cyclopropylmethoxy)[1,4′-bipiperidine]-1-carboxylate

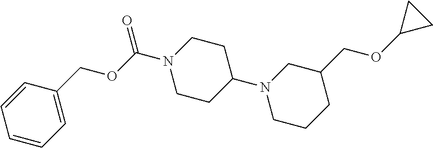 [SMILES: O=C(OCc1ccccc1)N1CCC(N2CCCC(COC3CC3)C2)CC1]

Under argon, rac-benzyl 3-hydroxy[1,4′-bipiperidine]-1′-carboxylate (250 mg, 785 μmol) was initially charged in 5 ml of THF, and the mixture was cooled with an ice bath to 0° C. At this temperature, sodium hydride (47.1 mg, purity 60%, 1.18 mmol) was added and the mixture was stirred at room temperature for 30 min. Subsequently, (bromomethyl)cyclopropane (110 μl, 1.2 mmol) was added and the reaction mixture was stirred at 60° C. overnight. (Bromomethyl)cyclopropane (110 μl, 1.2 mmol) and sodium hydride (47.1 mg, purity 60%, 1.18 mmol) were added and the mixture was stirred at 60° C. for a further 24 h. Subsequently, the product was isolated by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined and concentrated on a rotary evaporator, and the residue was dried under high vacuum. This gave 68.0 mg (purity 68%, 16% of theory) of the target compound.

LC-MS (Methode 1): Rₜ=1.25 min; MS (ESIpos): m/z=373 [M+H]⁺.

### Example 32A

### rac-Benzyl 3-[(cyclobutyloxy)methyl][1,4′-bipiperidine]-1-carboxylate

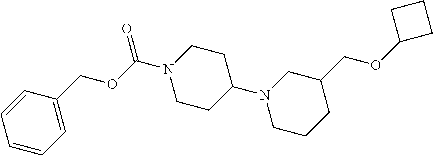 [SMILES: O=C(OCc1ccccc1)N1CCC(N2CCCC(COC3CCC3)C2)CC1]

Under argon, cyclobutanol (72 μl, 910 μmol) was initially charged in 5 ml of DMF, and the mixture was cooled with an ice bath to 0° C. At this temperature, sodium hydride (36.5 mg, purity 60%, 913 μmol) was added and the mixture was stirred at room temperature for 30 min. Subsequently, rac-benzyl 3-{[(methylsulfonyl)oxy]methyl}[1,4′-bipiperidine]-1-carboxylate (250 mg, 609 μmol) was added and the reaction mixture was stirred at 60° C. overnight. Water was then added, and the reaction mixture was extracted with ethyl acetate. The organic phase was washed with water and sat. NaCl solution, dried over Na₂SO₄, filtered and concentrated on a rotary evaporator. The residue was dried under high vacuum. This gave 290 mg (purity 46%, 57% of theory) of the target compound.

LC-MS (Methode 4): Rₜ=0.73 min; MS (ESIpos): m/z=387 [M+H]⁺.

### Example 33A

### rac-Benzyl 3-[(cyclopropylmethoxy)methyl][1,4′-bipiperidine]-1-carboxylate

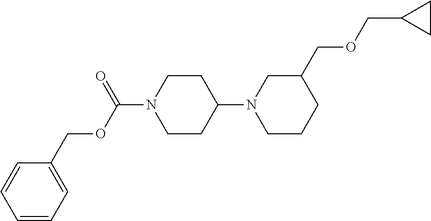 [SMILES: O=C(OCc1ccccc1)N1CCC(N2CCCC(COCC3CC3)C2)CC1]

Under argon, sodium hydride (268 mg, purity 60%, 6.70 mmol) was initially charged in 25 ml of DMF, and the mixture was cooled with an ice bath to 0° C. At this temperature, cyclopropylmethanol (540 μl, 6.7 mmol) was added and the mixture was stirred at room temperature for 30 min. Subsequently, rac-benzyl 3-{[(methylsulfonyl)oxy]methyl}[1,4′-bipiperidine]-1-carboxylate (2.50 g, 6.09 mmol) was added and the reaction mixture was stirred at 55° C. overnight. Cyclopropylmethanol (540 μl, 6.7 mmol) and sodium hydride (268 mg, purity 60%, 6.70 mmol) were added and the mixture was stirred at 55° C. for a further 24 h. Water was then added, and the reaction mixture was extracted with ethyl acetate. The organic phase was washed with water and sat. NaCl solution, dried over Na₂SO₄, filtered and concentrated on a rotary evaporator. The residue was purified by preparative HPLC (instrument: Waters Prep LC/MS System, column: Phenomenex Kinetex C18 5 μm 100×30 mm; mobile phase A: water, mobile phase B: acetonitrile, mobile phase C: 2% strength formic acid in water, mobile phase D: acetonitrile/water (80% by volume/20% by volume); total flow rate: 80 ml/min; room temperature, wavelength 200-400 nm, complete injection; gradient profile: mobile phase A 0 to 2 min 63 ml, mobile phase B 0 to 2 min 7 ml, mobile phase A 2 to 10 min from 63 ml to 39 ml and mobile phase B from 7 ml to 31 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 241 mg (purity 78%, 8% of theory) of the target compound.

LC-MS (Methode 1): Rₜ=1.27 min; MS (ESIpos): m/z=387 [M+H]⁺.

### Example 34A

### tert-Butyl 4-[(3R)-3-methylpiperidin-1-yl]azepane-1-carboxylate

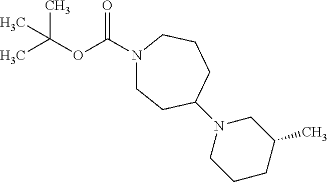 [SMILES: C[C@@H]1CCCN(C2CCCN(C(=O)OC(C)(C)C)CC2)C1]

Acetic acid (72 μl, 1.3 mmol) was added to a solution of tert-butyl 4-oxoazepane-1-carboxylate (179 mg, 840 μmol) and (3R)-3-methylpiperidine (167 mg, 1.68 mmol) in 5 ml of dichloromethane, and the mixture was stirred at room temperature. After 5 h, sodium triacetoxyborohydride (214 mg, 1.01 mmol) was added to the reaction and the mixture was stirred at room temperature overnight. Subsequently, sat. NaHCO₃ solution was added and the reaction mixture was extracted with dichloromethane. The organic phase was washed with water and dried over Na₂SO₄. The drying agent was filtered off with suction, the filtrate was concentrated on a rotary evaporator and the residue was dried under high vacuum. This gave 215 mg of a mixture which was reacted further without further purification and analysis.

### Example 35A

### diamix-Benzyl 3-({[-2,2-difluorocyclopropyl]methoxy}methyl)[1,4′-bipiperidine]-1′-carboxylate

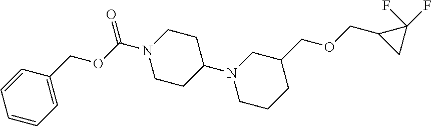 [SMILES: O=C(OCc1ccccc1)N1CCC(N2CCCC(COCC3CC3(F)F)C2)CC1]

Under argon, rac-(2,2-difluorocyclopropyl)methanol (98.7 mg, 913 μmol) was initially charged in 5 ml of DMF, and the mixture was cooled with an ice bath to 0° C. At this temperature, sodium hydride (36.5 mg, purity 60%, 913 μmol) was added and the mixture was stirred at room temperature for 30 min. Subsequently, rac-benzyl 3-{[(methylsulfonyl)oxy]methyl}[1,4′-bipiperidine]-1-carboxylate (250 mg, 609 μmol) was added and the reaction mixture was stirred at 60° C. overnight. Water was added and the reaction mixture was extracted with ethyl acetate. The organic phase was washed with water and sat. NaCl solution, dried over Na₂SO₄, filtered and concentrated on a rotary evaporator. The residue was dried under high vacuum. This gave 343 mg (purity 56%, 74% of theory) of the target compound.

LC-MS (Methode 1): Rₜ=1.32 min; MS (ESIpos): m/z=423 [M+H]⁺.

### Example 36A

### rac-Benzyl 3-{[(3,3-difluorocyclobutyl)methoxy]methyl}[1,4′-bipiperidine]-1′-carboxylate

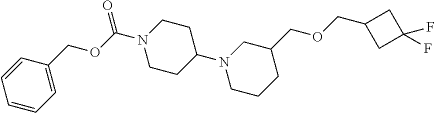 [SMILES: O=C(OCc1ccccc1)N1CCC(N2CCCC(COCC3CC(F)(F)C3)C2)CC1]

Under argon,(3,3-difluorocyclobutyl)methanol (112 mg, 913 μmol) was initially charged in 5 ml of DMF, and the mixture was cooled with an ice bath to 0° C. At this temperature, sodium hydride (36.5 mg, purity 60%, 913 μmol) was added and the mixture was stirred at room temperature for 30 min. Subsequently, rac-benzyl 3-{[(methylsulfonyl)oxy]methyl}[1,4′-bipiperidine]-1′-carboxylate (250 mg, 609 μmol) was added and the reaction mixture was stirred at 60° C. After 6 h, water was added and the reaction mixture was extracted with ethyl acetate. The organic phase was washed with water and sat. NaCl solution, dried over Na₂SO₄, filtered and concentrated on a rotary evaporator. The residue was dried under high vacuum. This gave 287 mg (purity 33%, 36% of theory) of the target compound.

LC-MS (Methode 1): Rₜ=1.44 min; MS (ESIpos): m/z=437 [M+H]⁺.

### Example 37A

### 3-(Difluoromethyl)-1,4′-bipiperidine dihydrochloride (racemate)

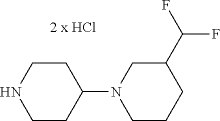 [SMILES: FC(F)C1CCCN(C2CCNCC2)C1]

1.35 g (3.83 mmol) of benzyl 3-(difluoromethyl)[1,4′-bipiperidine]-1-carboxylate (racemate) were dissolved in 100 ml of ethanol and hydrogenated using an H-Cube (ThalesNano H-Cube Pro™-1.7).

Reaction Conditions:

catalyst: Pd/C 10%; solvent: ethanol; cartridge pressure: 1 bar of hydrogen; flow rate: 1 ml/min; temperature: 50° C.

After complete conversion, 4 N HCl (in dioxane) was added and the reaction mixture was concentrated to dryness. The residue obtained was dried under reduced pressure at room temperature overnight. This gave 1,107 g (3.80 mmol, 99% of theory) of the target compound as a white solid. The target compound was reacted further without further purification.

GC-MS (Methode 3): Rₜ=4.87 min; m/z=218 (M−2HCl)⁺.

### Example 38A

### 3-[(3,3-Difluorocyclobutyl)methoxy]-1,4′-bipiperidine (racemate)

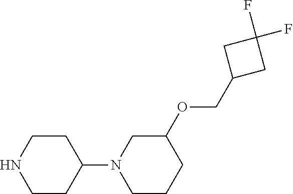 [SMILES: FC1(F)CC(COC2CCCN(C3CCNCC3)C2)C1]

2.7 g (6.39 mmol) of benzyl 3-[(3,3-difluorocyclobutyl)methoxy][1,4′-bipiperidine]-1′-carboxylate (racemate) were dissolved in 90 ml of ethanol and hydrogenated using an H-Cube (ThalesNano H-Cube Pro™-1.7).

Reaction Conditions:

catalyst: Pd/C 10%; solvent: ethanol; cartridge pressure: 50 bar of hydrogen; flow rate: 1 ml/min; temperature: 50° C.

After the reaction had gone to completion, the reaction mixture was concentrated to dryness. The residue obtained was dried under reduced pressure at room temperature overnight. This gave 1.27 g (4.40 mmol, 69% of theory) of the target compound as a yellow oil. The target compound was reacted further without further purification.

GC-MS (Methode 3): Rₜ=6.42 min; m/z=288 (M)⁺.

Analogously to Examples 37A and 38A, the following compounds of Examples 39A to 41A were prepared from the starting materials stated in each case:

| Example | Name/Structure/Starting materials | Analytical data |
|---|---|---|
| 39A | 3-(trifluoromethyl)-1,4′-bipiperidine dihydrochloride (racemate) 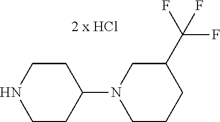 [SMILES: FC(F)(F)C1CCCN(C2CCNCC2)C1] | GC-MS (Methode 3): Rₜ = 4.33 min; m/z = 236 (M − 2HCl)⁺. |
| | from benzyl 3-(trifluoromethyl)[1,4′- | |
| | bipiperidine]-1′-carboxylate (racemate) | |
| 40A | 3-(fluoromethyl)-1,4′-bipiperidine dihydrochloride (racemate) 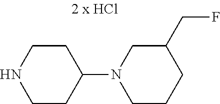 [SMILES: FCC1CCCN(C2CCNCC2)C1] | GC-MS (Methode 3): Rₜ = 5.07 min; m/z = 200 (M − 2HCl)⁺. |
| | from benzyl 3-(fluoromethyl)[1,4′-bipiperidine]- | |
| | 1′-carboxylate (racemate) | |
| 41A | 3-(cyclopropylmethyl)-1,4′-bipiperidine (racemate) 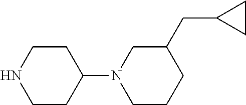 [SMILES: C1CC(CC2CC2)CN(C2CCNCC2)C1] | GC-MS (Methode 3): Rₜ = 5.81 min; m/z = 222 (M)⁺. |
| | from benzyl 3-(cyclopropylmethyl)[1,4′- | |
| | bipiperidine]-1′-carboxylate (racemate) | |

### Example 42A

### rac-3-(Methoxymethyl)-1,4′-bipiperidine dihydrochloride

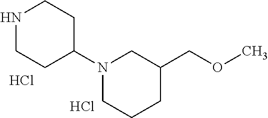

rac-Benzyl 3-(methoxymethyl)[1,4′-bipiperidine]-1-carboxylate (145 mg, 419 μmol) was initially charged in 5 ml of THF, and palladium (50.0 mg; 10% on activated carbon) was added under argon. The mixture was then hydrogenated under a hydrogen atmosphere overnight. The catalyst was filtered off through kieselguhr and washed with THF. Hydrochloric acid in diethyl ether (310 μl, 2.0 M, 630 μmol) was added to the filtrate, and the precipitated solid was filtered off with suction, washed with diethyl ether and dried under high vacuum. This gave 92.0 mg (purity 76%, 59% of theory) of the target compound.

GC-MS (Methode 3): Rₜ=5.45 min; MS (ESIpos): m/z=212 [M−HCl]⁺.

### Example 43A

### diamix-(3R)-3′-Fluoro-3-methyl-1,4′-bipiperidine dihydrochloride

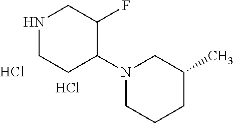

Synthesis Method 1:

diamix-Benzyl (3R)-3′-fluoro-3-methyl[1,4′-bipiperidine]-1′-carboxylate (5.13 g, purity 55%, 8.40 mmol) was initially charged in 250 ml of THF, and palladium (382 mg; 10% on activated carbon) was added under argon. The mixture was then hydrogenated under a hydrogen atmosphere overnight. The catalyst was filtered off through kieselguhr and washed with THF. Hydrochloric acid in diethyl ether (6.3 ml, 2.0 M, 13 mmol) was added to the filtrate and the mixture was concentrated on a rotary evaporator. The residue was stirred with dichloromethane, and the solid was filtered off with suction, washed with dichloromethane and dried under high vacuum. This gave 2.31 g (100% of theory) of the target compound.

LC-MS (Methode 4): MS (ESIpos): m/z=200 [M−2HCl]⁺.

Synthesis Method 2:

4 M Hydrochloric acid in 1,4-dioxane (22 ml, 4.0 M, 88 mmol) was added to a solution of diamix-tert-butyl (3R)-3′-fluoro-3-methyl[1,4′-bipiperidine]-1′-carboxylate (5.30 g, 17.6 mmol) in 250 ml of dichloromethane, and the mixture was stirred at room temperature for 48 h. The precipitated solid was filtered off with suction, washed with dichloromethane and dried in a vacuum drying cabinet at 40° C. overnight. This gave 3.47 g (purity 100%, 72% of theory) of the target compound.

GC-MS (Methode 3): MS (ESIpos): m/z=200 [M−2HCl].

### Example 44A

### rac-3-[(2,2,2-Trifluoroethoxy)methyl]-1,4′-bipiperidine dihydrochloride

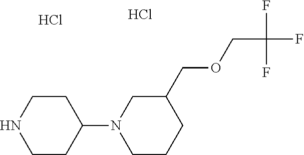 [SMILES: FC(F)(F)COCC1CCCN(C2CCNCC2)C1 ; Cl ; Cl]

rac-Benzyl 3-[(2,2,2-trifluoroethoxy)methyl][1,4′-bipiperidine]-1-carboxylate (218 mg, purity 81%, 526 μmol) was initially charged in 12 ml of THF, and palladium (63 mg; 10% on activated carbon) was added under argon. The mixture was then hydrogenated under a hydrogen atmosphere. After 3.5 h the catalyst was filtered off through kieselguhr and washed with THF. Hydrochloric acid in diethyl ether (390 μl, 2.0 M, 790 μmol) was added to the filtrate and the mixture was concentrated on a rotary evaporator. This gave 164 mg (purity 74%, 66% of theory) of the target compound.

GC-MS (Methode 3): R₁=5.26 min; MS (full ms): m/z=280 [M−2HCl]⁺.

### Example 45A

### rac-3-({[1-(Fluoromethyl)cyclopropyl]methoxy}methyl)-1,4′-bipiperidine dihydrochloride

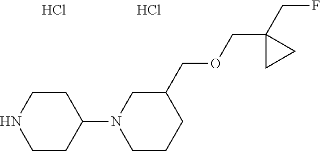 [SMILES: FCC1(COCC2CCCN(C3CCNCC3)C2)CC1 ; Cl ; Cl]

rac-Benzyl 3-({[1-(fluoromethyl)cyclopropyl]methoxy}methyl)[1,4′-bipiperidine]-1-carboxylate (204 mg, purity 40%, 487 μmol) was initially charged in 10 ml of THF, and palladium (58 mg; 10% on activated carbon) was added under argon. The mixture was then hydrogenated under a hydrogen atmosphere. After 2 h the catalyst was filtered off through kieselguhr and washed with THF. Hydrochloric acid in diethyl ether (370 μl, 2.0 M, 740 μmol) was added to the filtrate and the mixture was concentrated on a rotary evaporator. This gave 133 mg of a mixture which was reacted without further purification and analysis.

### Example 46A

### rac-3-({[1-(Difluoromethyl)cyclopropyl]methoxy}methyl)-1,4′-bipiperidine dihydrochloride

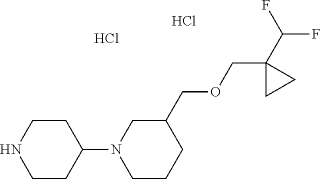 [SMILES: FC(F)C1(COCC2CCCN(C3CCNCC3)C2)CC1 ; Cl ; Cl]

rac-Benzyl 3-({[1-(difluoromethyl)cyclopropyl]methoxy}methyl)[1,4′-bipiperidine]-1-carboxylate (197 mg, purity 51%, 451 μmol) was initially charged in 10 ml of THF, and palladium (54 mg; 10% on activated carbon) was added under argon. The mixture was then hydrogenated under a hydrogen atmosphere. After 1.5 h the catalyst was filtered off through kieselguhr and washed with THF. Hydrochloric acid in diethyl ether (374 μl, 2.0 M, 680 μmol) was added to the filtrate and the mixture was concentrated on a rotary evaporator. This gave 112 mg of a mixture which was reacted without further purification and analysis.

### Example 47A

### rac-3-({[1-(Trifluoromethyl)cyclopropyl]methoxy}methyl)-1,4′-bipiperidine dihydrochloride

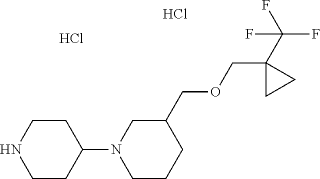 [SMILES: FC(F)(F)C1(COCC2CCCN(C3CCNCC3)C2)CC1 ; Cl ; Cl]

rac-Benzyl 3-({[1-(trifluoromethyl)cyclopropyl]methoxy}methyl)[1,4′-bipiperidine]-1-carboxylate (212 mg, purity 58%, 466 μmol) was initially charged in 10 ml of THF, and palladium (56 mg; 10% on activated carbon) was added under argon. The mixture was then hydrogenated under a hydrogen atmosphere. After 1.5 h the catalyst was filtered off through kieselguhr and washed with THF. Hydrochloric acid in diethyl ether (350 μl, 2.0 M, 700 μmol) was added to the filtrate and the mixture was concentrated on a rotary evaporator. This gave 129 mg of a mixture which was reacted further without further purification and analysis.

### Example 48A

### 3,3-Dimethyl-1,4′-bipiperidine dihydrochloride

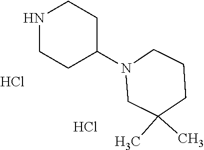

Benzyl 3,3-dimethyl[1,4′-bipiperidine]-1-carboxylate (260 mg, purity 81%, 637 μmol) was initially charged in 18 ml of THF, and palladium (27 mg; 10% on activated carbon, 255 μmol) was added under argon. The mixture was then hydrogenated under a hydrogen atmosphere overnight. The catalyst was filtered off through kieselguhr and washed with THF. Hydrochloric acid in diethyl ether (478 μl, 2.0 M, 956 μmol) was added to the filtrate and the mixture was concentrated on a rotary evaporator. The residue was stirred with dichloromethane, concentrated and dried under high vacuum. This gave 180 mg of a mixture which was reacted further without further purification and analysis.

### Example 49A

### 5-(Piperidin-4-yl)-5-azaspiro[2.5]octane dihydrochloride

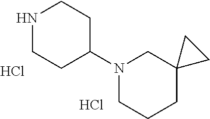 [SMILES: Cl ; C1CN(C2CCNCC2)CC2(C1)CC2 ; Cl]

Benzyl 4-(5-azaspiro[2.5]octan-5-yl)piperidine-1-carboxylate (368 mg, purity 40%, 1.12 mmol) was initially charged in 32 ml of THF, and palladium (51 mg, 10% on activated carbon) was added under argon. The mixture was then hydrogenated under a hydrogen atmosphere overnight. The catalyst was filtered off through kieselguhr and washed with THF. Hydrochloric acid in diethyl ether (840 μl, 2.0 M, 1.7 mmol) was added to the filtrate and the mixture was concentrated on a rotary evaporator. The residue was stirred with dichloromethane. The precipitated solid was filtered off with suction, washed with dichloromethane and dried under high vacuum. This gave 185 mg of a mixture which was reacted further without further purification and analysis.

### Example 50A

### rac-1,1-Difluoro-5-(piperidin-4-yl)-5-azaspiro[2.5]octane dihydrochloride

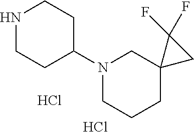 [SMILES: FC1(F)CC12CCCN(C1CCNCC1)C2 ; Cl ; Cl]

rac-Benzyl 4-(1,1-difluoro-5-azaspiro[2.5]octan-5-yl)piperidine-1-carboxylate (405 mg, purity 61%, 1.11 mmol) was initially charged in 32 ml of THF, and palladium (51 mg, 10% on activated carbon) was added under argon. The mixture was then hydrogenated under a hydrogen atmosphere overnight. The catalyst was filtered off through kieselguhr and washed with THF. Hydrochloric acid in diethyl ether (840 μl, 2.0 M, 1.7 mmol) was added to the filtrate and the mixture was concentrated on a rotary evaporator. The residue was stirred with dichloromethane, concentrated on a rotary evaporator and dried under high vacuum. This gave 280 mg of a mixture which was reacted further without further purification and analysis.

### Example 51A

### rac-3-(Cyclopropylmethoxy)-1,4′-bipiperidine dihydrochloride

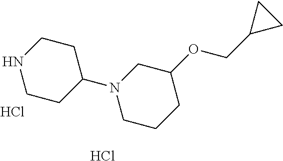 [SMILES: Cl ; C1CC(OCC2CC2)CN(C2CCNCC2)C1 ; Cl]

rac-Benzyl 3-(cyclopropylmethoxy)[1,4′-bipiperidine]-1-carboxylate (68.0 mg, purity 68%, 124 μmol) was initially charged in 5 ml of THF, and palladium (22 mg; 10% on activated carbon) was added under argon. The mixture was then hydrogenated under a hydrogen atmosphere overnight. The catalyst was filtered off through kieselguhr and washed with THF. Hydrochloric acid in diethyl ether (93 μl, 2.0 M, 186 μmol) was added to the filtrate and the mixture was concentrated on a rotary evaporator. The residue was stirred with dichloromethane, concentrated and dried under high vacuum. This gave 51 mg of a mixture which was reacted further without further purification and analysis.

### Example 52A

### rac-3-[(Cyclobutyloxy)methyl]-1,4′-bipiperidine dihydrochloride

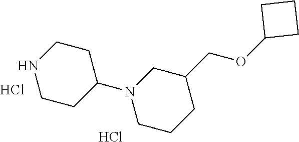 [SMILES: Cl ; C1CC(OCC2CCCN(C3CCNCC3)C2)C1 ; Cl]

rac-Benzyl 3-[(cyclobutyloxy)methyl][1,4′-bipiperidine]-1-carboxylate (290 mg, purity 46%, 386 μmol) was initially charged in 15 ml of THF, and palladium (41 mg; 10% on activated carbon) was added under argon. The mixture was then hydrogenated under a hydrogen atmosphere overnight. The catalyst was filtered off through kieselguhr and washed with THF. Hydrochloric acid in diethyl ether (259 μl, 2.0 M, 518 μmol) was added to the filtrate and the mixture was concentrated on a rotary evaporator. This gave 225 mg of a mixture which was reacted further without further purification and analysis.

### Example 53A

### rac-3-[(Cyclopropylmethoxy)methyl]-1,4′-bipiperidine dihydrochloride

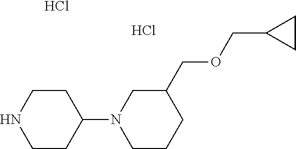 [SMILES: Cl ; C1CC(COCC2CC2)CN(C2CCNCC2)C1 ; Cl]

rac-Benzyl 3-[(cyclopropylmethoxy)methyl][1,4′-bipiperidine]-1′-carboxylate (241 mg, purity 78%, 486 μmol) was initially charged in 20 ml of THF, and palladium (58 mg; 10% on activated carbon) was added under argon. The mixture was then hydrogenated under a hydrogen atmosphere overnight. The catalyst was filtered off through kieselguhr and washed with THF. Hydrochloric acid in diethyl ether (360 μl, 2.0 M, 730 μmol) was added to the filtrate and the mixture was concentrated on a rotary evaporator. This gave 155 mg of a mixture which was reacted further without further purification and analysis.

### Example 54A

### 4-[(3R)-3-Methylpiperidin-1-yl]azepane dihydrochloride

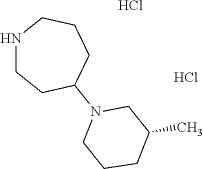

4 M Hydrochloric acid in 1,4-dioxane (2.2 ml, 4.0 M, 8.6 mmol) was added to a solution of tert-butyl 4-[(3R)-3-methylpiperidin-1-yl]azepane-1-carboxylate (215 mg) in 5.4 ml of dichloromethane, and the mixture was stirred at room temperature. After 2 h, the reaction mixture was concentrated on a rotary evaporator and the residue was dried under high vacuum. This gave 237 mg of a mixture which was reacted further without further purification and analysis.

### Example 55A

### diamix-3-[(3-Fluorobutoxy)methyl]-1,4′-bipiperidine dihydrochloride

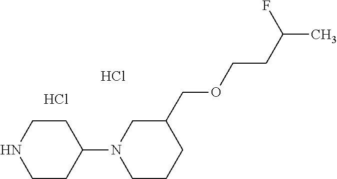 [SMILES: CC(F)CCOCC1CCCN(C2CCNCC2)C1 ; Cl ; Cl]

diamix-Benzyl 3-({[-2,2-difluorocyclopropyl]methoxy}methyl)[1,4′-bipiperidine]-1′-carboxylate (343 mg, purity 56%, 446 μmol) was initially charged in 25 ml of THF, and palladium (53 mg; 10% on activated carbon) was added under argon. The mixture was then hydrogenated under a hydrogen atmosphere overnight. The catalyst was filtered off through kieselguhr and washed with THF. Hydrochloric acid in diethyl ether (330 μl, 2.0 M, 670 μmol) was added to the filtrate and the mixture was concentrated on a rotary evaporator. This gave 218 mg of a mixture which was reacted further without further purification and analysis.

### Example 56A

### rac-3-{[(3,3-Difluorocyclobutyl)methoxy]methyl}-1,4′-bipiperidine dihydrochloride

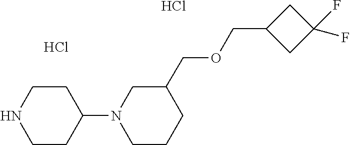 [SMILES: FC1(F)CC(COCC2CCCN(C3CCNCC3)C2)C1 ; Cl ; Cl]

rac-Benzyl 3-{[(3,3-difluorocyclobutyl)methoxy]methyl}[1,4′-bipiperidine]-1-carboxylate (287 mg, purity 33%, 217 μmol) was initially charged in 15 ml of THF, and palladium (26 mg; 10% on activated carbon) was added under argon. The mixture was then hydrogenated under a hydrogen atmosphere overnight. The catalyst was filtered off through kieselguhr and washed with THF. Hydrochloric acid in diethyl ether (163 μl, 2.0 M, 325 μmol) was added to the filtrate and the mixture was concentrated on a rotary evaporator. This gave 286 mg of a mixture which was reacted further without further purification and analysis.

### Example 57A

### Methyl 2-[(3R)-3-methyl[1,4′-bipiperidin]-1′-yl]-1,3-thiazole-5-carboxylate

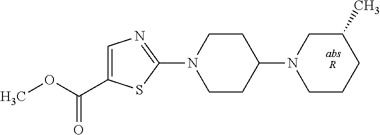

5 g (22.52 mmol) of methyl 2-bromo-1,3-thiazole-5-carboxylate, 4.926 g (22.52 mmol) of 1-(3,5-difluoropyridin-2-yl)methanamine dihydrochloride and 9.4 ml (67.55 mmol) of triethylamine in 30 ml of 2-propanol were heated to boiling point (oil bath temperature ˜100° C.) and stirred at this temperature overnight. After cooling of the reaction mixture, the solution was concentrated to dryness using a rotary evaporator. This gave 14.29 g (crude product, purity ˜34%) of the target product and the triethylamine salts. The mixture was reacted further without further purification.

LC-MS (Methode 4): Rₜ=0.51 min; m/z=324 (M+H)⁺.

### Example 58A

### 2-[(3R)-3-Methyl[1,4′-bipiperidin]-1′-yl]-1,3-thiazole-5-carboxylic acid dihydrochloride

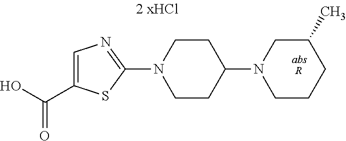

14.29 g of the mixture of methyl 2-[(3R)-3-methyl[1,4′-bipiperidin]-1′-yl]-1,3-thiazole-5-carboxylate and triethylamine salts were dissolved in water, and 221 ml of a 1 N NaOH solution were added. A brown oil separated off, which was dissolved by addition of 50 ml of THF. The reaction mixture was then heated to 60° C. and stirred at this temperature for one hour. After cooling of the reaction mixture to room temperature, the solution was concentrated to dryness on a rotary evaporator, taken up in water and acidified with concentrated hydrochloric acid. The solution was then once more concentrated to dryness. This gave 20.54 g of a beige solid which was purified by column chromatography.

Conditions: The separation was carried out using 1 g portions. RP column Chromatorex C18, 10 μm; 125×30 mm, acetonitrile/water (+0.05% formic acid) 5/95→gradient over 20 min→acetonitrile/water (+0.05% formic acid) 95/5, flow rate 75 ml/min.

Finally, product-containing fractions were combined and concentrated to dryness under reduced pressure and dried. This gave 4.75 g (12.42 mmol, 83% of theory) of the target compound as a light-beige solid.

LC-MS (Methode 1): Rₜ=0.54 min; m/z=310 (M+H−2HCl)⁺.

### Example 59A

### 3-[(3R)-3-Methyl[1,4′-bipiperidin]-1′-yl]-1,2,4-oxadiazole-5-carboxylic acid

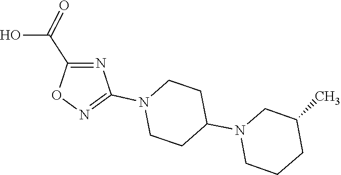 [SMILES: C[C@@H]1CCCN(C2CCN(c3noc(C(=O)O)n3)CC2)C1]

Ethyl 3-bromo-1,2,4-oxadiazole-5-carboxylate (100 mg, 452 μmol) and (3R)-3-methyl-1,4′-bipiperidine dihydrochloride (173 mg, 679 μmol) were stirred in 2 ml of sodium carbonate solution (2.0 ml, 2.0 M, 4.0 mmol) at 120° C. After 30 min, the reaction mixture was acidified with 2 N hydrochloric acid and purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined and concentrated and the residue was dried under high vacuum. This gave 25 mg (purity 60%, 11% of theory) of the target compound.

LC-MS (Methode 1): Rₜ=0.47 min; MS (ESIpos): m/z=295 [M+H]⁺.

### Example 60A

### rac-3-[(2,2-Difluorocyclopropyl)methoxy]pyridine hydrochloride

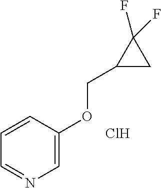 [SMILES: FC1(F)CC1COc1cccnc1 ; Cl]

Triphenylphosphine (2.43 g, 9.25 mmol) was added to a solution of pyridin-3-ol (677 mg, 7.12 mmol) in 25 ml of THF and the mixture was cooled in an ice bath to 0° C. At this temperature, diisopropyl azodicarboxylate (1.3 ml, 9.3 mmol) was added and the mixture was stirred at 0° C. for 5 min. Subsequently, a solution of rac-2,2-difluorocyclopropanemethanol (1.00 g, 9.25 mmol) in 5 ml of THE was added dropwise to the mixture. The ice bath was then removed and the mixture was stirred at room temperature overnight. Water was added and the reaction mixture was extracted with ethyl acetate. The organic phase was washed with sat. NaCl solution, dried over Na₂SO₄, filtered and concentrated. The oily residue was stirred with 75 ml of cyclohexane for 30 min. The precipitated solid was filtered off and the filtrate was concentrated to afford a residue. The residue was dissolved in 50 ml of MTBE, and 5 ml of hydrochloric acid (4N in 1,4-dioxane) were added. The precipitated solid was filtered off with suction, washed with MTBE and dried under high vacuum. This gave 698 mg (purity 93%, 41% of theory) of the target compound.

LC-MS (Methode 4): Rₜ=0.40 min; MS (ESIpos): m/z=186 [M−HCl]⁺.

### Example 61A

### diamix-3-[(2,2-Difluorocyclopropyl)methoxy]piperidine sulfate hydrochloride

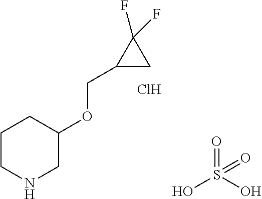 [SMILES: FC1(F)CC1COC1CCCNC1 ; Cl ; O=S(=O)(O)O]

Under argon, rac-3-[(2,2-difluorocyclopropyl)methoxy]pyridine hydrochloride (698 mg, purity 93%, 2.93 mmol) was dissolved in 35 ml of ethanol. Sulfuric acid (168 μl, 3.15 mmol) and platinum(IV) oxide (179 mg, 0.79 mmol) were added and the mixture was hydrogenated under a hydrogen atmosphere overnight. The catalyst was filtered off through Celite and washed with ethanol. The filtrate was concentrated by evaporation and the residue was dried in high vacuum. This gave 761 mg (74% of theory) of the target compound.

LC-MS (Methode 5): MS (ESIpos): m/z=192 [M−HCl—H₂SO₄]⁺.

### Example 62A

### 3-(Cyclobutyloxy)pyridine hydrochloride

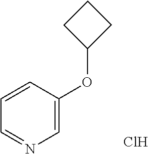 [SMILES: c1cncc(OC2CCC2)c1 ; Cl]

Triphenylphosphine (7.17 g, 27.3 mmol) was added to a solution of pyridin-3-ol (2.00 g, 21.0 mmol) in 70 ml of THF and the mixture was cooled in an ice bath to 0° C. At this temperature, diisopropyl azodicarboxylate (3.9 ml, 27 mmol) was added and the mixture was stirred at 0° C. for 5 min. Subsequently, a solution of cyclobutanol (2.1 ml, 27 mmol) in 10 ml of THF was added dropwise to the mixture. The ice bath was then removed and the mixture was stirred at room temperature over the weekend. Water was added and the reaction mixture was extracted with ethyl acetate. The organic phase was washed with sat. NaCl solution, dried over Na₂SO₄, filtered and concentrated. The oily residue was stirred with 150 ml of cyclohexane for 30 min. The solid was filtered off and the filtrate was concentrated to afford a residue. The residue was dissolved in 100 ml of MTBE, and 5 ml of hydrochloric acid (4N in 1,4-dioxane) were added. The precipitated solid was filtered off with suction, washed with MTBE and dried under high vacuum. This gave 2.02 g (purity 51%, 26% of theory) of the target compound.

LC-MS (Methode 5): Rₜ=1.34 min; MS (ESIpos): m/z=150 [M−HCl]⁺.

### Example 63A

### rac-3-(Cyclobutyloxy)piperidine sulfate hydrochloride

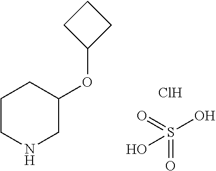 [SMILES: C1CC(OC2CCCNC2)C1 ; O=S(=O)(O)O ; Cl]

Under argon, 3-(cyclobutyloxy)pyridine hydrochloride (2.0 g, purity 51%, 5.51 mmol) was dissolved in 95 ml of ethanol. Sulfuric acid (550 μl, 10 mmol) and platinum(IV) oxide (612 mg, 2.6 mmol) were added and the mixture was hydrogenated under a hydrogen atmosphere overnight. The catalyst was filtered off through Celite and washed with ethanol. The filtrate was concentrated by evaporation and the residue was dried in high vacuum. This gave 2.52 g (157% of theory) of the target compound.

LC/MS (Methode 4): MS (ESIpos): m/z=156 [M−HCl—H₂SO₄]⁺.

### Example 64A

### 3-[(3,3-Difluorocyclobutyl)oxy]pyridine hydrochloride

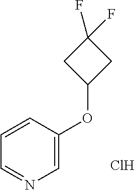 [SMILES: FC1(F)CC(Oc2cccnc2)C1 ; Cl]

Triphenylphosphine (2.43 g, 9.25 mmol) was added to a solution of pyridin-3-ol (677 mg, 7.12 mmol) in 25 ml of THF and the mixture was cooled in an ice bath to 0° C. At this temperature, diisopropyl azodicarboxylate (1.3 ml, 9.3 mmol) was added and the mixture was stirred at 0° C. for 5 min. Subsequently, a solution of 3,3-difluorocyclobutanol (1.00 g, 9.25 mmol) in 5 ml of THF was added dropwise to the mixture. The ice bath was then removed and the mixture was stirred at room temperature overnight. The reaction mixture was stirred at 80° C. for 5 h and then extracted between water and ethyl acetate. The organic phase was washed with sat. NaCl solution, dried over Na₂SO₄, filtered and concentrated. The oily residue was stirred with 150 ml of cyclohexane for 30 min. The precipitated solid was filtered off and the filtrate was concentrated to afford a residue. The residue was dissolved in 100 ml of MTBE, and 5 ml of hydrochloric acid (4N in 1,4-dioxane) were added. The precipitated solid was filtered off with suction, washed with MTBE and dried under high vacuum. This gave 289 mg (purity 94%, 17% of theory) of the target compound.

LC-MS (Methode 4): Rₜ=1.01 min; MS (ESIpos): m/z=186 [M−HCl]⁺.

### Example 65A

### rac-3-[(3,3-Difluorocyclobutyl)oxy]piperidine sulfate hydrochloride

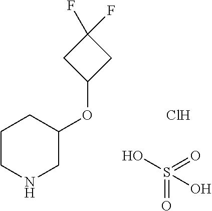 [SMILES: FC1(F)CC(OC2CCCNC2)C1 ; O=S(=O)(O)O ; Cl]

Under argon, 3-[(3,3-difluorocyclobutyl)oxy]pyridine hydrochloride (298 mg, 1.34 mmol) was dissolved in 12 ml of ethanol. Sulfuric acid (72 μl, 1.3 mmol) and platinum(IV) oxide (76.3 mg, 336 μmol) were added and the mixture was hydrogenated under a hydrogen atmosphere for 3 h. The catalyst was filtered off through Celite and washed with ethanol. The filtrate was concentrated by evaporation and the residue was dried in high vacuum. This gave 297 mg (68% of theory) of the target compound.

LC/MS (Methode 4): MS (ESIpos): m/z=192 [M−HCl—H₂SO₄]⁺.

### Example 66A

### 2-Chloro-N-[(3,5-difluoropyridin-2-yl)methyl]-1,3-oxazole-4-carboxamide

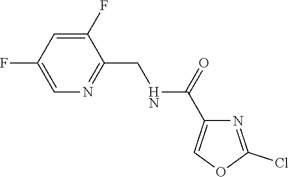 [SMILES: O=C(NCc1ncc(F)cc1F)c1coc(Cl)n1]

N,N-Diisopropylethylamine (680 μl, 3.9 mmol) and propylphosphonic anhydride (1.0 ml, 50% in ethyl acetate, 1.7 mmol) were added to a solution of 2-bromo-1,3-oxazole-4-carboxylic acid (250 mg, 1.30 mmol) and 1-(3,5-difluoropyridin-2-yl)methanamine dihydrochloride (283 mg, 1.30 mmol) in 10 ml of acetonitrile, and the mixture was stirred at room temperature overnight. The reaction mixture was concentrated and the residue was taken up in ethyl acetate and washed with sat. NaHCO₃ solution, water and sat. NaCl solution. The organic phase was dried over Na₂SO₄. The drying agent was filtered off and the filtrate was concentrated. The residue was applied to Isolute® and the mixture was purified by column chromatography (Biotage® Isolera One; column: Snap Ultra 10 g; Cy/EA gradient: 8% EA-66% EA; flow rate 36 ml/min). The product-containing fractions were combined and concentrated and the residue was dried under high vacuum. This gave 193 mg (46% of theory, purity 84%) of the target compound, which was reacted further without further purification.

LC-MS (Methode 1): Rₜ=1.32 min; MS (ESIpos): m/z=274 [M+H]⁺.

### Example 67A

### 2-Bromo-N-(5-chloro-2-fluorobenzyl)-1,3-thiazole-5-carboxamide

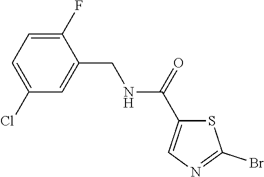 [SMILES: O=C(NCc1cc(Cl)ccc1F)c1cnc(Br)s1]

N,N-Diisopropylethylamine (630 μl, 3.6 mmol) and propylphosphonic anhydride (930 μl, 50% in ethyl acetate, 1.6 mmol) were added to a solution of 2-bromo-1,3-thiazole-5-carboxylic acid (250 mg, 1.20 mmol) and 1-(5-chloro-2-fluorophenyl)methanamine (192 mg, 1.20 mmol) in 10 ml of acetonitrile, and the mixture was stirred at room temperature overnight. The reaction mixture was concentrated and the residue was taken up in ethyl acetate and washed with sat. NaHCO₃ solution, water and sat. NaCl solution. The organic phase was dried over Na₂SO₄. The drying agent was filtered off and the filtrate was concentrated. The residue was applied to Isolute® and the mixture was purified by column chromatography (Biotage® Isolera One; column: Snap Ultra 10 g; Cy/EA gradient: 8% EA-66% EA; flow rate 36 ml/min). The product-containing fractions were combined and concentrated and the residue was dried under high vacuum. This gave 106 mg (purity 96%, 24% of theory) of the target compound.

LC-MS (Methode 1): Rₜ=1.85 min; MS (ESIpos): m/z=348 [M+H]⁺.

### Example 68A

### Benzyl (3R)-3-hydroxy[1,4′-bipiperidine]-1′-carboxylate

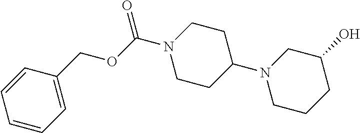 [SMILES: O=C(OCc1ccccc1)N1CCC(N2CCC[C@@H](O)C2)CC1]

Triethylamine (3.0 ml, 21 mmol) and acetic acid (740 μl, 13 mmol) were added to a solution of benzyl 4-oxopiperidine-1-carboxylate (2.00 g, 8.57 mmol) and (3R)-piperidin-3-ol hydrochloride (2.36 g, 17.1 mmol) in 100 ml of dichloromethane, and the mixture was stirred at room temperature for 1 h. Subsequently, sodium triacetoxyborohydride (2.18 g, 10.3 mmol) was added to the mixture and the mixture was stirred at room temperature for 48 h. Sat. NaHCO₃ solution was added and the reaction mixture was extracted with dichloromethane. The organic phase was washed with water and dried over Na₂SO₄. The drying agent was filtered off and the filtrate was concentrated. The residue was applied to Isolute® and the mixture was purified by column chromatography (Biotage® Isolera One; column: Snap Ultra 50 g; DCM/MeOH gradient: 2% MeOH-20% MeOH; flow rate 100 ml/min). The product-containing fractions were combined and concentrated and the residue was dried under high vacuum. This gave 1.79 g (purity 100%, 66% of theory) of the target compound.

LC-MS (Methode 1): Rₜ=0.87 min; MS (ESIpos): m/z=319 [M+H]⁺.

### Example 69A

### Benzyl (3R)-3-(cyclopropylmethoxy)[1,4′-bipiperidine]-1′-carboxylate

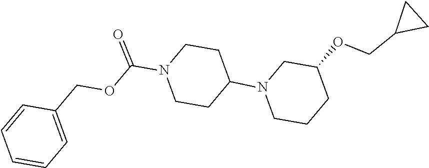 [SMILES: O=C(OCc1ccccc1)N1CCC(N2CCC[C@@H](OCC3CC3)C2)CC1]

Under argon, benzyl (3R)-3-hydroxy[1,4′-bipiperidine]-1-carboxylate (1.79 g, 5.62 mmol) was initially charged in 40 ml of THF, and the mixture was cooled with an ice bath to 0° C. At this temperature, sodium hydride (337 mg, purity 60%, 8.43 mmol) was added and the mixture was stirred at room temperature for 30 min. Subsequently, (bromomethyl)cyclopropane (820 μl, 8.4 mmol) was added and the reaction mixture was stirred at 60° C. overnight. (Bromomethyl)cyclopropane (820 μl, 8.4 mmol) and sodium hydride (337 mg, purity 60%, 8.43 mmol) were added and the mixture was stirred at 60° C. for a further 24 h. Water was added and the reaction mixture was extracted with ethyl acetate. The organic phase was washed with water and sat. NaCl solution and dried over Na₂SO₄. The drying agent was filtered off and the filtrate was concentrated. The product was purified by preparative HPLC (instrument: Waters Prep LC/MS System, column: Phenomenex Kinetex C18 5 μm 100×30 mm. mobile phase A: water, mobile phase B: acetonitrile, mobile phase C: 2% strength formic acid in water, mobile phase D: acetonitrile/water (80% by volume/20% by volume) total flow rate: 80 ml/min, room temperature, wavelength 200-400 nm, complete injection. Gradient profile: mobile phase A 0 to 2 min 63 ml, mobile phase B 0 to 2 min 7 ml, mobile phase A 2 to 10 min from 63 ml to 39 ml and mobile phase B from 7 ml to 31 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 100.0 mg (purity 100%, 4.8% of theory) of the target compound.

LC-MS (Methode 1): R₄=1.19 min; MS (ESIpos): m/z=373 [M+H]⁺.

### Example 70A

### (3R)-3-(Cyclopropylmethoxy)-1,4′-bipiperidine dihydrochloride

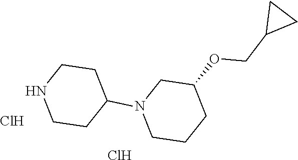 [SMILES: Cl ; C1C[C@@H](OCC2CC2)CN(C2CCNCC2)C1 ; Cl]

Benzyl (3R)-3-(cyclopropylmethoxy)[1,4′-bipiperidine]-1′-carboxylate (100 mg, 268 μmol) was initially charged in 7.5 ml of THF, and palladium (32.1 mg; 10% on activated carbon) was added under argon. The mixture was then hydrogenated under a hydrogen atmosphere for 2 h. The catalyst was filtered off through kieselguhr and washed with THF. Hydrochloric acid in diethyl ether (200 μl, 2.0 M, 400 μmol) was added to the filtrate and the mixture was concentrated on a rotary evaporator. The residue was stirred with dichloromethane, concentrated and dried under high vacuum. This gave 66 mg of a mixture which was reacted further without further purification and analysis.

### Example 71A

### rac-2-Bromo-N-[1-(2,5-difluorophenyl)ethyl]-1,3-thiazole-5-carboxamide

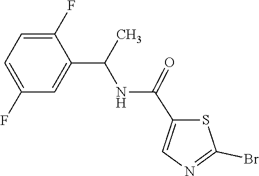 [SMILES: CC(NC(=O)c1cnc(Br)s1)c1cc(F)ccc1F]

N,N-Diisopropylethylamine (630 μl, 3.6 mmol) and propylphosphonic anhydride (930 μl, 50% in ethyl acetate, 1.6 mmol) were added to a solution of 2-bromo-1,3-thiazole-5-carboxylic acid (250 mg, 1.20 mmol) and rac-1-(2,5-difluorophenyl)ethanamine (189 mg, 1.20 mmol) in 10 ml of acetonitrile, and the mixture was stirred at room temperature overnight. The reaction mixture was concentrated and the residue was taken up in ethyl acetate and washed with sat. NaHCO₃ solution, water and sat. NaCl solution. The organic phase was dried over Na₂SO₄. The drying agent was filtered off and the filtrate was concentrated. The residue was applied to Isolute® and the mixture was purified by column chromatography (Biotage® Isolera One; column: Snap Ultra 10 g; Cy/EA gradient: 8% EA-66% EA; flow rate 36 ml/min). The product-containing fractions were combined and concentrated and the residue was dried under high vacuum. This gave 148 mg (purity 100%, 35% of theory) of the target compound.

LC-MS (Methode 1): Rₜ=1.81 min; MS (ESIpos): m/z=346 [M+H]⁺.

### Example 72A

### Ethyl 4-(2-chlorophenyl)-2-[(3R)-3-methyl[1,4′-bipiperidin]-1′-yl]-1,3-thiazole-5-carboxylate

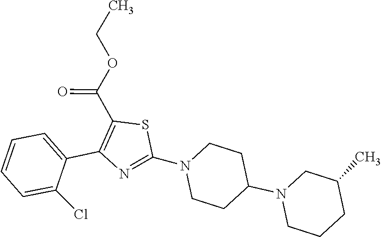 [SMILES: CCOC(=O)c1sc(N2CCC(N3CCC[C@@H](C)C3)CC2)nc1-c1ccccc1Cl]

Ethyl 2-bromo-4-(2-chlorophenyl)-1,3-thiazole-5-carboxylate (150 mg, 433 μmol) and (3R)-3-methyl-1,4′-bipiperidine dihydrochloride (166 mg, 649 μmol) were combined and stirred at 120° C. in sodium carbonate solution (870 μl, 2.0 M, 1.7 mmol) for 30 min. The reaction mixture was then diluted with water and extracted with dichloromethane. The organic phase was dried over Na₂SO₄ and filtered and the filtrate was concentrated on a rotary evaporator. The residue was dried under high vacuum. This gave 199 mg (purity 95%, 98% of theory) of the target compound.

LC-MS (Methode 1): Rₜ=1.34 min; MS (ESIpos): m/z=449 [M+H]⁺.

### Example 82A

### diamix-5-(3-Fluoropiperidin-4-yl)-5-azaspiro[2.5]octane dihydrochloride

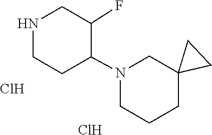 [SMILES: Cl ; FC1CNCCC1N1CCCC2(CC2)C1 ; Cl]

4 M hydrochloric acid in 1,4-dioxane (720 μl, 4.0 M, 2.9 mmol) was added to a solution of diamix-tert-butyl 4-(5-azaspiro[2.5]octan-5-yl)-3-fluoropiperidine-1-carboxylate (179 mg, 573 μmol) in 8 ml of dichloromethane, and the mixture was stirred at room temperature overnight. Subsequently, the reaction mixture was concentrated on a rotary evaporator and the residue was dried under high vacuum. This gave 162 mg of a mixture which was reacted further without further purification and analysis.

### Example 73A

### 4-(2-Chlorophenyl)-2-[(3R)-3-methyl[1,4′-bipiperidin]-1′-yl]-1,3-thiazole-5-carboxylic acid

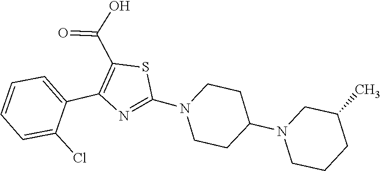 [SMILES: C[C@@H]1CCCN(C2CCN(c3nc(-c4ccccc4Cl)c(C(=O)O)s3)CC2)C1]

Ethyl 4-(2-chlorophenyl)-2-[(3R)-3-methyl[1,4′-bipiperidin]-1′-yl]-1,3-thiazole-5-carboxylate (199 mg, 444 μmol) was dissolved in 10 ml of THF. Aqueous sodium hydroxide solution (4 ml, 2.0 M, 8 mmol) was added to the solution and the mixture was stirred at room temperature for 5 days. The THF was removed on a rotary evaporator and the residue was acidified with hydrochloric acid. The precipitated solid was filtered off and dried under high vacuum. This gave 160 mg (purity 98%, 84% of theory) of the target compound.

LC-MS (Methode 1): Rₜ=0.97 min; MS (ESIpos): m/z=420 [M+H]⁺.

### Example 74A

### 4-Bromo-2-[(3R)-3-methyl[1,4′-bipiperidin]-1′-yl]-1,3-thiazole-5-carboxylic acid

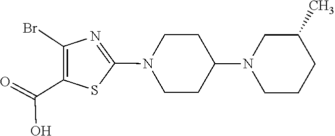 [SMILES: C[C@@H]1CCCN(C2CCN(c3nc(Br)c(C(=O)O)s3)CC2)C1]

2,4-Dibromo-1,3-thiazole-5-carboxylic acid (150 mg, 523 μmol) and (3R)-3-methyl-1,4′-bipiperidine dihydrochloride (133 mg, 523 μmol) were combined and stirred at 120° C. in sodium carbonate solution (1.0 ml, 2.0 M, 2.1 mmol) for 1 h. Subsequently, the reaction mixture was concentrated to dryness and stirred with DCM/MeOH 5:1. The insoluble salts were filtered off with suction. The filtrate was concentrated by evaporation and the residue was dried in high vacuum. This gave 240 mg (purity 100%, 118% of theory) of the target compound.

LC-MS (Methode 1): Rₜ=0.70 min; MS (ESIpos): m/z=388 [M+H]⁺.

### Example 75A

### 2-Bromo-4-chloro-N-[(3,5-difluoropyridin-2-yl)methyl]-1,3-thiazole-5-carboxamide

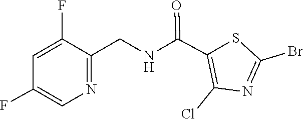 [SMILES: O=C(NCc1ncc(F)cc1F)c1sc(Br)nc1Cl]

N,N-Diisopropylethylamine (720 μl, 4.1 mmol) and propylphosphonic anhydride (800 μl, 50% in ethyl acetate, 1.3 mmol) were added to a solution of 2-bromo-4-chloro-1,3-thiazole-5-carboxylic acid (250 mg, 1.03 mmol) and 1-(3,5-difluoropyridin-2-yl)methanamine dihydrochloride (291 mg, 1.34 mmol) in 14 ml of acetonitrile, and the mixture was stirred at room temperature for 2 h. The reaction mixture was concentrated and the residue was taken up in ethyl acetate and washed with sat. NaHCO₃ solution, water and sat. NaCl solution. The organic phase was dried over Na₂SO₄. The drying agent was filtered off and the filtrate was concentrated. The residue was dried under high vacuum. This gave 250 mg (purity 95%, 62% of theory) of the target compound.

LC-MS (Methode 1): Rₜ=1.79 min; MS (ESIpos): m/z=367 [M+H]⁺.

### Example 76A

### 2-Bromo-4-cyclopropyl-N-[(3,5-difluoropyridin-2-yl)methyl]-1,3-thiazole-5-carboxamide

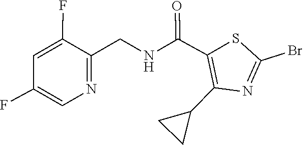 [SMILES: O=C(NCc1ncc(F)cc1F)c1sc(Br)nc1C1CC1]

N,N-Diisopropylethylamine (560 μl, 3.2 mmol) and propylphosphonic anhydride (620 μl, 50% in ethyl acetate, 1.0 mmol) were added to a solution of 2-bromo-4-cyclopropyl-1,3-thiazole-5-carboxylic acid (200 mg, 806 μmol) and 1-(3,5-difluoropyridin-2-yl)methanamine dihydrochloride (227 mg, 1.05 mmol) in 11 ml of acetonitrile, and the mixture was stirred at room temperature for 1 h. The reaction mixture was concentrated and the residue was taken up in ethyl acetate and washed with sat. NaHCO₃ solution, water and sat. NaCl solution. The organic phase was dried over Na₂SO₄. The drying agent was filtered off and the filtrate was concentrated. The residue was dried under high vacuum. This gave 239 mg (purity 78%, 62% of theory) of the target compound.

LC-MS (Methode 1): Rₜ=1.87 min; MS (ESIpos): m/z=373 [M+H]⁺.

### Example 77A

### 2-Bromo-4-ethyl-1,3-thiazole-5-carboxylic acid

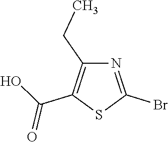

Methyl 2-bromo-4-ethyl-1,3-thiazole-5-carboxylate (150 mg, 600 μmol) was dissolved in 3 ml of THF. Aqueous sodium hydroxide solution (3 ml, 2.0 M, 6 mmol) was added to the solution and the mixture was stirred at room temperature overnight. The THF was removed on a rotary evaporator and the residue was acidified with 2 Nhydrochloric acid. The precipitated solid was filtered off and dried under high vacuum. This gave 100 mg (purity 98%, 69% of theory) of the target compound.

LC-MS (Methode 1): Rₜ=1.30 min; MS (ESIpos): m/z=235 [M+H]⁺.

### Example 78A

### 2-Bromo-N-[(3,5-difluoropyridin-2-yl)methyl]-4-ethyl-1,3-thiazole-5-carboxamide

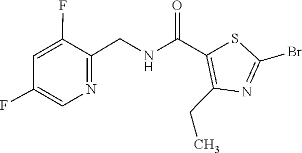 [SMILES: CCc1nc(Br)sc1C(=O)NCc1ncc(F)cc1F]

N,N-Diisopropylethylamine (300 μl, 1.7 mmol) and propylphosphonic anhydride (330 μl, 50% in ethyl acetate, 550 μmol) were added to a solution of 2-bromo-4-ethyl-1,3-thiazole-5-carboxylic acid (100 mg, 424 μmol) and 1-(3,5-difluoropyridin-2-yl)methanamine dihydrochloride (120 mg, 550 μmol) in 5.7 ml of acetonitrile, and the mixture was stirred at room temperature for 2 h. The reaction mixture was concentrated and the residue was taken up in ethyl acetate and washed with sat. NaHCO₃ solution, water and sat. NaCl solution. The organic phase was dried over Na₂SO₄. The drying agent was filtered off and the filtrate was concentrated. The residue was dried under high vacuum. This gave 150 mg (purity 95%, 93% of theory) of the target compound.

LC-MS (Methode 4): Rₜ=0.86 min; MS (ESIpos): m/z=364 [M+H]⁺.

### Example 79A

### diamix-tert-Butyl 4-(1,1-difluoro-5-azaspiro[2.5]octan-5-yl)-3-fluoropiperidine-1-carboxylate

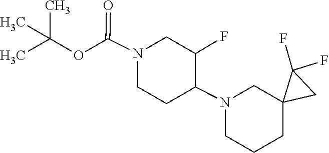 [SMILES: CC(C)(C)OC(=O)N1CCC(N2CCCC3(C2)CC3(F)F)C(F)C1]

N,N-Diisopropylethylamine (570 μl, 3.3 mmol) was added to a solution of rac-1,1-difluoro-5-azaspiro[2.5]octane hydrochloride (600 mg, 3.27 mmol) in 15 ml of 1,2-dichloroethane, and the mixture was stirred for 5 min, after which rac-tert-butyl 3-fluoro-4-oxopiperidine-1-carboxylate (355 mg, 1.63 mmol) and acetic acid (140 μl, 2.5 mmol) were added to the mixture. The mixture was then stirred at room temperature. After 5 h, sodium triacetoxyborohydride (416 mg, 1.96 mmol) was added to the mixture and the mixture was stirred at room temperature overnight. Sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: Phenomenex Kinetex C18 5 μm 100×30 mm. Mobile phase A: water, mobile phase B: acetonitrile, mobile phase C: 2% strength formic acid in water, mobile phase D: acetonitrile/water (80% by volume/20% by volume) total flow rate: 80 ml/min, room temperature, wavelength 200-400 nm, complete injection. Gradient profile: mobile phase A 0 to 2 min 70 ml, mobile phase B 0 to 2 min 0 ml, mobile phase A 2 to 10 min from 70 ml to 55 ml and mobile phase B from 0 ml to 15 ml, 10 to 12 min 0 ml of mobile phase A and 70 ml of mobile phase B. Mobile phase C and mobile phase D constant flow rate of 5 ml/min each over the entire running time). The product-containing fractions were combined and concentrated and the residue was dried under high vacuum. This gave 264 mg (purity 100%, 46% of theory) of the target compound.

LC-MS (Methode 4): Rₜ=0.56 min; MS (ESIpos): m/z=349 [M+H]⁺.

### Example 80A

### diamix-1,1-Difluoro-5-(3-fluoropiperidin-4-yl)-5-azaspiro[2.5]octane dihydrochloride

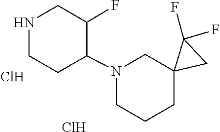 [SMILES: Cl ; FC1CNCCC1N1CCCC2(C1)CC2(F)F ; Cl]

4 M hydrochloric acid in 1,4-dioxane (950 μl, 4.0 M, 3.8 mmol) was added to a solution of diamix-tert-butyl 4-(1,1-difluoro-5-azaspiro[2.5]octan-5-yl)-3-fluoropiperidine-1-carboxylate (264 mg, 760 μmol) in 10 ml of dichloromethane, and the mixture was stirred at room temperature overnight. Subsequently, the reaction mixture was concentrated on a rotary evaporator and the residue was dried under high vacuum. This gave 246 mg of a mixture which was reacted further without further purification and analysis.

### Example 81A

### diamix-tert-Butyl 4-(5-azaspiro[2.5]octan-5-yl)-3-fluoropiperidine-1-carboxylate

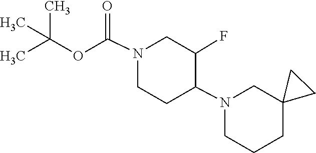 [SMILES: CC(C)(C)OC(=O)N1CCC(N2CCCC3(CC3)C2)C(F)C1]

N,N-Diisopropylethylamine (410 μl, 2.4 mmol) was added to a solution of 5-azaspiro[2.5]octane hydrochloride (350 mg, 2.37 mmol) in 10 ml of 1,2-dichloroethane, and the mixture was stirred for 5 min, after which rac-tert-butyl 3-fluoro-4-oxopiperidine-1-carboxylate (257 mg, 1.19 mmol) and acetic acid (100 μl, 1.8 mmol) were added to the mixture. The mixture was then stirred at room temperature. After 5 h, sodium triacetoxyborohydride (416 mg, 1.96 mmol) was added to the mixture and the mixture was stirred at room temperature overnight. Sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: Phenomenex Kinetex C18 5 μm 100×30 mm. mobile phase A: water, mobile phase B: acetonitrile, mobile phase C: 2% strength formic acid in water, mobile phase D: acetonitrile/water (80% by volume/20% by volume) total flow rate: 80 ml/min, room temperature, wavelength 200-400 nm, complete injection. Gradient profile: mobile phase A 0 to 2 min 70 ml, mobile phase B 0 to 2 min 0 ml, mobile phase A 2 to 10 min from 70 ml to 55 ml and mobile phase B from 0 ml to 15 ml, 10 to 12 min 0 ml of mobile phase A and 70 ml of mobile phase B. Mobile phase C and mobile phase D constant flow rate of 5 ml/min each over the entire running time). The product-containing fractions were combined and concentrated and the residue was dried under high vacuum. This gave 179 mg (purity 100%, 48% of theory) of the target compound.

LC-MS (Methode 4): Rₜ=0.53 min; MS (ESIpos): m/z=313 [M+H]⁺.

### Example 82A

### Ethyl 5-[(3R)-3-methyl[1,4′-bipiperidin]-1′-yl]-1,3,4-thiadiazole-2-carboxylate

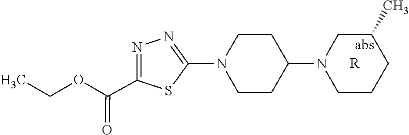

3.67 ml (21.09 mmol) of N,N-diisopropylethylamine were added to 1 g (4.22 mmol) of ethyl 5-bromo-1,3,4-thiadiazole-2-carboxylate and 1.077 g (4.22 mmol) of 1-(3,5-difluoropyridin-2-yl)methanamine dihydrochloride in 25 ml of acetonitrile, and the mixture was heated to 80° C. and stirred at this temperature overnight. After cooling of the reaction mixture, the solution was diluted with ethyl acetate and washed with water. The organic phase was finally separated off and the organic solution obtained was then filtered through hydrophobic filters (pleated filter MN 616 WA ¼, D=12.5 cm), dried and concentrated to dryness under reduced pressure. This gave 1.29 g (3.81 mmol, 90% of theory) of the target compound as a red solid.

¹H NMR (600 MHz, DMSO-d6) δ[ppm]: 0.77-0.87 (m, 4H, including at 0.82 (d, 3H)), 1.30 (t, 3H), 1.34-1.46 (m, 1H), 1.48-1.67 (m, 5H), 1.72-1.85 (m, 3H), 2.06 (br. t, 1H), 2.48-2.58 (m, 1H, partially obscured by DMSO), 2.74 (br. t, 2H), 3.24 (td, 2H), 3.98 (br. d, 2H), 4.34 (q, 2H).

LC-MS (Methode 1): Rₜ=0.82 min; m/z=339 (M+H)⁺.

### Example 83A

### 5-[(3R)-3-Methyl[1,4′-bipiperidin]-1′-yl]-1,3,4-thiadiazole-2-carboxylic acid

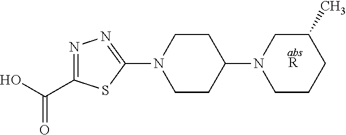

1.52 g (4.49 mmol) of ethyl 5-[(3R)-3-methyl[1,4′-bipiperidin]-1′-yl]-1,3,4-thiadiazole-2-carboxylate were dissolved in 8 ml of THF, 538 mg (22.45 mmol) of lithium hydroxide were added and 5 ml of water were then added to the reaction solution. The reaction solution was then stirred at room temperature for several hours. After complete conversion, the reaction solution was adjusted to pH 7 with 1 N HCl and concentrated to dryness on a rotary evaporator. This gave 2.95 g of an amber oil which was purified by column chromatography.

Conditions: The separation was carried out using portions of about 1 g. RP column Chromatorex C18, 10 μm; 125×30 mm, acetonitrile/water 10/90→gradient over 38 min 4 acetonitrile/water 90/10, flow rate 75 ml/min.

Finally, product-containing fractions were combined and concentrated to dryness under reduced pressure and dried. This gave 487 mg (1.57 mmol, 35% of theory) of the target compound as a white solid.

LC-MS (Methode 1): Rₜ=0.39 min; m/z=311 (M+H)⁺.

### WORKING EXAMPLES

### Example 1

### N-[(3,5-Difluoropyridin-2-yl)methyl]-2-[(3R)-3-methyl[1,4′-bipiperidin]-1′-yl]-1,3-thiazole-5-carboxamide

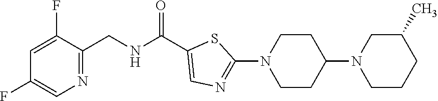 [SMILES: C[C@@H]1CCCN(C2CCN(c3ncc(C(=O)NCc4ncc(F)cc4F)s3)CC2)C1]

13 g (38.91 mmol) of 2-bromo-N-[(3,5-difluoropyridin-2-yl)methyl]-1,3-thiazole-5-carboxamide, 8.51 g (38.91 mmol) of (3R)-3-methyl-1,4′-bipiperidine hydrochloride (1:1) (CAS Registry Number 1799475-27-6) and 20.62 g (194.53 mmol) of sodium carbonate in 200 ml of water were heated to 120° C. and stirred at this temperature overnight. After cooling of the reaction mixture, the solution was extracted with ethyl acetate. The separated organic phase was subsequently filtered through a hydrophobic filter (pleated filter MN 616 WA ¼, D=12.5 cm), dried and concentrated to dryness on a rotary evaporator. The residue obtained was taken up in acetonitrile, heated to 80° C. and, with stirring, slowly cooled back to room temperature. The precipitated solid was filtered off with suction and washed with acetonitrile. The residue was then once more taken up in acetonitrile and recrystallized again. This gave 10.75 g (24.68 mmol, 63% of theory) of the target compound as a light-beige solid. The two mother liquors were combined and concentrated to dryness on a rotary evaporator. The residue obtained was purified further by column chromatography on silica gel (Isolera Biotage SNAP-Ultra 100 g column, mobile phase: dichloromethane→gradient over 20 CV (CV=column volumes)→dichloromethane/methanol 9:1). The product fractions obtained were then combined, concentrated on a rotary evaporator and recrystallized from acetonitrile. This gave a further 3.28 g (7.48 mmol, 19% of theory) of the target compound as a light-beige solid.

¹H-NMR (600 MHz, DMSO-d₆, S/ppm): 0.76-0.86 (m, 4H, including at 0.82 (d, 3H)), 1.34-1.66 (m, 6H), 1.71-1.81 (m, 3H), 2.01-2.09 (m, 1H), 2.44-2.56 (m, 1H, partially obscured by DMSO), 2.69-2.77 (m, 2H), 3.04 (td, 2H), 3.93 (br. d, 2H), 4.53 (br. d, 2H), 7.83 (s, 1H), 7.88-7.95 (m, 1H), 8.47 (d, 1H), 8.71 (t, 1H).

LC-MS (Methode 4): Rₜ=0.50 min; m/z=436 (M+H)⁺.

[α]_{D}²⁰=−8.06° (c=0.430, methanol).

### Example 2

### N-[(3,5-Difluoropyridin-2-yl)methyl]-2-[4-(3,4-dihydroisoquinolin-2(1H)-yl)piperidin-1-yl]-1,3-thiazole-5-carboxamide

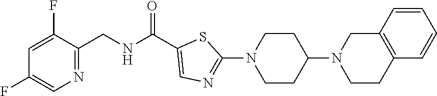 [SMILES: O=C(NCc1ncc(F)cc1F)c1cnc(N2CCC(N3CCc4ccccc4C3)CC2)s1]

60 mg (0.18 mmol) of 2-bromo-N-[(3,5-difluoropyridin-2-yl)methyl]-1,3-thiazole-5-carboxamide, 51 mg (0.18 mmol) of 2-(piperidin-4-yl)-1,2,3,4-tetrahydroisoquinoline dihydrochloride and 95 mg (0.9 mmol) of sodium carbonate in 1 ml of water in a closed vessel were heated to 160° C. and stirred at this temperature for 30 min. After cooling of the reaction mixture, water was added and the solution was extracted with dichloromethane. The separated organic phase was subsequently filtered through a hydrophobic filter (pleated filter MN 616 WA ¼, D=12.5 cm), dried and concentrated to dryness on a rotary evaporator. The residue obtained was purified further by column chromatography on silica gel (Isolera Biotage SNAP-Ultra 10 g column, mobile phase: ethyl acetate→gradient over 5 CV (CV=column volumes)→ethyl acetate/methanol 95:5). The product fractions obtained were then combined and concentrated to dryness on a rotary evaporator. This gave 62.7 mg (0.13 mmol, 74% of theory) of the target compound as a yellow solid.

¹H-NMR (600 MHz, DMSO-d₆, δ/ppm): 1.55-1.65 (m, 2H), 1.86-1.94 (m, 2H), 2.67-2.73 (m, 1H), 2.73-2.81 (m, 4H), 3.12 (br. t, 2H), 3.70 (s, 2H), 3.97 (br. d, 2H), 4.53 (br. d, 2H), 7.01-7.12 (m, 4H), 7.85 (s, 1H), 7.93 (td, 1H), 8.48 (d, 1H), 8.76 (t, 1H).

LC-MS (Methode 1): Rₜ=0.97 min; m/z=470 (M+H)⁺.

### Example 3

### 2-[3-(Cyclopropylmethyl)[1,4′-bipiperidin]-1′-yl]-N-[(3,5-difluoropyridin-2-yl)methyl]-1,3-thiazole-5-carboxamide (racemate)

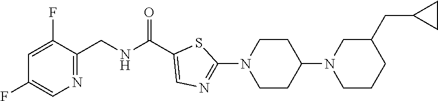 [SMILES: O=C(NCc1ncc(F)cc1F)c1cnc(N2CCC(N3CCCC(CC4CC4)C3)CC2)s1]

32 mg (0.10 mmol) of 2-bromo-N-[(3,5-difluoropyridin-2-yl)methyl]-1,3-thiazole-5-carboxamide, 22 mg (0.10 mmol) of 3-(cyclopropylmethyl)-1,4′-bipiperidine (racemate) and 31 mg (0.29 mmol) of sodium carbonate in 1 ml of water in a closed vessel were heated to 120° C. and stirred at this temperature for 30 min. After cooling of the reaction mixture the solution was extracted with dichloromethane. The separated organic phase was subsequently filtered through a hydrophobic filter (pleated filter MN 616 WA ¼, D=12.5 cm), dried and concentrated to dryness on a rotary evaporator. The residue obtained was purified using the following method.

Method 7: Instrument: Waters Prep LC/MS System, column: XBridge C18 5 μm 100×30 mm

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time.

This gave 40.8 mg (0.09 mmol, 88% of theory) of the target compound as a white lyophylisate.

¹H-NMR (400 MHz, DMSO-d₆, δ/ppm): −0.07-0.03 (m, 2H), 0.34-0.43 (m, 2H), 0.60-0.73 (m, 1H), 0.80-0.94 (m, 1H), 0.99-1.14 (m, 2H), 1.32-1.65 (m, 5H), 1.68-1.91 (m, 4H), 2.02-2.14 (m, 1H), 2.44-2.59 (m, 1H, partially obscured by DMSO), 2.73 (br. d, 1H), 2.83 (br. d, 1H), 3.04 (br. t, 2H), 3.94 (br. d, 2H), 4.52 (br. d, 2H), 7.83 (s, 1H), 7.87-7.96 (m, 1H), 8.47 (d, 1H), 8.71 (t, 1H).

LC-MS (Methode 1): Rₜ=1.13 min; m/z=476 (M+H)⁺.

Analogously to Examples 1 to 3, the following compounds of Examples 4 to 14 were prepared from the starting materials stated in each case:

| Example | Name/Structure/Starting materials | Analytical data |
|---|---|---|
| 4 | 2-[3-(difluoromethyl)[1,4′-bipiperidin]-1′-yl]-N- [(3,5-difluoropyridin-2-yl)methyl]-1,3-thiazole- 5-carboxamide (racemate) 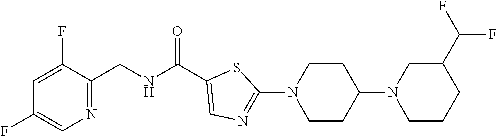 [SMILES: O=C(NCc1ncc(F)cc1F)c1cnc(N2CCC(N3CCCC(C(F)F)C3)CC2)s1] from 2-bromo-N-[(3,5-difluoropyridin-2- | ¹H-NMR (600 MHz, DMSO-d₆, δ/ppm): 1.11-1.21 (m, 1H), 1.37- 1.53 (m, 3H), 1.62-1.72 (m, 2H), 1.73-1.81 (m, 2H), 1.88-1.98 (m, 1H), 2.10-2.21 (m, 2H), 2.46-2.60 (m, 1H, partially obscured by DMSO), 2.72 (br. d, 1H), 2.79 (br. d, 1H), 3.05 (td, 2H), 3.94 (br. d, 2H), 4.53 (br. d, 2H), 5.82-6.06 (m, 1H), 7.84 (s, 1H), 7.93 (td, 1H), 8.47 (d, 1H), 8.75 (t, 1H). LC-MS (Methode 5): Rₜ = 1.51 min; m/z = 472 (M + H)⁺. |
| | yl)methyl]-1,3-thiazole-5-carboxamide and 3- | |
| | (difluoromethyl)-1,4′-bipiperidine | |
| | dihydrochloride (racemate) | |
| 5 | N-[(3,5-difluoropyridin-2-yl)methyl]-2-[3- (trifluoromethyl)[1,4′-bipiperidin]-1′-yl]-1,3- thiazole-5-carboxamide (racemate) 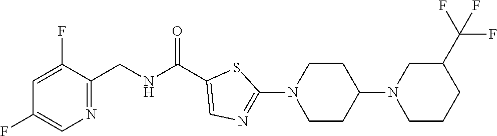 [SMILES: O=C(NCc1ncc(F)cc1F)c1cnc(N2CCC(N3CCCC(C(F)(F)F)C3)CC2)s1] from 2-bromo-N-[(3,5-difluoropyridin-2- | ¹H-NMR (500 MHz, DMSO-d₆, δ/ppm): 1.15-1.27 (m, 1H), 1.38- 1.56 (m, 3H), 1.65-1.73 (m, 1H), 1.74-1.82 (m, 2H), 1.82-1.88 (m, 1H), 2.06-2.20 (m, 2H), 2.32-2.44 (m, 1H), 2.57-2.66 (m, 1H), 2.81 (br. d, 1H), 2.96 (br. d, 1H), 3.00- 3.10 (m, 2H), 3.95 (br. d, 2H), 4.53 (br. d, 2H), 7.83 (s, 1H), 7.88-7.95 (m, 1H), 8.47 (d, 1H), 8.71 (t, 1H). LC-MS (Methode 5): Rₜ = 1.63 min; m/z = 490 (M + H)⁺. |
| | yl)methyl]-1,3-thiazole-5-carboxamide and 3- | |
| | (trifluoromethyl)-1,4′-bipiperidine | |
| | dihydrochloride (racemate) | |
| 6 | N-[(3,5-difluoropyridin-2-yl)methyl]-2-[3- (fluoromethyl)[1,4′-bipiperidin]-1′-yl]-1,3- thiazole-5-carboxamide (racemate) 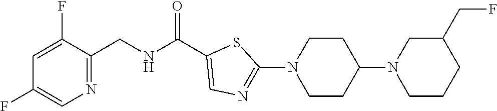 [SMILES: O=C(NCc1ncc(F)cc1F)c1cnc(N2CCC(N3CCCC(CF)C3)CC2)s1] from 2-bromo-N-[(3,5-difluoropyridin-2- yl)methyl]-1,3-thiazole-5-carboxamide and 3- (fluoromethyl)-1,4′-bipiperidine dihydrochloride | ¹H-NMR (500 MHz, DMSO-d₆, δ/ppm): 0.95-1.07 (m, 1H), 1.37- 1.54 (m, 3H), 1.61 (br. d, 2H), 1.73-1.91 (m, 3H), 2.02 (t, 1H), 2.15 (t, 1H), 2.47-2.57 (m, 1H, partially obscured by DMSO), 2.68-2.75 (m, 1H), 2.80 (br. d, 1H), 3.01-3.10 (m, 2H), 3.94 (br. d, 2H), 4.21-4.29 (m, 1H), 4.31- 4.39 (m, 1H), 4.53 (br. d, 2H), 7.83 (s, 1H), 7.88-7.94 (m, 1H), 8.47 (d, 1H), 8.71 (t, 1H). LC-MS (Methode 5): Rₜ = 1.48 min; m/z = 454 (M + H)⁺. |
| | (racemate) | |
| 7 | 2-{3-[(3,3-difluorocyclobutyl)methoxy][1,4′- bipiperidin]-1′-yl}-N-[(3,5-difluoropyridin-2- yl)methyl]-1,3-thiazole-5-carboxamide (racemate) 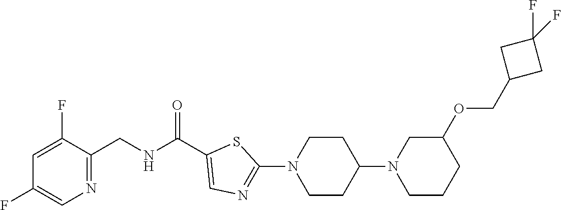 [SMILES: O=C(NCc1ncc(F)cc1F)c1cnc(N2CCC(N3CCCC(OCC4CC(F)(F)C4)C3)CC2)s1] | ¹H-NMR (500 MHz, DMSO-d₆, δ/ppm): 1.02-1.12 (m, 1H), 1.30- 1.40 (m, 1H), 1.44-1.54 (m, 2H), 1.60-1.67 (m, 1H), 1.73-1.80 (m, 2H), 1.87-1.93 (m, 1H), 1.98 (br. t, 1H), 2.06-2.14 (m, 1H), 2.24-2.35 (m, 3H), 2.48-2.62 (m, 3H, partially obscured by DMSO), 2.62-2.68 (m, 1H), 2.95 (br. d, 1H), 3.04 (br. t, 2H), 3.24-3.30 (m, 1H), 3.41-3.50 (m, 2H), 3.94 (br. d, 2H), 4.53 (br. d, 2H), 7.83 (s, 1H), 7.88-7.95 (m, 1H), 8.47 (d, 1H), 8.71 (t, 1H). LC-MS (Methode 1): Rₜ = 1.11 min; m/z = 542 (M + H)⁺. |
| | from 2-bromo-N-[(3,5-difluoropyridin-2- | |
| | yl)methyl]-1,3-thiazole-5-carboxamide and 3- | |
| | [(3,3-difluorocyclobutyl)methoxy]-1,4′- | |
| | bipiperidine (racemate) | |
| 8 | N-[(3,5-difluoropyridin-2-yl)methyl]-4-methyl-2- [(3R)-3-methyl[1,4′-bipiperidin]-1′-yl]-1,3- thiazole-5-carboxamide 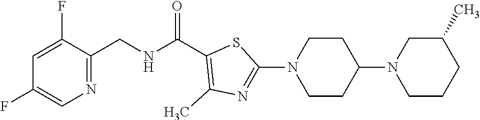 [SMILES: Cc1nc(N2CCC(N3CCC[C@@H](C)C3)CC2)sc1C(=O)NCc1ncc(F)cc1F] from 2-bromo-N-[(3,5-difluoropyridin-2- | ¹H-NMR (500 MHz, DMSO-d₆, δ/ppm): 0.76-0.88 (m, 4H, including at 0.82 (d, 3H)), 1.34- 1.67 (m, 6H), 1.71-1.82 (m, 3H), 2.05 (br. t, 1H), 2.38 (s, 3H), 2.44- 2.56 (m, 1H, partially obscured by DMSO), 2.70-2.78 (m, 2H), 3.02 (br. t, 2H), 3.90 (br. d, 2H), 4.50 (br. d, 2H), 7.86-7.93 (m, 1H), 8.01 (t, 1H), 8.46 (d, 1H). LC-MS (Methode 1): Rₜ = 0.98 min; m/z = 450 (M + H)⁺. |
| | yl)methyl]-4-methyl-1,3-thiazole-5-carboxamide | |
| | and (3R)-3-methyl-1,4′-bipiperidine | |
| | dihydrochloride | |
| 9 | N-[(3,5-difluoropyridin-2-yl)methyl]-5-methyl-2- [(3R)-3-methyl[1,4′-bipiperidin]-1′-yl]-1,3- thiazole-4-carboxamide 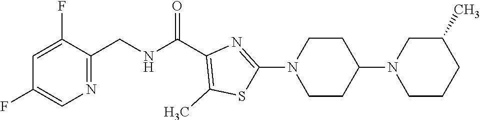 [SMILES: Cc1sc(N2CCC(N3CCC[C@@H](C)C3)CC2)nc1C(=O)NCc1ncc(F)cc1F] from 2-bromo-N-[(3,5-difluoropyridin-2- | ¹H-NMR (400 MHz, DMSO-d₆, δ/ppm): 0.73-0.92 (m, 4H, including at 0.82 (d, 3H)), 1.32- 1.68 (m, 6H), 1.70-1.86 (m, 3H), 1.97-2.14 (m, 1H), 2.38 (s, 3H), 2.44-2.58 (m, 1H, partially obscured by DMSO), 2.69-2.82 (m, 2H), 3.03 (br. t, 2H), 3.90 (br. d, 2H), 4.50 (br. d, 2H), 7.86-7.95 (m, 1H), 8.02 (br. t, 1H), 8.46 (d, 1H). LC-MS (Methode 1): Rₜ = 0.92 min; m/z = 450 (M + H)⁺. |
| | yl)methyl]-5-methyl-1,3-thiazole-4-carboxamide | |
| | and (3R)-3-methyl-1,4′-bipiperidine | |
| | dihydrochloride | |
| 10 | N-[(3,5-difluoropyridin-2-yl)methyl]-2-[(3R)-3- methyl[1,4′-bipiperidin]-1′-yl]-1,3-thiazole-4- carboxamide 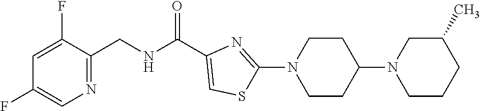 [SMILES: C[C@@H]1CCCN(C2CCN(c3nc(C(=O)NCc4ncc(F)cc4F)cs3)CC2)C1] from 2-bromo-N-[(3,5-difluoropyridin-2- yl)methyl]-1,3-thiazole-4-carboxamide and (3R)- | ¹H-NMR (600 MHz, DMSO-d₆, δ/ppm): 0.76-0.87 (m, 4H, including at 0.82 (d, 3H)), 1.36- 1.45 (m, 1H), 1.46-1.55 (m, 3H), 1.56-1.67 (m, 2H), 1.73-1.84 (m, 3H), 2.06 (br. t, 1H), 2.44-2.56 (m, 1H, partially obscured by DMSO), 2.71-2.80 (m, 2H), 3.02 (td, 2H), 3.97 (br. d, 2H), 4.58 (d, 2H), 7.38 (s, 1H), 7.89-7.95 (m, 1H), 8.46 (d, 1H), 8.48 (t, 1H). LC-MS (Methode 4): Rₜ = 0.56 min; m/z = 436 (M + H)⁺. |
| | 3-methyl-1,4′-bipiperidine dihydrochloride | |
| 11 | N-[(3,5-difluoropyridin-2-yl)methyl]-2-[(3R)-3- methyl[1,4′-bipiperidin]-1′-yl]-4- (trifluoromethyl)-1,3-thiazole-5-carboxamide 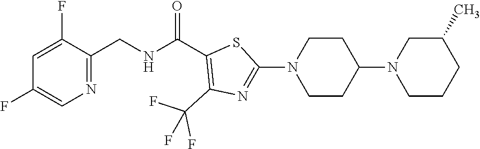 [SMILES: C[C@@H]1CCCN(C2CCN(c3nc(C(F)(F)F)c(C(=O)NCc4ncc(F)cc4F)s3)CC2)C1] | ¹H-NMR (600 MHz, DMSO-d₆, δ/ppm): 0.77-0.86 (m, 4H, including at 0.82 (d, 3H)), 1.34- 1.45 (m, 1H), 1.45-1.55 (m, 3H), 1.55-1.66 (m, 2H), 1.75 (t, 1H), 1.80 (br. d, 2H), 2.05 (td, 1H), 2.45-2.56 (m, 1H, partially obscured by DMSO), 2.69-2.79 (m, 2H), 3.09 (td, 2H), 3.88 (br. d, 2H), 4.52 (br. d, 2H), 7.89-7.97 (m, 1H), 8.47 (d, 1H), 8.90 (t, 1H). LC-MS (Methode 1): Rₜ = 1.24 min; m/z = 504 (M + H)⁺ |
| | from 2-bromo-N-[(3,5-difluoropyridin-2- | |
| | yl)methyl]-4-(trifluoromethyl)-1,3-thiazole-5- | |
| | carboxamide and (3R)-3-methyl-1,4′-bipiperidine | |
| | dihydrochloride | |
| 12 | N-[(3,5-difluoropyridin-2-yl)methyl]-5-ethyl-2- [(3R)-3-methyl[1,4′-bipiperidin]-1′-yl]-1,3- thiazole-4-carboxamide 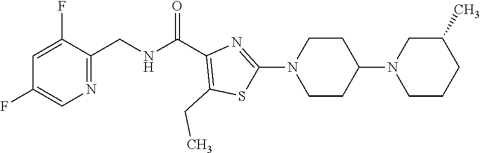 [SMILES: CCc1sc(N2CCC(N3CCC[C@@H](C)C3)CC2)nc1C(=O)NCc1ncc(F)cc1F] from 2-bromo-N-[(3,5-difluoropyridin-2- | 1H-NMR (600 MHz, DMSO-d₆, δ/ppm): 0.77-0.87 (m, 4H, including at 0.82 (d, 3H)), 1.13 (t, 3H), 1.35-1.45 (m, 1H), 1.45-1.56 (m, 3H), 1.56-1.67 (m, 2H), 1.71- 1.80 (m, 3H), 2.06 (br. t, 1H), 2.42-2.52 (m, 1H, partially obscured by DMSO), 2.75 (br. t, 2H), 2.96 (td, 2H), 3.10 (q, 2H), 3.91 (br. d, 2H), 4.56 (d, 2H), 7.88-7.95 (m, 1H), 8.44 (t, 1H), 8.46 (d, 1H). LC-MS (Methode 4): Rₜ = 0.68 min; m/z = 464 (M + H)⁺. |
| | yl)methyl]-5-ethyl-1,3-thiazole-4-carboxamide | |
| | and (3R)-3-methyl-1,4′-bipiperidine | |
| | dihydrochloride | |
| 13 | N-[(3,5-difluoropyridin-2-yl)methyl]-2-[(3R)-3- methyl[1,4′-bipiperidin]-1′-yl]-1,3-oxazole-4- carboxamide 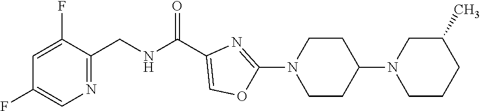 [SMILES: C[C@@H]1CCCN(C2CCN(c3nc(C(=O)NCc4ncc(F)cc4F)co3)CC2)C1] from 2-bromo-N-[(3,5-difluoropyridin-2- | ¹H-NMR (600 MHz, DMSO-d₆, δ/ppm): 0.76-0.88 (m, 4H, including at 0.82 (d, 3H)), 1.33- 1.67 (m, 6H), 1.71-1.82 (m, 3H), 2.01-2.10 (m, 1H), 2.40-2.56 (m, 1H, partially obscured by DMSO), 2.69-2.79 (m, 2H), 2.97 (br. t, 2H), 3.99 (br. d, 2H), 4.56 (d, 2H), 7.89-7.95 (m, 1H), 8.02 (s, 1H), 8.24 (t, 1H), 8.47 (d, 1H). LC-MS (Methode 4): Rₜ = 0.52 min; m/z = 420 (M + H)⁺. |
| | yl)methyl]-1,3-oxazole-4-carboxamide and (3R)- | |
| | 3-methyl-1,4′-bipiperidine dihydrochloride | |
| 14 | N-[(3,5-difluoropyridin-2-yl)methyl]-5-methyl-2- [(3R)-3-methyl[1,4′-bipiperidin]-1′-yl]-1,3- oxazole-4-carboxamide 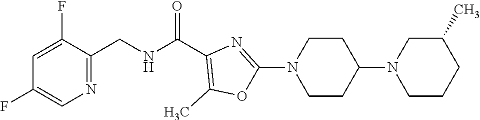 [SMILES: Cc1oc(N2CCC(N3CCC[C@@H](C)C3)CC2)nc1C(=O)NCc1ncc(F)cc1F] from 2-bromo-N-[(3,5-difluoropyridin-2- | ¹H-NMR (600 MHz, DMSO-d₆, δ/ppm): 0.77-0.87 (m, 4H, including at 0.82 (d, 3H)), 1.35- 1.67 (m, 6H), 1.76 (br. d, 3H), 2.05 (br. t, 1H), 2.36-2.58 (m, 4H, partially obscured by DMSO, including at 2.50 (br. s, 3H)), 2.70- 2.80 (m, 2H), 2.92 (br. t, 2H), 3.94 (br. d, 2H), 4.54 (br. d, 2H), 7.91 (br. t, 1H), 8.09 (br. t, 1H), 8.47 (br. s, 1H). LC-MS (Methode 4): Rₜ = 0.57 min; m/z = 434 (M + H)⁺. |
| | yl)methyl]-5-methyl-1,3-oxazole-4-carboxamide | |
| | and (3R)-3-methyl-1,4′-bipiperidine | |
| | dihydrochloride | |

### Example 15

### N-[(3,5-difluoropyridin-2-yl)methyl]-2-[(3R)-3-methoxy[1,4′-bipiperidin]-1′-yl]-1,3-thiazole-5-carboxamide

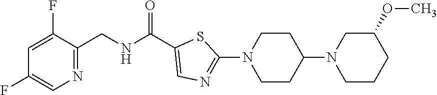 [SMILES: CO[C@@H]1CCCN(C2CCN(c3ncc(C(=O)NCc4ncc(F)cc4F)s3)CC2)C1]

100 mg (0.28 mmol) of N-[(3,5-difluoropyridin-2-yl)methyl]-2-(4-oxopiperidin-1-yl)-1,3-thiazole-5-carboxamide were dissolved in 5 ml of dichloromethane, and 65 mg (0.57 mmol) of (3R)-3-methoxypiperidine and 24 μl (0.43 mmol) of glacial acetic acid were added. 72 mg (0.34 mmol) of sodium acetoxyborohydride were then metered in and stirring of the reaction solution was then continued at room temperature overnight. Subsequently, the reaction mixture was diluted with dichloromethane and washed with sodium hydrogencarbonate solution. The organic phase was finally separated off and the organic solution obtained was then filtered through hydrophobic filters (pleated filter MN 616 WA ¼, D=12.5 cm), dried and concentrated to dryness under reduced pressure. The residue obtained was purified using the following method.

Method 8:

Instrument: Waters Prep LC/MS System, column: XBridge C18 5 μm 100×30 mm

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 63 ml, mobile phase B 0 to 2 min 7 ml, mobile phase A 2 to 10 min from 63 ml to 39 ml and mobile phase B from 7 ml to 31 ml, 10 to 12 min 0 ml of mobile phase A and 70 ml of mobile phase B. Mobile phase C and mobile phase D constant flow rate of 5 ml/min each over the entire running time.

This gave 62 mg (0.14 mmol, 48% of theory) of the target compound as a white lyophylisate.

¹H-NMR (600 MHz, DMSO-d₆, δ/ppm): 1.00-1.11 (m, 1H), 1.30-1.40 (m, 1H), 1.43-1.54 (m, 2H), 1.59-1.66 (m, 1H), 1.77 (br. d, 2H), 1.86-1.93 (m, 1H), 1.98 (t, 1H), 2.11 (t, 1H), 2.47-2.58 (m, 1H, partially obscured by DMSO), 2.64 (br. d, 1H), 2.94 (br. d, 1H), 3.04 (br. t, 2H), 3.12-3.19 (m, 1H), 3.23 (s, 3H), 3.94 (br. d, 2H), 4.53 (br. d, 2H), 7.83 (s, 1H), 7.91 (td, 1H), 8.47 (d, 1H), 8.71 (t, 1H).

LC-MS (Methode 1): Rₜ=0.83 min; m/z=452 (M+H)⁺.

### Example 16

### 2-[3-(Difluoromethoxy)[1,4′-bipiperidin]-1′-yl]-N-[(3,5-difluoropyridin-2-yl)methyl]-1,3-thiazole-5-carboxamide (racemate)

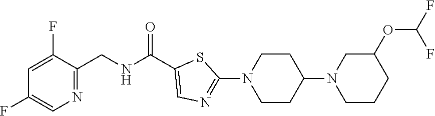 [SMILES: O=C(NCc1ncc(F)cc1F)c1cnc(N2CCC(N3CCCC(OC(F)F)C3)CC2)s1]

100 mg (0.28 mmol) of N-[(3,5-difluoropyridin-2-yl)methyl]-2-(4-oxopiperidin-1-yl)-1,3-thiazole-5-carboxamide were dissolved in 5 ml of dichloromethane, and 86 mg (0.57 mmol) of 3-(difluoromethoxy)piperidine (racemate) and 24 μl (0.43 mmol) of glacial acetic acid were added. 72 mg (0.34 mmol) of sodium acetoxyborohydride were then metered in and stirring of the reaction solution was then continued at room temperature overnight. Subsequently, the reaction mixture was diluted with dichloromethane and washed with sodium hydrogencarbonate solution. The organic phase was finally separated off and the organic solution obtained was then filtered through hydrophobic filters (pleated filter MN 616 WA ¼, D=12.5 cm), dried and concentrated to dryness under reduced pressure. The residue obtained was purified using the following method.

Method 9:

Instrument: Waters Prep LC/MS System, column: XBridge C18 5 μm 100×30 mm

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time.

This gave 60 mg (0.12 mmol, 44% of theory) of the target compound as a white lyophylisate.

¹H-NMR (600 MHz, DMSO-d₆, δ/ppm): 1.27-1.36 (m, 1H), 1.36-1.53 (m, 3H), 1.62-1.69 (m, 1H), 1.73-1.81 (m, 2H), 1.85-1.93 (m, 1H), 2.13-2.25 (m, 2H), 2.54-2.67 (m, 2H), 2.90 (br. d, 1H), 3.05 (br. t, 2H), 3.94 (br. d, 2H), 4.01-4.08 (m, 1H), 4.53 (d, 2H), 6.57-6.88 (m, 1H), 7.83 (s, 1H), 7.91 (t, 1H), 8.47 (d, 1H), 8.72 (t, 1H).

LC-MS (Methode 1): Rₜ=0.91 min; m/z=488 (M+H)⁺.

### Example 17

### N-[(3,5-Difluoropyridin-2-yl)methyl]-2-(3-ethyl[1,4′-bipiperidin]-1′-yl)-1,3-thiazole-5-carboxamide (racemate)

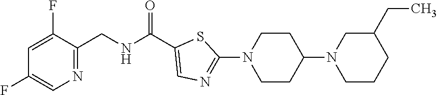 [SMILES: CCC1CCCN(C2CCN(c3ncc(C(=O)NCc4ncc(F)cc4F)s3)CC2)C1]

100 mg (0.28 mmol) of N-[(3,5-difluoropyridin-2-yl)methyl]-2-(4-oxopiperidin-1-yl)-1,3-thiazole-5-carboxamide were dissolved in 5 ml of dichloromethane, and 64 mg (0.57 mmol) of 3-ethylpiperidine (racemate) and 24 μl (0.43 mmol) of glacial acetic acid were added. 72 mg (0.34 mmol) of sodium acetoxyborohydride were then metered in and stirring of the reaction solution was then continued at room temperature overnight. Subsequently, the reaction mixture was diluted with dichloromethane and washed with sodium hydrogencarbonate solution. The organic phase was finally separated off and the organic solution obtained was then filtered through hydrophobic filters (pleated filter MN 616 WA ¼, D=12.5 cm), dried and concentrated to dryness under reduced pressure. The residue obtained was purified using the following method.

Method 7:

Instrument: Waters Prep LC/MS System, column: XBridge C18 5 μm 100×30 mm

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time.

This gave 46 mg (0.10 mmol, 36% of theory) of the target compound as a white lyophylisate.

¹H-NMR (600 MHz, DMSO-d₆, δ/ppm): 0.76-0.87 (m, 4H, including at 0.85 (t, 3H)), 1.09-1.25 (m, 2H), 1.26-1.34 (m, 1H), 1.34-1.43 (m, 1H), 1.44-1.53 (m, 2H), 1.55-1.62 (m, 1H), 1.65-1.71 (m, 1H), 1.73-1.83 (m, 3H), 2.08 (br. t 1H), 2.46-2.56 (m, 1H, partially obscured by DMSO), 2.70-2.79 (m, 2H), 3.04 (br. t, 2H), 3.94 (br. d, 2H), 4.53 (br. d, 2H), 7.82 (s, 1H), 7.89 (br. t, 1H), 8.46 (d, 1H), 8.67 (t, 1H).

LC-MS (Methode 1): Rₜ=0.99 min; m/z=450 (M+H)⁺.

### Example 18

### 2-[(3R)-3-Methyl[1,4′-bipiperidin]-1′-yl]-N-{[4-(trifluoromethyl)pyridin-2-yl]methyl}-1,3-thiazole-5-carboxamide

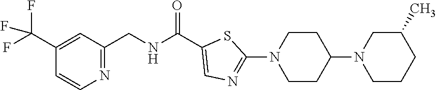 [SMILES: C[C@@H]1CCCN(C2CCN(c3ncc(C(=O)NCc4cc(C(F)(F)F)ccn4)s3)CC2)C1]

0.46 ml (2.62 mmol) of N,N-diisopropylethylamine was added to 200 mg (0.52 mmol) of 2-[(3R)-3-methyl[1,4′-bipiperidin]-1′-yl]-1,3-thiazole-5-carboxylic acid dihydrochloride and 122 mg (0.58 mmol) of 1-[4-(trifluoromethyl)pyridin-2-yl]methanamine hydrochloride (1:1) in 20 ml of acetonitrile, and 0.34 ml (0.58 mmol) of a 50% strength solution of T3P (2,4,6-tripropyl-1,3,5,2,4,6-trioxatriphosphorinane 2,4,6-trioxide) in ethyl acetate was then added dropwise to the reaction solution at room temperature. After the addition had ended, the reaction solution was stirred at room temperature overnight. The reaction mixture was then extracted with water and with dichloromethane. The organic phase was finally separated off and the organic solution obtained was then filtered through hydrophobic filters (pleated filter MN 616 WA ¼, D=12.5 cm), dried and concentrated to dryness under reduced pressure. The residue obtained was purified using the following method.

Method 7:

Instrument: Waters Prep LC/MS System, column: XBridge C18 5 μm 100×30 mm

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time.

This gave 55 mg (0.12 mmol, 23% of theory) of the target compound as a white lyophylisate.

¹H-NMR (400 MHz, DMSO-d₆, δ/ppm): 0.74-0.89 (m, 4H, including at 0.82 (d, 3H)), 1.34-1.68 (m, 6H), 1.70-1.84 (m, 3H), 1.99-2.11 (m, 1H), 2.44-2.58 (m, 1H, partially obscured by DMSO), 2.69-2.80 (m, 2H), 3.06 (td, 2H), 3.95 (br. d, 2H), 4.59 (d, 2H), 7.62 (s, 1H), 7.67 (d, 1H), 7.87 (s, 1H), 8.81 (d, 1H), 8.89 (t, 1H).

LC-MS (Methode 1): Rₜ=1.05 min; m/z=469 (M+H)⁺.

### Example 19

### 2-[(3R)-3-Methyl[1,4′-bipiperidin]-1′-yl]-N-[3-(trifluoromethyl)benzyl]-1,3-thiazole-5-carboxamide

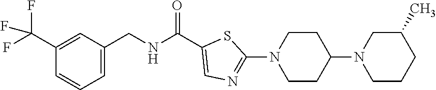 [SMILES: C[C@@H]1CCCN(C2CCN(c3ncc(C(=O)NCc4cccc(C(F)(F)F)c4)s3)CC2)C1]

100 mg (0.26 mmol) of 2-[(3R)-3-methyl[1,4′-bipiperidin]-1′-yl]-1,3-thiazole-5-carboxylic acid dihydrochloride were dissolved in 10 ml of dichloromethane, 56 mg (0.42 mmol) of 1-chloro-N,N,2-trimethylprop-1-en-1-amine were added and the mixture was stirred at room temperature for 30 min. Subsequently, 60 μl of pyridine and then 46 mg (0.26 mmol) of 1-[3-(trifluoromethyl)phenyl]methanamine were metered into the reaction solution and the mixture was stirred at room temperature overnight. After addition of water, the resulting precipitate was filtered off with suction. The biphasic filtrate obtained was separated off and the resulting organic phase was filtered through hydrophobic filters (pleated filter MN 616 WA ¼, D=12.5 cm), dried and concentrated to dryness under reduced pressure. The residue obtained was purified using the following method.

Method 11:

Instrument: Abimed Gilson 305; column: Reprosil C18 10 μm, 250 mm×30 mm; mobile phase A: water, mobile phase B: acetonitrile; gradient: 0-3 min 10% B, 3-27 min 10% B→95% B, 27-34.5 min 95% B, 34.5-35.5 min 95% B→10% B, 35.5-36.5 min 10% B; flow rate: 50 ml/min; room temperature; UV detection: 210 nm.

This gave 45 mg (0.10 mmol, 37% of theory) of the target compound.

¹H-NMR (600 MHz, DMSO-d₆, δ/ppm): 0.78-0.91 (m, 4H, including at 0.83 (d, 3H)), 1.37-1.69 (m, 6H), 1.73-1.94 (m, 3H), 2.05-2.23 (m, 1H), 2.56-2.67 (m, 1H), 2.73-2.90 (m, 2H), 3.06 (br. t, 2H), 3.96 (br. d, 2H), 4.48 (d, 2H), 7.54-7.65 (m, 4H), 7.84 (s, 1H), 8.84 (t, 1H).

LC-MS (Methode 1): Rₜ=1.31 min; m/z=467 (M+H)⁺.

### Example 20

### N-[(3-Fluoropyridin-2-yl)methyl]-2-[(3R)-3-methyl[1,4′-bipiperidin]-1′-yl]-1,3-thiazole-5-carboxamide

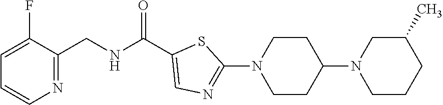 [SMILES: C[C@@H]1CCCN(C2CCN(c3ncc(C(=O)NCc4ncccc4F)s3)CC2)C1]

0.18 ml (1.05 mmol) of N,N-diisopropylethylamine was added to 100 mg (0.26 mmol) of 2-[(3R)-3-methyl[1,4′-bipiperidin]-1′-yl]-1,3-thiazole-5-carboxylic acid dihydrochloride and 47 mg (0.29 mmol) of 1-(3-fluoropyridin-2-yl)methanamine hydrochloride (1:1) in 10 ml of acetonitrile, and 0.17 ml (0.29 mmol) of a 50% strength solution of T3P (2,4,6-tripropyl-1,3,5,2,4,6-trioxatriphosphorinane 2,4,6-trioxide) in ethyl acetate was then metered into the reaction solution at room temperature. After the addition had ended, the reaction solution was stirred at room temperature overnight. The reaction mixture was then extracted with water and with dichloromethane. The organic phase was finally separated off and the organic solution obtained was then filtered through hydrophobic filters (pleated filter MN 616 WA ¼, D=12.5 cm), dried and concentrated to dryness under reduced pressure. The residue obtained was purified using the following method.

Method 9:

Instrument: Waters Prep LC/MS System, column: XBridge C18 5 μm 100×30 mm

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time.

This gave 5.4 mg (0.01 mmol, 5% of theory) of the target compound as a white lyophylisate.

¹H-NMR (400 MHz, DMSO-d₆, δ/ppm): 0.75-0.89 (m, 4H, including at 0.82 (d, 3H)), 1.33-1.68 (m, 6H), 1.71-1.83 (m, 3H), 2.05 (br. t, 1H), 2.44-2.58 (m, 1H, partially obscured by DMSO), 2.69-2.80 (m, 2H), 3.05 (td, 2H), 3.94 (br. d, 2H), 4.56 (dd, 2H), 7.36-7.43 (m, 1H), 7.64-7.72 (m, 1H), 7.84 (s, 1H), 8.38 (dt, 1H), 8.69 (t, 1H).

LC-MS (Methode 4): Rₜ=0.48 min; m/z=418 (M+H)⁺.

### Example 21

### N-(5-Chloro-2-fluorobenzyl)-2-[(3R)-3-methyl[1,4′-bipiperidin]-1′-yl]-1,3-thiazole-5-carboxamide

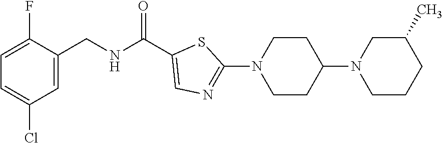 [SMILES: C[C@@H]1CCCN(C2CCN(c3ncc(C(=O)NCc4cc(Cl)ccc4F)s3)CC2)C1]

0.18 ml (1.05 mmol) of N,N-diisopropylethylamine was added to 100 mg (0.26 mmol) of 2-[(3R)-3-methyl[1,4′-bipiperidin]-1′-yl]-1,3-thiazole-5-carboxylic acid dihydrochloride and 46 mg (0.29 mmol) of 1-(5-chloro-2-fluorophenyl)methanamine in 10 ml of acetonitrile, and 0.17 ml (0.29 mmol) of a 50% strength solution of T3P (2,4,6-tripropyl-1,3,5,2,4,6-trioxatriphosphorinane 2,4,6-trioxide) in ethyl acetate was then metered into the reaction solution at room temperature. After the addition had ended, the reaction solution was stirred at room temperature overnight. The reaction mixture was then extracted with water and with dichloromethane. The organic phase was finally separated off and the organic solution obtained was then filtered through hydrophobic filters (pleated filter MN 616 WA ¼, D=12.5 cm), dried and concentrated to dryness under reduced pressure. The residue obtained was purified using the following method.

Method 7:

Instrument: Waters Prep LC/MS System, column: XBridge C18 5 μm 100×30 mm

Mobile phase A: water, mobile phase B: acetonitrile, mobile phase C: 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time.

This gave 45 mg of a mixture which was purified further by column chromatography on silica gel (Isolera Biotage SNAP-Ultra 10 g column; mobile phase: cyclohexane/ethyl acetate 8:2→gradient over 15 CV (CV=column volumes)→cyclohexane/ethyl acetate 2:8). This gave 16 mg (0.04 mmol, 14% of theory) of the target compound as a beige solid.

¹H-NMR (600 MHz, DMSO-d₆, δ/ppm): 0.76-0.87 (m, 4H, including at 0.82 (d, 3H)), 1.35-1.67 (m, 6H), 1.72-1.82 (m, 3H), 2.05 (br. t, 1H), 2.45-2.57 (m, 1H, partially obscured by DMSO), 2.74 (br. t, 2H), 3.05 (td, 2H), 3.94 (br. d, 2H), 4.41 (d, 2H), 7.26 (t, 1H), 7.33-7.40 (m, 2H), 7.85 (s, 1H), 8.76 (t, 1H).

LC-MS (Methode 4): Rₜ=0.68 min; m/z=451/453 (M+H)⁺.

### Example 22

### 2-[(3R)-3-Methyl[1,4′-bipiperidin]-1′-yl]-N-[4-(trifluoromethyl)benzyl]-1,3-thiazole-5-carboxamide

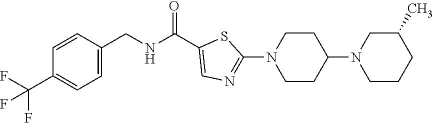 [SMILES: C[C@@H]1CCCN(C2CCN(c3ncc(C(=O)NCc4ccc(C(F)(F)F)cc4)s3)CC2)C1]

0.22 ml (1.23 mmol) of N,N-diisopropylethylamine was added to 200 mg (0.31 mmol, purity 59%) of 2-[(3R)-3-methyl[1,4′-bipiperidin]-1′-yl]-1,3-thiazole-5-carboxylic acid dihydrochloride and 59 mg (0.34 mmol) of 1-[4-(trifluoromethyl)phenyl]methanamine in 10 ml of acetonitrile, and 0.2 ml (0.34 mmol) of a 50% strength solution of T3P (2,4,6-tripropyl-1,3,5,2,4,6-trioxatriphosphorinane 2,4,6-trioxide) in ethyl acetate was then metered into the reaction solution at room temperature. After the addition had ended, the reaction solution was stirred at room temperature overnight. The reaction mixture was then extracted with water and with dichloromethane. The organic phase was finally separated off and the organic solution obtained was then filtered through hydrophobic filters (pleated filter MN 616 WA ¼, D=12.5 cm), dried and concentrated to dryness under reduced pressure. The residue obtained was purified using the following method.

Method 10:

Instrument: Waters Prep LC/MS System, column: XBridge C18 5 μm 100×30 mm

Mobile phase A: water, mobile phase B: acetonitrile, mobile phase C 2% ammonia in water, mobile phase D: acetonitrile/water (80% by volume/20% by volume) total flow rate: 80 ml/min, room temperature, wavelength 200-400 nm, At-Column Injection (complete injection)

Gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time.

This gave 25 mg (0.05 mmol, 17% of theory) of the target compound as a white lyophylisate.

¹H-NMR (400 MHz, DMSO-d₆, δ/ppm): 0.74-0.89 (m, 4H, including at 0.82 (d, 3H)), 1.33-1.68 (m, 6H), 1.71-1.83 (m, 3H), 2.00-2.10 (m, 1H), 2.45-2.57 (m, 1H, partially obscured by DMSO), 2.70-2.79 (m, 2H), 3.06 (td, 2H), 3.94 (br. d, 2H), 4.47 (d, 2H), 7.50 (d, 2H), 7.70 (d, 2H), 7.84 (s, 1H), 8.83 (t, 1H).

LC-MS (Methode 1): Rₜ=1.27 min; m/z=467 (M+H)⁺.

Analogously to Examples 18 to 22, the following compounds of Examples 23 to 37 were prepared from the starting materials stated in each case:

| Example | Name/Structure/Starting material | Analytical data |
|---|---|---|
| 23 | N-[(5-chloro-3-fluoropyridin-2-yl)methyl]-2- [(3R)-3-methyl[1,4′-bipiperidin]-1′-yl]-1,3- thiazole-5-carboxamide 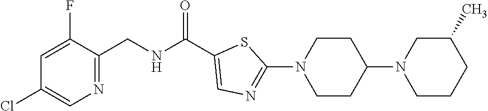 [SMILES: C[C@@H]1CCCN(C2CCN(c3ncc(C(=O)NCc4ncc(Cl)cc4F)s3)CC2)C1] from 2-[(3R)-3-methyl[1,4′-bipiperidin]-1′-yl]- 1,3-thiazole-5-carboxylic acid dihydrochloride | ¹H-NMR (400 MHz, DMSO-d₆, δ/ppm): 0.74-0.88 (m, 4H, including at 0.82 (d, 3H)), 1.32- 1.67 (m, 6H), 1.71-1.82 (m, 3H), 2.00-2.10 (m, 1H), 2.44-2.58 (m, 1H, partially obscured by DMSO), 2.69-2.79 (m, 2H), 3.04 (td, 2H), 3.93 (br. d, 2H), 4.53 (dd, 2H), 7.83 (s, 1H), 8.06 (dd, 1H), 8.48 (d, 1H), 8.73 (t, 1H). LC-MS (Methode 1): Rₜ = 1.04 min; m/z = 452/454 (M + H)⁺. |
| | and 1-(5-chloro-3-fluoropyridin-2- | |
| | yl)methanamine hydrochloride (1:1) | |
| 24 | 2-[(3R)-3-methyl[1,4′-bipiperidin]-1′-yl]-N-[(3- methylpyridin-2-yl)methyl]-1,3-thiazole-5- carboxamide 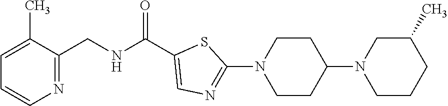 [SMILES: Cc1cccnc1CNC(=O)c1cnc(N2CCC(N3CCC[C@@H](C)C3)CC2)s1] from 2-[(3R)-3-methyl[1,4′-bipiperidin]-1′-yl]- 1,3-thiazole-5-carboxylic acid dihydrochloride | ¹H-NMR (400 MHz, DMSO-d6, δ/ppm): 0.75-0.88 (m, 4H, including at 0.82 (d, 3H)), 1.33- 1.68 (m, 6H), 1.70-1.83 (m, 3H), 2.00-2.11 (m, 1H), 2.31 (s, 3H), 2.43-2.58 (m, 1H, partially obscured by DMSO), 2.69-2.80 (m, 2H), 3.04 (td, 2H), 3.94 (br. d, 2H), 4.50 (d, 2H), 7.21 (dd, 1H), 7.57 (dd, 1H), 7.86 (s, 1H), 8.35 (dd, 1H), 8.54 (t, 1H). LC-MS (Methode 1): Rₜ = 0.66 min; m/z = 414 (M + H)⁺. |
| | and 1-(3-methylpyridin-2-yl)methanamine | |
| 25 | 2-[(3R)-3-methyl[1,4′-bipiperidin]-1′-yl]-N-[(4- methylpyridin-2-yl)methyl]-1,3-thiazole-5- carboxamide 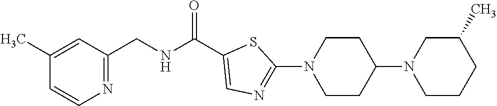 from 2-[(3R)-3-methyl[1,4′-bipiperidin]-1′-yl]- | ¹H-NMR (600 MHz, DMSO-d₆, δ/ppm): 0.77-0.86 (m, 4H, including at 0.82 (d, 3H)), 1.35- 1.67 (m, 6H), 1.71-1.81 (m, 3H), 2.05 (br. t, 1H), 2.29 (s, 3H), 2.46- 2.53 (m, 1H, partially obscured by DMSO), 2.74 (br. t, 2H), 3.05 (td, 2H), 3.95 (br. d, 2H), 4.44 (d, 2H), 7.09 (d, 1H), 7.11 (s, 1H), 7.86 (s, 1H), 8.35 (d, 1H), 8.78 (t, 1H). LC-MS (Methode 1): Rₜ = 0.63 min; m/z = 414 (M + H)⁺. |
| | 1,3-thiazole-5-carboxylic acid dihydrochloride | |
| | and 1-(4-methylpyridin-2-yl)methanamine | |
| 26 | N-[(3-chloropyridin-2-yl)methyl]-2-[(3R)-3- methyl[1,4′-bipiperidin]-1′-yl]-1,3-thiazole-5- carboxamide 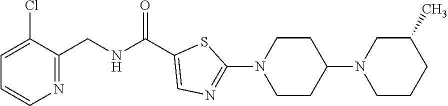 [SMILES: C[C@@H]1CCCN(C2CCN(c3ncc(C(=O)NCc4ncccc4Cl)s3)CC2)C1] from 2-[(3R)-3-methyl[1,4′-bipiperidin]-1′-yl]- 1,3-thiazole-5-carboxylic acid dihydrochloride | ¹H-NMR (400 MHz, DMSO-d₆, δ/ppm): 0.75-0.88 (m, 4H, including at 0.82 (d, 3H)), 1.33- 1.68 (m, 6H), 1.71-1.83 (m, 3H), 2.05 (br. t, 1H), 2.44-2.57 (m, 1H, partially obscured by DMSO), 2.69-2.80 (m, 2H), 3.05 (br. t, 2H), 3.94 (br. d, 2H), 4.60 (d, 2H), 7.36 (dd, 1H), 7.86 (s, 1H), 7.92 (dd, 1H), 8.50 (dd, 1H), 8.64 (t, 1H). LC-MS (Methode 1): Rₜ = 0.93 min; m/z = 434/436 (M + H)⁺. |
| | and 1-(3-chloropyridin-2-yl)methanamine | |
| | hydrochloride (1:1) | |
| 27 | N-[(3-fluoropyridin-2-yl)methyl]-N-methyl-2- [(3R)-3-methyl[1,4′-bipiperidin]-1′-yl]-1,3- thiazole-5-carboxamide 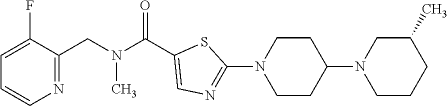 [SMILES: C[C@@H]1CCCN(C2CCN(c3ncc(C(=O)N(C)Cc4ncccc4F)s3)CC2)C1] from 2-[(3R)-3-methyl[1,4′-bipiperidin]-1′-yl]- 1,3-thiazole-5-carboxylic acid dihydrochloride | ¹H-NMR (400 MHz, DMSO-d₆, δ/ppm): 0.74-0.88 (m, 4H, including at 0.82 (d, 3H)), 1.32- 1.67 (m, 6H), 1.70-1.83 (m, 3H), 2.05 (br. t, 1H), 2.43-2.58 (m, 1H, partially obscured by DMSO), 2.69-2.79 (m, 2H), 3.04 (td, 2H), 3.16 (br. s, 3H), 3.94 (br. d, 2H), 4.86 (s, 2H), 7.38-7.46 (m, 1H), 7.59 (s, 1H), 7.68-7.77 (m, 1H), 8.37-8.45 (m, 1H). LC-MS (Methode 1): Rₜ = 0.91 min; m/z = 432 (M + H)⁺. |
| | and (3-fluoropyridin-2-yl)-N-methylmethanamine | |
| 28 | 2-[(3R)-3-methyl[1,4′-bipiperidin]-1′-yl]-N-{[6- (trifluoromethyl)pyridin-2-yl]methyl}-1,3- thiazole-5-carboxamide 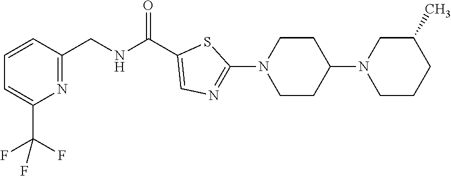 [SMILES: C[C@@H]1CCCN(C2CCN(c3ncc(C(=O)NCc4cccc(C(F)(F)F)n4)s3)CC2)C1] | ¹H-NMR (400 MHz, DMSO-d₆, δ/ppm): 0.75-0.88 (m, 4H, including at 0.82 (d, 3H)), 1.33- 1.68 (m, 6H), 1.71-1.83 (m, 3H), 2.05 (br. t, 1H), 2.45-2.58 (m, 1H, partially obscured by DMSO), 2.69-2.79 (m, 2H), 3.06 (td, 2H), 3.95 (br. d, 2H), 4.54 (d, 2H), 7.61 (d, 1H), 7.79 (d, 1H), 7.87 (s, 1H), 8.07 (t, 1H), 8.95 (t, 1H). LC-MS (Methode 1): Rₜ = 1.09 min; m/z = 468 (M + H)⁺. |
| | from 2-[(3R)-3-methyl[1,4′-bipiperidin]-1′-yl]- | |
| | 1,3-thiazole-5-carboxylic acid dihydrochloride | |
| | and 1-[6-(trifluoromethyl)pyridin-2- | |
| | yl]methanamine hydrochloride (1:1) | |
| 29 | N-[(5-chloropyridin-2-yl)methyl]-2-[(3R)-3- | LC-MS (Methode 1): |
| | methyl[1,4′-bipiperidin]-1′-yl]-1,3-thiazole-5- | Rₜ = 0.91 min; m/z = 434/436 |
| | carboxamide | (M + H)⁺. |
| | from 2-[(3R)-3-methyl[1,4′-bipiperidin]-1′-yl]- | |
| | 1,3-thiazole-5-carboxylic acid dihydrochloride | |
| | and 1-(5-chloropyridin-2-yl)methanamine | |
| 30 | N-[1-(2,5-difluorophenyl)ethyl]-2-[(3R)-3- methyl[1,4′-bipiperidin]-1′-yl]-1,3-thiazole-5- carboxamide (diastereomer mixture) 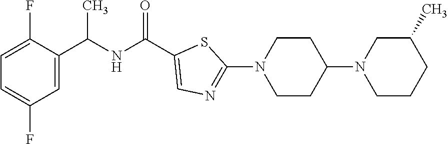 [SMILES: CC(NC(=O)c1cnc(N2CCC(N3CCC[C@@H](C)C3)CC2)s1)c1cc(F)ccc1F] | ¹H-NMR (600 MHz, DMSO-d₆, δ/ppm): 0.76-0.86 (m, 4H, including at 0.82 (d, 3H)), 1.32- 1.66 (m, 9H, including at 1.42 (d, 3H)), 1.71-1.81 (m, 3H), 2.05 (td, 1H), 2.46-2.56 (m, 1H, partially obscured by DMSO), 2.73 (br. t, 2H), 3.01-3.09 (m, 2H), 3.90-3.99 (m, 2H), 5.22-5.29 (m, 1H), 7.09- 7.16 (m, 1H), 7.19-7.26 (m, 2H), 7.92 (s, 1H), 8.55 (d, 1H). LC-MS (Methode 1): Rₜ = 1.22 min; m/z = 449 (M + H)⁺. |
| | from 2-[(3R)-3-methyl[1,4′-bipiperidin]-1′-yl]- | |
| | 1,3-thiazole-5-carboxylic acid dihydrochloride | |
| | and 1-(2,5-difluorophenyl)ethanamine (racemate) | |
| 31 | N-[(3-chloro-5-fluoropyridin-2-yl)methyl]-2- | ¹H-NMR (400 MHz, DMSO-d₆, |
| | [(3R)-3-methyl[1,4′-bipiperidin]-1′-yl]-1,3- | δ/ppm): 0.75-0.88 (m, 4H, |
| | thiazole-5-carboxamide | including at 0.82 (d, 3H)), 1.33- |
| | from 2-[(3R)-3-methyl[1,4′-bipiperidin]-1′-yl]- | 1.68 (m, 6H), 1.71-1.83 (m, 3H), |
| | 1,3-thiazole-5-carboxylic acid dihydrochloride | 2.06 (br. t, 1H), 2.44-2.57 (m, 1H, |
| | and 1-(3-chloro-5-fluoropyridin-2- | partially obscured by DMSO), |
| | yl)methanamine hydrochloride (1:1) | 2.69-2.80 (m, 2H), 3.05 (br. t, 2H), |
| | | 3.94 (br. d, 2H), 4.57 (d, 2H), 7.85 |
| | | (s, 1H), 8.09 (dd, 1H), 8.57 (d, |
| | | 1H), 8.66 (t, 1H). |
| | | LC-MS (Methode 1): |
| | | Rₜ = 1.02 min; m/z = 452/454 |
| | | (M + H)⁺. |
| 32 | 2-[(3R)-3-methyl[1,4′-bipiperidin]-1′-yl]-N-{[6- | ¹H-NMR (400 MHz, DMSO-d₆, |
| | (trifluoromethoxy)pyridin-2-yl]methyl}-1,3- | δ/ppm): 0.75-0.89 (m, 4H, |
| | thiazole-5-carboxamide | including at 0.82 (d, 3H)), 1.33- |
| | from 2-[(3R)-3-methyl[1,4′-bipiperidin]-1′-yl]- | 1.68 (m, 6H), 1.71-1.83 (m, 3H), |
| | 1,3-thiazole-5-carboxylic acid dihydrochloride | 2.05 (br. t, 1H), 2.43-2.57 (m, 1H, |
| | and 1-[6-(trifluoromethoxy)pyridin-2- | partially obscured by DMSO), |
| | yl]methanamine | 2.69-2.79 (m, 2H), 3.06 (br. t, 2H), |
| | | 3.95 (br. d, 2H), 4.44 (d, 2H), 7.16 |
| | | (d, 1H), 7.33 (d, 1H), 7.86 (s, 1H), |
| | | 7.99 (t, 1H), 8.87 (t, 1H). |
| | | LC-MS (Methode 4): |
| | | Rₜ = 0.65 min; m/z = 484 (M + H)⁺. |
| 33 | N-(4-chlorobenzyl)-2-[(3R)-3-methyl[1,4′- bipiperidin]-1′-yl]-1,3-thiazole-5- carboxamide 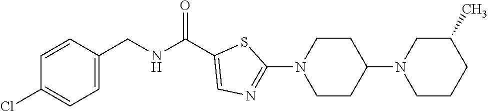 [SMILES: C[C@@H]1CCCN(C2CCN(c3ncc(C(=O)NCc4ccc(Cl)cc4)s3)CC2)C1] from 2-[(3R)-3-methyl[1,4′-bipiperidin]-1′-yl]- 1,3-thiazole-5-carboxylic acid dihydrochloride | ¹H-NMR (400 MHz, DMSO-d₆, δ/ppm): 0.75-0.88 (m, 4H, including at 0.82 (d, 3H)), 1.33- 1.68 (m, 6H), 1.71-1.83 (m, 3H), 2.00-2.10 (m, 1H), 2.44-2.57 (m, 1H, partially obscured by DMSO), 2.69-2.79 (m, 2H), 3.05 (br. td, 2H), 3.94 (br. d, 2H), 4.37 (d, 2H), 7.27-7.33 (m, 2H), 7.35-7.42 (m, 2H), 7.82 (s, 1H), 8.75 (t, 1H). LC-MS (Methode 1): Rₜ = 1.18 min; m/z = 433/435 (M + H)⁺. |
| | and 1-(4-chlorophenyl)methanamine | |
| 34 | N-(2-chloro-5-fluorobenzyl)-2-[(3R)-3- | ¹H-NMR (600 MHz, DMSO-d₆, |
| | methyl[1,4′-bipiperidin]-1′-yl]-1,3-thiazole-5- | δ/ppm): 0.76-0.87 (m, 4H, |
| | carboxamide | including at 0.82 (d, 3H)), 1.35- |
| | from 2-[(3R)-3-methyl[1,4′-bipiperidin]-1′-yl]- | 1.67 (m, 6H), 1.72-1.83 (m, 3H), |
| | 1,3-thiazole-5-carboxylic acid dihydrochloride | 2.05 (td, 1H), 2.46-2.57 (m, 1H, |
| | and 1-(2-chloro-5-fluorophenyl)methanamine | partially obscured by DMSO), |
| | | 2.74 (br. t, 2H), 3.06 (td, 2H), 3.96 |
| | | (br. d, 2H), 4.44 (d, 2H), 7.13 (dd, |
| | | 1H), 7.18 (td, 1H), 7.51 (dd, 1H), |
| | | 7.88 (s, 1H), 8.77 (t, 1H). |
| | | LC-MS (Methode 1): |
| | | Rₜ = 1.23 min; m/z = 451/453 |
| | | (M + H)⁺. |
| 35 | N-(4-methylbenzyl)-2-[(3R)-3-methyl[1,4′- bipiperidin]-1′-yl]-1,3-thiazole-5- carboxamide 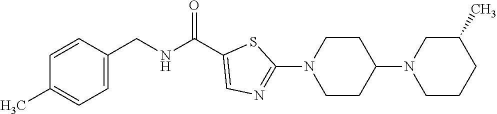 from 2-[(3R)-3-methyl[1,4′-bipiperidin]-1′-yl]- | ¹H-NMR (600 MHz, DMSO-d₆, δ/ppm): 0.76-0.87 (m, 4H, including at 0.82 (d, 3H)), 1.34- 1.66 (m, 6H), 1.71-1.81 (m, 3H), 2.05 (br. t, 1H), 2.27 (s, 3H), 2.45- 2.56 (m, 1H, partially obscured by DMSO), 2.73 (br. t, 2H), 3.04 (td, 2H), 3.94 (br. d, 2H), 4.34 (d, 2H), 7.15 (q, 4H), 7.81 (s, 1H), 8.68 (t, 1H). LC-MS (Methode 1): Rₜ = 1.19 min; m/z = 413 (M + H)⁺. |
| | 1,3-thiazole-5-carboxylic acid dihydrochloride | |
| | and 1-(4-methylphenyl)methanamine | |
| 36 | N-(3-methylbenzyl)-2-[(3R)-3-methyl[1,4′- bipiperidin]-1′-yl]-1,3-thiazole-5- carboxamide 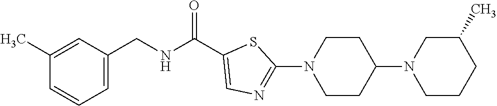 from 2-[(3R)-3-methyl[1,4′-bipiperidin]-1′-yl]- | ¹H-NMR (600 MHz, DMSO-d₆, δ/ppm): 0.76-0.87 (m, 4H, including at 0.82 (d, 3H)), 1.35- 1.67 (m, 6H), 1.71-1.81 (m, 3H), 2.05 (br. t, 1H), 3.02 (s, 3H), 2.45- 2.56 (m, 1H, partially obscured by DMSO), 2.74 (br. t, 2H), 3.04 (td, 2H), 3.94 (br. d, 2H), 4.35 (d, 2H), 7.03-7.11 (m, 3H), 7.20 (t, 1H), 7.82 (s, 1H), 8.69 (t, 1H). LC-MS (Methode 1): Rₜ = 1.19 min; m/z = 413 (M + H)⁺. |
| | 1,3-thiazole-5-carboxylic acid dihydrochloride | |
| | and 1-(3-methylphenyl)methanamine | |
| 37 | N-(2-methylbenzyl)-2-[(3R)-3-methyl[1,4′- bipiperidin]-1′-yl]-1,3-thiazole-5- carboxamide 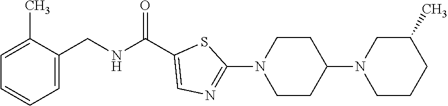 [SMILES: Cc1ccccc1CNC(=O)c1cnc(N2CCC(N3CCC[C@@H](C)C3)CC2)s1] from 2-[(3R)-3-methyl[1,4′-bipiperidin]-1′-yl]- | ¹H-NMR (600 MHz, DMSO-d₆, δ/ppm): 0.77-0.86 (m, 4H, including at 0.82 (d, 3H)), 1.35- 1.66 (m, 6H), 1.72-1.81 (m, 3H), 2.05 (br. t, 1H), 3.05 (s, 3H), 2.46- 2.55 (m, 1H, partially obscured by DMSO), 2.74 (br. t, 2H), 3.04 (td, 2H), 3.94 (br. d, 2H), 4.37 (d, 2H), 7.13-7.18 (m, 3H), 7.19-7.24 (m, 1H), 7.85 (s, 1H), 8.58 (t, 1H). LC-MS (Methode 1): Rₜ = 1.16 min; m/z = 413 (M + H)⁺. |
| | 1,3-thiazole-5-carboxylic acid dihydrochloride | |
| | and 1-(2-methylphenyl)methanamine | |

### Example 38 and Example 39

### 2-[3-(Difluoromethyl)[1,4′-bipiperidin]-1′-yl]-N-[(3,5-difluoropyridin-2-yl)methyl]-1,3-thiazole-5-carboxamide (enantiomers 1 and 2)

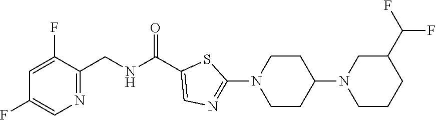 [SMILES: O=C(NCc1ncc(F)cc1F)c1cnc(N2CCC(N3CCCC(C(F)F)C3)CC2)s1]

203 mg (0.43 mmol) of the racemic 2-[3-(difluoromethyl)[1,4′-bipiperidin]-1′-yl]-N-[(3,5-difluoropyridin-2-yl)methyl]-1,3-thiazole-5-carboxamide (Example 4) were separated into the enantiomers by preparative HPLC on a chiral phase [column: Daicel Chiralpak AY-H, 5 μm, 250 mm×20 mm; mobile phase: 2-propanol+0.2% diethylamine/n-heptane 50:50; flow rate: 20 ml/min; UV detection: 220 nm; temperature: 40° C.]:

### Example 38 (enantiomer 1)

### 2-[(3S)-3-(Difluoromethyl)[1,4′-bipiperidin]-1′-yl]-N-[(3,5-difluoropyridin-2-yl)methyl]-1,3-thiazole-5-carboxamide

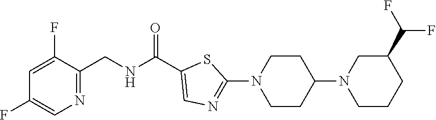 [SMILES: O=C(NCc1ncc(F)cc1F)c1cnc(N2CCC(N3CCC[C@H](C(F)F)C3)CC2)s1]

yield: 97 mg

Rₜ=4.93 min; chemical purity>99%; >99% ee

[column: Chiraltek AY-3, 3 μm, 100 mm×4.6 mm; mobile phase: isohexane/2-propanol+0.2% diethylamine 20:80; flow rate: 1 ml/min; temperature: 25° C.; UV detection: 220 nm].

LC-MS (Methode 5): Rₜ=1.52 min; m/z=472 (M+H)⁺.

### Example 39 (enantiomer 2)

### 2-[(3R)-3-(Difluoromethyl)[1,4′-bipiperidin]-1′-yl]-N-[(3,5-difluoropyridin-2-yl)methyl]-1,3-thiazole-5-carboxamide

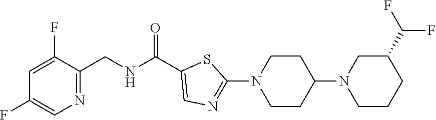 [SMILES: O=C(NCc1ncc(F)cc1F)c1cnc(N2CCC(N3CCC[C@@H](C(F)F)C3)CC2)s1]

yield: 101 mg

Rₜ=6.03 min; chemical purity>96%; >94% ee

[column: Chiraltek AY-3, 3 μm, 100 mm×4.6 mm; mobile phase: isohexane/2-propanol+0.2% diethylamine 20:80; flow rate: 1 ml/min; temperature: 25° C.; UV detection: 220 nm].

LC-MS (Methode 5): Rₜ=1.52 min; m/z=472 (M+H)⁺.

¹H-NMR (600 MHz, DMSO-d₆, δ/ppm): 1.11-1.22 (m, 1H), 1.37-1.54 (m, 3H), 1.62-1.72 (m, 2H), 1.73-1.81 (m, 2H), 1.88-1.99 (m, 1H), 2.10-2.21 (m, 2H), 2.47-2.60 (m, 1H, partially obscured by DMSO), 2.72 (br. d, 1H), 2.79 (br. d, 1H), 3.05 (br. t, 2H), 3.94 (br. d, 2H), 4.53 (br. d, 2H), 5.82-6.06 (m, 1H), 7.84 (s, 1H), 7.93 (td, 1H), 8.47 (d, 1H), 8.75 (t, 1H).

### Example 40 and Example 41

### N-[(3,5-difluoropyridin-2-yl)methyl]-2-[3-(fluoromethyl)[1,4′-bipiperidin]-1′-yl]-1,3-thiazole-5-carboxamide (enantiomers 1 and 2)

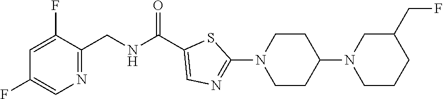 [SMILES: O=C(NCc1ncc(F)cc1F)c1cnc(N2CCC(N3CCCC(CF)C3)CC2)s1]

144 mg (0.32 mmol) of the racemic N-[(3,5-difluoropyridin-2-yl)methyl]-2-[3-(fluoromethyl)[1,4′-bipiperidin]-1′-yl]-1,3-thiazole-5-carboxamide (Example 6) were separated into the enantiomers by preparative HPLC on a chiral phase [column: Daicel Chiralpak IG, 5 μm, 250 mm×20 mm; mobile phase: ethanol; flow rate: 15 ml/min; UV detection: 220 nm; temperature: 70° C.]:

### Example 40 (enantiomer 1)

### N-[(3,5-Difluoropyridin-2-yl)methyl]-2-[(3S)-3-(fluoromethyl)[1,4′-bipiperidin]-1′-yl]-1,3-thiazole-5-carboxamide

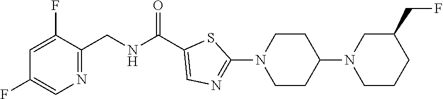 [SMILES: O=C(NCc1ncc(F)cc1F)c1cnc(N2CCC(N3CCC[C@H](CF)C3)CC2)s1]

yield: 71 mg

Rₜ=10.94 min; chemical purity 99%; 99% ee

[column: Daicel Chiralcel IG, 5 μm, 250 mm×4.6 mm; mobile phase: ethanol+0.2% diethylamine; flow rate: 1 ml/min; temperature: 40° C.; UV detection: 235 nm].

LC-MS (Methode 1): Rₜ=0.85 min; m/z=454 (M+H)⁺.

### Example 41 (enantiomer 2)

### N-[(3,5-Difluoropyridin-2-yl)methyl]-2-[(3R)-3-(fluoromethyl)[1,4′-bipiperidin]-1′-yl]-1,3-thiazole-5-carboxamide

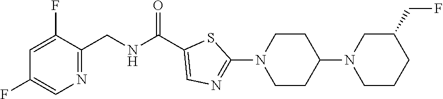 [SMILES: O=C(NCc1ncc(F)cc1F)c1cnc(N2CCC(N3CCC[C@@H](CF)C3)CC2)s1]

yield: 70 mg

Rₜ=12.21 min; chemical purity 99%; 99% ee

[column: Daicel Chiralcel IG, 5 μm, 250 mm×4.6 mm; mobile phase: ethanol+0.2% diethylamine; flow rate: 1 ml/min; temperature: 40° C.; UV detection: 235 nm].

LC-MS (Methode 1): Rₜ=0.84 min; m/z=454 (M+H)⁺.

¹H-NMR (400 MHz, DMSO-d₆, δ/ppm): 0.94-1.10 (m, 1H), 1.35-1.55 (m, 3H), 1.61 (br. d, 2H), 1.72-1.92 (m, 3H), 2.03 (t, 1H), 2.16 (br. t, 1H), 2.47-2.57 (m, 1H, partially obscured by DMSO), 2.65-2.76 (m, 1H), 2.80 (br. d, 1H), 3.05 (br. t, 2H), 3.94 (br. d, 2H), 4.19-4.29 (m, 1H), 4.31-4.41 (m, 1H), 4.53 (br. d, 2H), 7.83 (s, 1H), 7.87-7.96 (m, 1H), 8.47 (d, 1H), 8.71 (t, 1H).

### Example 42 and Example 43

### N-[(3,5-Difluoropyridin-2-yl)methyl]-2-[3-(trifluoromethyl)[1,4′-bipiperidin]-1′-yl]-1,3-thiazole-5-carboxamide (enantiomers 1 and 2)

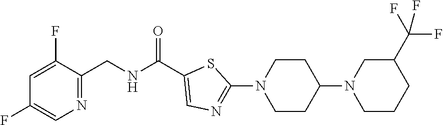 [SMILES: O=C(NCc1ncc(F)cc1F)c1cnc(N2CCC(N3CCCC(C(F)(F)F)C3)CC2)s1]

143 mg (0.29 mmol) of N-[(3,5-difluoropyridin-2-yl)methyl]-2-[3-(trifluoromethyl)[1,4′-bipiperidin]-1′-yl]-1,3-thiazole-5-carboxamide (Example 5) were separated into the enantiomers by preparative HPLC on a chiral phase [column: Daicel Chiralpak IG, 5 μm, 250 mm×20 mm; mobile phase: ethanol; flow rate: 15 ml/min; UV detection: 220 nm; temperature: 40° C.]:

### Example 42 (enantiomer 1)

### N-[(3,5-Difluoropyridin-2-yl)methyl]-2-[(3S)-3-(trifluoromethyl)[1,4′-bipiperidin]-1′-yl]-1,3-thiazole-5-carboxamide

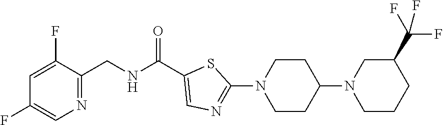 [SMILES: O=C(NCc1ncc(F)cc1F)c1cnc(N2CCC(N3CCC[C@H](C(F)(F)F)C3)CC2)s1]

yield: 67 mg

Rₜ=11.22 min; chemical purity 99%; 99% ee

[column: Daicel Chiralcel IG, 5 μm, 250 mm×4.6 mm; mobile phase: ethanol+0.2% diethylamine; flow rate: 1 ml/min; temperature: 50° C.; UV detection: 235 nm].

LC-MS (Methode 1): Rₜ=0.97 min; m/z=490 (M+H)⁺.

### Example 43 (enantiomer 2)

### N-[(3,5-Difluoropyridin-2-yl)methyl]-2-[(3R)-3-(trifluoromethyl)[1,4′-bipiperidin]-1′-yl]-1,3-thiazole-5-carboxamide

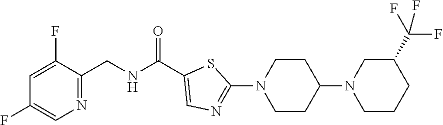 [SMILES: O=C(NCc1ncc(F)cc1F)c1cnc(N2CCC(N3CCC[C@@H](C(F)(F)F)C3)CC2)s1]

yield: 67 mg

Rₜ=11.87 min; chemical purity 99%; >96% ee

[column: Daicel Chiralcel IG, 5 μm, 250 mm×4.6 mm; mobile phase: ethanol+0.2% diethylamine; flow rate: 1 ml/min; temperature: 50° C.; UV detection: 235 nm].

LC-MS (Methode 1): Rₜ=0.96 min; m/z=490 (M+H)⁺.

¹H-NMR (500 MHz, DMSO-d₆, δ/ppm): 1.14-1.27 (m, 1H), 1.39-1.57 (m, 3H), 1.65-1.73 (m, 1H), 1.74-1.82 (m, 2H), 1.82-1.88 (m, 1H), 2.06-2.20 (m, 2H), 2.32-2.44 (m, 1H), 2.61 (br. t, 1H), 2.81 (br. d, 1H), 2.96 (br. d, 1H), 3.05 (td, 2H), 3.95 (br. d, 2H), 4.53 (br. d, 2H), 7.83 (s, 1H), 7.88-7.94 (m, 1H), 8.47 (d, 1H), 8.71 (t, 1H).

### Example 44 and Example 45

### 2-{3-[(3,3-Difluorocyclobutyl)methoxy][1,4′-bipiperidin]-1′-yl}-N-[(3,5-difluoropyridin-2-yl)methyl]-1,3-thiazole-5-carboxamide (enantiomers 1 and 2)

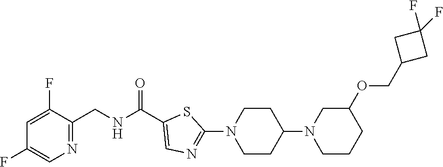 [SMILES: O=C(NCc1ncc(F)cc1F)c1cnc(N2CCC(N3CCCC(OCC4CC(F)(F)C4)C3)CC2)s1]

251 mg (0.46 mmol) of 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide (Example 7) were separated into the enantiomers by preparative HPLC on a chiral phase [column: Daicel Chiralcel OD-H, 5 μm, 250 mm×20 mm; mobile phase: n-heptane/2-propanol+0.2% diethylamine 50:50; flow rate: 20 ml/min; UV detection: 220 nm; temperature: 30° C.]:

### Example 44 (enantiomer 1)

### 2-{(3R)-3-[(3,3-Difluorocyclobutyl)methoxy][1,4′-bipiperidin]-1′-yl}-N-[(3,5-difluoropyridin-2-yl)methyl]-1,3-thiazole-5-carboxamide

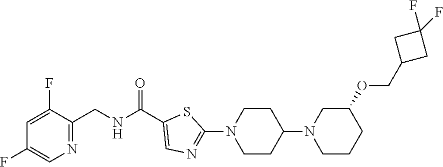 [SMILES: O=C(NCc1ncc(F)cc1F)c1cnc(N2CCC(N3CCC[C@@H](OCC4CC(F)(F)C4)C3)CC2)s1]

yield: 93 mg

Rₜ=1.50 min; chemical purity>99%; 99% ee

[column: Phenomenex Cellulose-1, 3 μm, 50 mm×4.6 mm; mobile phase: n-heptane/2-propanol+0.2% diethylamine); flow rate: 1 ml/min; temperature: 25° C.; UV detection: 220 nm].

LC-MS (Methode 4): Rₜ=0.63 min; m/z=542 (M+H)⁺.

### Example 45 (enantiomer 2)

### 2-{(3S)-3-[(3,3-Difluorocyclobutyl)methoxy][1,4′-bipiperidin]-1′-yl}-N-[(3,5-difluoropyridin-2-yl)methyl]-1,3-thiazole-5-carboxamide

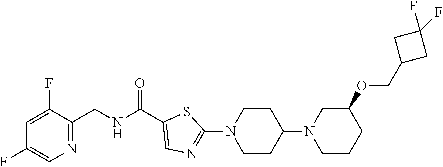 [SMILES: O=C(NCc1ncc(F)cc1F)c1cnc(N2CCC(N3CCC[C@H](OCC4CC(F)(F)C4)C3)CC2)s1]

yield: 86 mg

Rₜ=2.21 min; chemical purity>99%; 99% ee

[column: Phenomenex Cellulose-1, 3 μm, 50 mm×4.6 mm; mobile phase: n-heptane/2-propanol+0.2% diethylamine); flow rate: 1 ml/min; temperature: 25° C.; UV detection: 220 nm].

LC-MS (Methode 4): Rₜ=0.62 min; m/z=542 (M+H)⁺.

¹H-NMR (400 MHz, DMSO-d₆, δ/ppm): 1.02-1.12 (m, 1H), 1.30-1.42 (m, 1H), 1.42-1.56 (m, 2H), 1.58-1.68 (m, 1H), 1.72-1.83 (m, 2H), 1.85-1.94 (m, 1H), 1.99 (br. t, 1H), 2.10 (br. t, 1H), 2.21-2.38 (m, 3H), 2.48-2.62 (m, 3H, partially obscured by DMSO), 2.62-2.70 (m, 1H), 2.95 (br. d, 1H), 3.04 (br. t, 2H), 3.22-3.34 (m, 1H, partially obscured by H₂O), 3.40-3.51 (m, 2H), 3.95 (br. d, 2H), 4.53 (br. d, 2H), 7.83 (s, 1H), 7.87-7.95 (m, 1H), 8.47 (d, 1H), 8.71 (t, 1H).

### Example 46 and Example 47

### N-[1-(2,5-Difluorophenyl)ethyl]-2-[(3R)-3-methyl[1,4′-bipiperidin]-1′-yl]-1,3-thiazole-5-carboxamide (diastereomers 1 and 2)

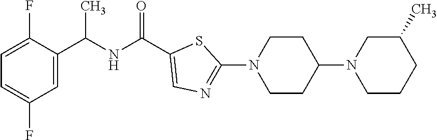 [SMILES: CC(NC(=O)c1cnc(N2CCC(N3CCC[C@@H](C)C3)CC2)s1)c1cc(F)ccc1F]

51 mg (0.11 mmol) of the diastereomer mixture N-[1-(2,5-difluorophenyl)ethyl]-2-[(3R)-3-methyl[1,4′-bipiperidin]-1′-yl]-1,3-thiazole-5-carboxamide (Example 30) were separated into the diastereomers by preparative HPLC on a chiral phase [column: Daicel Chiralcel OX-H 5 μm, 250 mm×20 mm; mobile phase: n-heptane/ethanol 50:50; flow rate: 20 ml/min; UV detection: 220 nm; temperature: 40° C.]:

### Example 46 (diastereomer 1)

yield: 20 mg

Rₜ=1.32 min; chemical purity>99%; 99% ee

[column: Daicel Chiralpak OX-3, 3 μm, 50 mm×4.6 mm; mobile phase: n-heptane/ethanol+0.2% diethylamine; flow rate: 1 ml/min; temperature: 25° C.; UV detection: 220 nm].

LC-MS (Methode 1): Rₜ=1.22 min; m/z=449 (M+H)⁺.

¹H-NMR (600 MHz, DMSO-d₆, δ/ppm): 0.76-0.87 (m, 4H, including at 0.82 (d, 3H)), 1.34-1.66 (m, 9H, including at 1.42 (d, 3H)), 1.70-1.84 (m, 3H), 2.00-2.12 (m, 1H), 2.44-2.56 (m, 1H, partially obscured by DMSO), 2.68-2.80 (m, 2H), 3.00-3.09 (m, 2H), 3.95 (br. t, 2H), 5.21-5.29 (m, 1H), 7.09-7.16 (m, 1H), 7.19-7.25 (m, 2H), 7.92 (s, 1H), 8.56 (d, 1H).

### Example 47 (diastereomer 2)

yield: 19 mg

Rₜ=1.78 min; chemical purity>99%; 99% ee

[column: Daicel Chiralpak OX-3, 3 μm, 50 mm×4.6 mm; mobile phase: n-heptane/ethanol+0.2% diethylamine; flow rate: 1 ml/min; temperature: 25° C.; UV detection: 220 nm].

LC-MS (Methode 1): Rₜ=1.19 min; m/z=449 (M+H)⁺.

¹H-NMR (600 MHz, DMSO-d₆, δ/ppm): 0.76-0.89 (m, 4H, including at 0.82 (d, 3H)), 1.34-1.67 (m, 9H, including at 1.42 (d, 3H)), 1.72-1.84 (m, 3H), 2.00-2.12 (m, 1H), 2.44-2.60 (m, 1H, partially obscured by DMSO), 2.69-2.81 (m, 2H), 3.05 (br. t, 2H), 3.89-4.00 (m, 2H), 5.21-5.29 (m, 1H), 7.09-7.16 (m, 1H), 7.18-7.26 (m, 2H), 7.92 (s, 1H), 8.56 (d, 1H).

### Example 48

### rac-N-[(3,5-Difluoropyridin-2-yl)methyl]-2-[3-(methoxymethyl)[1,4′-bipiperidin]-1′-yl]-1,3-thiazole-5-carboxamide

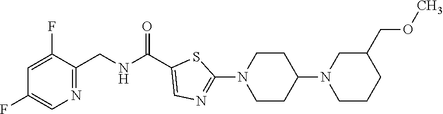 [SMILES: COCC1CCCN(C2CCN(c3ncc(C(=O)NCc4ncc(F)cc4F)s3)CC2)C1]

2-Bromo-N-[(3,5-difluoropyridin-2-yl)methyl]-1,3-thiazole-5-carboxamide (124 mg, 370 μmol) and rac-3-(methoxymethyl)-1,4′-bipiperidine dihydrochloride (123 mg, purity 75%, 285 μmol) were combined and stirred at 120° C. in 2 ml of sodium carbonate solution (2.0 ml, 2.0 M, 4.0 mmol) for 1 h. The reaction mixture was then concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 60.0 mg (purity 100%, 35% of theory) of the target compound.

LC-MS (Methode 4): Rₜ=0.51 min; MS (ESIpos): m/z=466 [M+H]⁺.

¹H-NMR (600 MHz, DMSO-d6) δ[ppm]: 0.903 (0.47), 0.918 (0.53), 1.389 (0.42), 1.409 (0.44), 1.432 (0.44), 1.445 (0.53), 1.452 (0.88), 1.460 (0.62), 1.465 (0.64), 1.472 (0.94), 1.480 (0.56), 1.578 (1.12), 1.596 (1.00), 1.716 (0.49), 1.755 (1.11), 1.774 (0.96), 1.878 (0.66), 1.895 (1.06), 1.912 (0.56), 2.091 (0.43), 2.106 (0.78), 2.109 (0.78), 2.124 (0.42), 2.483 (0.43), 2.520 (0.42), 2.706 (0.61), 2.724 (0.57), 2.795 (0.63), 2.809 (0.61), 3.018 (0.74), 3.023 (0.88), 3.040 (1.54), 3.043 (1.52), 3.060 (0.87), 3.064 (0.76), 3.129 (0.51), 3.144 (1.48), 3.157 (1.78), 3.159 (1.83), 3.169 (1.56), 3.175 (0.63), 3.184 (0.52), 3.200 (16.00), 3.920 (1.12), 3.941 (1.06), 4.521 (2.22), 4.530 (2.22), 7.828 (5.37), 7.893 (0.59), 7.897 (0.63), 7.910 (0.90), 7.913 (0.94), 7.925 (0.60), 7.929 (0.62), 8.465 (2.32), 8.468 (2.28), 8.701 (0.73), 8.710 (1.47), 8.720 (0.71).

### Example 49

### N-[(3,5-Difluoropyridin-2-yl)methyl]-3-[(3R)-3-methyl[1,4′-bipiperidin]-1′-yl]-1,2,4-oxadiazole-5-carboxamide

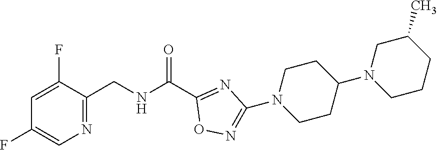 [SMILES: C[C@@H]1CCCN(C2CCN(c3noc(C(=O)NCc4ncc(F)cc4F)n3)CC2)C1]

N,N-Diisopropylethylamine (44 μl, 250 mmol) and propylphosphonic anhydride (66 μl, 50% in ethyl acetate, 110 μmol) were added to a solution of 3-[(3R)-3-methyl[1,4′-bipiperidin]-1′-yl]-1,2,4-oxadiazole-5-carboxylic acid (25.0 mg, 84.9 μmol) and 1-(3,5-difluoropyridin-2-yl)methanamine dihydrochloride (24.0 mg, 110 μmol) in 1 ml of acetonitrile, and the mixture was stirred at room temperature. After 1.5 h, the reaction mixture was concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 7.00 mg (purity 100%, 20% of theory) of the target compound.

LC-MS (Methode 1): Rₜ=0.96 min; MS (ESIpos): m/z=421 [M+H]⁺.

¹H-NMR (600 MHz, DMSO-d6) δ[ppm]: 0.780 (0.59), 0.786 (0.66), 0.813 (14.94), 0.824 (16.00), 0.841 (0.69), 0.847 (0.57), 1.370 (0.56), 1.377 (0.45), 1.391 (1.47), 1.411 (1.58), 1.431 (1.38), 1.437 (1.22), 1.457 (2.49), 1.472 (2.70), 1.493 (1.64), 1.498 (1.66), 1.510 (1.34), 1.516 (1.25), 1.522 (1.29), 1.527 (1.13), 1.567 (1.91), 1.583 (1.19), 1.588 (1.52), 1.618 (1.61), 1.639 (1.55), 1.744 (2.47), 1.760 (5.97), 1.778 (4.64), 2.040 (1.21), 2.055 (2.23), 2.074 (1.19), 2.449 (1.19), 2.467 (2.20), 2.487 (1.30), 2.732 (2.07), 2.746 (3.74), 2.763 (1.77), 2.931 (2.53), 2.949 (4.76), 2.969 (2.54), 3.905 (3.81), 3.927 (3.64), 4.586 (6.49), 4.596 (6.41), 7.930 (1.47), 7.934 (1.53), 7.949 (2.60), 7.962 (1.51), 7.966 (1.50), 8.476 (5.87), 8.479 (5.69), 9.631 (1.76), 9.641 (3.44), 9.651 (1.75).

### Example 50

### diamix-N-[(3,5-Difluoropyridin-2-yl)methyl]-2-[(3R)-3′-fluoro-3-methyl[1,4′-bipiperidin]-1′-yl]-1,3-thiazole-5-carboxamide

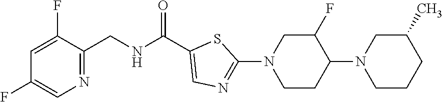 [SMILES: C[C@@H]1CCCN(C2CCN(c3ncc(C(=O)NCc4ncc(F)cc4F)s3)CC2F)C1]

2-Bromo-N-[(3,5-difluoropyridin-2-yl)methyl]-1,3-thiazole-5-carboxamide (200 mg, 599 μmol) and diamix-(3R)-3′-fluoro-3-methyl-1,4′-bipiperidine dihydrochloride (142 mg, 519 μmol) were combined and stirred at 120° C. in 1.2 ml of sodium carbonate solution (1.2 ml, 2.0 M, 2.4 mmol) for 30 min. The reaction mixture was then concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 192 mg (purity 100%, 70% of theory) of the target compound.

LC-MS (Methode 4): Rₜ=0.54 min; MS (ESIpos): m/z=454 [M+H]⁺.

¹H-NMR (600 MHz, DMSO-d6) δ[ppm]: 0.807 (8.04), 0.818 (8.54), 0.823 (9.19), 0.833 (9.26), 0.845 (1.26), 0.851 (1.27), 0.865 (0.57), 0.872 (0.48), 1.375 (0.72), 1.396 (0.88), 1.425 (0.72), 1.513 (0.76), 1.524 (0.78), 1.550 (1.22), 1.572 (1.24), 1.578 (1.30), 1.600 (1.01), 1.624 (1.99), 1.639 (1.94), 1.647 (1.92), 1.795 (1.24), 1.817 (0.99), 1.923 (0.96), 1.932 (0.79), 1.940 (1.73), 1.948 (1.32), 1.958 (1.00), 1.964 (0.66), 2.226 (1.04), 2.245 (1.98), 2.264 (1.01), 2.424 (0.59), 2.653 (0.51), 2.730 (2.22), 2.744 (2.48), 2.801 (1.20), 2.813 (1.28), 3.129 (1.00), 3.134 (1.13), 3.154 (1.85), 3.169 (1.17), 3.214 (0.84), 3.226 (1.61), 3.235 (1.14), 3.247 (1.52), 3.261 (0.83), 3.286 (0.43), 3.705 (1.26), 3.726 (1.18), 4.117 (0.76), 4.123 (0.88), 4.136 (1.42), 4.144 (1.43), 4.156 (0.80), 4.162 (0.74), 4.527 (5.54), 4.536 (5.52), 4.691 (0.60), 4.698 (0.88), 4.705 (1.12), 4.713 (0.79), 4.719 (0.57), 4.773 (0.59), 4.779 (0.81), 4.787 (1.13), 4.794 (0.85), 4.801 (0.57), 7.844 (16.00), 7.899 (1.65), 7.903 (1.77), 7.916 (2.25), 7.918 (2.38), 7.931 (1.68), 7.935 (1.72), 8.468 (6.33), 8.472 (6.30), 8.754 (1.79), 8.764 (3.76), 8.773 (1.86).

### Example 51

### ent-N-[(3,5-Difluoropyridin-2-yl)methyl]-2-[(3R)-3′-fluoro-3-methyl[1,4′-bipiperidin]-1′-yl]-1,3-thiazole-5-carboxamide (enantiomer 1)

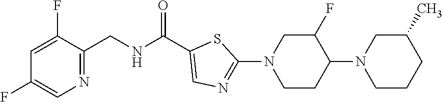 [SMILES: C[C@@H]1CCCN(C2CCN(c3ncc(C(=O)NCc4ncc(F)cc4F)s3)CC2F)C1]

190 mg of diamix-N-[(3,5-difluoropyridin-2-yl)methyl]-2-[(3R)-3′-fluoro-3-methyl[1,4′-bipiperidin]-1′-yl]-1,3-thiazole-5-carboxamide were separated into the stereoisomers by chiral HPLC (preparative HPLC: column Daicel® Chiralpak IA, 5 μm, 250×20 mm; mobile phase: 100% ethanol+0.2% diethylamine; flow rate 20 ml/min; temperature 60° C., detection: 220 nm). The stereoisomer having a retention time of 7.873 min (HPLC: column Daicel® Chiralpak IE 5 μm, flow rate 1 ml/min; mobile phase: 100% ethanol+0.2% diethylamine; temperature 60° C.; detection: 220 nm) was collected. Removal of the solvents gave 88 mg (99% ee) of the title compound.

LC-MS (Methode 1): Rₜ=0.93 min; MS (ESIpos): m/z=454 [M+H]⁺.

¹H.NMR (500 MHz, DMSO-d6) δ[ppm]: S 8.72 (t, 1H), 8.47 (d, 1H), 7.94-7.89 (m, 1H), 7.82 (s, 1H), 5.10 (d, 1H), 4.53 (d, 2H), 4.18 (m, 1H), 4.00 (m, 1H), 3.32 (dd, 1H), 3.18-3.11 (m, 1H), 2.82 (m, 2H), 2.70-2.57 (m, 1H), 2.20-2.14 (m, 1H), 1.94-1.83 (m, 2H), 1.70-1.51 (m, 4H), 1.43-1.33 (m, 1H), 0.88-0.78 (m, 1H), 0.82 (d, 3H).

### Example 52

### ent-N-[(3,5-Difluoropyridin-2-yl)methyl]-2-[(3R)-3′-fluoro-3-methyl[1,4′-bipiperidin]-1′-yl]-1,3-thiazole-5-carboxamide (enantiomer 2)

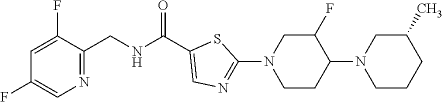 [SMILES: C[C@@H]1CCCN(C2CCN(c3ncc(C(=O)NCc4ncc(F)cc4F)s3)CC2F)C1]

190 mg of diamix-N-[(3,5-difluoropyridin-2-yl)methyl]-2-[(3R)-3′-fluoro-3-methyl[1,4′-bipiperidin]-1′-yl]-1,3-thiazole-5-carboxamide were separated into the stereoisomers by chiral HPLC (preparative HPLC: column Daicel® Chiralpak IA, 5 μm, 250×20 mm; mobile phase: 100% ethanol+0.2% diethylamine; flow rate 20 ml/min; temperature 60° C., detection: 220 nm). The stereoisomer having a retention time of 10.179 min (HPLC: column Daicel® Chiralpak IE 5 μm, flow rate 1 ml/min; mobile phase: 100% ethanol+0.2% diethylamine; temperature 60° C.; detection: 220 nm) was collected. Removal of the solvents gave 91 mg (99% ee) of the title compound.

LC-MS (Methode 1): Rₜ=0.93 min; MS (ESIpos): m/z=454 [M+H]⁺.

¹H.NMR (500 MHz, DMSO-d6) δ[ppm]: δ 8.72 (t, 1H), 8.47 (d, 1H), 7.94-7.89 (m, 1H), 7.82 (s, 1H), 5.10 (d, 1H), 4.53 (d, 2H), 4.18 (m, 1H), 4.00 (m, 1H), 3.32 (dd, 1H), 3.19-3.12 (m, 1H), 2.82 (d br, 2H), 2.70-2.57 (m, 1H), 2.21-2.15 (m, 1H), 1.94-1.84 (m, 2H), 1.70-1.56 (m, 3H), 1.53-1.38 (m, 2H), 0.88-0.78 (m, 1H), 0.81 (d, 3H).

### Example 53

### rac-N-[(3,5-Difluoropyridin-2-yl)methyl]-2-[4-(4-methylazepan-1-yl)piperidin-1-yl]-1,3-thiazole-5-carboxamide

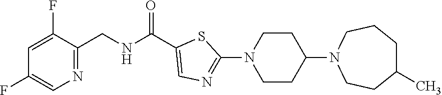 [SMILES: CC1CCCN(C2CCN(c3ncc(C(=O)NCc4ncc(F)cc4F)s3)CC2)CC1]

N,N-Diisopropylethylamine (49 μl, 280 μmol) and acetic acid (9.7 μl, 170 μmol) were added in succession to a solution of N-[(3,5-difluoropyridin-2-yl)methyl]-2-(4-oxopiperidin-1-yl)-1,3-thiazole-5-carboxamide (50.0 mg, 142 μmol) and rac-4-methylazepane (32.1 mg, 284 μmol) in 2.5 ml of dichloromethane, and the mixture was stirred at room temperature overnight. Subsequently, sodium triacetoxyborohydride (45.1 mg, 213 μmol) was added and stirring of the mixture at room temperature was continued. After 2 h, sat. NaHCO₃ solution was added and the reaction mixture was extracted with dichloromethane. The organic phase was concentrated on a rotary evaporator and the residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 43.0 mg (100% purity, 67% of theory) of the title compound.

LC-MS (Methode 1): Rₜ=0.98 min; MS (ESIpos): m/z=450 [M+H]⁺.

¹H-NMR (600 MHz, DMSO-d6) δ[ppm]: 0.874 (16.00), 0.886 (15.94), 1.166 (1.35), 1.173 (2.14), 1.180 (1.44), 1.189 (2.16), 1.195 (1.69), 1.206 (1.49), 1.223 (2.09), 1.240 (2.28), 1.257 (1.07), 1.262 (0.94), 1.417 (1.40), 1.431 (3.73), 1.438 (3.97), 1.451 (4.78), 1.457 (4.63), 1.471 (3.48), 1.478 (2.98), 1.491 (1.13), 1.499 (0.91), 1.566 (1.97), 1.572 (1.71), 1.590 (2.05), 1.609 (1.83), 1.632 (4.40), 1.642 (4.13), 1.649 (3.78), 1.727 (2.59), 1.747 (4.36), 1.766 (2.28), 2.519 (3.82), 2.525 (2.88), 2.567 (1.76), 2.574 (1.84), 2.588 (3.16), 2.594 (2.42), 2.603 (2.38), 2.610 (2.22), 2.636 (3.23), 2.645 (6.11), 2.653 (6.12), 2.664 (4.76), 2.675 (3.65), 2.684 (1.53), 3.020 (3.04), 3.038 (5.45), 3.059 (3.16), 3.327 (0.99), 3.921 (4.02), 3.941 (3.84), 4.523 (7.77), 4.532 (7.71), 7.819 (13.98), 7.877 (1.85), 7.881 (1.92), 7.895 (3.21), 7.897 (3.22), 7.909 (1.86), 7.913 (1.83), 8.458 (6.41), 8.462 (6.13), 8.662 (2.36), 8.671 (4.40), 8.680 (2.29).

### Example 54

### rac-N-[(3,5-Difluoropyridin-2-yl)methyl]-2-[4-(3-methylazepan-1-yl)piperidin-1-yl]-1,3-thiazole-5-carboxamide

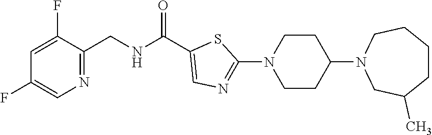 [SMILES: CC1CCCCN(C2CCN(c3ncc(C(=O)NCc4ncc(F)cc4F)s3)CC2)C1]

N,N-Diisopropylethylamine (49 μl, 280 μmol) and acetic acid (9.7 μl, 170 μmol) were added in succession to a solution of N-[(3,5-difluoropyridin-2-yl)methyl]-2-(4-oxopiperidin-1-yl)-1,3-thiazole-5-carboxamide (50.0 mg, 142 μmol) and rac-3-methylazepane hydrochloride (42.5 mg, 284 μmol) in 2.5 ml of dichloromethane, and the mixture was stirred at room temperature overnight. Subsequently, sodium triacetoxyborohydride (45.1 mg, 213 μmol) was added and stirring of the mixture at room temperature was continued. After 2 h, sat. NaHCO₃ solution was added and the reaction mixture was extracted with dichloromethane. The organic phase was concentrated on a rotary evaporator and the residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 40.0 mg (purity 100%, 63% of theory) of the target compound.

LC-MS (Methode 1): Rₜ=0.97 min; MS (ESIpos): m/z=450 [M+H]⁺

¹H-NMR (600 MHz, DMSO-d6) δ[ppm]: 0.813 (15.55), 0.823 (16.00), 1.127 (0.63), 1.144 (1.47), 1.151 (1.29), 1.168 (1.44), 1.409 (0.90), 1.422 (3.44), 1.428 (3.08), 1.440 (5.99), 1.449 (4.90), 1.461 (3.41), 1.468 (2.80), 1.481 (1.05), 1.561 (1.44), 1.573 (1.70), 1.589 (0.97), 1.610 (3.22), 1.629 (4.80), 1.637 (3.85), 1.648 (2.15), 1.738 (3.86), 1.757 (3.39), 2.188 (2.20), 2.202 (2.24), 2.210 (2.45), 2.224 (2.31), 2.569 (0.93), 2.578 (1.13), 2.591 (2.08), 2.600 (2.09), 2.609 (1.71), 2.630 (1.60), 2.639 (4.89), 2.644 (4.73), 2.660 (4.71), 2.664 (4.78), 2.683 (1.26), 3.018 (2.77), 3.035 (5.01), 3.039 (4.91), 3.056 (2.76), 3.256 (0.45), 3.933 (3.56), 3.953 (3.40), 4.524 (7.13), 4.533 (7.07), 7.819 (13.92), 7.880 (1.63), 7.883 (1.71), 7.896 (2.87), 7.899 (2.96), 7.911 (1.68), 7.915 (1.70), 8.460 (6.36), 8.463 (6.29), 8.662 (2.12), 8.672 (4.27), 8.681 (2.16).

### Example 55

### diamix-N-[1-(3,5-Difluoropyridin-2-yl)ethyl]-2-[(3R)-3-methyl[1,4′-bipiperidin]-1′-yl]-1,3-thiazole-5-carboxamide

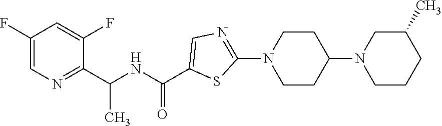 [SMILES: CC(NC(=O)c1cnc(N2CCC(N3CCC[C@@H](C)C3)CC2)s1)c1ncc(F)cc1F]

N,N-Diisopropylethylamine (182 μl, 105 μmol) and propylphosphonic anhydride (86 μl, 50% in ethyl acetate, 290 μmol) were added to a solution of 2-[(3R)-3-methyl[1,4′-bipiperidin]-1′-yl]-1,3-thiazole-5-carboxylic acid dihydrochloride (100 mg, 262 μmol) and rac-1-(3,5-difluoropyridin-2-yl)ethanamine (45.5 mg, 288 μmol) in 5 ml of acetonitrile, and the mixture was stirred at room temperature overnight. The reaction mixture was concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 12.0 mg (purity 100%, 10% of theory) of the target compound.

LC-MS (Methode 1): Rₜ=1.02 min; MS (ESIpos): m/z=450 [M+H]⁺.

¹H-NMR (600 MHz, DMSO-d6) δ[ppm]: 0.788 (0.72), 0.811 (14.96), 0.822 (16.00), 0.843 (0.68), 1.389 (1.55), 1.409 (1.64), 1.440 (14.70), 1.452 (14.49), 1.480 (2.90), 1.502 (2.05), 1.564 (1.99), 1.586 (1.51), 1.615 (1.65), 1.636 (1.56), 1.735 (1.85), 1.753 (4.97), 1.779 (3.32), 2.032 (1.18), 2.049 (2.19), 2.069 (1.17), 2.423 (0.65), 2.466 (1.28), 2.653 (0.49), 2.716 (2.04), 2.731 (3.74), 2.748 (1.88), 3.015 (2.36), 3.036 (4.36), 3.057 (2.38), 3.224 (0.42), 3.249 (0.65), 3.316 (0.89), 3.913 (2.65), 5.317 (0.57), 5.329 (2.00), 5.341 (3.01), 5.353 (1.96), 7.861 (1.44), 7.876 (2.73), 7.893 (1.49), 7.912 (11.30), 8.468 (5.59), 8.531 (3.80), 8.543 (3.75).

### Example 56

### N-[(5-Chloro-1,3-thiazol-2-yl)methyl]-2-[(3R)-3-methyl[1,4′-bipiperidin]-1′-yl]-1,3-thiazole-5-carboxamide

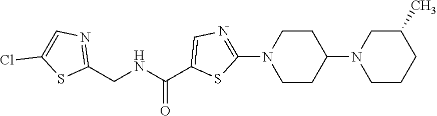 [SMILES: C[C@@H]1CCCN(C2CCN(c3ncc(C(=O)NCc4ncc(Cl)s4)s3)CC2)C1]

N,N-Diisopropylethylamine (230 μl, 1.3 mmol) and propylphosphonic anhydride (86 μl, 50% in ethyl acetate, 290 μmol) were added to a solution of 2-[(3R)-3-methyl[1,4′-bipiperidin]-1′-yl]-1,3-thiazole-5-carboxylic acid dihydrochloride (100 mg, 262 μmol) and 1-(5-chloro-1,3-thiazol-2-yl)methanamine hydrochloride (53.2 mg, 288 μmol) in 5 ml of acetonitrile, and the mixture was stirred at room temperature overnight. The reaction mixture was concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 14.0 mg (purity 100%, 12% of theory) of the target compound.

LC-MS (Methode 1): Rₜ=1.00 min; MS (ESIpos): m/z=440 [M+H]⁺.

¹H-NMR (400 MHz, DMSO-d6) δ[ppm]: −0.149 (0.78), 0.146 (0.87), 0.811 (14.60), 0.827 (16.00), 1.475 (2.13), 1.498 (2.88), 1.605 (1.71), 1.729 (1.52), 1.756 (3.69), 1.802 (2.53), 2.051 (1.90), 2.366 (1.52), 2.710 (2.65), 3.041 (2.14), 3.067 (3.51), 3.098 (1.95), 3.937 (2.72), 3.966 (2.56), 4.573 (8.03), 4.588 (7.85), 7.731 (15.89), 7.837 (15.31), 9.094 (1.71), 9.108 (3.31), 9.122 (1.68).

### Example 57

### N-[(5-Fluoro-2-thienyl)methyl]-2-[(3R)-3-methyl[1,4′-bipiperidin]-1′-yl]-1,3-thiazole-5-carboxamide

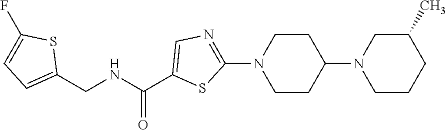 [SMILES: C[C@@H]1CCCN(C2CCN(c3ncc(C(=O)NCc4ccc(F)s4)s3)CC2)C1]

N,N-Diisopropylethylamine (180 μl, 1.0 mmol) and propylphosphonic anhydride (86 μl, 50% in ethyl acetate, 290 μmol) were added to a solution of 2-[(3R)-3-methyl[1,4′-bipiperidin]-1′-yl]-1,3-thiazole-5-carboxylic acid dihydrochloride (100 mg, 262 μmol) and 1-(5-fluoro-2-thienyl)methanamine (37.7 mg, 288 μmol) in 5 ml of acetonitrile, and the mixture was stirred at room temperature overnight. The reaction mixture was concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 12.0 mg (purity 100%, 11% of theory) of the target compound.

LC-MS (Methode 1): Rₜ=1.09 min; MS (ESIpos): m/z=423 [M+H]⁺.

¹H-NMR (600 MHz, DMSO-d6) δ[ppm]: 0.783 (0.52), 0.790 (0.59), 0.813 (15.03), 0.824 (16.00), 0.843 (0.57), 0.850 (0.47), 1.370 (0.50), 1.391 (1.25), 1.411 (1.35), 1.431 (0.57), 1.454 (0.72), 1.474 (1.98), 1.494 (2.47), 1.511 (1.80), 1.529 (0.96), 1.540 (0.58), 1.565 (1.59), 1.571 (1.23), 1.582 (0.96), 1.587 (1.28), 1.616 (1.32), 1.637 (1.24), 1.737 (1.79), 1.754 (3.23), 1.771 (4.08), 1.788 (2.51), 2.036 (1.05), 2.050 (1.91), 2.054 (1.88), 2.069 (1.04), 2.471 (1.13), 2.477 (0.78), 2.722 (1.66), 2.734 (3.05), 2.752 (1.45), 3.031 (1.84), 3.035 (2.16), 3.052 (3.73), 3.055 (3.70), 3.072 (2.12), 3.077 (1.85), 3.257 (0.59), 3.278 (0.99), 3.927 (2.78), 3.948 (2.65), 4.394 (4.22), 4.398 (4.54), 4.404 (4.54), 4.408 (4.29), 6.512 (3.08), 6.516 (3.37), 6.518 (3.69), 6.522 (3.52), 6.660 (2.25), 6.666 (4.14), 6.672 (2.16), 7.780 (13.01), 8.786 (1.58), 8.796 (3.27), 8.806 (1.66).

### Example 58

### 2-[(3R)-3-Methyl[1,4′-bipiperidin]-1′-yl]-N-(pyridin-4-ylmethyl)-1,3-thiazole-5-carboxamide

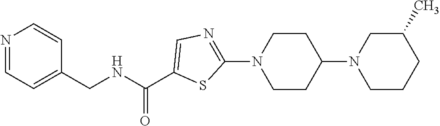 [SMILES: C[C@@H]1CCCN(C2CCN(c3ncc(C(=O)NCc4ccncc4)s3)CC2)C1]

N,N-Diisopropylethylamine (180 μl, 1.0 mmol) and propylphosphonic anhydride (86 μl, 50% in ethyl acetate, 290 μmol) were added to a solution of 2-[(3R)-3-methyl[1,4′-bipiperidin]-1′-yl]-1,3-thiazole-5-carboxylic acid dihydrochloride (100 mg, 262 μmol) and 1-(pyridin-4-yl)methanamine (31.1 mg, 288 μmol) in 5 ml of acetonitrile, and the mixture was stirred at room temperature overnight. The reaction mixture was concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 7.00 mg (purity 100%, 7% of theory) of the target compound.

LC-MS (Methode 1): Rₜ=0.48 min; MS (ESIneg): m/z=398 [M−H]⁻.

¹H-NMR (400 MHz, DMSO-d6) δ[ppm]: 0.776 (0.54), 0.796 (1.55), 0.812 (14.81), 0.828 (16.00), 0.855 (0.65), 0.865 (0.55), 0.950 (1.20), 0.966 (1.16), 1.356 (0.44), 1.387 (1.16), 1.417 (1.35), 1.446 (1.24), 1.474 (2.25), 1.499 (2.83), 1.525 (1.95), 1.534 (1.73), 1.563 (1.86), 1.604 (1.82), 1.645 (1.30), 1.731 (1.87), 1.758 (4.55), 1.783 (2.68), 1.796 (2.56), 2.030 (1.05), 2.052 (1.88), 2.058 (1.85), 2.080 (1.06), 2.366 (0.57), 2.473 (1.30), 2.725 (2.30), 2.741 (2.70), 3.031 (2.07), 3.057 (3.71), 3.088 (2.13), 3.932 (2.97), 3.965 (2.79), 4.401 (6.43), 4.416 (6.43), 7.269 (4.34), 7.280 (4.58), 7.849 (13.88), 8.505 (1.83), 8.800 (1.58), 8.815 (3.27), 8.830 (1.59).

### Example 59

### rac-N-[(3,5-Difluoropyridin-2-yl)methyl]-2-{3-[(2,2,2-trifluoroethoxy)methyl][1,4′-bipiperidin]-1′-yl}-1,3-thiazole-5-carboxamide

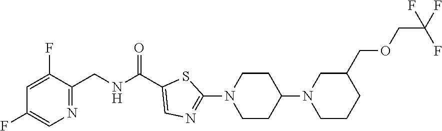 [SMILES: O=C(NCc1ncc(F)cc1F)c1cnc(N2CCC(N3CCCC(COCC(F)(F)F)C3)CC2)s1]

2-Bromo-N-[(3,5-difluoropyridin-2-yl)methyl]-1,3-thiazole-5-carboxamide (118 mg, 353 μmol) and rac-3-[(2,2,2-trifluoroethoxy)methyl]-1,4′-bipiperidine dihydrochloride (164 mg, purity 75%, 348 μmol) were combined and stirred at 120° C. in 2 ml of sodium carbonate solution (2 ml, 2.0 M, 4 mmol) for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 56.0 mg (purity 100%, 30% of theory) of the target compound.

LC-MS (Methode 5): Rₜ=1.64 min; MS (ESIpos): m/z=534 [M+H]⁺.

¹H-NMR (600 MHz, DMSO-d6) δ[ppm]: 0.937 (0.65), 0.955 (1.56), 0.970 (1.59), 0.988 (0.67), 1.381 (0.58), 1.402 (1.31), 1.420 (1.45), 1.431 (1.19), 1.439 (1.22), 1.452 (2.08), 1.458 (1.80), 1.465 (1.92), 1.471 (3.02), 1.478 (1.98), 1.485 (1.91), 1.492 (2.15), 1.505 (0.81), 1.512 (0.59), 1.589 (3.51), 1.605 (3.09), 1.767 (4.45), 1.952 (1.98), 1.969 (3.12), 1.986 (1.74), 2.133 (1.36), 2.148 (2.51), 2.166 (1.33), 2.513 (2.55), 2.689 (1.93), 2.707 (1.83), 2.776 (2.08), 2.791 (2.00), 3.029 (2.57), 3.049 (4.90), 3.070 (2.56), 3.425 (0.45), 3.443 (7.66), 3.454 (8.96), 3.925 (3.82), 3.947 (3.63), 3.976 (3.33), 3.992 (9.56), 4.008 (9.29), 4.023 (3.00), 4.525 (7.17), 4.534 (7.14), 7.824 (16.00), 7.877 (1.74), 7.881 (1.86), 7.897 (2.97), 7.909 (1.76), 7.913 (1.81), 8.458 (6.85), 8.462 (6.81), 8.666 (2.27), 8.676 (4.58), 8.685 (2.26).

### Example 60

### rac-N-[(3,5-Difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide

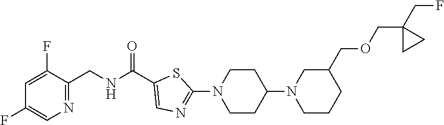 [SMILES: O=C(NCc1ncc(F)cc1F)c1cnc(N2CCC(N3CCCC(COCC4(CF)CC4)C3)CC2)s1]

2-Bromo-N-[(3,5-difluoropyridin-2-yl)methyl]-1,3-thiazole-5-carboxamide (75.5 mg, 226 μmol) and rac-3-({[1-(fluoromethyl)cyclopropyl]methoxy}methyl)-1,4′-bipiperidine dihydrochloride (133 mg) were combined and stirred at 120° C. in 2 ml of sodium carbonate solution (2 ml, 2.0 M, 4 mmol) for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 10.5 mg (purity 100%, 9% of theory) of the target compound.

LC-MS (Methode 5): Rₜ=1.65 min; MS (ESIpos): m/z=538 [M+H]⁺.

¹H-NMR (600 MHz, DMSO-d6) δ[ppm]: 0.500 (0.62), 0.507 (0.85), 0.526 (0.86), 0.531 (1.03), 1.601 (0.40), 3.051 (0.52), 3.242 (1.08), 3.253 (1.11), 3.279 (2.71), 3.289 (16.00), 3.923 (0.41), 3.943 (0.40), 4.219 (0.96), 4.301 (0.97), 4.524 (0.77), 4.533 (0.76), 7.824 (1.56), 8.459 (0.67), 8.463 (0.68), 8.675 (0.48).

### Example 61

### rac-2-[3-({[1-(Difluoromethyl)cyclopropyl]methoxy}methyl)[1,4′-bipiperidin]-1′-yl]-N-[(3,5-difluoropyridin-2-yl)methyl]-1,3-thiazole-5-carboxamide

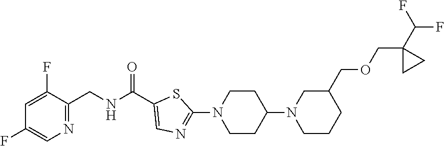 [SMILES: O=C(NCc1ncc(F)cc1F)c1cnc(N2CCC(N3CCCC(COCC4(C(F)F)CC4)C3)CC2)s1]

2-Bromo-N-[(3,5-difluoropyridin-2-yl)methyl]-1,3-thiazole-5-carboxamide (100 mg, 300 μmol) and rac-3-({[1-(difluoromethyl)cyclopropyl]methoxy}methyl)-1,4′-bipiperidine dihydrochloride (112 mg) were combined and stirred at 120° C. in 2 ml of sodium carbonate solution (2 ml, 2.0 M, 4 mmol) for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 49.8 mg (purity 100%, 30% of theory) of the target compound.

LC-MS (Methode 5): Rₜ=1.71 min; MS (ESIpos): m/z=556 [M+H]⁺.

¹H-NMR (600 MHz, DMSO-d6) δ[ppm]: 0.608 (6.75), 0.611 (6.72), 0.725 (3.50), 0.733 (9.17), 0.735 (8.79), 0.743 (2.54), 0.904 (0.57), 0.921 (1.21), 0.936 (1.35), 0.955 (0.59), 1.372 (0.53), 1.391 (1.17), 1.411 (1.22), 1.437 (0.65), 1.444 (0.72), 1.456 (1.67), 1.466 (1.85), 1.476 (2.47), 1.485 (1.95), 1.496 (1.74), 1.515 (0.52), 1.587 (2.51), 1.592 (2.61), 1.599 (2.32), 1.722 (1.44), 1.739 (1.01), 1.759 (2.61), 1.781 (2.25), 1.909 (1.70), 1.926 (2.76), 1.943 (1.48), 2.109 (1.17), 2.125 (2.16), 2.142 (1.16), 2.486 (1.43), 2.522 (1.19), 2.699 (1.73), 2.718 (1.60), 2.791 (1.81), 2.806 (1.75), 3.029 (2.22), 3.050 (4.07), 3.071 (2.19), 3.237 (7.84), 3.248 (8.49), 3.384 (0.66), 3.403 (16.00), 3.422 (0.65), 3.922 (3.21), 3.943 (3.05), 4.524 (6.21), 4.533 (6.24), 5.805 (2.61), 5.901 (5.22), 5.996 (2.47), 7.824 (12.56), 7.878 (1.45), 7.882 (1.55), 7.897 (2.60), 7.910 (1.53), 7.914 (1.57), 8.458 (5.72), 8.462 (5.73), 8.666 (1.94), 8.675 (3.99), 8.684 (2.01).

### Example 62

### rac-N-[(3,5-Difluoropyridin-2-yl)methyl]-2-[3-({[1-(trifluoromethyl)cyclopropyl]methoxy}methyl)[1,4′-bipiperidin]-1′-yl]-1,3-thiazole-5-carboxamide

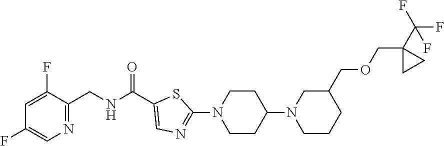 [SMILES: O=C(NCc1ncc(F)cc1F)c1cnc(N2CCC(N3CCCC(COCC4(C(F)(F)F)CC4)C3)CC2)s1]

2-Bromo-N-[(3,5-difluoropyridin-2-yl)methyl]-1,3-thiazole-5-carboxamide (110 mg, 329 μmol) and rac-3-({[1-(trifluoromethyl)cyclopropyl]methoxy}methyl)-1,4′-bipiperidine dihydrochloride (129 mg) were combined and stirred at 120° C. in 2 ml of sodium carbonate solution (2 ml, 2.0 M, 4 mmol) for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 56.0 mg (purity 100%, 30% of theory) of the target compound.

LC-MS (Methode 5): Rₜ=1.78 min; MS (ESIpos): m/z=574 [M+H]⁺.

¹H-NMR (600 MHz, DMSO-d6) δ[ppm]: 0.808 (7.29), 0.908 (0.91), 0.926 (1.41), 0.934 (3.81), 0.942 (9.64), 0.953 (2.74), 1.393 (1.17), 1.411 (1.23), 1.432 (0.90), 1.440 (0.99), 1.452 (1.48), 1.460 (2.20), 1.470 (2.07), 1.480 (2.36), 1.500 (0.99), 1.584 (2.93), 1.600 (2.53), 1.721 (1.36), 1.757 (2.85), 1.779 (2.43), 1.907 (1.65), 1.925 (2.68), 1.941 (1.46), 2.110 (1.18), 2.125 (2.13), 2.144 (1.12), 2.482 (1.29), 2.519 (1.31), 2.699 (1.69), 2.717 (1.59), 2.788 (1.78), 2.804 (1.71), 3.031 (2.12), 3.052 (3.86), 3.073 (2.08), 3.233 (0.60), 3.252 (5.22), 3.262 (7.02), 3.456 (0.42), 3.475 (16.00), 3.495 (0.43), 3.920 (3.15), 3.942 (3.00), 4.524 (6.08), 4.533 (6.04), 7.824 (11.55), 7.879 (1.35), 7.883 (1.50), 7.897 (2.52), 7.911 (1.45), 7.914 (1.45), 8.459 (5.46), 8.462 (5.45), 8.666 (1.84), 8.675 (3.81), 8.685 (1.89).

### Example 63

### N-[(3,5-Difluoropyridin-2-yl)methyl]-2-(3,3-dimethyl[1,4′-bipiperidin]-1′-yl)-1,3-thiazole-5-carboxamide

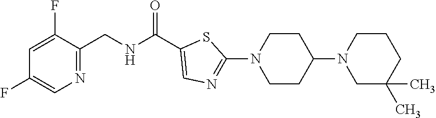 [SMILES: CC1(C)CCCN(C2CCN(c3ncc(C(=O)NCc4ncc(F)cc4F)s3)CC2)C1]

2-Bromo-N-[(3,5-difluoropyridin-2-yl)methyl]-1,3-thiazole-5-carboxamide (50.0 mg, 150 μmol) and 3,3-dimethyl-1,4′-bipiperidine dihydrochloride (52.3 mg) were initially charged in 1 ml of water. Sodium carbonate (63.4 mg, 599 μmol) was added and the mixture was stirred at 120° C. for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 37.0 mg (purity 100%, 55% of theory) of the target compound.

LC-MS (Methode 1): Rₜ=0.95 min; MS (ESIpos): m/z=450 [M+H]⁺.

¹H-NMR (600 MHz, DMSO-d6) δ[ppm]: 0.882 (16.00), 1.150 (0.79), 1.160 (1.14), 1.170 (0.89), 1.468 (1.40), 1.479 (1.32), 1.488 (1.33), 1.729 (0.86), 1.747 (0.73), 2.097 (2.01), 2.392 (0.76), 2.473 (0.61), 3.034 (0.54), 3.038 (0.63), 3.056 (1.10), 3.076 (0.63), 3.080 (0.54), 3.902 (0.87), 3.923 (0.82), 4.520 (1.58), 4.529 (1.56), 7.826 (3.86), 7.894 (0.42), 7.898 (0.44), 7.911 (0.63), 7.913 (0.67), 7.926 (0.43), 7.930 (0.43), 8.464 (1.64), 8.468 (1.59), 8.699 (0.51), 8.709 (1.03), 8.719 (0.50).

### Example 64

### 2-[4-(5-Azaspiro[2.5]octan-5-yl)piperidin-1-yl]-N-[(3,5-difluoropyridin-2-yl)methyl]-1,3-thiazole-5-carboxamide

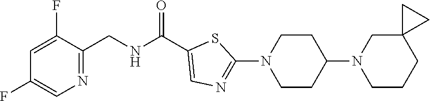 [SMILES: O=C(NCc1ncc(F)cc1F)c1cnc(N2CCC(N3CCCC4(CC4)C3)CC2)s1]

2-Bromo-N-[(3,5-difluoropyridin-2-yl)methyl]-1,3-thiazole-5-carboxamide (200 mg, 599 μmol) and 5-(piperidin-4-yl)-5-azaspiro[2.5]octane dihydrochloride (180 mg) were initially charged in 2 ml of water. Sodium carbonate (254 mg, 2.39 mmol) was added and the mixture was stirred at 120° C. for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 108 mg (purity 100%, 40% of theory) of the target compound.

LC-MS (Methode 1): Rₜ=0.90 min; MS (ESIpos): m/z=448 [M+H]⁺.

¹H.NMR (500 MHz, DMSO-d6) δ[ppm]: δ 8.70 (t, 1H), 8.46 (d, 1H), 7.94-7.89 (m, 1H), 7.82 (s, 1H), 4.52 (d, 2H), 3.90 (d br, 2H), 3.08-3.02 (m, 2H), 2.47-2.40 (m, 3H), 2.19 (s, 2H) 1.77 (d br, 2H), 1.57 (m, 2H), 1.50-1.40 (m, 2H), 1.24 (t, 2H), 0.28-0.21 (m, 4H).

### Example 65

### rac-2-[4-(1,1-Difluoro-5-azaspiro[2.5]octan-5-yl)piperidin-1-yl]-N-[(3,5-difluoropyridin-2-yl)methyl]-1,3-thiazole-5-carboxamide

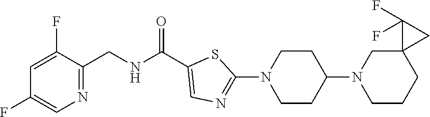 [SMILES: O=C(NCc1ncc(F)cc1F)c1cnc(N2CCC(N3CCCC4(C3)CC4(F)F)CC2)s1]

2-Bromo-N-[(3,5-difluoropyridin-2-yl)methyl]-1,3-thiazole-5-carboxamide (100 mg, 299 μmol) and rac-1,1-difluoro-5-(piperidin-4-yl)-5-azaspiro[2.5]octane dihydrochloride (104 mg) were initially charged in 1 ml of water. Sodium carbonate (127 mg, 1.20 mmol) was added and the mixture was stirred at 120° C. for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 62.0 mg (purity 100%, 43% of theory) of the target compound.

LC-MS (Methode 5): Rₜ=1.56 min; MS (ESIpos): m/z=484 [M+H]⁺.

¹H.NMR (500 MHz, DMSO-d6) δ[ppm]: δ 8.71 (t, 1H), 8.46 (d, 1H), 7.94-7.89 (m, 1H), 7.83 (s, 1H), 4.52 (d, 2H), 3.92 (d br, 2H), 3.10-3.02 (m, 2H), 2.67-2.57 (m, 3H), 2.44-2.37 (m, 2H), 1.78 (t br, 2H), 1.60 (m, 1H), 1.53-1.42 (m, 5H), 1.26-1.14 (m, 2H).

### Example 66

### rac-2-[3-(Cyclobutylmethoxy)[1,4′-bipiperidin]-1′-yl]-N-[(3,5-difluoropyridin-2-yl)methyl]-1,3-thiazole-5-carboxamide

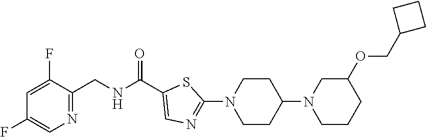 [SMILES: O=C(NCc1ncc(F)cc1F)c1cnc(N2CCC(N3CCCC(OCC4CCC4)C3)CC2)s1]

N,N-Diisopropylethylamine (49 μl, 280 μmol) and acetic acid (12 μl, 210 μmol) were added in succession to a solution of N-[(3,5-difluoropyridin-2-yl)methyl]-2-(4-oxopiperidin-1-yl)-1,3-thiazole-5-carboxamide (50.0 mg, 142 μmol) and rac-3-(cyclobutylmethoxy)piperidine hydrochloride (58.4 mg, 284 μmol) in 5 ml of dichloromethane, and the mixture was stirred at room temperature overnight. Subsequently, sodium triacetoxyborohydride (36.1 mg, 170 μmol) was added and stirring of the mixture at room temperature was continued. After 1.5 h, more sodium triacetoxyborohydride (36.1 mg, 170 μmol) was added and stirring of the mixture at room temperature was continued. After 2 h, sat. NaHCO₃ solution was added and the reaction mixture was extracted with dichloromethane. The organic phase was concentrated on a rotary evaporator and the residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 5.00 mg (purity 100%, 7% of theory) of the target compound.

LC-MS (Methode 1): Rₜ=1.22 min; MS (ESIpos): m/z=506 [M+H]⁺.

¹H-NMR (600 MHz, DMSO-d6) δ[ppm]: 1.009 (0.53), 1.016 (0.59), 1.029 (1.45), 1.036 (1.37), 1.053 (1.53), 1.066 (0.71), 1.074 (0.56), 1.316 (0.60), 1.337 (1.44), 1.357 (1.50), 1.376 (0.65), 1.453 (0.95), 1.472 (2.73), 1.492 (2.83), 1.511 (1.08), 1.610 (1.84), 1.632 (1.99), 1.643 (3.00), 1.657 (3.90), 1.672 (3.26), 1.685 (1.32), 1.759 (3.93), 1.781 (4.11), 1.796 (2.12), 1.808 (3.21), 1.822 (4.29), 1.836 (2.48), 1.840 (1.45), 1.849 (0.87), 1.854 (0.98), 1.867 (0.44), 1.890 (1.64), 1.904 (1.62), 1.919 (1.99), 1.931 (3.81), 1.945 (6.23), 1.953 (3.44), 1.961 (4.23), 1.974 (1.09), 2.059 (1.35), 2.073 (2.48), 2.092 (1.33), 2.403 (1.00), 2.415 (2.19), 2.427 (2.80), 2.440 (2.11), 2.452 (0.94), 2.564 (1.15), 2.652 (2.37), 2.669 (1.88), 2.942 (1.96), 2.954 (1.82), 3.018 (2.55), 3.038 (4.84), 3.058 (2.54), 3.205 (1.36), 3.214 (1.73), 3.221 (2.34), 3.229 (1.64), 3.237 (1.32), 3.244 (0.74), 3.293 (0.74), 3.354 (1.90), 3.365 (2.08), 3.370 (5.42), 3.382 (7.32), 3.394 (5.38), 3.399 (2.01), 3.410 (1.64), 3.929 (3.33), 3.949 (3.21), 4.520 (6.95), 4.530 (6.93), 7.828 (16.00), 7.894 (1.82), 7.898 (1.94), 7.913 (2.92), 7.926 (1.83), 7.930 (1.88), 8.465 (7.15), 8.468 (7.02), 8.701 (2.24), 8.711 (4.57), 8.721 (2.24).

### Example 67

### rac-2-[3-(Cyclopropylmethoxy)[1,4′-bipiperidin]-1′-yl]-N-[(3,5-difluoropyridin-2-yl)methyl]-1,3-thiazole-5-carboxamide

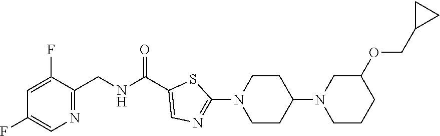 [SMILES: O=C(NCc1ncc(F)cc1F)c1cnc(N2CCC(N3CCCC(OCC4CC4)C3)CC2)s1]

2-Bromo-N-[(3,5-difluoropyridin-2-yl)methyl]-1,3-thiazole-5-carboxamide (60.8 mg, 182 μmol) and rac-3-(cyclopropylmethoxy)-1,4′-bipiperidine dihydrochloride (50.0 mg) were combined and stirred at 120° C. in 2 ml of sodium carbonate solution (2 ml, 2.0 M, 4 mmol) for 1 hour. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 20.0 mg (purity 100%, 22% of theory) of the target compound.

LC-MS (Methode 1): Rₜ=1.06 min; MS (ESIpos): m/z=492 [M+H]⁺.

¹H-NMR (600 MHz, DMSO-d6) δ[ppm]: 0.114 (2.20), 0.122 (7.19), 0.129 (7.41), 0.137 (2.33), 0.410 (2.11), 0.416 (6.34), 0.419 (6.12), 0.430 (6.52), 0.433 (6.08), 0.439 (1.85), 0.923 (1.53), 0.935 (2.28), 0.947 (1.44), 1.024 (0.64), 1.038 (1.55), 1.060 (1.59), 1.075 (0.78), 1.316 (0.66), 1.336 (1.50), 1.357 (1.60), 1.376 (0.70), 1.451 (1.00), 1.471 (2.74), 1.482 (2.39), 1.491 (2.85), 1.499 (1.88), 1.511 (1.16), 1.610 (1.97), 1.633 (1.67), 1.758 (3.91), 1.777 (3.38), 1.886 (1.71), 1.900 (1.62), 1.926 (2.05), 1.942 (3.48), 1.959 (2.11), 2.062 (1.42), 2.077 (2.57), 2.095 (1.40), 2.423 (0.62), 2.520 (1.90), 2.558 (1.22), 2.652 (2.62), 2.669 (2.02), 2.943 (2.00), 2.956 (1.92), 3.018 (2.70), 3.037 (5.11), 3.057 (2.70), 3.240 (13.81), 3.251 (14.04), 3.264 (2.67), 3.271 (1.87), 3.280 (1.60), 3.288 (1.56), 3.344 (0.84), 3.927 (3.48), 3.946 (3.30), 4.520 (7.38), 4.529 (7.39), 7.827 (16.00), 7.895 (1.83), 7.899 (1.91), 7.915 (3.15), 7.927 (1.89), 7.931 (1.94), 8.465 (7.46), 8.469 (7.11), 8.702 (2.42), 8.711 (4.84), 8.721 (2.39).

### Example 68

### rac-2-{3-[(Cyclobutyloxy)methyl][1,4′-bipiperidin]-1′-yl}-N-[(3,5-difluoropyridin-2-yl)methyl]-1,3-thiazole-5-carboxamide

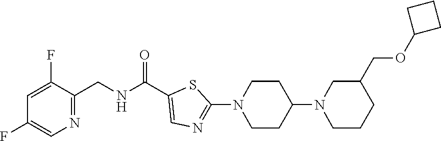 [SMILES: O=C(NCc1ncc(F)cc1F)c1cnc(N2CCC(N3CCCC(COC4CCC4)C3)CC2)s1]

2-Bromo-N-[(3,5-difluoropyridin-2-yl)methyl]-1,3-thiazole-5-carboxamide (100 mg, 299 μmol) and rac-3-[(cyclobutyloxy)methyl]-1,4′-bipiperidine dihydrochloride (144 mg) were combined and stirred at 120° C. in 2 ml of sodium carbonate solution (2 ml, 2.0 M, 4 mmol) for 1 hour. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength: 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 38.0 mg (purity 100%, 25% of theory) of the target compound.

LC-MS (Methode 1): Rₜ=1.11 min; MS (ESIpos): m/z=506 [M+H]⁺.

¹H-NMR (600 MHz, DMSO-d6) δ[ppm]: 0.880 (0.72), 0.897 (1.70), 0.913 (1.85), 0.932 (0.78), 1.361 (0.72), 1.381 (1.75), 1.387 (1.75), 1.404 (2.77), 1.418 (3.45), 1.435 (3.99), 1.443 (2.72), 1.450 (3.84), 1.463 (3.80), 1.483 (2.61), 1.574 (4.23), 1.588 (6.01), 1.604 (4.37), 1.622 (1.30), 1.659 (1.93), 1.753 (6.08), 1.761 (6.58), 1.768 (6.61), 1.774 (6.32), 1.804 (0.59), 1.883 (2.18), 1.900 (3.68), 1.917 (1.93), 2.099 (5.66), 2.113 (7.26), 2.131 (2.89), 2.522 (1.55), 2.691 (2.43), 2.709 (2.25), 2.788 (2.57), 2.802 (2.45), 3.022 (3.24), 3.041 (6.13), 3.061 (3.30), 3.084 (4.70), 3.098 (4.88), 3.101 (5.01), 3.111 (4.67), 3.117 (2.12), 3.127 (1.53), 3.294 (0.66), 3.357 (0.67), 3.793 (0.85), 3.805 (3.00), 3.818 (4.35), 3.829 (2.93), 3.842 (0.82), 3.924 (4.69), 3.945 (4.39), 4.520 (8.81), 4.529 (8.73), 7.827 (16.00), 7.896 (1.92), 7.911 (3.69), 7.926 (1.86), 8.464 (7.34), 8.467 (7.27), 8.700 (2.70), 8.709 (5.37), 8.719 (2.66).

### Example 69

### rac-2-{3-[(Cyclopropylmethoxy)methyl][1,4′-bipiperidin]-1′-yl}-N-[(3,5-difluoropyridin-2-yl)methyl]-1,3-thiazole-5-carboxamide

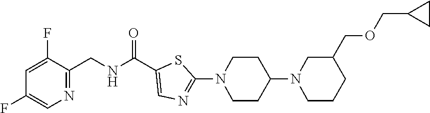 [SMILES: O=C(NCc1ncc(F)cc1F)c1cnc(N2CCC(N3CCCC(COCC4CC4)C3)CC2)s1]

2-Bromo-N-[(3,5-difluoropyridin-2-yl)methyl]-1,3-thiazole-5-carboxamide (50.9 mg, 152 μmol) and rac-3-[(cyclopropylmethoxy)methyl]-1,4′-bipiperidine dihydrochloride (44.0 mg) were combined and stirred at 120° C. in 2 ml of sodium carbonate solution (2 ml, 2.0 M, 4 mmol) for 1 hour. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 15.0 mg (purity 100%, 19% of theory) of the target compound.

LC-MS (Methode 1): Rₜ=1.12 min; MS (ESIpos): m/z=506 [M+H]⁺.

¹H-NMR (600 MHz, DMSO-d6) δ[ppm]: 0.118 (2.25), 0.126 (9.10), 0.134 (9.17), 0.142 (2.22), 0.419 (2.38), 0.426 (7.33), 0.427 (7.44), 0.439 (7.57), 0.441 (7.33), 0.448 (1.98), 0.889 (0.73), 0.904 (1.72), 0.922 (2.00), 0.947 (2.51), 0.958 (2.65), 0.969 (1.73), 0.978 (0.81), 1.369 (0.75), 1.388 (1.75), 1.409 (1.76), 1.428 (1.26), 1.435 (1.20), 1.447 (2.46), 1.467 (3.67), 1.487 (2.56), 1.507 (0.71), 1.573 (2.43), 1.579 (2.59), 1.593 (4.27), 1.609 (2.19), 1.705 (1.94), 1.766 (3.63), 1.894 (2.27), 1.911 (3.77), 1.928 (1.94), 2.099 (1.66), 2.114 (3.06), 2.132 (1.60), 2.526 (1.44), 2.701 (2.49), 2.719 (2.26), 2.802 (2.62), 2.817 (2.51), 3.026 (3.23), 3.045 (6.22), 3.064 (3.23), 3.147 (0.41), 3.165 (13.68), 3.176 (13.45), 3.191 (1.65), 3.206 (5.08), 3.219 (9.08), 3.228 (5.08), 3.234 (1.95), 3.244 (1.30), 3.296 (0.60), 3.923 (4.82), 3.944 (4.45), 4.521 (8.96), 4.530 (8.75), 7.827 (16.00), 7.893 (2.03), 7.896 (2.11), 7.911 (3.75), 7.924 (2.05), 7.928 (2.02), 8.464 (8.06), 8.467 (7.70), 8.700 (2.76), 8.710 (5.37), 8.719 (2.62).

### Example 70

### rac-N-[(3,5-Difluoropyridin-2-yl)methyl]-2-[3-ethoxy[1,4′-bipiperidin]-1′-yl]-1,3-thiazole-5-carboxamide

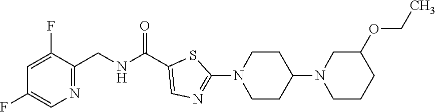 [SMILES: CCOC1CCCN(C2CCN(c3ncc(C(=O)NCc4ncc(F)cc4F)s3)CC2)C1]

Acetic acid (12 μl, 210 μmol) was added to a solution of N-[(3,5-difluoropyridin-2-yl)methyl]-2-(4-oxopiperidin-1-yl)-1,3-thiazole-5-carboxamide (50.0 mg, 142 μmol) and rac-3-ethoxypiperidine (36.7 mg, 284 μmol) in 5 ml of dichloromethane, and the mixture was stirred at room temperature overnight. Subsequently, sodium triacetoxyborohydride (36.1 mg, 170 μmol) was added and stirring of the mixture at room temperature was continued. After 4 h, sat. NaHCO₃ solution was added and the reaction mixture was extracted with dichloromethane. The organic phase was concentrated on a rotary evaporator and the residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 15.0 mg (purity 100%, 23% of theory) of the target compound.

LC-MS (Methode 1): Rₜ=0.94 min; MS (ESIpos): m/z=466 [M+H]⁺.

¹H-NMR (600 MHz, DMSO-d6) δ[ppm]: 1.021 (0.44), 1.035 (0.92), 1.042 (0.86), 1.057 (8.38), 1.068 (16.00), 1.080 (8.12), 1.320 (0.40), 1.342 (0.87), 1.360 (0.89), 1.472 (1.63), 1.492 (1.70), 1.511 (0.65), 1.611 (1.11), 1.617 (0.92), 1.627 (0.80), 1.634 (1.00), 1.759 (2.28), 1.778 (1.99), 1.887 (0.97), 1.901 (0.93), 1.935 (1.27), 1.951 (2.00), 1.967 (1.30), 2.062 (0.81), 2.076 (1.56), 2.080 (1.49), 2.095 (0.84), 2.523 (1.00), 2.561 (0.76), 2.652 (1.54), 2.670 (1.13), 2.938 (1.14), 2.949 (1.07), 3.019 (1.52), 3.038 (2.87), 3.058 (1.56), 3.221 (0.46), 3.228 (0.88), 3.235 (1.05), 3.244 (1.46), 3.251 (1.03), 3.259 (0.83), 3.266 (0.45), 3.346 (0.70), 3.351 (0.76), 3.423 (0.98), 3.427 (1.06), 3.434 (1.21), 3.438 (4.04), 3.450 (6.06), 3.461 (4.04), 3.465 (1.20), 3.473 (1.09), 3.477 (0.96), 3.927 (1.96), 3.948 (1.87), 4.521 (4.14), 4.530 (4.13), 7.828 (11.05), 7.895 (1.16), 7.899 (1.24), 7.912 (1.70), 7.914 (1.80), 7.927 (1.19), 7.931 (1.24), 8.465 (4.56), 8.469 (4.52), 8.702 (1.37), 8.712 (2.80), 8.721 (1.38).

### Example 71

### N-[(3,5-Difluoropyridin-2-yl)methyl]-2-{4-[(3R)-3-methylpiperidin-1-yl]azepan-1-yl}-1,3-thiazole-5-carboxamide

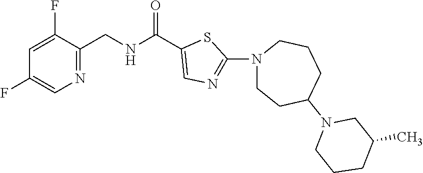 [SMILES: C[C@@H]1CCCN(C2CCCN(c3ncc(C(=O)NCc4ncc(F)cc4F)s3)CC2)C1]

2-Bromo-N-[(3,5-difluoropyridin-2-yl)methyl]-1,3-thiazole-5-carboxamide (70.0 mg, 209 μmol) and 4-[(3R)-3-methylpiperidin-1-yl]azepane dihydrochloride (48.8 mg) were initially charged in 1 ml of water. Sodium carbonate (88.8 mg, 838 μmol) was added and the mixture was stirred at 120° C. for 1 h. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 38.0 mg (purity 100%, 40% of theory) of the target compound.

LC-MS (Methode 5): Rₜ=1.80 min; MS (ESIpos): m/z=450 [M+H]⁺.

¹H-NMR (600 MHz, DMSO-d6) δ[ppm]: 0.763 (0.66), 0.790 (11.01), 0.794 (11.81), 0.800 (12.77), 0.805 (11.44), 1.354 (1.54), 1.368 (3.05), 1.373 (2.83), 1.387 (2.70), 1.409 (1.20), 1.488 (1.52), 1.534 (2.35), 1.556 (2.32), 1.593 (2.70), 1.614 (2.01), 1.688 (1.57), 1.705 (1.69), 1.722 (0.79), 1.740 (1.27), 1.757 (4.10), 1.773 (4.04), 1.790 (2.51), 1.894 (3.01), 2.065 (1.69), 2.383 (1.59), 2.399 (2.60), 2.417 (1.27), 2.599 (4.31), 2.615 (3.21), 3.354 (1.52), 3.378 (2.77), 3.397 (2.73), 3.655 (1.75), 4.519 (7.77), 4.528 (7.74), 7.825 (16.00), 7.893 (1.86), 7.897 (1.91), 7.909 (3.18), 7.924 (1.95), 7.928 (1.92), 8.463 (7.52), 8.467 (7.26), 8.646 (2.37), 8.656 (4.74), 8.665 (2.33).

### Example 72

### 2-[(3R)-3-methyl[1,4′-bipiperidin]-1′-yl]-N-[(6-methylpyridin-3-yl)methyl]-1,3-thiazole-5-carboxamide

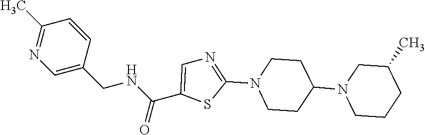

N,N-Diisopropylethylamine (180 μl, 1.0 mmol) and propylphosphonic anhydride (86 μl, 50% in ethyl acetate, 290 μmol) were added to a solution of 2-[(3R)-3-methyl[1,4′-bipiperidin]-1′-yl]-1,3-thiazole-5-carboxylic acid dihydrochloride (100 mg, 262 μmol) and 1-(6-methylpyridin-3-yl)methanamine (35.1 mg, 288 μmol) in 5 ml of acetonitrile, and the mixture was stirred at room temperature overnight. The reaction mixture was concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 19.0 mg (purity 100%, 18% of theory) of the target compound.

LC-MS (Methode 1): Rₜ=0.55 min; MS (ESIneg): m/z=412 [M−H]⁻.

¹H-NMR (600 MHz, DMSO-d6) δ[ppm]: 0.799 (0.74), 0.811 (7.56), 0.822 (8.04), 1.388 (0.65), 1.408 (0.66), 1.466 (1.03), 1.490 (1.23), 1.508 (0.93), 1.519 (0.60), 1.525 (0.54), 1.564 (0.83), 1.580 (0.51), 1.586 (0.67), 1.615 (0.70), 1.636 (0.67), 1.732 (0.88), 1.750 (1.63), 1.767 (2.09), 1.783 (1.34), 2.032 (0.52), 2.046 (0.96), 2.050 (0.94), 2.065 (0.52), 2.431 (16.00), 2.470 (0.64), 2.720 (0.87), 2.734 (1.57), 2.751 (0.74), 3.026 (1.11), 3.044 (1.97), 3.064 (1.13), 3.924 (1.42), 3.945 (1.35), 4.349 (3.74), 4.359 (3.72), 7.196 (2.33), 7.210 (2.52), 7.556 (1.53), 7.560 (1.54), 7.570 (1.45), 7.573 (1.43), 7.795 (6.86), 8.366 (2.40), 8.370 (2.40), 8.711 (0.92), 8.721 (1.84), 8.731 (0.92).

### Example 73

### N-Benzyl-2-[(3R)-3-methyl[1,4′-bipiperidin]-1′-yl]-1,3-thiazole-5-carboxamide

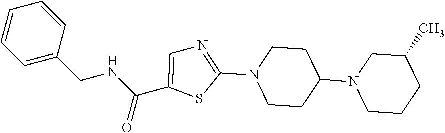 [SMILES: C[C@@H]1CCCN(C2CCN(c3ncc(C(=O)NCc4ccccc4)s3)CC2)C1]

N,N-Diisopropylethylamine (100 μl, 580 μmol) and propylphosphonic anhydride (47 μl, 50% in ethyl acetate, 160 μmol) were added to a solution of 2-[(3R)-3-methyl[1,4′-bipiperidin]-1′-yl]-1,3-thiazole-5-carboxylic acid hydrochloride (50.0 mg, 145 μmol) and 1-phenylmethanamine (17 μl, 160 μmol) in 5 ml of acetonitrile, and the mixture was stirred at room temperature. After 30 min, the reaction mixture was concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 25.0 mg (purity 100%, 43% of theory) of the target compound.

LC-MS (Methode 1): Rₜ=1.04 min; MS (ESIpos): m/z=399 [M+H]⁺.

¹H-NMR (600 MHz, DMSO-d6) δ[ppm]: 0.785 (0.53), 0.791 (0.62), 0.815 (14.94), 0.826 (16.00), 0.844 (0.63), 0.851 (0.53), 1.372 (0.52), 1.393 (1.35), 1.407 (0.92), 1.413 (1.45), 1.433 (0.62), 1.458 (0.78), 1.477 (2.21), 1.496 (2.75), 1.513 (2.00), 1.531 (1.04), 1.542 (0.63), 1.567 (1.74), 1.572 (1.34), 1.583 (1.05), 1.589 (1.39), 1.617 (1.47), 1.638 (1.39), 1.739 (1.91), 1.757 (3.65), 1.773 (4.17), 1.788 (2.83), 2.038 (1.12), 2.053 (2.08), 2.056 (2.04), 2.071 (1.14), 2.471 (1.17), 2.477 (0.82), 2.724 (1.87), 2.736 (3.43), 2.754 (1.63), 3.030 (2.28), 3.048 (4.18), 3.068 (2.31), 3.929 (3.15), 3.951 (2.98), 4.387 (7.95), 4.397 (7.93), 7.225 (1.22), 7.237 (3.31), 7.248 (2.13), 7.277 (4.54), 7.289 (8.70), 7.310 (6.60), 7.322 (7.31), 7.336 (2.52), 7.822 (11.60), 8.684 (1.68), 8.694 (3.35), 8.704 (1.70).

### Example 74

### diamix-N-[(3,5-Difluoropyridin-2-yl)methyl]-2-[3-({[3-fluorobutyl]oxy}methyl)[1,4′-bipiperidin]-1′-yl]-1,3-thiazole-5-carboxamide

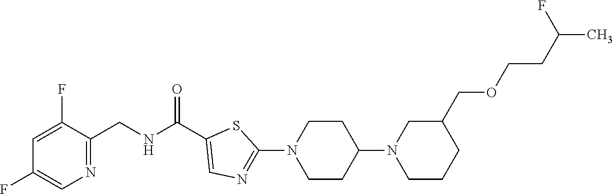 [SMILES: CC(F)CCOCC1CCCN(C2CCN(c3ncc(C(=O)NCc4ncc(F)cc4F)s3)CC2)C1]

2-Bromo-N-[(3,5-difluoropyridin-2-yl)methyl]-1,3-thiazole-5-carboxamide (100 mg, 299 μmol) and diamix-3-[(3-fluorobutoxy)methyl]-1,4′-bipiperidine dihydrochloride (92.4 mg) were combined and stirred at 120° C. in 2 ml of sodium carbonate solution (2 ml, 2.0 M, 4 mmol) for 1 hour. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B.

Mobile phase C and mobile phase D constant flow rate of 5 ml/min each over the entire running time). The product-containing fractions were combined and lyophilized. This gave 45.0 mg (purity 100%, 29% of theory) of the target compound.

LC-MS (Methode 1): Rₜ=1.10 min; MS (ESIpos): m/z=526 [M+H]⁺.

¹H-NMR (600 MHz, DMSO-d6) δ[ppm]: 0.894 (0.51), 0.910 (1.21), 0.927 (1.31), 0.946 (0.55), 1.231 (1.01), 1.249 (9.88), 1.259 (9.97), 1.289 (9.92), 1.300 (9.69), 1.367 (0.53), 1.387 (1.24), 1.407 (1.28), 1.426 (0.92), 1.434 (0.92), 1.445 (1.68), 1.455 (1.93), 1.465 (2.52), 1.476 (2.04), 1.485 (1.80), 1.493 (0.81), 1.504 (0.53), 1.574 (1.89), 1.589 (2.71), 1.595 (2.68), 1.698 (1.15), 1.710 (1.65), 1.721 (1.89), 1.729 (1.76), 1.734 (1.74), 1.742 (2.56), 1.752 (3.56), 1.758 (3.71), 1.768 (3.79), 1.771 (3.66), 1.777 (3.74), 1.780 (3.74), 1.787 (2.97), 1.799 (1.97), 1.808 (0.76), 1.900 (1.21), 1.915 (2.14), 1.931 (1.08), 2.101 (1.15), 2.115 (2.11), 2.133 (1.13), 2.485 (1.36), 2.522 (1.14), 2.698 (1.78), 2.716 (1.64), 2.788 (1.77), 2.804 (1.72), 3.023 (2.35), 3.043 (4.47), 3.064 (2.31), 3.181 (0.85), 3.197 (1.94), 3.209 (5.46), 3.221 (4.27), 3.231 (1.98), 3.237 (1.28), 3.247 (0.88), 3.368 (0.58), 3.378 (1.01), 3.384 (1.18), 3.387 (0.79), 3.394 (1.92), 3.401 (3.11), 3.411 (4.27), 3.421 (2.19), 3.428 (1.41), 3.431 (1.34), 3.441 (1.12), 3.444 (0.79), 3.457 (0.56), 3.921 (3.36), 3.943 (3.18), 4.521 (6.49), 4.530 (6.43), 4.687 (0.71), 4.698 (0.98), 4.708 (0.96), 4.718 (0.66), 4.769 (0.74), 4.780 (1.12), 4.790 (1.10), 4.800 (0.71), 7.828 (16.00), 7.892 (1.77), 7.896 (1.83), 7.909 (2.66), 7.911 (2.78), 7.924 (1.78), 7.928 (1.78), 8.463 (7.00), 8.467 (6.72), 8.701 (2.17), 8.710 (4.39), 8.720 (2.11).

### Example 75

### rac-2-(3-{[(3,3-Difluorocyclobutyl)methoxy]methyl}[1,4′-bipiperidin]-1′-yl)-N-[(3,5-difluoropyridin-2-yl)methyl]-1,3-thiazole-5-carboxamide

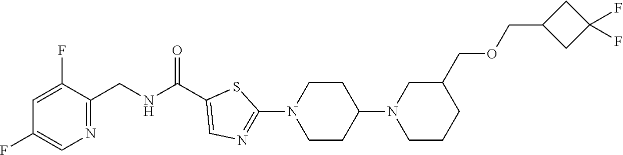 [SMILES: O=C(NCc1ncc(F)cc1F)c1cnc(N2CCC(N3CCCC(COCC4CC(F)(F)C4)C3)CC2)s1]

2-Bromo-N-[(3,5-difluoropyridin-2-yl)methyl]-1,3-thiazole-5-carboxamide (100 mg, 299 μmol) and rac-3-{[(3,3-difluorocyclobutyl)methoxy]methyl}-1,4′-bipiperidine dihydrochloride (286 mg) were combined and stirred at 120° C. in 2 ml of sodium carbonate solution (2 ml, 2.0 M, 4 mmol) for 1 hour. The reaction mixture was then diluted with water and extracted with dichloromethane. The organic phase was dried over Na₂SO₄, the drying agent was filtered off and the filtrate was concentrated on a rotary evaporator. The residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 42.0 mg (purity 100%, 25% of theory) of the target compound.

LC-MS (Methode 1): Rₜ=1.20 min; MS (ESIpos): m/z=556 [M+H]⁺.

¹H-NMR (600 MHz, DMSO-d6) δ[ppm]: 0.911 (0.78), 0.928 (1.72), 0.945 (1.85), 0.963 (0.81), 1.374 (0.78), 1.394 (1.76), 1.414 (1.76), 1.434 (1.28), 1.441 (1.17), 1.454 (2.40), 1.466 (2.42), 1.473 (3.58), 1.486 (2.51), 1.493 (2.58), 1.596 (3.83), 1.731 (2.04), 1.759 (4.07), 1.917 (2.39), 1.935 (3.91), 1.952 (2.04), 2.110 (1.73), 2.126 (3.13), 2.144 (1.58), 2.261 (0.89), 2.274 (1.32), 2.295 (3.70), 2.317 (5.95), 2.336 (3.61), 2.485 (2.03), 2.521 (1.62), 2.564 (2.23), 2.574 (2.41), 2.578 (2.59), 2.587 (3.86), 2.601 (2.66), 2.611 (2.16), 2.701 (2.47), 2.719 (2.29), 2.789 (2.63), 2.804 (2.46), 3.029 (3.08), 3.050 (5.72), 3.070 (3.05), 3.231 (0.68), 3.247 (9.82), 3.258 (11.25), 3.358 (0.88), 3.381 (7.63), 3.389 (5.10), 3.405 (0.86), 3.921 (4.65), 3.943 (4.34), 4.524 (8.79), 4.534 (8.71), 7.823 (16.00), 7.878 (1.95), 7.882 (2.02), 7.897 (3.64), 7.910 (1.95), 7.914 (2.00), 8.458 (7.56), 8.462 (7.46), 8.665 (2.65), 8.674 (5.27), 8.684 (2.62).

### Example 76

### N-[(3-Fluoropyridin-4-yl)methyl]-2-[(3R)-3-methyl[1,4′-bipiperidin]-1′-yl]-1,3-thiazole-5-carboxamide

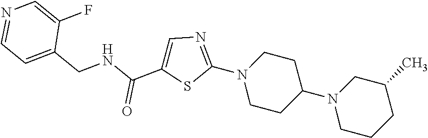 [SMILES: C[C@@H]1CCCN(C2CCN(c3ncc(C(=O)NCc4ccncc4F)s3)CC2)C1]

N,N-Diisopropylethylamine (180 μl, 1.0 mmol) and propylphosphonic anhydride (86 μl, 50% in ethyl acetate, 290 μmol) were added to a solution of 2-[(3R)-3-methyl[1,4′-bipiperidin]-1′-yl]-1,3-thiazole-5-carboxylic acid hydrochloride (100 mg, 262 μmol) and 1-(3-fluoropyridin-4-yl)methanamine (36.3 mg, 288 μmol) in 5 ml of acetonitrile, and the mixture was stirred at room temperature overnight. The reaction mixture was then concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 21.0 mg (purity 100%, 19% of theory) of the target compound.

LC-MS (Methode 1): Rₜ=0.82 min; MS (ESIneg): m/z=416 [M−H].

¹H-NMR (600 MHz, DMSO-d6) δ[ppm]: 0.788 (0.58), 0.814 (15.04), 0.825 (16.00), 0.842 (0.70), 1.371 (0.56), 1.392 (1.36), 1.412 (1.46), 1.432 (0.62), 1.473 (2.29), 1.493 (2.88), 1.511 (2.09), 1.568 (1.79), 1.589 (1.42), 1.618 (1.54), 1.640 (1.49), 1.738 (1.72), 1.756 (3.14), 1.773 (4.60), 1.792 (2.86), 2.037 (1.07), 2.056 (2.00), 2.071 (1.12), 2.425 (0.56), 2.520 (1.70), 2.653 (0.50), 2.726 (1.87), 2.738 (3.35), 2.757 (1.60), 3.042 (2.38), 3.059 (4.22), 3.080 (2.39), 3.287 (0.93), 3.937 (3.04), 3.959 (2.92), 4.466 (7.56), 4.476 (7.60), 7.336 (2.33), 7.345 (3.35), 7.355 (2.45), 7.859 (12.63), 8.383 (4.52), 8.391 (4.59), 8.511 (6.60), 8.513 (6.51), 8.819 (1.88), 8.829 (3.81), 8.839 (1.85).

### Example 77

### rac-N-[(3,5-Difluoropyridin-2-yl)methyl]-2-[3-(2,2,2-trifluoroethoxy)[1,4′-bipiperidin]-1′-yl]-1,3-thiazole-5-carboxamide

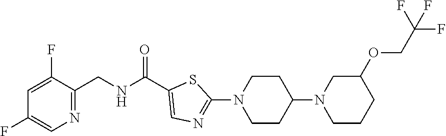 [SMILES: O=C(NCc1ncc(F)cc1F)c1cnc(N2CCC(N3CCCC(OCC(F)(F)F)C3)CC2)s1]

Acetic acid (12 μl, 210 μmol) was added to a solution of N-[(3,5-difluoropyridin-2-yl)methyl]-2-(4-oxopiperidin-1-yl)-1,3-thiazole-5-carboxamide (50.0 mg, 142 μmol) and rac-3-(2,2,2-trifluoroethoxy)piperidine (52.0 mg, 284 μmol) in 5 ml of dichloromethane, and the mixture was stirred at room temperature overnight. Subsequently, sodium triacetoxyborohydride (36.1 mg, 170 μmol) was added and stirring of the mixture at room temperature was continued. After 1.5 h, more sodium triacetoxyborohydride (36.1 mg, 170 μmol) was added and stirring of the mixture at room temperature was continued. After 2 h, sat. NaHCO₃ solution was added and the reaction mixture was extracted with dichloromethane. The organic phase was concentrated on a rotary evaporator and the residue was dissolved in DMSO and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 7.00 mg (purity 100%, 9% of theory) of the target compound.

LC-MS (Methode 1): Rₜ=1.06 min; MS (ESIpos): m/z=520 [M+H]⁺.

¹H-NMR (600 MHz, DMSO-d6) δ[ppm]: 1.124 (1.34), 1.147 (1.39), 1.160 (0.64), 1.343 (1.36), 1.365 (1.39), 1.475 (1.80), 1.496 (2.67), 1.516 (1.84), 1.637 (1.77), 1.658 (1.46), 1.758 (3.69), 1.777 (3.15), 1.916 (1.56), 2.015 (1.74), 2.032 (2.94), 2.048 (1.77), 2.106 (1.29), 2.120 (2.39), 2.138 (1.28), 2.423 (0.93), 2.565 (2.35), 2.584 (0.93), 2.640 (1.95), 2.652 (2.04), 2.658 (1.76), 2.969 (1.90), 2.981 (1.80), 3.025 (2.41), 3.045 (4.81), 3.065 (2.50), 3.282 (1.41), 3.289 (0.62), 3.345 (1.02), 3.350 (0.92), 3.447 (1.28), 3.455 (1.66), 3.462 (2.19), 3.470 (1.56), 3.478 (1.17), 3.934 (3.16), 3.953 (3.03), 4.042 (1.46), 4.049 (1.60), 4.058 (4.17), 4.065 (4.12), 4.073 (4.05), 4.081 (3.91), 4.096 (1.27), 4.520 (6.74), 4.529 (6.68), 7.828 (16.00), 7.895 (1.76), 7.900 (1.83), 7.915 (2.87), 7.927 (1.78), 7.931 (1.82), 8.465 (6.78), 8.469 (6.85), 8.703 (2.10), 8.712 (4.35), 8.722 (2.21).

### Example 78

### N-[(4,6-Dimethylpyridin-3-yl)methyl]-2-[(3R)-3-methyl[1,4′-bipiperidin]-1′-yl]-1,3-thiazole-5-carboxamide

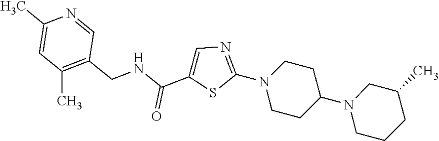

N,N-Diisopropylethylamine (180 μl, 1.0 mmol) and propylphosphonic anhydride (86 μl, 50% in ethyl acetate, 290 μmol) were added to a solution of 2-[(3R)-3-methyl[1,4′-bipiperidin]-1′-yl]-1,3-thiazole-5-carboxylic acid hydrochloride (100 mg, 262 μmol) and 1-(4,6-dimethylpyridin-3-yl)methanamine (39.2 mg, 288 μmol) in 5 ml of acetonitrile, and the mixture was stirred at room temperature overnight. The reaction mixture was then concentrated on a rotary evaporator and the residue was dissolved in DMSO, filtered and purified by preparative HPLC (instrument: Waters Prep LC/MS System, column: XBridge C18 5 μm 100×30 mm; mobile phase A: water, mobile phase B: acetonitrile, mobile phase C: 2% ammonia in water, mobile phase D: acetonitrile/water (80% by volume/20% by volume); total flow rate: 80 ml/min; room temperature; wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 8.00 mg (purity 100%, 7% of theory) of the target compound.

LC-MS (Methode 1): Rₜ=0.53 min; MS (ESIneg): m/z=426 [M−H]⁻.

¹H-NMR (400 MHz, DMSO-d6) δ[ppm]: −0.149 (0.57), 0.146 (0.57), 0.808 (8.12), 0.825 (8.74), 1.382 (0.62), 1.413 (0.76), 1.422 (0.69), 1.465 (1.17), 1.486 (1.46), 1.494 (1.54), 1.513 (0.97), 1.559 (0.97), 1.601 (0.93), 1.640 (0.67), 1.724 (1.01), 1.751 (2.71), 1.776 (1.44), 1.786 (1.36), 2.023 (0.58), 2.045 (0.99), 2.073 (0.56), 2.263 (16.00), 2.327 (0.71), 2.366 (1.24), 2.386 (15.84), 2.459 (0.67), 2.669 (0.76), 2.674 (0.57), 2.710 (2.03), 2.736 (1.41), 3.013 (1.10), 3.039 (1.91), 3.070 (1.13), 3.294 (2.40), 3.916 (1.56), 3.949 (1.50), 4.352 (3.75), 4.366 (3.82), 7.051 (3.86), 7.802 (8.23), 8.243 (4.42), 8.518 (0.89), 8.532 (1.87), 8.546 (0.89).

### Example 79

### N-[(4-Chloro-1-methyl-1H-pyrazol-5-yl)methyl]-2-[(3R)-3-methyl[1,4′-bipiperidin]-1′-yl]-1,3-thiazole-5-carboxamide

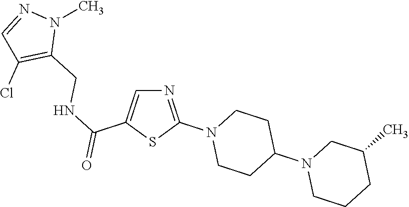 [SMILES: C[C@@H]1CCCN(C2CCN(c3ncc(C(=O)NCc4c(Cl)cnn4C)s3)CC2)C1]

30.9 mg (0.10 mmol) of 2-[(3R)-3-methyl[1,4′-bipiperidin]-1′-yl]-1,3-thiazole-5-carboxylic acid, 53.2 mg (0.14 mmol) of HATU and 50 μl of 4-methylmorpholine were dissolved in 0.7 ml of DMF and stirred at RT for 30 min. The solution was then added to 29.2 mg (0.20 mmol) of 1-(4-chloro-1-methyl-1H-pyrazol-5-yl)methanamine, which had been initially charged into a well of a 96-well multititre plate, and the multititre plate was sealed and shaken at RT overnight. 0.2 ml of water were then added, the mixture was filtered and the filtrate was separated into its components by preparative LC-MS using one of the following methods:

Prep. Lc-Ms Methods:
MS instrument: Waters, HPLC instrument: Waters (column Waters X-Bridge C18, 19 mm×50 mm, 5 μm, mobile phase A: water+0.375% ammonia, mobile phase B: acetonitrile (ULC)+0.375% ammonia with gradient; flow rate: 40 ml/min; UV detection: DAD; 210-400 nm).
or alternatively:
MS instrument: Waters, HPLC instrument: Waters (column Phenomenex Luna 5μ C18(2) 100A, AXIA Tech. 50×21.2 mm, mobile phase A: water+0.0375% formic acid, mobile phase B: acetonitrile (ULC)+0.0375% formic acid with gradient; flow rate: 40 ml/min; UV detection: DAD; 210-400 nm).

In this way, 27.7 mg (63% of theory, 96% purity) of the title compound were obtained.

LC-MS (Methode 6, ESIpos): Rₜ=0.69 min; m/z=437 (M+H)⁺.

¹H-NMR (500 MHz, DMSO-d₆, δ/ppm): 0.90 (d, 3H), 1.03-1.15 (m, 1H), 1.60-1.90 (m, 6H), 2.05-2.14 (m, 2H), 2.56-2.65 (m, 1H), 2.80-2.91 (m, 1H), 3.12 (br. t, 2H), 3.33 (br. d, 1H), 3.36-3.51 (m, 1H, partially obscured by H₂O), 3.82 (s, 3H), 4.08 (br. d, 2H), 4.45 (d, 2H), 7.49 (s, 1H), 7.85 (s, 1H), 8.68 (t, 1H), 8.96-9.04 (m, 1H).

In a parallel-synthetic manner analogously to Example 79, the following compounds of Examples 80 to 98 were prepared from 2-[(3R)-3-methyl[1,4′-bipiperidin]-1′-yl]-1,3-thiazole-5-carboxylic acid and the appropiate amine or its salt:

| Example | IUPAC name/structure (yield; purity) | LC-MS (Method 6) |
|---|---|---|
| 80 | 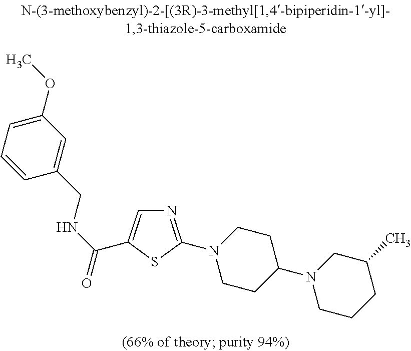 [SMILES: COc1cccc(CNC(=O)c2cnc(N3CCC(N4CCC[C@@H](C)C4)CC3)s2)c1] | Rₜ = 0.73 min; m/z = 429 (M + H)⁺ |
| 81 | 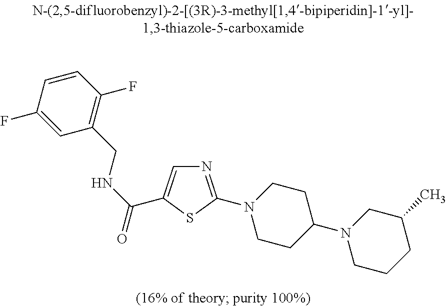 [SMILES: C[C@@H]1CCCN(C2CCN(c3ncc(C(=O)NCc4cc(F)ccc4F)s3)CC2)C1] | Rₜ = 0.75 min; m/z = 435 (M + H)⁺ |
| 82 | 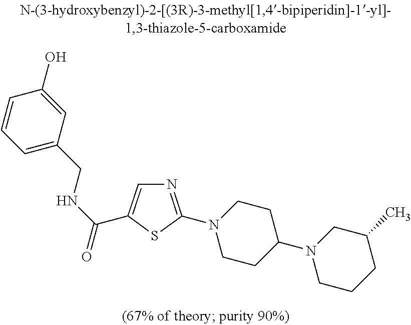 [SMILES: C[C@@H]1CCCN(C2CCN(c3ncc(C(=O)NCc4cccc(O)c4)s3)CC2)C1] | Rₜ = 0.66 min; m/z = 415 (M + H)⁺ |
| 83 | 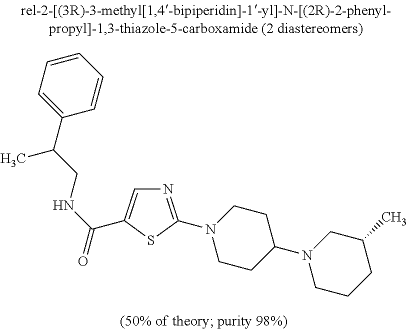 [SMILES: CC(CNC(=O)c1cnc(N2CCC(N3CCC[C@@H](C)C3)CC2)s1)c1ccccc1] | Rₜ = 0.76 min; m/z = 427 (M + H)⁺ |
| 84 | 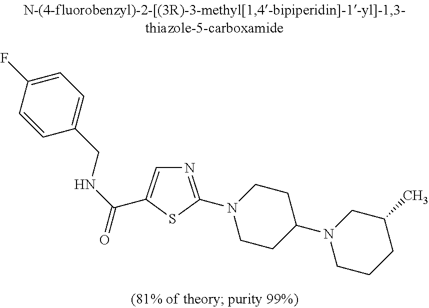 [SMILES: C[C@@H]1CCCN(C2CCN(c3ncc(C(=O)NCc4ccc(F)cc4)s3)CC2)C1] | Rₜ = 0.73 min; m/z = 417 (M + H)⁺ |
| 85 | 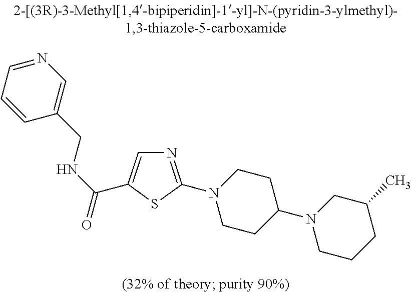 [SMILES: C[C@@H]1CCCN(C2CCN(c3ncc(C(=O)NCc4cccnc4)s3)CC2)C1] | Rₜ = 0.63 min; m/z = 400 (M + H)⁺ |
| 86 | 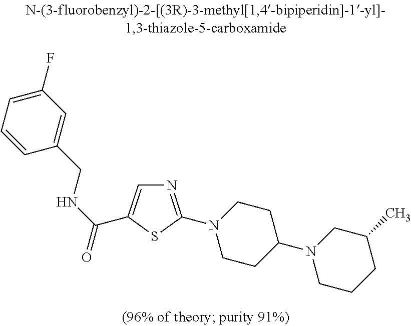 [SMILES: C[C@@H]1CCCN(C2CCN(c3ncc(C(=O)NCc4cccc(F)c4)s3)CC2)C1] | Rₜ = 0.72 min; m/z = 417 (M + H)⁺ |
| 87 | 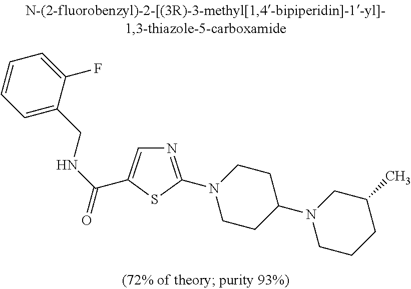 [SMILES: C[C@@H]1CCCN(C2CCN(c3ncc(C(=O)NCc4ccccc4F)s3)CC2)C1] | Rₜ = 0.73 min; m/z = 417 (M + H)⁺ |
| 88 | 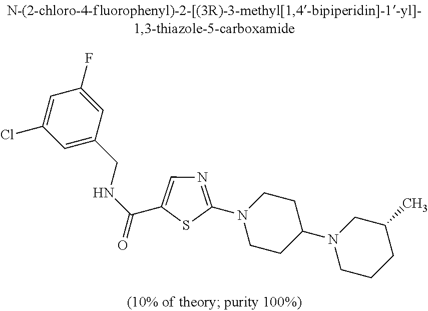 | Rₜ = 0.76 min; m/z = 437 (M + H)⁺ |
| 89 | 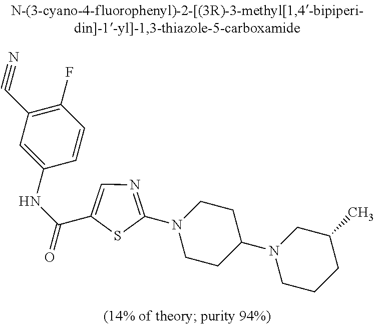 [SMILES: C[C@@H]1CCCN(C2CCN(c3ncc(C(=O)Nc4ccc(F)c(C#N)c4)s3)CC2)C1] | Rₜ = 0.77 min; m/z = 428 (M + H)⁺ |
| 90 | 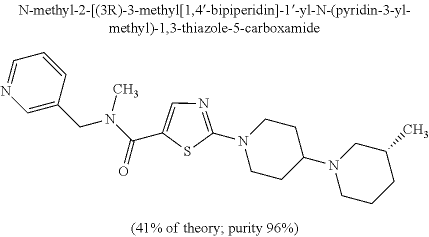 [SMILES: C[C@@H]1CCCN(C2CCN(c3ncc(C(=O)N(C)Cc4cccnc4)s3)CC2)C1] | Rₜ = 0.60 min; m/z = 414 (M + H)⁺ |
| 91 | 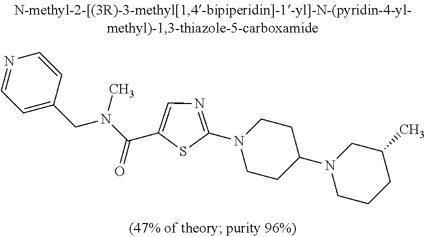 [SMILES: C[C@@H]1CCCN(C2CCN(c3ncc(C(=O)N(C)Cc4ccncc4)s3)CC2)C1] | Rₜ = 0.57 min; m/z = 414 (M + H)⁺ |
| 92 | 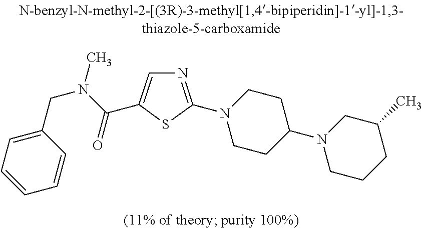 [SMILES: C[C@@H]1CCCN(C2CCN(c3ncc(C(=O)N(C)Cc4ccccc4)s3)CC2)C1] | Rₜ = 0.75 min; m/z = 413 (M + H)⁺ |
| 93 | 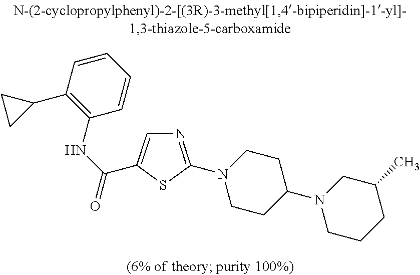 [SMILES: C[C@@H]1CCCN(C2CCN(c3ncc(C(=O)Nc4ccccc4C4CC4)s3)CC2)C1] | Rₜ = 0.87 min; m/z = 425 (M + H)⁺ |
| 94 | 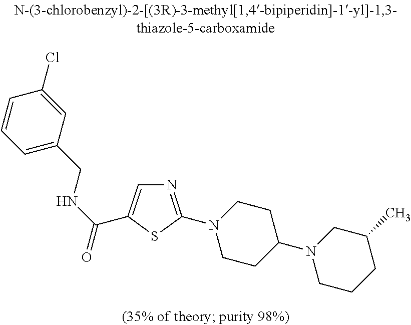 [SMILES: C[C@@H]1CCCN(C2CCN(c3ncc(C(=O)NCc4cccc(Cl)c4)s3)CC2)C1] | Rₜ = 0.85 min; m/z = 433 (M + H)⁺ |
| 95 | 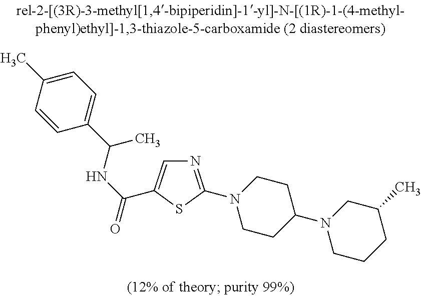 | Rₜ = 0.87 min; m/z = 427 (M + H)⁺ |
| 96 | 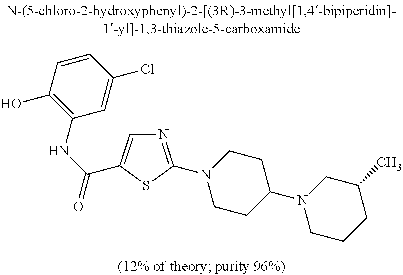 [SMILES: C[C@@H]1CCCN(C2CCN(c3ncc(C(=O)Nc4cc(Cl)ccc4O)s3)CC2)C1] | Rₜ = 0.85 min; m/z = 435 (M + H)⁺ |
| 97 | 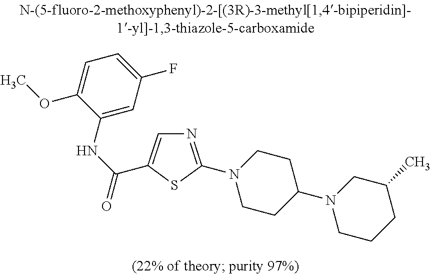 | Rₜ = 0.87 min; m/z = 433 (M + H)⁺ |
| 98 | 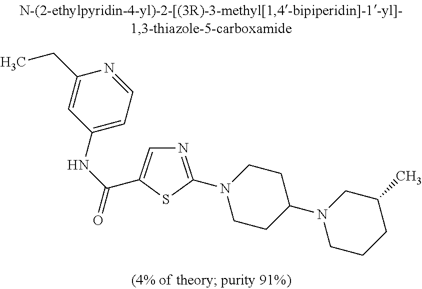 [SMILES: CCc1cc(NC(=O)c2cnc(N3CCC(N4CCC[C@@H](C)C4)CC3)s2)ccn1] | Rₜ = 0.65 min; m/z = 414 (M + H)⁺ |

### Example 99

### ent-N-[(3,5-Difluoropyridin-2-yl)methyl]-2-[3-(methoxymethyl) [1,4′-bipiperidin]-1′-yl]-1,3-thiazole-5-carboxamide (enantiomer 1)

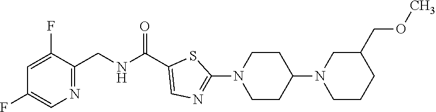 [SMILES: COCC1CCCN(C2CCN(c3ncc(C(=O)NCc4ncc(F)cc4F)s3)CC2)C1]

45 mg of rac-N-[(3,5-difluoropyridin-2-yl)methyl]-2-[3-(methoxymethyl)[1,4′-bipiperidin]-1′-yl]-1,3-thiazole-5-carboxamide were separated into the enantiomers by chiral HPLC (preparative HPLC: column Daicel@ Chiralpak IG, 5 μm, 250×20 mm; mobile phase: 100% ethanol+0.2% diethylamine; flow rate 15 ml/min; temperature 55° C., detection: 220 nm). The enantiomer having a retention time of 10.838 min (HPLC: column Daicel© Chiralpak IE 5 μm, flow rate 1 ml/min; mobile phase: 100% ethanol+0.2% diethylamine; temperature 60° C.; detection: 220 nm) was collected. Removal of the solvents gave 23 mg (99% ee) of the title compound.

LC-MS (Methode 1): Rₜ=0.89 min; MS (ESIpos): m/z=466 [M+H]⁺.

¹H-NMR (600 MHz, DMSO-d6) δ[ppm]: 0.911 (0.66), 0.927 (0.72), 1.392 (0.58), 1.410 (0.63), 1.436 (0.52), 1.457 (1.21), 1.477 (1.28), 1.497 (0.51), 1.582 (1.53), 1.598 (1.39), 1.719 (0.66), 1.758 (1.52), 1.778 (1.33), 1.887 (0.89), 1.904 (1.48), 1.921 (0.77), 2.097 (0.59), 2.114 (1.12), 2.132 (0.59), 2.707 (0.80), 2.726 (0.79), 2.796 (0.89), 2.809 (0.83), 3.026 (1.13), 3.044 (2.13), 3.065 (1.14), 3.136 (0.55), 3.151 (1.82), 3.164 (3.39), 3.173 (1.89), 3.189 (0.57), 3.203 (16.00), 3.919 (1.63), 3.941 (1.53), 4.522 (2.97), 4.531 (2.96), 7.822 (5.28), 7.879 (0.69), 7.897 (1.23), 7.910 (0.69), 8.459 (2.58), 8.462 (2.46), 8.663 (0.90), 8.673 (1.76), 8.682 (0.91).

### Example 100

### ent-N-[(3,5-Difluoropyridin-2-yl)methyl]-2-[3-(methoxymethyl)[1,4′-bipiperidin]-1′-yl]-1,3-thiazole-5-carboxamide (enantiomer 2)

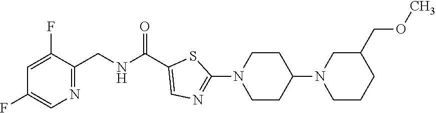 [SMILES: COCC1CCCN(C2CCN(c3ncc(C(=O)NCc4ncc(F)cc4F)s3)CC2)C1]

45 mg of rac-N-[(3,5-difluoropyridin-2-yl)methyl]-2-[3-(methoxymethyl)[1,4′-bipiperidin]-1′-yl]-1,3-thiazole-5-carboxamide were separated into the enantiomers by chiral HPLC (preparative HPLC: column Daicel® Chiralpak IG, 5 μm, 250×20 mm; mobile phase: 100% ethanol+0.2% diethylamine; flow rate 15 ml/min; temperature 55° C., detection: 220 nm). The enantiomer having a retention time of 11.879 min (HPLC: column Daicel® Chiralpak IE 5 μm, flow rate 1 ml/min; mobile phase: 100% ethanol+0.2% diethylamine; temperature 60° C.; detection: 220 nm) was collected. Removal of the solvents gave 19 mg (99% ee) of the title compound.

LC-MS (Methode 1): Rₜ=0.87 min; MS (ESIpos): m/z=466 [M+H]⁺.

¹H-NMR (600 MHz, DMSO-d6) δ[ppm]: 0.912 (0.54), 0.927 (0.59), 1.393 (0.47), 1.411 (0.49), 1.437 (0.45), 1.458 (0.99), 1.469 (0.70), 1.478 (1.03), 1.497 (0.43), 1.582 (1.26), 1.599 (1.14), 1.720 (0.53), 1.759 (1.25), 1.778 (1.08), 1.887 (0.77), 1.904 (1.24), 1.921 (0.65), 2.098 (0.48), 2.113 (0.90), 2.132 (0.48), 2.521 (0.54), 2.708 (0.69), 2.725 (0.65), 2.795 (0.72), 2.809 (0.70), 3.027 (0.95), 3.044 (1.74), 3.065 (0.96), 3.136 (0.52), 3.151 (1.62), 3.164 (3.04), 3.173 (1.73), 3.179 (0.62), 3.189 (0.51), 3.203 (16.00), 3.920 (1.30), 3.942 (1.23), 4.523 (2.44), 4.532 (2.44), 7.822 (5.22), 7.878 (0.60), 7.882 (0.63), 7.897 (1.01), 7.910 (0.61), 7.914 (0.60), 8.459 (2.33), 8.462 (2.25), 8.664 (0.75), 8.673 (1.50), 8.683 (0.73).

### Example 101

### ent-2-{3-[(Cyclobutyloxy)methyl][1,4′-bipiperidin]-1′-yl}-N-[(3,5-difluoropyridin-2-yl)methyl]-1,3-thiazole-5-carboxamide (enantiomer 1)

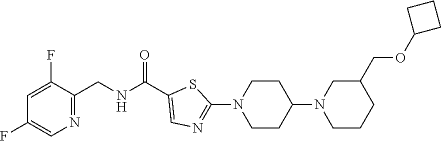 [SMILES: O=C(NCc1ncc(F)cc1F)c1cnc(N2CCC(N3CCCC(COC4CCC4)C3)CC2)s1]

28 mg of rac-2-{3-[(cyclobutyloxy)methyl][1,4′-bipiperidin]-1′-yl}-N-[(3,5-difluoropyridin-2-yl)methyl]-1,3-thiazole-5-carboxamide were separated into the enantiomers by chiral HPLC (preparative HPLC: column Daicel@ Chiralpak IG, 5 μm, 250×20 mm; mobile phase: 100% ethanol+0.2% diethylamine; flow rate 15 ml/min; temperature 35° C., detection: 220 nm). The enantiomer having a retention time of 13.192 min (HPLC: column Daicel© Chiralpak IG 5 μm, flow rate 1 ml/min; mobile phase: 100% ethanol+0.2% diethylamine; temperature 40° C.; detection: 220 nm) was collected. Removal of the solvents gave 11 mg (99% ee) of the title compound.

LC-MS (Methode 4): Rₜ=0.61 min; MS (ESIpos): m/z=506 [M+H]⁺.

¹H-NMR (600 MHz, DMSO-d6) δ[ppm]: 0.890 (0.70), 0.905 (1.48), 0.923 (1.66), 0.941 (0.75), 1.236 (0.70), 1.365 (0.68), 1.384 (1.52), 1.391 (1.55), 1.405 (2.41), 1.409 (2.43), 1.422 (3.30), 1.426 (2.37), 1.436 (2.51), 1.439 (3.65), 1.453 (3.11), 1.457 (3.28), 1.469 (3.44), 1.477 (2.22), 1.488 (2.22), 1.576 (3.79), 1.592 (5.01), 1.608 (3.25), 1.626 (1.13), 1.661 (1.66), 1.736 (0.87), 1.757 (5.34), 1.765 (5.46), 1.772 (5.55), 1.779 (5.30), 1.809 (0.51), 1.892 (2.15), 1.909 (3.40), 1.926 (1.85), 2.088 (1.66), 2.092 (2.09), 2.105 (5.13), 2.120 (5.86), 2.132 (1.97), 2.136 (2.23), 2.421 (0.40), 2.523 (1.40), 2.693 (2.11), 2.711 (1.92), 2.788 (2.15), 2.803 (2.11), 3.027 (2.76), 3.045 (5.15), 3.065 (2.86), 3.077 (1.68), 3.093 (4.48), 3.106 (7.14), 3.117 (4.69), 3.123 (1.81), 3.132 (1.31), 3.260 (0.75), 3.797 (0.82), 3.810 (2.77), 3.822 (3.96), 3.834 (2.63), 3.846 (0.73), 3.924 (4.03), 3.945 (3.80), 4.523 (7.43), 4.532 (7.38), 7.822 (16.00), 7.878 (1.81), 7.882 (1.92), 7.897 (3.16), 7.910 (1.88), 7.913 (1.87), 8.458 (7.01), 8.462 (6.89), 8.664 (2.34), 8.673 (4.66), 8.683 (2.34).

### Example 102

### ent-2-{3-[(Cyclobutyloxy)methyl][1,4′-bipiperidin]-1′-yl}-N-[(3,5-difluoropyridin-2-yl)methyl]-1,3-thiazole-5-carboxamide (enantiomer 2)

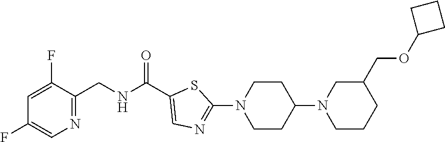 [SMILES: O=C(NCc1ncc(F)cc1F)c1cnc(N2CCC(N3CCCC(COC4CCC4)C3)CC2)s1]

28 mg of rac-2-{3-[(cyclobutyloxy)methyl][1,4′-bipiperidin]-1′-yl}-N-[(3,5-difluoropyridin-2-yl)methyl]-1,3-thiazole-5-carboxamide were separated into the enantiomers by chiral HPLC (preparative HPLC: column Daicel@ Chiralpak IG, 5 μm, 250×20 mm; mobile phase: 100% ethanol+0.2% diethylamine; flow rate 15 ml/min; temperature 35° C., detection: 220 nm). The enantiomer having a retention time of 15.649 min (HPLC: column Daicel© Chiralpak IG 5 μm, flow rate 1 ml/min; mobile phase: 100% ethanol+0.2% diethylamine; temperature 40° C.; detection: 220 nm) was collected. Removal of the solvents gave 15 mg (99% ee) of the title compound.

LC-MS (Methode 4): Rₜ=0.61 min; MS (ESIpos): m/z=506 [M+H]⁺.

¹H-NMR (600 MHz, DMSO-d6) δ[ppm]: 0.892 (0.70), 0.911 (1.47), 0.925 (1.61), 0.944 (0.75), 1.181 (0.58), 1.236 (0.75), 1.393 (1.83), 1.406 (2.67), 1.410 (2.79), 1.423 (3.49), 1.427 (2.71), 1.437 (2.88), 1.441 (3.93), 1.454 (3.44), 1.458 (3.62), 1.471 (3.83), 1.479 (2.52), 1.490 (2.38), 1.578 (4.21), 1.593 (5.37), 1.609 (3.64), 1.627 (1.42), 1.664 (1.80), 1.737 (1.27), 1.758 (5.60), 1.766 (5.97), 1.773 (5.87), 1.781 (5.68), 1.810 (0.82), 1.898 (1.60), 1.915 (2.56), 1.931 (1.34), 2.089 (2.00), 2.093 (2.40), 2.107 (5.04), 2.111 (4.26), 2.117 (4.59), 2.122 (4.87), 2.136 (2.03), 2.423 (0.43), 2.572 (0.60), 2.697 (1.87), 2.716 (1.71), 2.793 (2.04), 2.808 (1.91), 3.028 (2.85), 3.046 (5.10), 3.066 (3.05), 3.078 (2.04), 3.094 (4.70), 3.108 (6.61), 3.118 (4.75), 3.124 (2.06), 3.134 (1.53), 3.798 (0.78), 3.811 (2.64), 3.822 (3.74), 3.835 (2.51), 3.847 (0.68), 3.926 (3.99), 3.947 (3.69), 4.524 (7.26), 4.533 (7.00), 7.824 (16.00), 7.878 (2.01), 7.882 (2.03), 7.895 (2.96), 7.898 (2.98), 7.910 (1.94), 7.914 (1.86), 8.459 (7.08), 8.463 (6.55), 8.665 (2.41), 8.675 (4.52), 8.684 (2.21).

### Example 103

### rac-N-[(3,5-Difluoropyridin-2-yl)methyl]-2-(3-isopropyl[1,4′-bipiperidin]-1′-yl)-1,3-thiazole-5-carboxamide

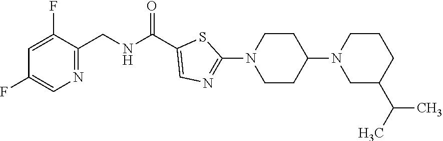 [SMILES: CC(C)C1CCCN(C2CCN(c3ncc(C(=O)NCc4ncc(F)cc4F)s3)CC2)C1]

N,N-Diisopropylethylamine (49 μl, 280 μmol) and acetic acid (9.7 μl, 170 μmol) were added in succession to a solution of N-[(3,5-difluoropyridin-2-yl)methyl]-2-(4-oxopiperidin-1-yl)-1,3-thiazole-5-carboxamide (50.0 mg, 142 μmol) and rac-3-isopropylpiperidine (36.1 mg, 284 μmol) in 3 ml of dichloromethane, and the mixture was stirred at room temperature 6 h. Subsequently, sodium triacetoxyborohydride (45.1 mg, 213 μmol) was added and stirring of the mixture at room temperature was continued. After 15 h, sat. NaHCO₃ solution was added and the reaction mixture was extracted with dichloromethane. The organic phase was concentrated on a rotary evaporator and the residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 23.0 mg (100% purity, 35% of theory) of the title compound.

LC-MS (Methode 5): Rₜ=1.85 min; MS (ESIpos): m/z=464 [M+H]⁺.

¹H-NMR (600 MHz, DMSO-d6) δ[ppm]: 0.837 (14.82), 0.843 (15.99), 0.848 (16.00), 0.854 (15.31), 0.877 (1.10), 0.883 (1.12), 0.897 (1.16), 0.903 (1.14), 0.917 (0.49), 0.923 (0.43), 1.160 (0.54), 1.171 (0.93), 1.177 (1.09), 1.183 (0.97), 1.188 (1.09), 1.195 (0.85), 1.206 (0.49), 1.334 (0.44), 1.354 (1.17), 1.375 (1.58), 1.387 (1.58), 1.398 (2.24), 1.409 (1.94), 1.420 (1.12), 1.441 (0.42), 1.448 (0.49), 1.461 (1.12), 1.467 (1.32), 1.486 (1.96), 1.506 (1.42), 1.525 (0.55), 1.533 (0.43), 1.600 (1.43), 1.606 (1.16), 1.616 (0.92), 1.622 (1.22), 1.627 (0.94), 1.647 (1.19), 1.668 (1.14), 1.765 (1.59), 1.778 (2.27), 1.792 (1.35), 1.866 (1.66), 1.883 (3.02), 1.901 (1.55), 2.024 (1.02), 2.038 (1.80), 2.042 (1.78), 2.057 (1.01), 2.524 (1.03), 2.733 (1.42), 2.751 (1.37), 2.770 (1.47), 2.786 (1.40), 3.020 (1.59), 3.026 (1.23), 3.041 (2.88), 3.057 (1.18), 3.063 (1.59), 3.931 (2.24), 3.948 (2.14), 4.523 (4.92), 4.532 (4.94), 7.821 (13.40), 7.879 (1.40), 7.883 (1.52), 7.895 (2.03), 7.898 (2.11), 7.910 (1.43), 7.914 (1.50), 8.459 (5.28), 8.462 (5.28), 8.662 (1.61), 8.672 (3.25), 8.681 (1.63).

### Example 104

### ent-N-[(3,5-Difluoropyridin-2-yl)methyl]-2-[4-(4-methylazepan-1-yl)piperidin-1-yl]-1,3-thiazole-5-carboxamide (enantiomer 1)

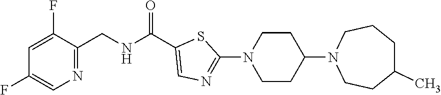 [SMILES: CC1CCCN(C2CCN(c3ncc(C(=O)NCc4ncc(F)cc4F)s3)CC2)CC1]

33 mg of rac-N-[(3,5-difluoropyridin-2-yl)methyl]-2-[4-(4-methylazepan-1-yl)piperidin-1-yl]-1,3-thiazole-5-carboxamide were separated into the enantiomers by chiral HPLC (preparative HPLC: column Daicel® Chiralpak AY-H, 5 μm, 250×20 mm; mobile phase: 70% n-heptane, mobile phase B: 30% ethanol+0.2% diethylamine in B; flow rate 15 ml/min; temperature 60° C., detection: 220 nm). The enantiomer having a retention time of 10.241 min (HPLC: column Daicel® Chiralpak AY-H 5 μm, flow rate 1 ml/min; mobile phase A: 70% n-Heptan, mobile phase B: 30% ethanol+0.2% diethylamine in B; temperature 60° C.; detection: 220 nm) was collected. Removal of the solvents gave 15 mg (99% ee) of the title compound.

LC-MS (Methode 1): Rₜ=0.98 min; MS (ESIpos): m/z=450 [M+H]⁺.

¹H-NMR (600 MHz, DMSO-d6) δ[ppm]: 0.876 (16.00), 0.887 (15.94), 1.181 (1.40), 1.200 (1.81), 1.223 (2.27), 1.240 (2.37), 1.257 (1.01), 1.446 (2.85), 1.460 (3.55), 1.479 (2.38), 1.572 (1.57), 1.595 (1.81), 1.613 (1.45), 1.619 (1.81), 1.642 (3.31), 1.648 (3.08), 1.655 (2.78), 1.756 (2.90), 2.422 (0.41), 2.611 (1.54), 2.668 (2.96), 3.023 (2.75), 3.040 (5.00), 3.061 (2.82), 3.926 (3.42), 3.946 (3.24), 4.523 (7.45), 4.532 (7.44), 7.820 (14.18), 7.879 (1.77), 7.882 (1.83), 7.895 (3.04), 7.910 (1.83), 7.914 (1.85), 8.458 (6.84), 8.462 (6.66), 8.663 (2.10), 8.672 (4.24), 8.682 (2.21).

### Example 105

### ent-N-[(3,5-Difluoropyridin-2-yl)methyl]-2-[4-(4-methylazepan-1-yl)piperidin-1-yl]-1,3-thiazole-5-carboxamide (enantiomer 2)

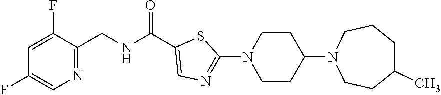 [SMILES: CC1CCCN(C2CCN(c3ncc(C(=O)NCc4ncc(F)cc4F)s3)CC2)CC1]

33 mg of rac-N-[(3,5-difluoropyridin-2-yl)methyl]-2-[4-(4-methylazepan-1-yl)piperidin-1-yl]-1,3-thiazole-5-carboxamide were separated into the enantiomers by chiral HPLC (preparative HPLC: column Daicel@ Chiralpak AY-H, 5 μm, 250×20 mm; mobile phase: 70% n-heptane, mobile phase B: 30% ethanol+0.2% diethylamine in B; flow rate 15 ml/min; temperature 60° C., detection: 220 nm). The enantiomer having a retention time of 10.783 min (HPLC: column Daicel© Chiralpak AY-H 5 μm, flow rate 1 ml/min; mobile phase A: 70% n-heptane, mobile phase B: 30% ethanol+0.2% diethylamine in B; temperature 60° C.; detection: 220 nm) was collected. Removal of the solvents gave 16 mg (99% ee) of the title compound.

LC-MS (Methode 1): Rₜ=0.98 min; MS (ESIpos): m/z=450 [M+H]⁺.

¹H-NMR (600 MHz, DMSO-d6) δ[ppm]: 0.876 (15.76), 0.887 (16.00), 1.181 (1.39), 1.201 (1.73), 1.223 (2.16), 1.240 (2.40), 1.257 (1.05), 1.444 (2.79), 1.458 (3.56), 1.477 (2.43), 1.572 (1.52), 1.596 (1.76), 1.641 (3.34), 1.647 (3.12), 1.655 (2.79), 1.754 (3.00), 2.610 (1.55), 2.664 (3.12), 3.023 (2.70), 3.040 (4.97), 3.060 (2.82), 3.258 (0.86), 3.324 (0.78), 3.924 (3.44), 3.944 (3.25), 4.522 (7.39), 4.531 (7.46), 7.819 (14.10), 7.879 (1.72), 7.882 (1.79), 7.895 (3.01), 7.910 (1.67), 7.914 (1.76), 8.458 (6.66), 8.462 (6.47), 8.663 (2.16), 8.672 (4.25), 8.682 (2.15).

### Example 106

### ent-N-[(3,5-Difluoropyridin-2-yl)methyl]-2-{3-[(2,2,2-trifluoroethoxy)methyl][1,4′-bipiperidin]-1′-yl}-1,3-thiazole-5-carboxamide (enantiomer 1)

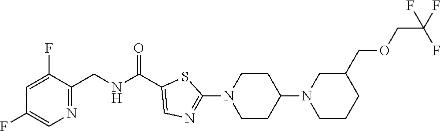 [SMILES: O=C(NCc1ncc(F)cc1F)c1cnc(N2CCC(N3CCCC(COCC(F)(F)F)C3)CC2)s1]

53 mg of rac-N-[(3,5-difluoropyridin-2-yl)methyl]-2-{3-[(2,2,2-trifluoroethoxy)methyl][1,4′-bipiperidin]-1′-yl}-1,3-thiazole-5-carboxamide were separated into the enantiomers by chiral HPLC (preparative HPLC: column Daicelo® Chiralpak AY-H, 5 μm, 250×20 mm; mobile phase A: 55% n-heptane, mobile phase B: 45% ethanol+0.2% diethylamine in B; flow rate 15 ml/min; temperature 60° C., detection: 220 nm). The enantiomer having a retention time of 5.622 min (HPLC: column Daicel® Chiralpak AY-H 5 μm, flow rate 1 ml/min; mobile phase A: 50% n-heptane, mobile phase B: 50% ethanol+0.2% diethylamine in B; temperature 70° C.; detection: 220 nm) was collected. Removal of the solvents gave 27 mg (99% ee) of the title compound.

LC-MS (Methode 1): Rₜ=1.09 min; MS (ESIpos): m/z=534 [M+H]⁺.

¹H-NMR (600 MHz, DMSO-d6) δ[ppm]: 0.957 (1.60), 0.972 (1.65), 0.991 (0.73), 1.405 (1.42), 1.422 (1.56), 1.433 (1.26), 1.453 (2.17), 1.474 (3.10), 1.493 (2.23), 1.591 (3.88), 1.609 (3.56), 1.771 (4.73), 1.974 (1.76), 2.155 (1.88), 2.697 (1.60), 2.780 (1.80), 2.796 (1.72), 3.030 (2.88), 3.051 (5.58), 3.071 (2.93), 3.322 (0.44), 3.426 (0.55), 3.443 (8.06), 3.454 (9.47), 3.926 (4.21), 3.948 (4.04), 3.977 (3.49), 3.993 (10.05), 4.008 (9.84), 4.024 (3.21), 4.524 (8.20), 4.533 (8.19), 7.823 (16.00), 7.879 (1.88), 7.882 (2.06), 7.898 (3.42), 7.910 (1.91), 7.914 (2.03), 8.458 (7.35), 8.462 (7.51), 8.667 (2.44), 8.676 (4.94), 8.685 (2.45).

### Example 107

### ent-N-[(3,5-Difluoropyridin-2-yl)methyl]-2-{3-[(2,2,2-trifluoroethoxy)methyl][1,4′-bipiperidin]-1′-yl}-1,3-thiazole-5-carboxamide (enantiomer 2)

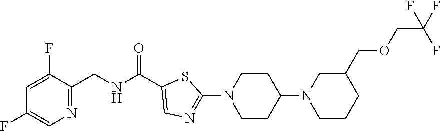 [SMILES: O=C(NCc1ncc(F)cc1F)c1cnc(N2CCC(N3CCCC(COCC(F)(F)F)C3)CC2)s1]

53 mg of rac-N-[(3,5-difluoropyridin-2-yl)methyl]-2-{3-[(2,2,2-trifluoroethoxy)methyl][1,4′-bipiperidin]-1′-yl}-1,3-thiazole-5-carboxamide were separated into the enantiomers by chiral HPLC (preparative HPLC: column Daicel® Chiralpak AY-H, 5 μm, 250×20 mm; mobile phase A: 55% n-heptane, mobile phase B: 45% ethanol+0.2% diethylamine in B; flow rate 15 ml/min; temperature 60° C., detection: 220 nm). The enantiomer having a retention time of 6.301 min (HPLC: column Daicel® Chiralpak AY-H 5 μm, flow rate 1 ml/min; mobile phase A: 50% n-heptane, mobile phase B: 50% ethanol+0.2% diethylamine in B; temperature 70° C.; detection: 220 nm) was collected. Removal of the solvents gave 25 mg (99% ee) of the title compound. LC-MS (Methode 1): Rₜ=1.08 min; MS (ESIpos): m/z=534 [M+H]⁺.

¹H-NMR (600 MHz, DMSO-d6) δ[ppm]: 0.956 (1.27), 0.972 (1.33), 1.403 (1.14), 1.422 (1.29), 1.433 (1.11), 1.453 (1.79), 1.473 (2.52), 1.493 (1.88), 1.592 (3.06), 1.608 (2.89), 1.770 (3.84), 1.973 (1.45), 2.154 (1.54), 2.693 (1.28), 2.780 (1.42), 2.794 (1.42), 3.030 (2.30), 3.050 (4.43), 3.071 (2.43), 3.426 (0.52), 3.443 (6.29), 3.454 (7.83), 3.926 (3.34), 3.947 (3.28), 3.977 (3.35), 3.992 (9.35), 4.008 (9.05), 4.024 (3.07), 4.524 (6.38), 4.532 (6.45), 7.823 (16.00), 7.878 (1.79), 7.882 (1.93), 7.895 (2.60), 7.897 (2.76), 7.910 (1.89), 7.914 (1.90), 8.458 (6.68), 8.462 (6.64), 8.666 (2.02), 8.676 (4.13), 8.685 (2.11).

### Example 108

### diamix-2-{3-[(2,2-Difluorocyclopropyl)methoxy][1,4′-bipiperidin]-1′-yl}-N-[(3,5-difluoropyridin-2-yl)methyl]-1,3-thiazole-5-carboxamide

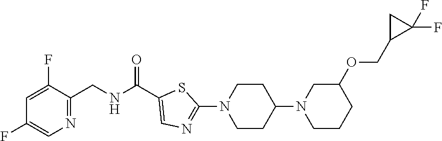 [SMILES: O=C(NCc1ncc(F)cc1F)c1cnc(N2CCC(N3CCCC(OCC4CC4(F)F)C3)CC2)s1]

N,N-Diisopropylethylamine (200 μl, 1.1 mmol) was added to a solution of diamix-3-[(2,2-difluorocyclopropyl)methoxy]piperidine sulfate hydrochloride (185 mg, 568 μmol) in 5 ml of dichloromethane, and the mixture was stirred for 5 min, after which N-[(3,5-difluoropyridin-2-yl)methyl-2-(4-oxopiperidin-1-yl)-1,3-thiazole-5-carboxamide (100 mg, 284 μmol) and acetic acid (19 μl, 340 μmol) were added to the mixture. The mixture was then stirred at room temperature. After 3 h, sodium triacetoxyborohydride (90.2 mg, 426 μmol) was added to the mixture and the mixture was stirred at room temperature overnight. Sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and concentrated and the residue was dried under high vacuum. This gave 10.0 mg (purity 100%, 7% of theory) of the target compound.

LC-MS (Methode 1): Rₜ=1.05 min; MS (ESIpos): m/z=528 [M+H]⁺.

¹H-NMR (600 MHz, DMSO-d6) δ[ppm]: 1.074 (1.61), 1.091 (1.50), 1.237 (1.72), 1.251 (1.61), 1.348 (1.50), 1.367 (1.61), 1.488 (2.47), 1.548 (1.93), 1.556 (1.40), 1.568 (1.83), 1.623 (1.83), 1.764 (3.97), 1.783 (3.54), 1.907 (2.58), 1.963 (1.61), 1.982 (2.79), 1.997 (1.61), 2.098 (1.83), 2.383 (0.97), 2.422 (1.29), 2.566 (1.40), 2.611 (0.86), 2.651 (2.79), 2.942 (2.04), 2.956 (1.93), 3.023 (2.79), 3.043 (5.26), 3.063 (2.79), 3.254 (1.40), 3.260 (0.64), 3.315 (3.76), 3.322 (3.97), 3.375 (1.07), 3.391 (2.58), 3.406 (2.79), 3.423 (1.40), 3.570 (2.04), 3.581 (1.93), 3.928 (3.65), 3.950 (3.44), 4.524 (7.73), 4.532 (7.84), 7.822 (16.00), 7.879 (1.93), 7.883 (2.15), 7.897 (3.22), 7.910 (2.04), 7.914 (2.04), 8.459 (7.30), 8.462 (7.41), 8.666 (2.36), 8.675 (4.83), 8.685 (2.36).

### Example 109

### rac-2-[3-(Cyclobutyloxy)[1,4′-bipiperidin]-1′-yl]-N-[(3,5-difluoropyridin-2-yl)methyl]-1,3-thiazole-5-carboxamide

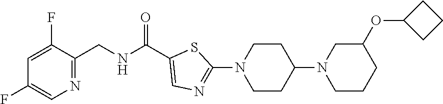 [SMILES: O=C(NCc1ncc(F)cc1F)c1cnc(N2CCC(N3CCCC(OC4CCC4)C3)CC2)s1]

N,N-Diisopropylethylamine (200 μl, 1.1 mmol) and acetic acid (19 μl, 340 μmol) were added in succession to a solution of N-[(3,5-difluoropyridin-2-yl)methyl]-2-(4-oxopiperidin-1-yl)-1,3-thiazole-5-carboxamide (100 mg, 284 μmol) and rac-3-(cyclobutyloxy)piperidine sulfate hydrochloride (164 mg, 568 μmol) in 5 ml of dichloromethane, and the mixture was stirred at room temperature for 5 h. Subsequently, sodium triacetoxyborohydride (90.2 mg, 426 μmol) was added and the mixture was stirred at room temperature overnight. Sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and concentrated and the residue was dried under high vacuum. This gave 10.0 mg (purity 100%, 7% of theory) of the target compound.

LC-MS (Methode 1): Rₜ=1.04 min; MS (ESIpos): m/z=492 [M+H]⁺.

¹H-NMR (600 MHz, DMSO-d6) δ[ppm]: 1.034 (0.72), 1.046 (1.63), 1.069 (1.63), 1.083 (0.81), 1.091 (0.68), 1.311 (0.68), 1.333 (1.54), 1.352 (1.72), 1.372 (1.08), 1.385 (1.04), 1.390 (1.58), 1.403 (2.76), 1.407 (1.72), 1.416 (1.72), 1.420 (3.07), 1.433 (1.99), 1.438 (2.26), 1.445 (1.31), 1.458 (2.98), 1.464 (3.12), 1.478 (3.30), 1.485 (3.12), 1.498 (1.45), 1.505 (1.27), 1.550 (0.90), 1.567 (2.53), 1.585 (2.71), 1.600 (2.85), 1.623 (1.76), 1.757 (4.84), 1.777 (6.37), 1.790 (4.07), 1.810 (3.30), 1.823 (2.53), 1.838 (1.76), 1.937 (2.21), 1.953 (3.66), 1.969 (2.26), 2.046 (1.49), 2.064 (2.71), 2.079 (1.54), 2.112 (3.44), 2.120 (3.39), 2.383 (0.45), 2.422 (0.59), 2.465 (0.50), 2.611 (0.54), 2.641 (2.26), 2.651 (1.94), 2.659 (2.12), 2.864 (2.08), 2.882 (1.94), 3.019 (2.89), 3.037 (5.24), 3.057 (2.94), 3.234 (1.63), 3.243 (2.12), 3.250 (2.85), 3.257 (3.12), 3.924 (3.98), 3.946 (3.84), 3.968 (0.90), 3.980 (2.71), 3.993 (3.80), 4.005 (2.62), 4.017 (0.77), 4.523 (7.73), 4.532 (7.73), 7.823 (16.00), 7.879 (1.90), 7.882 (2.08), 7.897 (3.30), 7.910 (1.99), 7.914 (2.03), 8.459 (7.28), 8.462 (7.37), 8.666 (2.44), 8.676 (4.79), 8.685 (2.44).

### Example 110

### rac-2-{3-[(3,3-Difluorocyclobutyl)oxy][1,4′-bipiperidin]-1′-yl}-N-[(3,5-difluoropyridin-2-yl)methyl]-1,3-thiazole-5-carboxamide

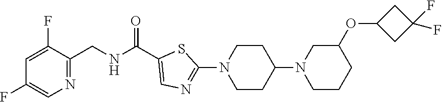 [SMILES: O=C(NCc1ncc(F)cc1F)c1cnc(N2CCC(N3CCCC(OC4CC(F)(F)C4)C3)CC2)s1]

N,N-Diisopropylethylamine (200 μl, 1.1 mmol) and acetic acid (19 μl, 340 μmol) were added in succession to a solution of N-[(3,5-difluoropyridin-2-yl)methyl]-2-(4-oxopiperidin-1-yl)-1,3-thiazole-5-carboxamide (100 mg, 284 μmol) and rac-3-[(3,3-difluorocyclobutyl)oxy]piperidine sulfate hydrochloride (185 mg, 568 μmol) in 5 ml of dichloromethane, and the mixture was stirred at room temperature for 5 h. Subsequently, sodium triacetoxyborohydride (90.2 mg, 426 μmol) was added and the mixture was stirred at room temperature overnight. Sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and concentrated and the residue was dried under high vacuum. This gave 30.0 mg (purity 100%, 20% of theory) of the target compound.

LC-MS (Methode 1): Rₜ=1.06 min; MS (ESIpos): m/z=528 [M+H]⁺.

¹H-NMR (600 MHz, DMSO-d6) δ[ppm]: 1.083 (0.83), 1.097 (2.04), 1.119 (2.12), 1.134 (0.94), 1.324 (0.86), 1.344 (1.99), 1.364 (2.10), 1.384 (0.88), 1.478 (3.86), 1.498 (4.08), 1.518 (1.52), 1.621 (2.46), 1.643 (2.15), 1.757 (5.57), 1.777 (4.80), 1.853 (2.32), 1.867 (2.21), 2.003 (2.54), 2.019 (4.36), 2.035 (2.65), 2.084 (1.85), 2.100 (3.42), 2.117 (1.88), 2.422 (2.26), 2.431 (2.76), 2.441 (2.59), 2.446 (2.68), 2.451 (2.73), 2.459 (2.87), 2.468 (2.12), 2.524 (2.07), 2.561 (1.68), 2.636 (2.87), 2.654 (2.79), 2.874 (4.00), 2.884 (5.49), 2.901 (3.70), 3.019 (3.59), 3.040 (6.90), 3.061 (3.56), 3.257 (0.66), 3.265 (0.69), 3.308 (2.37), 3.317 (2.76), 3.325 (3.06), 3.331 (2.48), 3.340 (1.74), 3.929 (5.08), 3.951 (4.86), 4.101 (2.37), 4.524 (9.90), 4.533 (9.74), 7.824 (16.00), 7.882 (2.21), 7.897 (4.14), 7.914 (2.18), 8.459 (7.92), 8.462 (8.17), 8.667 (2.84), 8.677 (5.71), 8.686 (2.92).

### Example 111

### diamix-N-[(3,5-Difluoropyridin-2-yl)methyl]-2-[(3R)-3′-fluoro-3-methyl[1,4′-bipiperidin]-1′-yl]-1,3-thiazole-4-carboxamide

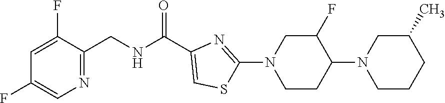 [SMILES: C[C@@H]1CCCN(C2CCN(c3nc(C(=O)NCc4ncc(F)cc4F)cs3)CC2F)C1]

2-Bromo-N-[(3,5-difluoropyridin-2-yl)methyl]-1,3-thiazole-5-carboxamide (100 mg, 299 μmol) and diamix-(3R)-3′-fluoro-3-methyl-1,4′-bipiperidine dihydrochloride (70.9 mg, 259 μmol) were combined and stirred at 120° C. in 2 ml of sodium carbonate solution (2 ml, 2.0 M, 4 mmol) for 1 h. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm, mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 78.0 mg (purity 100%, 57% of theory) of the target compound.

LC-MS (Methode 1): Rₜ=0.95 min; MS (ESIpos): m/z=454 [M+H]⁺.

¹H-NMR (600 MHz, DMSO-d6) δ[ppm]: 0.816 (10.43), 0.823 (12.35), 0.826 (12.52), 0.834 (10.93), 0.849 (1.65), 0.869 (0.67), 1.386 (0.84), 1.406 (1.13), 1.428 (1.10), 1.448 (0.90), 1.469 (0.43), 1.497 (0.61), 1.503 (0.75), 1.514 (0.75), 1.552 (0.84), 1.572 (2.20), 1.588 (1.25), 1.594 (1.45), 1.629 (1.77), 1.649 (1.68), 1.689 (1.68), 1.706 (1.97), 1.887 (1.48), 1.896 (1.86), 1.904 (2.87), 1.917 (2.26), 1.923 (2.52), 1.938 (1.57), 1.946 (1.48), 1.958 (0.55), 1.965 (0.43), 2.175 (1.28), 2.193 (2.38), 2.210 (1.19), 2.591 (1.04), 2.596 (1.01), 2.611 (1.25), 2.650 (1.25), 2.672 (1.01), 2.838 (3.19), 2.846 (3.16), 3.105 (1.42), 3.122 (2.61), 3.126 (2.52), 3.143 (1.42), 3.213 (2.09), 3.236 (2.17), 3.262 (0.78), 3.279 (3.51), 3.302 (2.87), 4.028 (1.68), 4.049 (1.59), 4.189 (1.10), 4.210 (2.03), 4.229 (1.01), 4.578 (7.30), 4.588 (7.30), 5.065 (2.26), 5.149 (2.29), 7.373 (16.00), 7.883 (1.88), 7.887 (2.00), 7.902 (2.96), 7.915 (1.88), 7.919 (1.94), 8.452 (7.65), 8.456 (8.70), 8.467 (4.12), 8.477 (2.09).

### Example 112

### diamix-N-[(3,5-Difluoropyridin-2-yl)methyl]-2-[(3R)-3′-fluoro-3-methyl[1,4′-bipiperidin]-1′-yl]-1,3-oxazole-4-carboxamide

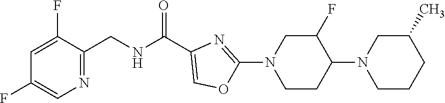 [SMILES: C[C@@H]1CCCN(C2CCN(c3nc(C(=O)NCc4ncc(F)cc4F)co3)CC2F)C1]

2-Chloro-N-[(3,5-difluoropyridin-2-yl)methyl]-1,3-oxazole-4-carboxamide (100 mg, 314 μmol) and diamix-(3R)-3′-fluoro-3-methyl-1,4′-bipiperidine dihydrochloride (86.5 mg, 317 μmol) were combined and stirred at 120° C. in 2 ml of sodium carbonate solution (2 ml, 2.0 M, 4 mmol) for 1 hour. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm, mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 81.0 mg (purity 100%, 51% of theory) of the target compound.

LC-MS (Methode 1): Rₜ=0.88 min; MS (ESIpos): m/z=438 [M+H]⁺.

¹H-NMR (600 MHz, DMSO-d6) δ[ppm]: 0.809 (11.35), 0.817 (14.02), 0.820 (14.57), 0.827 (12.10), 0.841 (2.08), 0.862 (0.75), 1.379 (1.01), 1.398 (1.40), 1.419 (1.40), 1.440 (1.11), 1.460 (0.55), 1.505 (0.91), 1.564 (2.73), 1.586 (1.85), 1.623 (2.24), 1.644 (4.13), 1.665 (2.50), 1.864 (1.53), 1.880 (3.45), 1.889 (3.32), 1.900 (2.57), 1.906 (2.57), 2.068 (5.46), 2.160 (1.56), 2.178 (3.09), 2.197 (1.63), 2.578 (1.40), 2.615 (1.46), 2.636 (1.33), 2.824 (4.33), 3.057 (1.72), 3.075 (3.28), 3.096 (1.76), 3.181 (2.67), 3.205 (2.83), 3.246 (2.83), 3.259 (1.01), 3.271 (3.77), 3.317 (0.52), 4.085 (2.37), 4.106 (2.28), 4.130 (1.63), 4.150 (2.67), 4.173 (1.46), 4.561 (9.04), 4.570 (9.01), 5.028 (2.86), 5.111 (2.89), 7.883 (2.02), 7.887 (2.05), 7.901 (3.64), 7.915 (2.05), 7.919 (2.02), 8.004 (16.00), 8.207 (2.47), 8.217 (4.81), 8.226 (2.37), 8.459 (7.93), 8.463 (7.61).

### Example 113

### diamix-N-(5-Chloro-2-fluorobenzyl)-2-[(3R)-3′-fluoro-3-methyl[1,4′-bipiperidin]-1′-yl]-1,3-thiazole-5-carboxamide

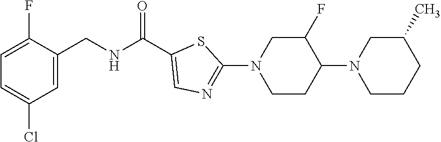 [SMILES: C[C@@H]1CCCN(C2CCN(c3ncc(C(=O)NCc4cc(Cl)ccc4F)s3)CC2F)C1]

2-Bromo-N-(5-chloro-2-fluorobenzyl)-1,3-thiazole-5-carboxamide (100 mg, 286 μmol) and diamix-(3R)-3′-fluoro-3-methyl-1,4′-bipiperidine dihydrochloride (67.7 mg, 248 μmol) were combined and stirred at 120° C. in 2 ml of sodium carbonate solution (2 ml, 2.0 M, 4 mmol) for 1 h. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm, mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 25.0 mg (purity 97%, 18% of theory) of the target compound.

LC-MS (Methode 1): Rₜ=1.17 min; MS (ESIpos): m/z=469 [M+H]⁺.

¹H-NMR (600 MHz, DMSO-d6) δ[ppm]: 0.809 (11.21), 0.818 (13.98), 0.820 (14.09), 0.828 (11.39), 0.843 (2.02), 0.863 (0.72), 1.122 (0.47), 1.381 (0.94), 1.401 (1.30), 1.423 (1.26), 1.443 (1.08), 1.464 (0.58), 1.496 (0.90), 1.565 (2.85), 1.587 (1.84), 1.623 (2.16), 1.645 (2.09), 1.681 (1.98), 1.699 (2.38), 1.884 (3.14), 1.892 (3.71), 1.909 (2.56), 1.927 (0.58), 2.162 (1.41), 2.180 (2.70), 2.199 (1.41), 2.384 (0.43), 2.422 (0.47), 2.607 (1.37), 2.622 (1.15), 2.665 (1.15), 2.682 (1.15), 2.823 (4.07), 3.143 (1.62), 3.160 (3.03), 3.181 (1.69), 3.241 (2.59), 3.265 (3.96), 3.307 (3.14), 3.332 (2.49), 3.411 (0.86), 4.001 (2.09), 4.024 (1.98), 4.174 (1.37), 4.195 (2.34), 4.217 (1.23), 4.405 (10.20), 4.414 (10.13), 5.058 (2.77), 5.140 (2.74), 7.231 (3.17), 7.247 (6.09), 7.262 (3.96), 7.352 (4.36), 7.362 (6.56), 7.375 (2.56), 7.382 (2.45), 7.822 (16.00), 8.713 (2.52), 8.722 (5.01). 8.732 (2.56).

### Example 114

### 2-[(3R)-3-(Cyclopropylmethoxy)[1,4′-bipiperidin]-1′-yl]-N-[(3,5-difluoropyridin-2-yl)methyl]-1,3-thiazole-5-carboxamide

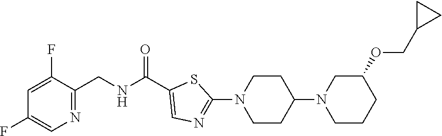 [SMILES: O=C(NCc1ncc(F)cc1F)c1cnc(N2CCC(N3CCC[C@@H](OCC4CC4)C3)CC2)s1]

2-Bromo-N-[(3,5-difluoropyridin-2-yl)methyl]-1,3-thiazole-5-carboxamide (80.2 mg, 240 μmol) and (3R)-3-(cyclopropylmethoxy)-1,4′-bipiperidine dihydrochloride (66.0 mg, 212 μmol) were combined and stirred at 120° C. in 2 ml of sodium carbonate solution (2 ml, 2.0 M, 4 mmol) for 1 hour. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm, mobile phase A: water, mobile phase B: acetonitrile, mobile phase C: 2% ammonia in water, mobile phase D: acetonitrile/water (80% by volume/20% by volume) total flow rate: 80 ml/min; room temperature, wavelength 200-400 nm, complete injection; gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 46.0 mg (purity 100%, 39% of theory) of the target compound.

LC-MS (Methode 1): Rₜ=1.01 min; MS (ESIpos): m/z=492 [M+H]⁺.

¹H-NMR (600 MHz, DMSO-d6) δ[ppm]: 0.116 (2.25), 0.124 (7.52), 0.131 (7.71), 0.140 (2.25), 0.411 (2.21), 0.417 (6.60), 0.420 (6.38), 0.431 (6.78), 0.433 (6.27), 0.440 (1.84), 0.915 (0.77), 0.926 (1.59), 0.937 (2.32), 0.948 (1.55), 1.023 (0.59), 1.030 (0.66), 1.044 (1.59), 1.067 (1.66), 1.080 (0.77), 1.088 (0.66), 1.320 (0.70), 1.339 (1.59), 1.359 (1.62), 1.380 (0.70), 1.455 (1.11), 1.474 (2.95), 1.486 (2.40), 1.494 (3.13), 1.514 (1.25), 1.613 (2.06), 1.636 (1.73), 1.762 (4.17), 1.781 (3.65), 1.885 (1.81), 1.900 (1.73), 1.933 (2.21), 1.949 (3.69), 1.965 (2.25), 2.067 (1.51), 2.081 (2.73), 2.099 (1.47), 2.422 (0.44), 2.521 (1.73), 2.557 (1.33), 2.652 (2.54), 2.671 (2.14), 2.943 (2.18), 2.955 (2.03), 3.021 (2.80), 3.040 (5.46), 3.060 (2.88), 3.243 (14.49), 3.255 (14.56), 3.268 (3.61), 3.320 (0.81), 3.927 (3.80), 3.946 (3.61), 4.523 (7.74), 4.532 (7.71), 7.822 (16.00), 7.878 (1.92), 7.882 (1.99), 7.895 (3.17), 7.910 (1.95), 7.914 (1.92), 8.459 (7.37), 8.462 (7.12), 8.665 (2.43), 8.675 (4.83), 8.684 (2.40).

### Example 115

### ent-2-{3-[(Cyclopropylmethoxy)methyl][1,4′-bipiperidin]-1′-yl}-N-[(3,5-difluoropyridin-2-yl)methyl]-1,3-thiazole-5-carboxamide (enantiomer 1)

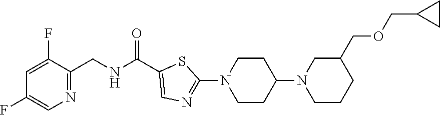 [SMILES: O=C(NCc1ncc(F)cc1F)c1cnc(N2CCC(N3CCCC(COCC4CC4)C3)CC2)s1]

67 mg of rac-2-{3-[(cyclopropylmethoxy)methyl][1,4′-bipiperidin]-1′-yl}-N-[(3,5-difluoropyridin-2-yl)methyl]-1,3-thiazole-5-carboxamide were separated into the enantiomers by chiral HPLC (preparative HPLC: column Daicel® Chiralpak AY-H, 5 μm, 250×20 mm; mobile phase A: 60% n-heptane, mobile phase B: 40% ethanol+0.2% diethylamine in B; flow rate 15 ml/min; temperature 55° C., detection: 220 nm). The enantiomer having a retention time of 8.062 min (HPLC: column Daicel® Chiralpak AY-H 5 μm, flow rate 1 ml/min; mobile phase A: 50% n-heptane, mobile phase B: 50% ethanol+0.2% diethylamine in B; temperature 55° C.; detection: 220 nm) was collected. Removal of the solvents gave 30 mg (99% ee) of the title compound.

LC-MS (Methode 1): Rₜ=1.07 min; MS (ESIpos): m/z=506 [M+H]⁺.

¹H-NMR (600 MHz, DMSO-d6) δ[ppm]: −0.146 (2.10), −0.024 (1.44), −0.017 (5.45), −0.009 (5.51), 0.275 (1.55), 0.282 (4.53), 0.285 (4.49), 0.288 (2.09), 0.295 (4.69), 0.298 (4.42), 0.305 (1.34), 0.755 (0.46), 0.773 (1.01), 0.791 (1.41), 0.794 (1.44), 0.804 (1.40), 0.807 (1.47), 0.815 (1.92), 0.823 (1.00), 0.826 (1.08), 0.835 (0.50), 1.230 (0.43), 1.249 (1.00), 1.269 (1.02), 1.290 (0.76), 1.298 (0.72), 1.310 (1.42), 1.322 (1.47), 1.330 (2.10), 1.338 (1.53), 1.350 (1.51), 1.369 (0.44), 1.433 (1.41), 1.439 (1.58), 1.451 (2.33), 1.467 (1.38), 1.566 (1.12), 1.617 (2.07), 1.623 (2.07), 1.764 (0.92), 1.781 (1.55), 1.798 (0.81), 1.965 (0.79), 1.983 (1.44), 2.000 (0.76), 2.352 (9.20), 2.355 (11.79), 2.357 (8.79), 2.369 (1.29), 2.394 (16.00), 2.580 (1.15), 2.662 (1.37), 2.678 (1.31), 2.885 (1.91), 2.904 (3.60), 2.923 (1.91), 3.026 (9.38), 3.037 (9.30), 3.053 (1.05), 3.069 (3.29), 3.080 (5.47), 3.089 (3.47), 3.095 (1.22), 3.105 (0.91), 3.143 (10.67), 3.780 (2.83), 3.801 (2.66), 4.379 (5.25), 4.388 (5.22), 7.679 (11.00), 7.732 (1.28), 7.735 (1.33), 7.748 (2.15), 7.750 (2.18), 7.763 (1.30), 7.767 (1.30), 8.313 (4.95), 8.316 (4.80), 8.520 (1.64), 8.529 (3.26), 8.538 (1.62).

### Example 116

### ent-2-{3-[(Cyclopropylmethoxy)methyl][1,4′-bipiperidin]-1′-yl}-N-[(3,5-difluoropyridin-2-yl)methyl]-1,3-thiazole-5-carboxamide (enantiomer 2)

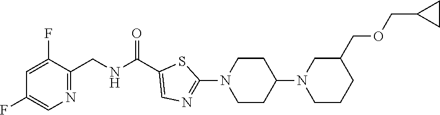 [SMILES: O=C(NCc1ncc(F)cc1F)c1cnc(N2CCC(N3CCCC(COCC4CC4)C3)CC2)s1]

67 mg of rac-2-{3-[(cyclopropylmethoxy)methyl][1,4′-bipiperidin]-1′-yl}-N-[(3,5-difluoropyridin-2-yl)methyl]-1,3-thiazole-5-carboxamide were separated into the enantiomers by chiral HPLC (preparative HPLC: column Daicel® Chiralpak AY-H, 5 μm, 250×20 mm; mobile phase A: 60% n-heptane, mobile phase B: 40% ethanol+0.2% diethylamine in B; flow rate 15 ml/min; temperature 55° C., detection: 220 nm). The enantiomer having a retention time of 8.740 min (HPLC: column Daicel® Chiralpak AY-H 5 μm, flow rate 1 ml/min; mobile phase A: 50% n-heptane, mobile phase B: 50% ethanol+0.2% diethylamine in B; temperature 55° C.; detection: 220 nm) was collected. Removal of the solvents gave 28 mg (99% ee) of the title compound.

LC-MS (Methode 1): Rₜ=1.07 min; MS (ESIpos): m/z=506 [M+H]⁺.

¹H-NMR (600 MHz, DMSO-d6) δ[ppm]: −0.146 (2.11), −0.024 (0.87), −0.017 (3.12), −0.015 (3.03), −0.009 (3.17), −0.007 (3.10), 0.275 (0.95), 0.282 (2.68), 0.285 (2.76), 0.288 (1.22), 0.292 (1.19), 0.295 (2.78), 0.298 (2.72), 0.305 (0.83), 0.774 (0.55), 0.791 (0.83), 0.793 (0.81), 0.804 (0.78), 0.807 (0.82), 0.815 (1.19), 0.823 (0.58), 0.826 (0.62), 1.250 (0.55), 1.270 (0.56), 1.291 (0.42), 1.298 (0.41), 1.310 (0.79), 1.322 (0.82), 1.330 (1.17), 1.338 (0.85), 1.350 (0.84), 1.452 (1.28), 1.467 (0.79), 1.567 (0.60), 1.623 (1.14), 1.766 (0.43), 1.783 (0.69), 1.984 (0.68), 2.351 (8.08), 2.354 (10.97), 2.357 (8.10), 2.369 (0.63), 2.393 (16.00), 2.581 (0.58), 2.662 (0.72), 2.679 (0.68), 2.886 (1.09), 2.904 (2.03), 2.924 (1.09), 3.026 (5.94), 3.037 (5.84), 3.054 (0.64), 3.069 (1.97), 3.081 (3.29), 3.089 (2.05), 3.095 (0.72), 3.105 (0.53), 3.141 (15.73), 3.779 (1.60), 3.801 (1.51), 4.378 (3.00), 4.387 (2.96), 7.678 (7.07), 7.732 (0.80), 7.736 (0.84), 7.749 (1.21), 7.751 (1.24), 7.764 (0.81), 7.768 (0.81), 8.312 (3.04), 8.316 (2.99), 8.519 (0.96), 8.529 (1.94), 8.538 (0.95).

### Example 117

### diamix-N-[1-(2,5-Difluorophenyl)ethyl]-2-[(3R)-3′-fluoro-3-methyl[1,4′-bipiperidin]-1′-yl]-1,3-thiazole-5-carboxamide

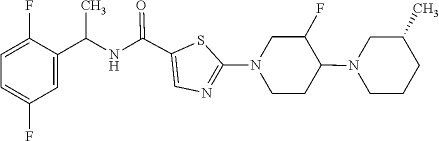 [SMILES: CC(NC(=O)c1cnc(N2CCC(N3CCC[C@@H](C)C3)C(F)C2)s1)c1cc(F)ccc1F]

rac-2-Bromo-N-[1-(2,5-difluorophenyl)ethyl]-1,3-thiazole-5-carboxamide (145 mg, 418 μmol) and diamix-(3R)-3′-fluoro-3-methyl-1,4′-bipiperidine dihydrochloride (98.9 mg, 362 μmol) were combined and stirred at 120° C. in 2 ml of sodium carbonate solution (2 ml, 2.0 M, 4 mmol) for 1 h. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 117 mg (purity 100%, 60% of theory) of the target compound.

LC-MS (Methode 1): Rₜ=1.18 min; MS (ESIpos): m/z=467 [M+H]⁺.

¹H-NMR (600 MHz, DMSO-d6) δ[ppm]: 0.808 (6.23), 0.816 (13.24), 0.826 (12.53), 0.841 (1.66), 0.861 (0.65), 1.378 (1.12), 1.398 (1.48), 1.417 (16.00), 1.429 (15.57), 1.544 (1.06), 1.563 (2.56), 1.580 (1.42), 1.585 (1.64), 1.622 (1.79), 1.643 (1.71), 1.675 (1.64), 1.693 (1.97), 1.863 (1.00), 1.872 (2.46), 1.888 (3.25), 1.905 (2.33), 2.157 (1.20), 2.176 (2.36), 2.194 (1.22), 2.617 (1.14), 2.655 (1.04), 2.676 (1.00), 2.805 (1.54), 2.820 (3.23), 3.131 (1.10), 3.153 (2.11), 3.175 (1.10), 3.232 (1.60), 3.257 (2.09), 3.322 (1.73), 3.998 (1.42), 4.018 (1.34), 4.194 (1.34), 5.053 (2.25), 5.135 (2.27), 5.228 (0.55), 5.240 (2.19), 5.251 (3.23), 5.263 (2.17), 5.276 (0.51), 7.099 (1.22), 7.113 (2.42), 7.120 (1.81), 7.127 (1.62), 7.133 (0.85), 7.195 (1.64), 7.203 (2.01), 7.211 (4.14), 7.218 (4.04), 7.226 (2.84), 7.233 (2.40), 7.902 (11.61), 7.914 (0.51), 8.535 (3.76), 8.547 (3.57).

### Example 118

### 4-(2-Chlorophenyl)-N-[(3,5-difluoropyridin-2-yl)methyl]-2-[(3R)-3-methyl[1,4′-bipiperidin]-1′-yl]-1,3-thiazole-5-carboxamide

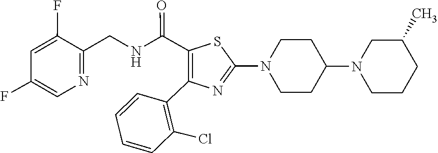 [SMILES: C[C@@H]1CCCN(C2CCN(c3nc(-c4ccccc4Cl)c(C(=O)NCc4ncc(F)cc4F)s3)CC2)C1]

N,N-Diisopropylethylamine (250 μl, 1.4 mmol) and propylphosphonic anhydride (280 μl, 50% in ethyl acetate, 460 μmol) were added to a solution of 4-(2-chlorophenyl)-2-[(3R)-3-methyl[1,4′-bipiperidin]-1′-yl]-1,3-thiazole-5-carboxylic acid (150 mg, 357 μmol) and 1-(3,5-difluoropyridin-2-yl)methanamine dihydrochloride (101 mg, 464 μmol) in 4.8 ml of acetonitrile, and the mixture was stirred at room temperature overnight. The reaction mixture was concentrated on a rotary evaporator and the residue was dissolved in DMSO, filtered and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature, wavelength 200-400 nm, complete injection; gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 19.0 mg (purity 100%, 10% of theory) of the target compound.

LC-MS (Methode 5): Rₜ=2.13 min; MS (ESIpos): m/z=546 [M+H]⁺.

¹H-NMR (600 MHz, DMSO-d6) δ[ppm]: 0.796 (0.63), 0.819 (15.20), 0.830 (16.00), 0.850 (0.61), 0.857 (0.57), 1.377 (0.51), 1.397 (1.27), 1.418 (1.39), 1.438 (0.57), 1.485 (0.85), 1.492 (1.06), 1.505 (2.64), 1.512 (3.04), 1.525 (3.40), 1.531 (3.30), 1.544 (1.54), 1.573 (1.65), 1.595 (1.35), 1.621 (1.37), 1.642 (1.31), 1.746 (1.78), 1.763 (3.06), 1.782 (3.83), 1.806 (2.62), 2.040 (1.06), 2.055 (1.90), 2.073 (1.06), 2.423 (0.40), 2.474 (1.12), 2.740 (1.75), 2.753 (3.19), 2.770 (1.50), 3.061 (2.13), 3.078 (3.80), 3.098 (2.16), 3.258 (0.53), 3.314 (0.63), 3.319 (0.53), 3.917 (2.75), 3.939 (2.62), 4.384 (5.88), 4.392 (5.81), 7.141 (1.88), 7.149 (3.80), 7.157 (1.86), 7.393 (1.10), 7.404 (3.34), 7.417 (3.30), 7.427 (4.23), 7.430 (5.09), 7.440 (2.18), 7.443 (1.73), 7.480 (1.46), 7.484 (1.25), 7.494 (3.15), 7.497 (2.71), 7.506 (2.41), 7.508 (2.30), 7.522 (5.28), 7.535 (2.37), 7.857 (1.42), 7.861 (1.52), 7.873 (2.37), 7.877 (2.47), 7.889 (1.48), 7.893 (1.52), 8.248 (5.85), 8.252 (5.81).

### Example 119

### 4-Bromo-N-[(3,5-difluoropyridin-2-yl)methyl]-2-[(3R)-3-methyl[1,4′-bipiperidin]-1′-yl]-1,3-thiazole-5-carboxamide

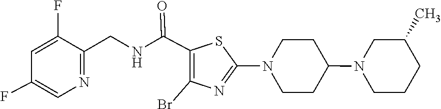 [SMILES: C[C@@H]1CCCN(C2CCN(c3nc(Br)c(C(=O)NCc4ncc(F)cc4F)s3)CC2)C1]

N,N-Diisopropylethylamine (180 μl, 1.0 mmol) and propylphosphonic anhydride (200 μl, 50% in ethyl acetate, 330 μmol) were added to a solution of 4-bromo-2-[(3R)-3-methyl[1,4′-bipiperidin]-1′-yl]-1,3-thiazole-5-carboxylic acid (100 mg, 258 μmol) and 1-(3,5-difluoropyridin-2-yl)methanamine dihydrochloride (72.7 mg, 335 μmol) in 4.0 ml of acetonitrile, and the mixture was stirred at room temperature overnight. The reaction mixture was concentrated on a rotary evaporator and the residue was dissolved in DMSO, filtered and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature, wavelength 200-400 nm, complete injection; gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 24.0 mg (purity 100%, 18% of theory) of the target compound.

LC-MS (Methode 5): Rₜ=2.00 min; MS (ESIneg): m/z=513 [M−H]⁻.

¹H-NMR (600 MHz, DMSO-d6) δ[ppm]: 0.785 (0.48), 0.791 (0.54), 0.805 (1.48), 0.815 (15.08), 0.826 (16.00), 0.845 (0.61), 0.851 (0.50), 1.371 (0.48), 1.391 (1.23), 1.411 (1.30), 1.425 (0.40), 1.432 (0.56), 1.458 (0.71), 1.479 (1.90), 1.495 (2.41), 1.500 (2.41), 1.512 (1.82), 1.518 (1.65), 1.529 (0.94), 1.541 (0.59), 1.567 (1.57), 1.573 (1.21), 1.583 (0.96), 1.589 (1.26), 1.617 (1.28), 1.638 (1.25), 1.737 (1.80), 1.754 (3.05), 1.772 (3.93), 1.795 (2.40), 2.035 (1.03), 2.050 (1.90), 2.054 (1.86), 2.069 (1.69), 2.482 (1.21), 2.519 (1.17), 2.722 (1.72), 2.734 (2.95), 2.751 (1.42), 3.063 (1.74), 3.068 (2.05), 3.085 (3.51), 3.088 (3.41), 3.105 (2.05), 3.110 (1.76), 3.318 (0.48), 3.876 (2.18), 3.898 (2.07), 4.591 (5.46), 4.600 (5.48), 7.911 (1.44), 7.915 (1.53), 7.928 (2.03), 7.930 (2.15), 7.943 (1.48), 7.947 (1.55), 8.178 (1.69), 8.187 (3.45), 8.196 (1.71), 8.478 (5.56), 8.482 (5.54).

### Example 120

### 4-Chloro-N-[(3,5-difluoropyridin-2-yl)methyl]-2-[(3R)-3-methyl[1,4′-bipiperidin]-1′-yl]-1,3-thiazole-5-carboxamide

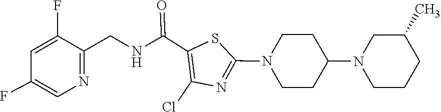 [SMILES: C[C@@H]1CCCN(C2CCN(c3nc(Cl)c(C(=O)NCc4ncc(F)cc4F)s3)CC2)C1]

2-Bromo-4-chloro-N-[(3,5-difluoropyridin-2-yl)methyl]-1,3-thiazole-5-carboxamide (100 mg, 271 μmol) and (3R)-3-methyl-1,4′-bipiperidine dihydrochloride (69.2 mg, 271 μmol) were combined and stirred at 120° C. in sodium carbonate solution (540 μl, 2.0 M, 1.1 mmol) for 1 h. The solid obtained was then filtered off with suction, washed with MTBE and dried under high vacuum. This gave 111 mg (purity 100%, 87% of theory) of the target compound.

LC-MS (Methode 5): Rₜ=1.96 min; MS (ESIpos): m/z=470 [M+H]⁺.

¹H-NMR (400 MHz, DMSO-d6) δ[ppm]: −0.149 (0.48), 0.146 (0.50), 0.773 (0.60), 0.810 (14.86), 0.826 (16.00), 0.852 (0.70), 0.862 (0.57), 1.352 (0.47), 1.383 (1.15), 1.413 (1.36), 1.443 (1.27), 1.472 (2.29), 1.495 (2.91), 1.504 (2.92), 1.522 (2.00), 1.531 (1.75), 1.560 (1.89), 1.602 (1.85), 1.641 (1.32), 1.725 (1.91), 1.751 (3.63), 1.775 (3.60), 1.797 (2.47), 2.023 (1.08), 2.046 (1.94), 2.052 (1.91), 2.074 (1.10), 2.328 (0.60), 2.367 (0.85), 2.670 (0.64), 2.674 (0.49), 2.710 (2.59), 2.719 (2.45), 2.736 (2.63), 3.055 (2.04), 3.080 (3.62), 3.111 (2.12), 3.868 (2.52), 3.900 (2.37), 4.580 (5.48), 4.593 (5.48), 7.910 (1.38), 7.916 (1.50), 7.935 (2.01), 7.938 (2.12), 7.941 (1.91), 7.957 (1.46), 7.963 (1.55), 8.146 (1.68), 8.159 (3.47), 8.173 (1.63), 8.483 (4.79), 8.489 (4.63).

### Example 121

### rac-N-[(3,5-Difluoropyridin-2-yl)methyl]-2-(3-propyl[1,4′-bipiperidin]-1′-yl)-1,3-thiazole-5-carboxamide

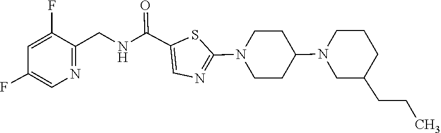 [SMILES: CCCC1CCCN(C2CCN(c3ncc(C(=O)NCc4ncc(F)cc4F)s3)CC2)C1]

N,N-Diisopropylethylamine (49 μl, 280 μmol) and acetic acid (9.7 μl, 170 μmol) were added in succession to a solution of N-[(3,5-difluoropyridin-2-yl)methyl]-2-(4-oxopiperidin-1-yl)-1,3-thiazole-5-carboxamide (50 mg, 142 μmol) and rac-3-propylpiperidine (36.1 mg, 284 μmol) in 3 ml of dichloromethane, and the mixture was stirred at room temperature for 6 h. Subsequently, sodium triacetoxyborohydride (45.1 mg, 213 μmol) was added and the mixture was stirred at room temperature overnight. Sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume), total flow rate: 80 ml/min; room temperature; wavelength 200-400 nm, complete injection. Gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time). The product-containing fractions were combined and concentrated and the residue was dried under high vacuum. This gave 9.00 mg (purity 100%, 14% of theory) of the target compound.

LC-MS (Methode 5): Rₜ=1.89 min; MS (ESIpos): m/z=464 [M+H]⁺.

¹H-NMR (600 MHz, DMSO-d6) δ[ppm]: 0.782 (0.42), 0.796 (0.99), 0.802 (1.00), 0.815 (1.07), 0.822 (1.05), 0.834 (7.50), 0.847 (16.00), 0.859 (8.04), 1.080 (0.68), 1.091 (0.99), 1.094 (0.85), 1.103 (1.64), 1.116 (1.70), 1.128 (1.13), 1.137 (1.31), 1.152 (1.62), 1.163 (1.21), 1.174 (0.77), 1.185 (0.44), 1.249 (0.74), 1.261 (2.17), 1.274 (3.33), 1.286 (2.73), 1.298 (1.24), 1.354 (0.40), 1.374 (1.15), 1.380 (0.89), 1.393 (1.72), 1.409 (1.32), 1.414 (1.31), 1.420 (1.00), 1.426 (0.70), 1.440 (0.48), 1.448 (0.57), 1.461 (1.18), 1.470 (1.54), 1.480 (1.76), 1.490 (1.64), 1.499 (1.26), 1.509 (0.64), 1.570 (1.35), 1.575 (1.08), 1.586 (0.84), 1.591 (1.10), 1.654 (1.11), 1.659 (1.08), 1.667 (0.72), 1.675 (1.11), 1.762 (2.32), 1.778 (3.07), 1.795 (2.67), 1.813 (1.30), 2.057 (0.93), 2.072 (1.64), 2.075 (1.62), 2.090 (0.89), 2.473 (0.92), 2.479 (0.63), 2.727 (1.42), 2.743 (2.48), 2.753 (1.62), 3.021 (1.72), 3.041 (3.32), 3.062 (1.72), 3.923 (2.55), 3.944 (2.45), 4.524 (4.71), 4.533 (4.70), 7.822 (12.11), 7.878 (1.31), 7.882 (1.39), 7.894 (1.90), 7.897 (1.97), 7.909 (1.32), 7.913 (1.36), 8.458 (5.03), 8.462 (4.90), 8.663 (1.52), 8.673 (3.08), 8.683 (1.51).

### Example 122

### 4-Cyclopropyl-N-[(3,5-difluoropyridin-2-yl)methyl]-2-[(3R)-3-methyl[1,4′-bipiperidin]-1′-yl]-1,3-thiazole-5-carboxamide

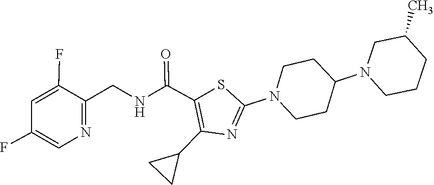 [SMILES: C[C@@H]1CCCN(C2CCN(c3nc(C4CC4)c(C(=O)NCc4ncc(F)cc4F)s3)CC2)C1]

2-Bromo-4-cyclopropyl-N-[(3,5-difluoropyridin-2-yl)methyl]-1,3-thiazole-5-carboxamide (100 mg, 267 μmol) and (3R)-3-methyl-1,4′-bipiperidine dihydrochloride (68.2 mg, 267 μmol) were combined and stirred at 120° C. in sodium carbonate solution (530 μl, 2.0 M, 1.1 mmol) for 1 h. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume), total flow rate: 80 ml/min; room temperature; wavelength 200-400 nm, complete injection. Gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 80.0 mg (purity 98%, 62% of theory) of the target compound.

LC-MS (Methode 5): Rₜ=2.11 min; MS (ESIpos): m/z=476 [M+H]⁺.

¹H-NMR (600 MHz, DMSO-d6) δ[ppm]: 0.787 (0.64), 0.801 (1.39), 0.812 (15.16), 0.823 (16.00), 0.834 (1.40), 0.841 (3.13), 0.846 (4.94), 0.850 (3.91), 0.855 (2.63), 0.860 (5.16), 0.868 (3.30), 0.872 (4.59), 0.876 (4.88), 0.880 (5.43), 0.884 (3.26), 0.892 (0.83), 1.366 (0.47), 1.387 (1.21), 1.407 (1.28), 1.431 (1.04), 1.444 (2.03), 1.451 (2.08), 1.464 (2.21), 1.471 (2.10), 1.485 (1.20), 1.491 (1.33), 1.502 (0.89), 1.508 (1.05), 1.519 (1.03), 1.526 (0.92), 1.564 (1.52), 1.580 (0.92), 1.585 (1.23), 1.615 (1.25), 1.636 (1.21), 1.733 (1.89), 1.750 (5.64), 1.768 (3.44), 2.029 (1.03), 2.044 (1.84), 2.048 (1.84), 2.063 (1.02), 2.423 (0.47), 2.442 (1.04), 2.461 (1.92), 2.479 (1.09), 2.652 (0.41), 2.715 (1.59), 2.728 (2.95), 2.746 (1.40), 2.772 (0.74), 2.781 (1.42), 2.786 (1.50), 2.794 (2.41), 2.802 (1.38), 2.807 (1.33), 2.816 (0.65), 2.974 (1.92), 2.991 (3.49), 3.012 (1.96), 3.264 (0.81), 3.321 (0.75), 3.826 (2.55), 3.847 (2.41), 4.507 (5.32), 4.516 (5.29), 7.868 (1.47), 7.872 (1.67), 7.885 (2.09), 7.887 (2.23), 7.888 (2.12), 7.900 (1.60), 7.903 (1.61), 7.955 (1.67), 7.964 (3.49), 7.973 (1.71), 8.452 (5.76), 8.455 (5.74).

### Example 123

### ent-N-[(3,5-Difluoropyridin-2-yl)methyl]-2-(3-ethoxy[1,4′-bipiperidin]-1′-yl)-1,3-thiazole-5-carboxamide (enantiomer 1)

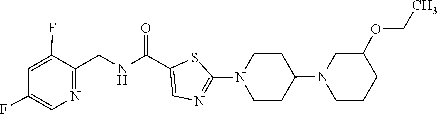 [SMILES: CCOC1CCCN(C2CCN(c3ncc(C(=O)NCc4ncc(F)cc4F)s3)CC2)C1]

97 mg of rac-N-[(3,5-difluoropyridin-2-yl)methyl]-2-(3-ethoxy[1,4′-bipiperidin]-1′-yl)-1,3-thiazole-5-carboxamide were separated into the enantiomers by chiral HPLC (preparative HPLC: column Daicel® Chiralpak ID, 5 μm, 250×20 mm; mobile phase A: 40% n-heptane, mobile phase B: 60% ethanol+0.2% diethylamine in B; flow rate 20 ml/min; temperature 50° C., detection: 220 nm). The enantiomer having a retention time of 2.336 min (HPLC: column Daicel® Chiralpak ID-3 3 μm, flow rate 1 ml/min; mobile phase A: 50% n-heptane, mobile phase B: 50% ethanol+0.2% diethylamine in B; detection: 220 nm) was collected. Removal of the solvents gave 38 mg (99% ee) of the title compound.

LC-MS (Methode 2): Rₜ=0.52 min; MS (ESIpos): m/z=466 [M+H]⁺.

¹H-NMR (600 MHz, DMSO-d6) δ[ppm]: 1.060 (8.60), 1.071 (16.00), 1.083 (8.26), 1.235 (0.59), 1.346 (1.07), 1.365 (1.08), 1.478 (2.05), 1.497 (2.16), 1.615 (1.29), 1.638 (1.12), 1.765 (2.83), 1.784 (2.51), 1.886 (1.30), 1.901 (1.26), 1.943 (0.94), 1.959 (1.62), 1.975 (0.96), 2.066 (0.82), 2.084 (1.44), 2.100 (0.79), 2.422 (0.44), 2.651 (1.43), 2.936 (1.37), 2.952 (1.34), 3.024 (2.23), 3.043 (4.12), 3.061 (2.16), 3.248 (1.59), 3.263 (1.29), 3.312 (0.54), 3.431 (1.22), 3.442 (3.89), 3.453 (5.34), 3.464 (4.14), 3.476 (1.31), 3.479 (1.09), 3.929 (2.79), 3.948 (2.67), 4.524 (6.31), 4.533 (6.25), 7.824 (13.37), 7.879 (1.72), 7.882 (1.81), 7.898 (2.60), 7.910 (1.70), 7.914 (1.78), 8.459 (6.60), 8.462 (6.49), 8.666 (1.78), 8.676 (3.41), 8.685 (1.73).

### Example 124

### ent-N-[(3,5-Difluoropyridin-2-yl)methyl]-2-(3-ethoxy[1,4′-bipiperidin]-1′-yl)-1,3-thiazole-5-carboxamide (enantiomer 2)

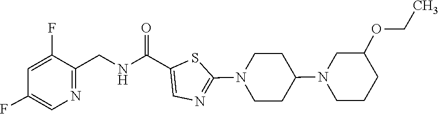 [SMILES: CCOC1CCCN(C2CCN(c3ncc(C(=O)NCc4ncc(F)cc4F)s3)CC2)C1]

97 mg of rac-N-[(3,5-difluoropyridin-2-yl)methyl]-2-(3-ethoxy[1,4′-bipiperidin]-1′-yl)-1,3-thiazole-5-carboxamide were separated into the enantiomers by chiral HPLC (preparative HPLC: column Daicel® Chiralpak ID, 5 μm, 250×20 mm; mobile phase A: 40% n-heptane, mobile phase B: 60% ethanol+0.2% diethylamine in B; flow rate 20 ml/min; temperature 50° C., detection: 220 nm). The enantiomer having a retention time of 4.263 min (HPLC: column Daicel® Chiralpak ID-3 3 μm, flow rate 1 ml/min; mobile phase A: 50% n-heptane, mobile phase B: 50% ethanol+0.2% diethylamine in B; detection: 220 nm) was collected. Removal of the solvents gave 37 mg (99% ee) of the title compound.

LC-MS (Methode 2): Rₜ=0.52 min; MS (ESIpos): m/z=466 [M+H]⁺.

¹H-NMR (600 MHz, DMSO-d6) δ[ppm]: 0.858 (0.50), 1.060 (8.69), 1.072 (16.00), 1.083 (8.44), 1.236 (1.50), 1.355 (1.34), 1.366 (1.33), 1.479 (2.47), 1.498 (2.65), 1.616 (1.60), 1.767 (3.37), 1.785 (3.03), 1.888 (1.68), 1.904 (1.66), 1.960 (1.76), 2.084 (1.62), 2.611 (0.50), 2.652 (1.56), 2.939 (1.58), 3.024 (2.76), 3.044 (5.20), 3.064 (2.72), 3.251 (2.20), 3.431 (1.40), 3.443 (4.13), 3.454 (5.81), 3.465 (4.31), 3.477 (1.43), 3.930 (3.48), 3.951 (3.36), 4.524 (8.10), 4.533 (8.05), 7.824 (12.08), 7.879 (1.78), 7.882 (1.96), 7.897 (3.40), 7.910 (1.75), 7.914 (1.92), 8.459 (7.08), 8.462 (7.27), 8.667 (2.01), 8.676 (3.85), 8.685 (1.99).

### Example 125

### ent-2-[3-(Cyclobutylmethoxy)[1,4′-bipiperidin]-1′-yl]-N-[(3,5-difluoropyridin-2-yl)methyl]-1,3-thiazole-5-carboxamide (enantiomer 1)

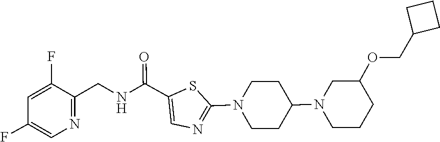 [SMILES: O=C(NCc1ncc(F)cc1F)c1cnc(N2CCC(N3CCCC(OCC4CCC4)C3)CC2)s1]

60 mg of rac-2-[3-(cyclobutylmethoxy)[1,4′-bipiperidin]-1′-yl]-N-[(3,5-difluoropyridin-2-yl)methyl]-1,3-thiazole-5-carboxamide were separated into the enantiomers by chiral HPLC (preparative HPLC: column Daicel@ Chiralpak IF, 5 μm, 250×20 mm; mobile phase A: 100% ethanol+0.2% diethylamine; flow rate 18 ml/min; temperature 70° C., detection: 220 nm). The enantiomer having a retention time of 9.999 min (HPLC: column Daicel© Chiralpak IF 5 μm, flow rate 1 ml/min; mobile phase A: 100% ethanol+0.2% diethylamine; temperature 70° C.; detection: 220 nm) was collected. Removal of the solvents gave 28 mg (99% ee) of the title compound.

LC-MS (Methode 1): Rₜ=1.17 min; MS (ESIpos): m/z=506 [M+H]⁺.

¹H-NMR (600 MHz, DMSO-d6) δ[ppm]: 1.040 (2.07), 1.059 (2.23), 1.146 (0.91), 1.158 (1.69), 1.171 (1.07), 1.234 (0.93), 1.341 (2.06), 1.360 (2.22), 1.478 (3.96), 1.497 (4.40), 1.615 (2.66), 1.645 (5.09), 1.661 (6.44), 1.675 (5.50), 1.690 (2.56), 1.765 (5.75), 1.784 (6.56), 1.809 (6.11), 1.824 (6.18), 1.837 (3.88), 1.856 (1.91), 1.888 (2.92), 1.904 (2.89), 1.922 (3.01), 1.936 (6.49), 1.950 (8.09), 2.081 (3.01), 2.405 (1.46), 2.418 (3.03), 2.430 (3.84), 2.442 (3.00), 2.455 (1.53), 2.654 (3.03), 2.941 (2.94), 2.954 (2.67), 3.022 (4.06), 3.041 (7.67), 3.061 (4.38), 3.225 (3.06), 3.357 (3.40), 3.373 (7.35), 3.384 (12.29), 3.396 (7.41), 3.411 (2.89), 3.929 (5.35), 3.950 (5.23), 4.523 (11.02), 4.532 (11.02), 7.823 (16.00), 7.878 (2.73), 7.895 (4.72), 7.910 (2.65), 8.458 (9.67), 8.664 (3.17), 8.674 (5.69), 8.683 (3.01).

### Example 126

### ent-2-[3-(Cyclobutylmethoxy)[1,4′-bipiperidin]-1′-yl]-N-[(3,5-difluoropyridin-2-yl)methyl]-1,3-thiazole-5-carboxamide (enantiomer 2)

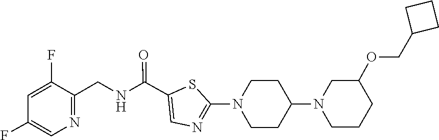 [SMILES: O=C(NCc1ncc(F)cc1F)c1cnc(N2CCC(N3CCCC(OCC4CCC4)C3)CC2)s1]

60 mg of rac-2-[3-(cyclobutylmethoxy)[1,4′-bipiperidin]-1′-yl]-N-[(3,5-difluoropyridin-2-yl)methyl]-1,3-thiazole-5-carboxamide were separated into the enantiomers by chiral HPLC (preparative HPLC: column Daicel® Chiralpak IF, 5 μm, 250×20 mm; mobile phase A: 100% ethanol+0.2% diethylamine; flow rate 18 ml/min; temperature 70° C., detection: 220 nm). The enantiomer having a retention time of 13.165 min (HPLC: column Daicel® Chiralpak IF 5 μm, flow rate 1 ml/min; mobile phase A: 100% ethanol+0.2% diethylamine; temperature 70° C.; detection: 220 nm) was collected. Removal of the solvents gave 28 mg (99% ee) of the title compound.

LC-MS (Methode 1): Rₜ=1.17 min; MS (ESIpos): m/z=506 [M+H]⁺.

¹H-NMR (600 MHz, DMSO-d6) δ[ppm]: 1.023 (0.51), 1.037 (1.19), 1.060 (1.31), 1.078 (0.73), 1.143 (1.12), 1.155 (2.25), 1.167 (1.19), 1.235 (0.77), 1.321 (0.52), 1.341 (1.20), 1.361 (1.25), 1.381 (0.56), 1.477 (2.28), 1.497 (2.44), 1.516 (0.97), 1.614 (1.60), 1.635 (1.78), 1.647 (2.81), 1.662 (3.59), 1.677 (2.98), 1.690 (1.31), 1.766 (3.39), 1.775 (2.10), 1.785 (3.78), 1.793 (2.73), 1.800 (2.08), 1.810 (3.38), 1.815 (1.70), 1.824 (4.53), 1.829 (1.22), 1.838 (2.43), 1.842 (1.48), 1.852 (0.85), 1.857 (0.97), 1.870 (0.53), 1.889 (1.42), 1.903 (1.41), 1.923 (1.61), 1.927 (1.22), 1.932 (1.92), 1.936 (3.89), 1.945 (3.31), 1.953 (4.52), 1.956 (4.37), 1.964 (2.83), 1.970 (2.58), 1.978 (1.21), 2.065 (1.01), 2.082 (1.80), 2.099 (0.98), 2.406 (0.92), 2.418 (1.98), 2.431 (2.54), 2.443 (1.88), 2.456 (0.85), 2.564 (0.87), 2.655 (1.60), 2.672 (1.48), 2.908 (0.92), 2.921 (0.98), 2.942 (1.72), 2.955 (1.58), 3.023 (2.34), 3.042 (4.41), 3.061 (2.35), 3.210 (1.08), 3.218 (1.46), 3.225 (1.90), 3.233 (1.43), 3.242 (1.19), 3.317 (0.46), 3.357 (1.62), 3.369 (1.91), 3.373 (5.39), 3.385 (10.05), 3.396 (5.32), 3.401 (1.89), 3.412 (1.57), 3.930 (3.09), 3.950 (2.94), 4.524 (6.50), 4.533 (6.41), 7.813 (0.48), 7.824 (16.00), 7.878 (1.87), 7.882 (1.93), 7.894 (2.71), 7.897 (2.72), 7.910 (1.85), 7.913 (1.87), 8.458 (7.25), 8.462 (6.95), 8.665 (2.10), 8.675 (4.20), 8.684 (2.03).

### Example 127

### rac-Formic acid N-[(3,5-difluoropyridin-2-yl)methyl]-2-[3-(2-fluoroethyl)[1,4′-bipiperidin]-1′-yl]-1,3-thiazole-5-carboxamide

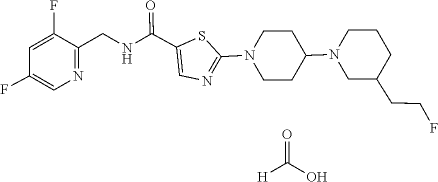 [SMILES: O=C(NCc1ncc(F)cc1F)c1cnc(N2CCC(N3CCCC(CCF)C3)CC2)s1 ; O=CO]

N,N-Diisopropylethylamine (49 μl, 280 μmol) and acetic acid (9.7 μl, 170 μmol) were added in succession to a solution of N-[(3,5-difluoropyridin-2-yl)methyl]-2-(4-oxopiperidin-1-yl)-1,3-thiazole-5-carboxamide (50 mg, 142 μmol) and rac-3-(2-fluoroethyl)piperidine (37.2 mg, 284 μmol) in 3 ml of dichloromethane, and the mixture was stirred at room temperature for 6 h. Subsequently, sodium triacetoxyborohydride (45.1 mg, 213 μmol) was added and the mixture was stirred at room temperature overnight. Sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: Phenomenex Kinetex C18 5 μm 100×30 mm. Mobile phase A: water, mobile phase B: acetonitrile, mobile phase C: 2% strength formic acid in water, mobile phase D: acetonitrile/water (80% by volume/20% by volume) total flow rate: 80 ml/min; room temperature; wavelength 200-400 nm, complete injection. Gradient profile: mobile phase A 0 to 2 min 63 ml, mobile phase B 0 to 2 min 7 ml, mobile phase A 2 to 10 min from 63 ml to 39 ml and mobile phase B from 7 ml to 31 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 8.3 mg (purity 90%, 62% of theory) of the target compound.

¹H-NMR (600 MHz, DMSO-d6) δ[ppm]: 0.891 (0.41), 0.908 (0.96), 0.929 (1.03), 0.943 (0.48), 1.380 (0.46), 1.401 (1.07), 1.421 (1.24), 1.440 (0.58), 1.471 (1.05), 1.485 (2.38), 1.491 (3.31), 1.504 (2.79), 1.511 (2.62), 1.524 (1.89), 1.532 (1.25), 1.542 (0.86), 1.552 (1.29), 1.562 (1.40), 1.586 (2.60), 1.592 (3.22), 1.598 (2.82), 1.609 (2.29), 1.613 (2.28), 1.620 (2.03), 1.630 (0.77), 1.681 (1.29), 1.702 (1.25), 1.774 (3.02), 1.794 (2.62), 1.901 (1.30), 1.917 (2.00), 1.934 (1.18), 2.135 (1.06), 2.150 (1.91), 2.154 (1.87), 2.168 (1.12), 2.520 (0.99), 2.564 (1.18), 2.652 (0.44), 2.735 (1.56), 2.754 (1.54), 2.781 (1.79), 2.799 (1.71), 3.028 (2.21), 3.048 (4.09), 3.068 (2.26), 3.102 (0.54), 3.480 (1.58), 3.563 (1.40), 3.934 (3.17), 3.955 (3.05), 4.430 (1.61), 4.438 (3.14), 4.448 (1.87), 4.509 (1.96), 4.519 (4.42), 4.525 (6.57), 4.533 (5.92), 7.824 (16.00), 7.865 (0.74), 7.879 (1.67), 7.883 (1.74), 7.895 (2.30), 7.898 (2.39), 7.910 (1.74), 7.914 (1.69), 8.171 (3.02), 8.459 (6.28), 8.463 (6.04), 8.668 (1.79), 8.678 (3.62), 8.687 (1.72).

### Example 128

### 2-([1,4′-Bipiperidin]-1′-yl)-N-[(3,5-difluoropyridin-2-yl)methyl]-1,3-thiazole-5-carboxamide

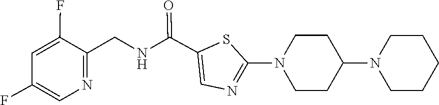 [SMILES: O=C(NCc1ncc(F)cc1F)c1cnc(N2CCC(N3CCCCC3)CC2)s1]

Acetic acid (9.7 μl, 170 μmol) was added to a solution of N-[(3,5-difluoropyridin-2-yl)methyl]-2-(4-oxopiperidin-1-yl)-1,3-thiazole-5-carboxamide (100.0 mg, 284 μmol) and piperidine (56 μl, 570 μmol) in 2 ml of dichloromethane, and the mixture was stirred at room temperature for 4 h. Subsequently, sodium triacetoxyborohydride (90.2 mg, 426 μmol) was added and the mixture was stirred at room temperature overnight. Subsequently, sat. NaHCO₃ solution was added and the reaction mixture was extracted with dichloromethane. The organic phase was concentrated on a rotary evaporator and the residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume). Total flow rate: 80 ml/min; room temperature; wavelength 200-400 nm, complete injection. Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 22.0 mg (100% purity, 18% of theory) of the title compound.

LC-MS (Methode 1): Rₜ=0.80 min; MS (ESIpos): m/z=422 [M+H]⁺.

¹H-NMR (600 MHz, DMSO-d6) δ[ppm]: 1.366 (3.44), 1.375 (2.92), 1.444 (1.06), 1.453 (3.16), 1.463 (7.91), 1.471 (9.96), 1.482 (6.13), 1.490 (4.09), 1.504 (1.17), 1.512 (1.00), 1.769 (3.09), 1.790 (2.71), 2.430 (5.90), 2.439 (8.35), 2.447 (6.39), 2.466 (1.66), 2.471 (2.35), 2.517 (0.56), 2.651 (0.41), 3.021 (2.01), 3.025 (2.37), 3.042 (4.07), 3.045 (4.08), 3.062 (2.33), 3.067 (2.09), 3.259 (0.66), 3.920 (3.18), 3.942 (3.09), 4.523 (5.69), 4.532 (5.70), 7.821 (16.00), 7.879 (1.63), 7.882 (1.78), 7.895 (2.33), 7.897 (2.41), 7.910 (1.70), 7.914 (1.76), 8.458 (6.16), 8.462 (6.16), 8.664 (1.76), 8.673 (3.62), 8.683 (1.83).

### Example 129

### N-[1-(3,5-Difluoropyridin-2-yl)cyclopropyl]-2-[(3R)-3-methyl[1,4′-bipiperidin]-1′-yl]-1,3-thiazole-5-carboxamide

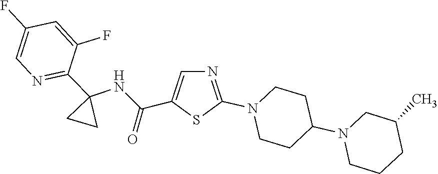 [SMILES: C[C@@H]1CCCN(C2CCN(c3ncc(C(=O)NC4(c5ncc(F)cc5F)CC4)s3)CC2)C1]

1-(3-Dimethylaminopropyl)-3-ethylcarbodiimide hydrochloride (32.6 mg, 170 μmol), 1-hydroxy-1H-benzotriazole hydrate (26.0 mg, 170 μmol) and N,N-diisopropylethylamine (110 μl, 650 μmol) were added to a solution of 2-[(3R)-3-methyl[1,4′-bipiperidin]-1′-yl]-1,3-thiazole-5-carboxylic acid dihydrochloride (50.0 mg, 131 μmol) in 2 ml of DMF and the mixture was stirred for 5 min, after which 1-(3,5-difluoropyridin-2-yl)cyclopropanamine hydrochloride (1:1) (29.7 mg, 144 μmol) was added. The mixture was then stirred at room temperature overnight. The reaction mixture was purified by preparative HPLC [instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 47 ml, mobile phase B 0 to 2 min 23 ml, mobile phase A 2 to 10 min from 47 ml to 23 ml and mobile phase B from 23 ml to 47 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 37.0 mg (100% purity, 61% of theory) of the title compound.

LC-MS (Methode 2): Rₜ=0.56 min; MS (ESIpos): m/z=462 [M+H]⁺.

¹H-NMR (600 MHz, DMSO-d6) δ[ppm]: 0.782 (0.51), 0.788 (0.60), 0.812 (15.17), 0.823 (16.00), 0.842 (0.59), 0.848 (0.53), 0.955 (0.47), 1.174 (2.26), 1.182 (6.55), 1.187 (6.16), 1.194 (2.43), 1.369 (0.51), 1.389 (1.31), 1.409 (1.42), 1.429 (0.62), 1.449 (0.85), 1.464 (2.23), 1.477 (4.28), 1.484 (9.56), 1.488 (8.40), 1.496 (3.72), 1.521 (1.41), 1.527 (1.17), 1.565 (1.73), 1.581 (1.06), 1.586 (1.39), 1.615 (1.46), 1.636 (1.39), 1.737 (1.83), 1.755 (5.18), 1.772 (2.86), 1.779 (2.96), 2.036 (1.15), 2.051 (2.05), 2.070 (1.12), 2.470 (1.22), 2.720 (1.75), 2.732 (3.29), 2.748 (1.72), 2.956 (0.44), 3.020 (2.17), 3.037 (3.83), 3.058 (2.15), 3.915 (2.81), 3.936 (2.67), 6.779 (0.67), 6.785 (0.65), 7.120 (0.64), 7.125 (0.60), 7.740 (1.36), 7.744 (1.44), 7.755 (1.57), 7.759 (2.67), 7.763 (1.55), 7.774 (1.38), 7.778 (1.38), 7.835 (11.84), 8.360 (5.23), 8.364 (4.87), 8.928 (5.56).

### Example 130

### N-[(3,5-Difluoropyridin-2-yl)methyl]-4-ethyl-2-[(3R)-3-methyl[1,4′-bipiperidin]-1′-yl]-1,3-thiazole-5-carboxamide

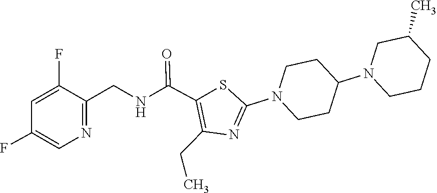 [SMILES: CCc1nc(N2CCC(N3CCC[C@@H](C)C3)CC2)sc1C(=O)NCc1ncc(F)cc1F]

2-Bromo-N-[(3,5-difluoropyridin-2-yl)methyl]-4-ethyl-1,3-thiazole-5-carboxamide (150 mg, 414 μmol) and (3R)-3-methyl-1,4′-bipiperidine dihydrochloride (106 mg, 414 μmol) were combined and stirred at 120° C. in sodium carbonate solution (830 μl, 2.0 M, 1.7 mmol) for 1 h. The reaction mixture was then purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 39 ml, mobile phase B 0 to 2 min 31 ml, mobile phase A 2 to 10 min from 39 ml to 15 ml and mobile phase B from 31 ml to 55 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 74.0 mg (purity 100%, 39% of theory) of the target compound.

LC-MS (Methode 2): Rₜ=0.60 min; MS (ESIpos): m/z=464 [M+H]⁺.

¹H-NMR (500 MHz, DMSO-d6) δ[ppm]: 0.799 (1.09), 0.813 (11.56), 0.827 (12.24), 0.847 (0.47), 1.091 (7.29), 1.106 (16.00), 1.121 (7.29), 1.388 (0.88), 1.395 (0.58), 1.412 (0.97), 1.438 (0.80), 1.447 (0.69), 1.464 (1.55), 1.472 (1.65), 1.488 (2.07), 1.495 (2.07), 1.510 (1.47), 1.521 (1.26), 1.530 (0.74), 1.544 (0.47), 1.565 (1.25), 1.571 (0.94), 1.584 (0.71), 1.591 (0.92), 1.598 (0.74), 1.615 (0.99), 1.641 (0.96), 1.733 (1.37), 1.754 (2.49), 1.774 (2.92), 1.793 (1.87), 2.029 (0.79), 2.047 (1.42), 2.052 (1.39), 2.069 (0.80), 2.453 (0.77), 2.459 (0.54), 2.469 (0.96), 2.475 (1.59), 2.482 (1.28), 2.523 (0.42), 2.727 (1.39), 2.740 (2.26), 2.760 (1.12), 2.789 (1.99), 2.804 (6.09), 2.819 (5.92), 2.834 (1.81), 2.998 (1.35), 3.003 (1.59), 3.024 (2.79), 3.028 (2.70), 3.048 (1.59), 3.891 (2.15), 3.917 (2.01), 4.488 (4.13), 4.499 (4.03), 7.879 (1.35), 7.883 (1.42), 7.897 (1.69), 7.899 (1.75), 7.901 (1.82), 7.903 (1.63), 7.917 (1.34), 7.921 (1.38), 7.989 (1.38), 8.000 (2.84), 8.011 (1.31), 8.452 (4.92), 8.456 (4.69).

### Example 131

### ent-2-[4-(1,1-Difluoro-5-azaspiro[2.5]octan-5-yl)piperidin-1-yl]-N-[(3,5-difluoropyridin-2-yl)methyl]-1,3-thiazole-5-carboxamide (enantiomer 1)

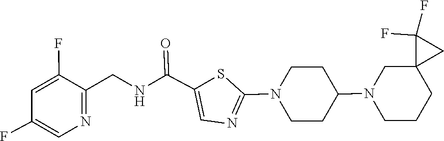 [SMILES: O=C(NCc1ncc(F)cc1F)c1cnc(N2CCC(N3CCCC4(C3)CC4(F)F)CC2)s1]

60 mg of rac-2-[4-(1,1-difluoro-5-azaspiro[2.5]octan-5-yl)piperidin-1-yl]-N-[(3,5-difluoropyridin-2-yl)methyl]-1,3-thiazole-5-carboxamide were separated into the enantiomers by chiral HPLC (preparative HPLC: column Daicel® Chiralpak ID, 5 μm, 250×20 mm; mobile phase A: 30% n-heptane, mobile phase B: 70% ethanol+0.2% diethylamine in B; flow rate 20 ml/min; temperature 40° C., detection: 220 nm). The enantiomer having a retention time of 1.927 min (HPLC: column Daicel® Chiralpak ID-3 3 μm, flow rate 1 ml/min; mobile phase A: 50% n-heptane, mobile phase B: 50% ethanol+0.2% diethylamine in B; detection: 220 nm) was collected. Removal of the solvents gave 23 mg (98% ee) of the title compound.

LC-MS (Methode 5): Rₜ=1.56 min; MS (ESIpos): m/z=484 [M+H]⁺.

¹H-NMR (600 MHz, DMSO-d6) δ[ppm]: 1.146 (0.84), 1.158 (2.52), 1.170 (2.50), 1.186 (1.37), 1.198 (1.48), 1.215 (2.36), 1.226 (2.39), 1.436 (0.92), 1.456 (3.88), 1.476 (8.04), 1.494 (7.15), 1.603 (2.19), 1.755 (2.31), 1.776 (4.13), 1.799 (1.99), 2.377 (2.43), 2.396 (3.21), 2.422 (2.44), 2.514 (4.21), 2.568 (1.52), 2.620 (1.95), 3.046 (3.16), 3.063 (5.74), 3.083 (3.23), 3.907 (3.91), 3.926 (3.72), 4.523 (8.18), 4.532 (8.18), 7.822 (16.00), 7.878 (1.98), 7.882 (2.00), 7.897 (3.37), 7.910 (2.00), 7.913 (1.97), 8.458 (7.68), 8.461 (7.29), 8.666 (2.49), 8.676 (4.92), 8.685 (2.41).

### Example 132

### ent-2-[4-(1,1-Difluoro-5-azaspiro[2.5]octan-5-yl)piperidin-1-yl]-N-[(3,5-difluoropyridin-2-yl)methyl]-1,3-thiazole-5-carboxamide (enantiomer 2)

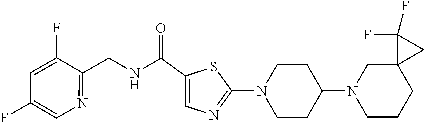 [SMILES: O=C(NCc1ncc(F)cc1F)c1cnc(N2CCC(N3CCCC4(C3)CC4(F)F)CC2)s1]

60 mg of rac-2-[4-(1,1-difluoro-5-azaspiro[2.5]octan-5-yl)piperidin-1-yl]-N-[(3,5-difluoropyridin-2-yl)methyl]-1,3-thiazole-5-carboxamide were separated into the enantiomers by chiral HPLC (preparative HPLC: column Daicel@ Chiralpak ID, 5 μm, 250×20 mm; mobile phase A: 30% n-heptane, mobile phase B: 70% ethanol+0.2% diethylamine in B; flow rate 20 ml/min; temperature 40° C., detection: 220 nm). The enantiomer having a retention time of 3.317 min (HPLC: column Daicel© Chiralpak ID-3 3 μm, flow rate 1 ml/min; mobile phase A: 50% n-heptane, mobile phase B: 50% ethanol+0.2% diethylamine in B; detection: 220 nm) was collected. Removal of the solvents gave 23 mg (99% ee) of the title compound.

LC-MS (Method 5): Rₜ=1.56 min; MS (ESIpos): m/z=484 [M+H]⁺.

¹H-NMR (600 MHz, DMSO-d6) δ[ppm]: 1.146 (0.75), 1.158 (1.98), 1.171 (1.91), 1.186 (1.08), 1.198 (1.15), 1.215 (1.81), 1.227 (1.85), 1.239 (0.96), 1.436 (0.80), 1.456 (2.96), 1.477 (5.97), 1.495 (5.35), 1.522 (1.38), 1.603 (1.63), 1.613 (1.24), 1.756 (1.75), 1.780 (3.00), 1.800 (1.48), 2.377 (1.82), 2.396 (2.41), 2.422 (1.92), 2.514 (3.06), 2.568 (1.08), 2.620 (1.43), 2.651 (0.41), 3.042 (2.15), 3.046 (2.46), 3.063 (4.28), 3.083 (2.49), 3.088 (2.11), 3.906 (2.87), 3.926 (2.70), 4.523 (6.11), 4.532 (6.07), 7.822 (16.00), 7.878 (1.73), 7.882 (1.81), 7.895 (2.55), 7.897 (2.67), 7.910 (1.78), 7.914 (1.86), 8.458 (6.46), 8.461 (6.36), 8.667 (1.89), 8.676 (3.89), 8.686 (1.94).

### Example 133

### rac-N-[(3,5-Difluoropyridin-2-yl)methyl]-2-(3-phenyl[1,4′-bipiperidin]-1′-yl)-1,3-thiazole-5-carboxamide

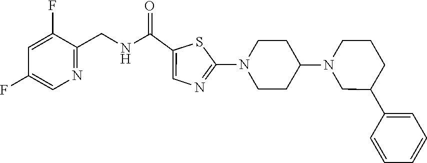 [SMILES: O=C(NCc1ncc(F)cc1F)c1cnc(N2CCC(N3CCCC(c4ccccc4)C3)CC2)s1]

N,N-Diisopropylethylamine (69 μl, 400 μmol) and acetic acid (14 μl, 240 μmol) were added in succession to a solution of N-[(3,5-difluoropyridin-2-yl)methyl]-2-(4-oxopiperidin-1-yl)-1,3-thiazole-5-carboxamide (70.0 mg, 199 μmol) and rac-3-phenylpiperidine (64.1 mg, 397 μmol) in 4.2 ml of dichloromethane, and the mixture was stirred at room temperature overnight. Subsequently, sodium triacetoxyborohydride (63.2 mg, 298 μmol) was added and the mixture was stirred at room temperature for 5 h. Subsequently, sat. NaHCO₃ solution was added and the reaction mixture was extracted with dichloromethane. The organic phase was washed with water and dried over Na₂SO₄. The drying agent was filtered off and the filtrate was concentrated. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 70 ml, mobile phase B 0 to 2 min 0 ml, mobile phase A 2 to 10 min from 70 ml to 0 ml and mobile phase B from 0 ml to 70 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 17.0 mg (purity 100%, 17% of theory) of the target compound.

LC-MS (Methode 5): Rₜ=1.74 min; MS (ESIpos): m/z=498 [M+H]⁺.

¹H-NMR (400 MHz, DMSO-d6) δ[ppm]: −0.149 (0.91), 0.146 (0.77), 1.378 (0.42), 1.400 (1.08), 1.408 (1.16), 1.430 (1.35), 1.439 (1.38), 1.461 (1.11), 1.496 (1.85), 1.510 (2.16), 1.528 (2.44), 1.560 (1.50), 1.704 (1.58), 1.736 (1.16), 1.796 (3.14), 1.819 (2.95), 2.073 (2.48), 2.157 (1.25), 2.185 (1.75), 2.201 (1.77), 2.228 (3.01), 2.255 (1.62), 2.328 (1.28), 2.367 (1.69), 2.524 (3.95), 2.574 (2.01), 2.601 (0.88), 2.666 (1.83), 2.670 (1.83), 2.693 (1.57), 2.711 (2.19), 2.856 (2.82), 2.883 (2.55), 3.015 (1.85), 3.045 (3.45), 3.075 (1.89), 3.921 (2.88), 3.954 (2.64), 4.514 (4.86), 4.527 (4.95), 7.166 (1.21), 7.172 (0.84), 7.182 (3.04), 7.193 (1.08), 7.199 (1.96), 7.204 (1.62), 7.241 (2.91), 7.257 (12.12), 7.263 (16.00), 7.280 (6.40), 7.299 (1.70), 7.820 (15.56), 7.881 (1.54), 7.887 (1.58), 7.906 (2.02), 7.910 (2.10), 7.929 (1.58), 7.935 (1.60), 8.173 (0.95), 8.460 (4.70), 8.465 (4.61), 8.685 (1.67), 8.699 (3.57), 8.713 (1.70).

### Example 134

### diamix-2-[4-(1,1-Difluoro-5-azaspiro[2.5]octan-5-yl)-3-fluoropiperidin-1-yl]-N-[(3,5-difluoropyridin-2-yl)methyl]-1,3-thiazole-5-carboxamide

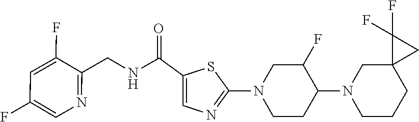 [SMILES: O=C(NCc1ncc(F)cc1F)c1cnc(N2CCC(N3CCCC4(C3)CC4(F)F)C(F)C2)s1]

2-Bromo-N-[(3,5-difluoropyridin-2-yl)methyl]-1,3-thiazole-5-carboxamide (100 mg, 299 μmol) and diamix-1,1-difluoro-5-(3-fluoropiperidin-4-yl)-5-azaspiro[2.5]octane dihydrochloride (96.1 mg, 299 μmol) were combined and stirred at 120° C. in 2 ml of sodium carbonate solution (2 ml, 2.0 M, 4 mmol) for 30 hours. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 46.0 mg (purity 100%, 31% of theory) of the target compound.

LC-MS (Methode 5): Rₜ=1.52 min; MS (ESIpos): m/z=502 [M+H]⁺.

¹H-NMR (400 MHz, DMSO-d6) δ[ppm]: −0.149 (0.40), 1.174 (2.35), 1.195 (4.08), 1.216 (2.33), 1.232 (0.76), 1.462 (1.17), 1.481 (2.78), 1.501 (4.70), 1.523 (1.86), 1.608 (1.52), 1.623 (1.37), 1.668 (1.19), 1.703 (1.07), 1.846 (0.95), 1.858 (1.09), 1.879 (1.18), 1.890 (1.21), 1.911 (0.72), 1.923 (0.63), 2.328 (0.44), 2.367 (0.65), 2.524 (3.86), 2.604 (2.69), 2.633 (1.49), 2.670 (1.14), 2.699 (2.14), 2.710 (2.28), 2.769 (0.60), 2.788 (0.77), 3.126 (1.00), 3.158 (1.99), 3.190 (1.15), 3.214 (1.44), 3.250 (1.52), 3.987 (1.42), 4.019 (1.34), 4.153 (0.87), 4.187 (1.56), 4.217 (0.79), 4.521 (5.39), 4.534 (5.43), 5.026 (1.17), 5.056 (0.66), 5.149 (1.18), 5.177 (0.67), 7.812 (16.00), 7.885 (1.58), 7.891 (1.73), 7.908 (2.00), 7.910 (2.18), 7.913 (2.27), 7.916 (2.13), 7.933 (1.66), 7.938 (1.74), 8.464 (5.05), 8.470 (5.00), 8.709 (1.84), 8.724 (3.89), 8.738 (1.87).

### Example 135

### diamix-2-[4-(5-Azaspiro[2.5]octan-5-yl)-3-fluoropiperidin-1-yl]-N-[(3,5-difluoropyridin-2-yl)methyl]-1,3-thiazole-5-carboxamide

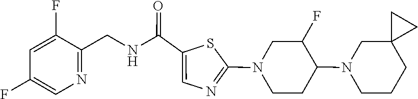 [SMILES: O=C(NCc1ncc(F)cc1F)c1cnc(N2CCC(N3CCCC4(CC4)C3)C(F)C2)s1]

2-Bromo-N-[(3,5-difluoropyridin-2-yl)methyl]-1,3-thiazole-5-carboxamide (100 mg, 299 μmol) and diamix-5-(3-fluoropiperidin-4-yl)-5-azaspiro[2.5]octane dihydrochloride (85.4 mg, 299 μmol) were combined and stirred at 120° C. in 2 ml of sodium carbonate solution (2 ml, 2.0 M, 4 mmol) for 30 hours. The reaction mixture was then diluted with water and extracted with dichloromethane. The organic phase was dried over Na₂SO₄ and filtered and the filtrate was concentrated on a rotary evaporator. The residue was dissolved in DMSO and purified by preparative HPLC (instrument: Waters Prep LC/MS System, column: XBridge C18 5 μm 100×30 mm. Mobile phase A: water, mobile phase B: acetonitrile, mobile phase C: 2% ammonia in water, mobile phase D: acetonitrile/water (80% by volume/20% by volume) total flow rate: 80 ml/min; room temperature; wavelength 200-400 nm, complete injection. Gradient profile: mobile phase A 0 to 2 min 55 ml, mobile phase B 0 to 2 min 15 ml, mobile phase A 2 to 10 min from 55 ml to 31 ml and mobile phase B from 15 ml to 39 ml, 10 to 12 min 0 ml of mobile phase A and 70 ml of mobile phase B. Mobile phase C and mobile phase D constant flow rate of 5 ml/min each over the entire running time). The product-containing fractions were combined and lyophilized. This gave 18.0 mg (purity 100%, 13% of theory) of the target compound.

LC-MS (Methode 5): Rₜ=1.52 min; MS (ESIpos): m/z=466 [M+H]⁺.

¹H-NMR (400 MHz, DMSO-d6) δ[ppm]: 0.236 (9.32), 0.259 (7.85), 0.278 (1.43), 0.294 (0.46), 1.209 (0.58), 1.227 (1.61), 1.242 (3.77), 1.257 (3.72), 1.271 (1.81), 1.290 (0.55), 1.561 (2.71), 1.571 (3.44), 1.585 (2.57), 1.669 (1.21), 1.694 (1.73), 1.787 (0.48), 1.797 (0.60), 1.818 (1.34), 1.828 (1.46), 1.849 (1.31), 1.860 (1.24), 1.880 (0.46), 2.073 (1.21), 2.269 (1.36), 2.297 (5.25), 2.313 (4.36), 2.328 (0.76), 2.339 (1.10), 2.367 (0.61), 2.577 (4.23), 2.589 (5.56), 2.602 (3.30), 2.635 (0.93), 2.644 (0.90), 2.666 (1.15), 2.710 (0.57), 3.110 (1.17), 3.136 (2.08), 3.142 (2.03), 3.167 (1.24), 3.199 (1.78), 3.235 (2.02), 3.968 (1.52), 4.001 (1.41), 4.142 (0.95), 4.172 (1.62), 4.206 (0.88), 4.520 (5.56), 4.533 (5.59), 5.026 (1.83), 5.148 (1.86), 7.810 (16.00), 7.884 (1.64), 7.890 (1.76), 7.909 (2.28), 7.913 (2.39), 7.915 (2.23), 7.932 (1.72), 7.938 (1.80), 8.164 (0.74), 8.463 (5.37), 8.469 (5.31), 8.706 (1.86), 8.720 (3.88), 8.734 (1.86).

Analogously to Examples 15 to 17, the following compounds of Examples 136 to 149 were prepared from the starting materials stated in each case:

| Example | Name/Structure/Starting materials | Analytical data |
|---|---|---|
| 136 | 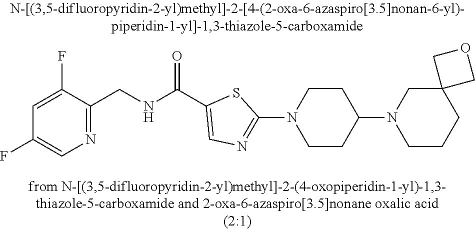 [SMILES: O=C(NCc1ncc(F)cc1F)c1cnc(N2CCC(N3CCCC4(COC4)C3)CC2)s1] | ¹H-NMR (600 MHz, DMSO-d₆, δ/ppm): 1.37-1.44 (m, 2H), 1.46- 1.56 (m, 2H), 1.57-1.65 (m, 2H), 1.78 (br. d, 2H), 2.37 (br. s, 2H), 2.52-2.63 (m, 3H, partially obscured by DMSO), 3.07 (td, 2H), 3.94 (br. d, 2H), 4.19 (s, 4H), 4.53 (d, 2H), 7.83 (s, 1H), 7.90 (td, 1H), 8.46 (d, 1H), 8.68 (t, 1H). LC-MS (Methode 1): Rt = 0.76 min; m/z = 464 (M + H)⁺. |
| 137 | 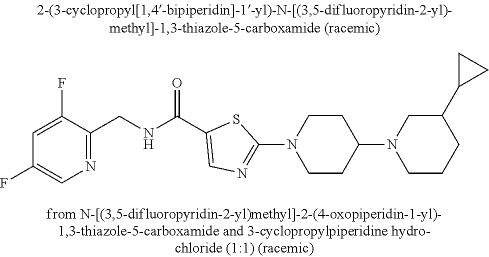 [SMILES: O=C(NCc1ncc(F)cc1F)c1cnc(N2CCC(N3CCCC(C4CC4)C3)CC2)s1] | ¹H-NMR (600 MHz, DMSO-d₆, δ/ppm): 0.01-0.08 (m, 2H), 0.29- 0.37 (m, 2H), 0.47-0.54 (m, 1H), 0.63-0.72 (m, 1H), 0.99 (qd, 1H), 1.28-1.38 (m, 1H), 1.42-1.54 (m, 2H), 1.56-1.63 (m, 1H), 1.66-1.72 (m, 1H), 1.74-1.81 (m, 2H), 1.99 (t, 1H), 2.05-2.12 (m, 1H), 2.47- 2.55 (m, 1H, partially obscured by DMSO), 2.71 (br. d, 1H), 2.80 (br. d, 1H), 3.04 (td, 2H), 3.93 (br. d, 2H), 4.52 (d, 2H), 7.82 (s, 1H), 7.89 (td, 1H), 8.46 (d, 1H), 8.67 (t, 1H). LC-MS (Methode 1): Rt = 1.02 min; m/z = 462 (M + H)⁺. |
| 138 | 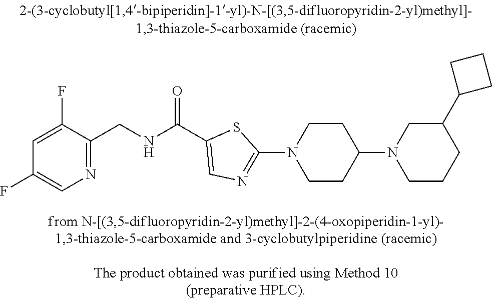 [SMILES: O=C(NCc1ncc(F)cc1F)c1cnc(N2CCC(N3CCCC(C4CCC4)C3)CC2)s1] | ¹H-NMR (600 MHz, DMSO-d₆, δ/ppm): 0.67-0.75 (m, 1H), 1.30- 1.40 (m, 2H), 1.41-1.52 (m, 2H), 1.54-1.83 (m, 9H), 1.88-1.95 (m, 2H), 1.97-2.10 (m, 2H), 2.44-2.52 (m, 1H, partially obscured by DMSO), 2.67 (br. d, 1H), 2.71 (br. d, 1H), 2.99-3.08 (m, 2H), 3.93 (br. d, 2H), 4.53 (d, 2H), 7.82 (s, 1H), 7.89 (td, 1H), 8.46 (d, 1H), 8.67 (t, 1H). LC-MS (Methode 1): Rt = 1.13 min; m/z = 476 (M + H)⁺. |
| 139 | 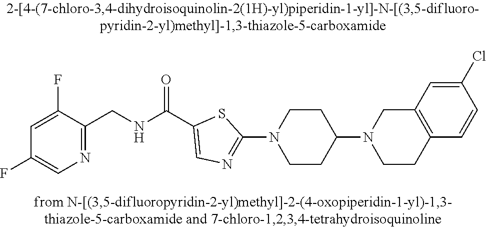 [SMILES: O=C(NCc1ncc(F)cc1F)c1cnc(N2CCC(N3CCc4ccc(Cl)cc4C3)CC2)s1] | ¹H-NMR (400 MHz, DMSO-d₆, δ/ppm): 1.51-1.66 (m, 2H), 1.89 (br. d, 2H), 2.65-2.80 (m, 5H), 3.12 (br. t, 2H), 3.70 (s, 2H), 3.96 (br. d, 2H), 4.53 (br. d, 2H), 7.07- 7.18 (m, 3H), 7.84 (s, 1H), 7.88- 7.95 (m, 1H), 8.47 (d, 1H), 8.72 (t, 1H). LC-MS (Methode 1): Rt = 1.10 min; m/z = 504/506 (M + H)⁺. |
| 140 | 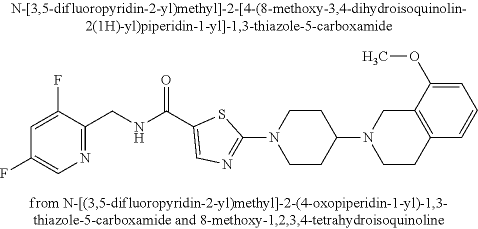 [SMILES: COc1cccc2c1CN(C1CCN(c3ncc(C(=O)NCc4ncc(F)cc4F)s3)CC1)CC2] | ¹H-NMR (400 MHz, DMSO-d₆, δ/ppm): 1.51-1.66 (m, 2H), 1.90 (br. d, 2H), 2.65-2.79 (m, 5H), 3.12 (br. t, 2H), 3.58 (s, 2H), 3.75 (s, 3H), 3.97 (br. d, 2H), 4.53 (br. d, 2H), 6.68 (d, 1H), 6.74 (d, 1H), 7.08 (t, 1H), 7.85 (s, 1H), 7.91 (ddd, 1H), 8.48 (d, 1H), 8.72 (t, 1H). LC-MS (Methode 1): Rt = 1.01 min; m/z = 500 (M + H)⁺. |
| 141 | 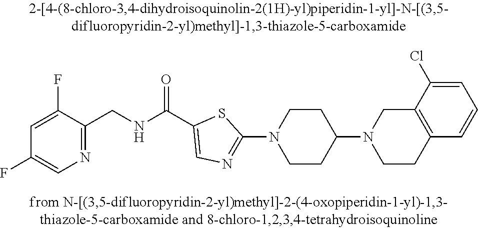 [SMILES: O=C(NCc1ncc(F)cc1F)c1cnc(N2CCC(N3CCc4cccc(Cl)c4C3)CC2)s1] | ¹H-NMR (400 MHz, DMSO-d₆, δ/ppm): 1.53-1.67 (m, 2H), 1.92 (br. d, 2H), 2.70-2.85 (m, 5H), 3.13 (br. t, 2H), 3.70 (s, 2H), 3.99 (br. d, 2H), 4.53 (br. d, 2H), 7.10 (d, 1H), 7.16 (t, 1H), 7.24 (d, 1H), 7.85 (s, 1H), 7.91 (ddd, 1H), 8.47 (d, 1H), 8.72 (t, 1H). LC-MS (Methode 1): Rt = 1.07 min; m/z = 504/506 (M + H)⁺. |
| 142 | 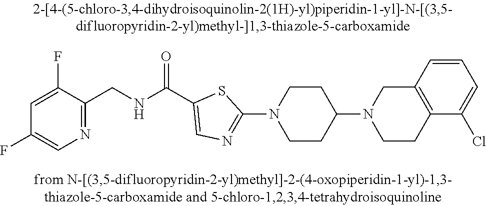 [SMILES: O=C(NCc1ncc(F)cc1F)c1cnc(N2CCC(N3CCc4c(Cl)cccc4C3)CC2)s1] | ¹H-NMR (400 MHz, DMSO-d₆, δ/ppm): 1.52-1.66 (m, 2H), 1.90 (br. d, 2H), 2.67-2.77 (m, 3H), 2.78-2.85 (m, 2H), 3.12 (br. t, 2H), 3.72 (s, 2H), 3.97 (br. d, 2H), 4.53 (br. d, 2H), 7.06 (d, 1H), 7.15 (t, 1H), 7.25 (d, 1H), 7.85 (s, 1H), 7.91 (ddd, 1H), 8.47 (d, 1H), 8.72 (t, 1H). LC-MS (Methode 1): Rt = 1.07 min; m/z = 504/506 (M + H)⁺. |
| 143 | 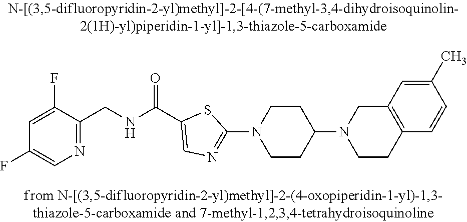 [SMILES: Cc1ccc2c(c1)CN(C1CCN(c3ncc(C(=O)NCc4ncc(F)cc4F)s3)CC1)CC2] | ¹H-NMR (400 MHz, DMSO-d₆, δ/ppm): 1.52-1.66 (m, 2H), 1.90 (br. d, 2H), 2.22 (s, 3H), 2.61-2.78 (m, 5H), 3.12 (br. t, 2H), 3.65 (s, 2H), 3.96 (br. d, 2H), 4.53 (br. d, 2H), 6.85 (s, 1H), 6.90 (d, 1H), 6.95 (d, 1H), 7.85 (s, 1H), 7.91 (ddd, 1H), 8.47 (d, 1H), 8.72 (t, 1H). LC-MS (Methode 1): Rt = 1.06 min; m/z = 484 (M + H)⁺. |
| 144 | 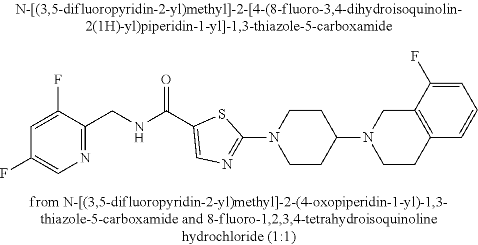 [SMILES: O=C(NCc1ncc(F)cc1F)c1cnc(N2CCC(N3CCc4cccc(F)c4C3)CC2)s1] | ¹H-NMR (400 MHz, DMSO-d₆, δ/ppm): 1.53-1.67 (m, 2H), 1.91 (br. d, 2H), 2.72-2.84 (m, 5H), 3.12 (br. t, 2H), 3.70 (s, 2H), 3.98 (br. d, 2H), 4.53 (br. d, 2H), 6.91- 6.98 (m, 2H), 7.11-7.19 (m, 1H), 7.85 (s, 1H), 7.91 (ddd, 1H), 8.47 (d, 1H), 8.72 (t, 1H). LC-MS (Methode 1): Rt = 1.00 min; m/z = 488 (M + H)⁺. |
| 145 | 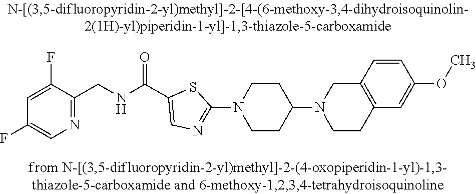 [SMILES: COc1ccc2c(c1)CCN(C1CCN(c3ncc(C(=O)NCc4ncc(F)cc4F)s3)CC1)C2] | ¹H-NMR (600 MHz, DMSO-d₆, δ/ppm): 1.53-1.64 (m, 2H), 1.89 (br. d, 2H), 2.65-2.71 (m, 1H), 2.71-2.78 (m, 4H), 3.12 (br. t, 2H), 3.63 (s, 2H), 3.69 (s, 3H), 3.95 (br. d, 2H), 4.53 (br. d, 2H), 6.64 (d, 1H), 6.67 (dd, 1H), 6.95 (d, 1H), 7.84 (s, 1H), 7.90 (ddd, 1H), 8.46 (d, 1H), 8.69 (t, 1H). LC-MS (Methode 1): Rt = 1.00 min; m/z = 500 (M + H)⁺. |
| 146 | 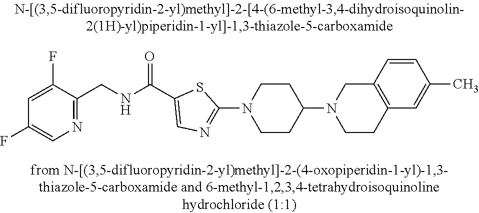 [SMILES: Cc1ccc2c(c1)CCN(C1CCN(c3ncc(C(=O)NCc4ncc(F)cc4F)s3)CC1)C2] | ¹H-NMR (400 MHz, DMSO-d₆, δ/ppm): 1.51-1.66 (m, 2H), 1.90 (br. d, 2H), 2.22 (s, 3H), 2.64-2.78 (m, 5H), 3.11 (br. t, 2H), 3.65 (s, 2H), 3.96 (br. d, 2H), 4.53 (br. d, 2H), 6.85-6.95 (m, 3H), 7.84 (s, 1H), 7.91 (ddd, 1H), 8.47 (d, 1H), 8.72 (t, 1H). LC-MS (Methode 1): Rt = 1.06 min; m/z = 484 (M + H)⁺. |
| 147 | 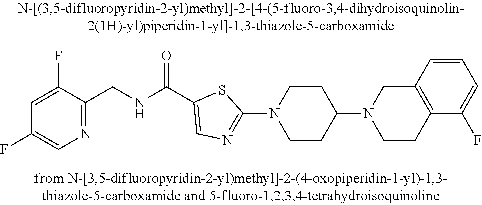 [SMILES: O=C(NCc1ncc(F)cc1F)c1cnc(N2CCC(N3CCc4c(F)cccc4C3)CC2)s1] | ¹H-NMR (600 MHz, DMSO-d₆, δ/ppm): 1.55-1.65 (m, 2H), 1.90 (br. d, 2H), 2.68-2.76 (m, 3H), 2.76-2.82 (m, 2H), 3.12 (br. t, 2H), 3.72 (s, 2H), 3.97 (br. d, 2H), 4.53 (d, 2H), 6.89-6.97 (m, 2H), 7.11- 7.16 (m, 1H), 7.84 (s, 1H), 7.90 (td, 1H), 8.46 (d, 1H), 8.69 (t, 1H). LC-MS (Methode 1): Rt = 1.00 min; m/z = 488 (M + H)⁺. |
| 148 | 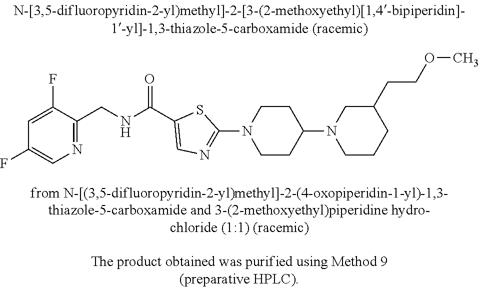 [SMILES: COCCC1CCCN(C2CCN(c3ncc(C(=O)NCc4ncc(F)cc4F)s3)CC2)C1] | ¹H-NMR (400 MHz, DMSO-d₆, δ/ppm): 0.77-0.92 (m, 1H), 1.25- 1.89 (m, 13H), 2.03-2.15 (m, 1H), 2.38-2.58 (m, 1H, partially obscured by DMSO), 2.64-2.78 (m, 2H), 3.04 (br. t, 2H), 3.20 (s, 3H), 3.94 (br. d, 2H), 4.53 (br. d, 2H), 7.83 (s, 1H), 7.87-7.95 (m, 1H), 8.47 (d, 1H), 8.71 (t, 1H). LC-MS (Methode 1): Rt = 0.90 min; m/z = 480 (M + H)⁺. |
| 149 | 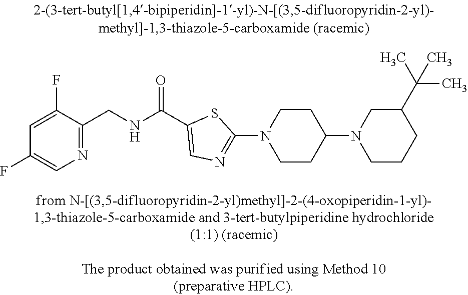 [SMILES: CC(C)(C)C1CCCN(C2CCN(c3ncc(C(=O)NCc4ncc(F)cc4F)s3)CC2)C1] | ¹H-NMR (600 MHz, DMSO-d₆, δ/ppm): 0.78-0.96 (m, 10H, including at 0.84 (s, 9H)), 1.18 (br. t, 1H), 1.30-1.40 (m, 1H), 1.43-1.56 (m, 2H), 1.64 (br. d, 1H), 1.70 (br. d, 1H), 1.75-1.82 (m, 2H), 1.86 (t, 1H), 1.98 (br. t, 1H), 2.48-2.55 (m, 1H, partially obscured by DMSO), 2.78 (br. d, 1H), 2.87 (br. d, 1H), 3.00-3.09 (m, 2H), 4.53 (br. d, 2H), 7.82 (s, 1H), 7.88 (td, 1H), 8.46 (d, 1H), 8.67 (t, 1H). LC-MS (Methode 1): Rt = 1.14 min; m/z = 478 (M + H)⁺. |

Analogously to Examples 18 to 22, the following compounds of Examples 150 to 152 were prepared from the starting materials stated in each case:

| Example | Name/Structure/Starting material | Analytical data |
|---|---|---|
| 150 | 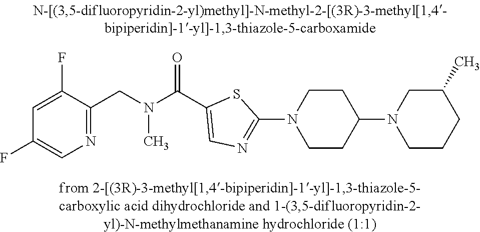 [SMILES: C[C@@H]1CCCN(C2CCN(c3ncc(C(=O)N(C)Cc4ncc(F)cc4F)s3)CC2)C1] | ¹H-NMR (600 MHz, DMSO-d₆, δ/ppm): 0.76-0.87 (m, 4H, including at 0.82 (d, 3H)), 1.35- 1.67 (m, 6H), 1.71-1.82 (m, 3H), 2.05 (br. t, 1H), 2.45-2.56 (m, 1H, partially obscured by DMSO), 2.74 (br. t, 2H), 3.05 (t, 2H), 3.16 (br. s, 3H), 3.94 (br. d, 2H), 4.83 (br. s, 2H), 7.59 (s, 1H), 7.93 (t, 1H), 8.48 (d, 1H). LC-MS (Methode 1): Rt = 0.99 min; m/z = 450 (M + H)⁺. |
| 151 | 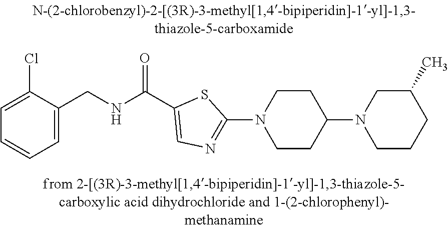 [SMILES: C[C@@H]1CCCN(C2CCN(c3ncc(C(=O)NCc4ccccc4Cl)s3)CC2)C1] | ¹H-NMR (600 MHz, DMSO-d₆, δ/ppm): 0.77-0.87 (m, 4H, including at 0.82 (d, 3H)), 1.34- 1.67 (m, 6H), 1.72-1.82 (m, 3H), 2.01-2.10 (m, 1H), 2.45-2.56 (m, 1H, partially obscured by DMSO), 2.74 (br. t, 2H), 3.06 (td, 2H), 3.95 (br. d, 2H), 4.46 (d, 2H), 7.26-7.37 (m, 3H), 7.41-7.49 (m, 1H), 7.87 (s, 1H), 7.87 (t, 1H). LC-MS (Methode 1): Rt = 1.17 min; m/z = 433/435 (M + H)⁺. |
| 152 | 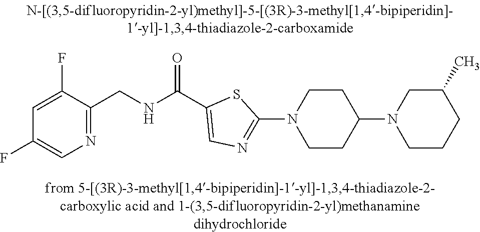 [SMILES: C[C@@H]1CCCN(C2CCN(c3ncc(C(=O)NCc4ncc(F)cc4F)s3)CC2)C1] | ¹H-NMR (600 MHz, DMSO-d₆, δ/ppm): 0.78-0.87 (m, 4H, including at 0.82 (d, 3H)), 1.36- 1.45 (m, 1H), 1.48-1.67 (m, 5H), 1.73-1.84 (m, 3H), 2.02-2.10 (m, 1H), 2.45-2.57 (m, 1H, partially obscured by DMSO), 2.70-2.78 (m, 2H), 3.20 (td, 2H), 3.94 (br. d, 2H), 4.59 (d, 2H), 7.91 (td, 1H), 8.46 (d, 1H), 9.11 (t, 1H). LC-MS (Methode 1): Rt = 0.94 min; m/z = 437 (M + H)⁺. |

B. Assessment of Pharmacological Efficacy of Compounds of Formula (I)

The pharmacological activity of the compounds of formula (I) can be demonstrated by in vitro and in vivo studies as known to the person skilled in the art. The application examples which follow describe the biological action of the compounds of the invention, without restricting the invention to these examples. Binding studies (B-1.) and activity studies (B-2.) were carried out for in vitro characterization of receptor/substance interaction and determination of biological activity, respectively.

B-1 In Vitro Radioligand Binding Studies for Determination of the Dissociation Constants K₁ at the Human Adrenoreceptor ADRA2C (Eurofins Panlabs Discovery Services, Taiwan, Ltd)

A competition assay based on [³H] rauwolscine as radioliganden was used to determine the binding affinity of the test substances at the human ADRA2C receptor.

To configure the competition assay, the equilibrium dissociation constant K_{d} of the radioligand [³H] rauwolscine was determined in a saturation experiment. To this end, homogenates of CHO-K1 cells recombinantly expressing the human ADRA2C receptor were incubated with increasing concentrations of the radiotracers for 1 h at 4° C. in binding buffer (50 mM Tris-HCl, 1 mM EDTA, pH 7.4). Unspecific binding was determined by addition of an excess of the not radioactively labelled ligand prazosin (10 μM). The radioactivity was determined in a scintiation counter.

The competition experiments were carried out in the presence of 0.5 nM [³H] rauwolscine and increasing concentrations of the test substances to be characterized under the conditions described above. The substance concentration which displaces 50% of the radiolabelled ligand is referred to as IC₅₀ value.

From the IC₅₀ value measured in the competition experiment and the Kₐ value from the saturation experiment, the equilibrium constant Ki of the inhibitor, which describes the affinity of the test substances to the receptor, was calculated using the Cheng Prusoff equation [Cheng Y, Prusoff WH. Relationship between the inhibition constant (Ki) and the concentration of inhibitor which causes 50 percent inhibition (I50) of an enzymatic reaction. Biochem Pharmacol. 22 (23): 3099-108. doi:10.1016/0006-2952(73)90196-2. PMID 4202581 PMID: 4202581].

$$K i = \frac{I C 50}{( 1 + \frac{L}{K d} )} \qquad Formula 1$$

Cheng Prusoff equation. Kᵢ=equilibrium constant of the inhibitor, IC₅₀=concentration which displaces 50% of the ligand, K_{d}=equilibrium constant of the ligand, L=concentration of the ligand

Table 1a below shows the binding affinity to the human ADRA2C receptor (Kᵢ [nM]) and the half-maximal inhibition of the human ADRA2C receptor (IC50 [nM]) of representative embodiments of the invention:

TABLE 1a

| Example | hARα_{2C} | hARα_{2C} |
|---|---|---|
| No. | IC₅₀ [nM] | Kᵢ [nM] |
| 1 | 54 | 24 |
| 100 | 5 | 2 |

The data in Table 1a show that the test substances listed both bind to the human ADRA2C receptor and block the biological activity of the human ADRA2C receptor. Accordingly, the results in Table 1 confirm the mechanism of action of the compounds according to the invention as ADRA2C inhibitors.

B-2. In Vitro Activity Assay to Determine the Inhibition of Recombinant ADRA2C

The human ADRA2C receptor belongs to the G protein(guanine-dependent protein)-coupled receptors, the main function of which is the transduction of signals into the interior of the cell.

The investigations of the inhibition of the recombinant human ADRA2C receptors were carried out with stabily transfected CHO-K1 cells coexpressing the G_{aq} protein and the calcium-sensitive photoprotein aequorin. In this recombinant system, binding of the agonists noradrenaline to the ADRA2C receptor leads, after activation of a signal cascade, to calcium release from intracellular stores, which is detected by the intracellular calcium sensor aequorin as a bioluminescent signal. The method is described in detail in the reference below. [Wunder F., Kalthof B., Muller T., Hueser J. Functional Cell-Based Assays in Microliter Volumes for Ultra-High Throughput Screening. Combinatorial Chemistry & High Throughput Screening, Volume 11, Number 7, 2008, pp. 495-504(10). doi.org/10.2174/138620708785204054]

The activity of the test substances was determined via their ability to inhibit the agonist-induced increase of the bioluminescence signal. The concentration which can block half of this signal increase is referred to as IC₅₀. The IC₅₀ value is calculated using the 4 parameter logistic function (Hill function):

$$Hill function$$

$$Y ( x ) = Bottom + \frac{Top - Bottom}{1 + 1 {0}^{( iogIC 50 + x ) - HillSlope}} \qquad Formula 2$$

Top=upper threshold, Bottom=lower threshold, Slope=slope, IC₅₀=turning point

Table 2 below lists the IC₅₀ values from this assay determined for individual working examples of the invention (some as mean values from multiple independent individual determinations):

TABLE 2a

| Example | ARα_{2C} |
|---|---|
| No. | IC₅₀ [nM] |
| 1 | 121 |
| 2 | 5.2 |
| 3 | 2.9 |
| 4 | 169 |
| 5 | 335 |
| 6 | 335 |
| 7 | 49.6 |
| 8 | 591 |
| 9 | 170 |
| 10 | 21.4 |
| 11 | 140 |
| 12 | 107 |
| 13 | 209 |
| 14 | 211 |
| 15 | 1850 |
| 16 | 2000 |
| 17 | 26.5 |
| 18 | 6800 |
| 19 | 690 |
| 20 | 110 |
| 21 | 7.2 |
| 22 | 640 |
| 23 | 17 |
| 24 | 1060 |
| 25 | 2400 |
| 26 | 280 |
| 27 | 310 |
| 28 | 890 |
| 29 | 96 |
| 31 | 89 |
| 32 | 640 |
| 33 | 15 |
| 34 | 640 |
| 35 | 1000 |
| 36 | 190 |
| 37 | 200 |
| 38 | 2340 |
| 39 | 135 |
| 41 | 243 |
| 43 | 26 |
| 44 | 41.2 |
| 46 | 8.3 |
| 47 | 25 |
| 48 | 17 |
| 49 | 56 |
| 50 | 73.5 |
| 51 | 150 |
| 52 | 6.7 |
| 53 | 110 |
| 54 | 230 |
| 55 | 150 |
| 56 | 240 |
| 57 | 830 |
| 58 | 870 |
| 59 | 11 |
| 60 | 74 |
| 61 | 87 |
| 62 | 130 |
| 63 | 570 |
| 64 | 1.4 |
| 65 | 4.5 |
| 66 | 1.5 |
| 67 | 2.2 |
| 68 | 4.3 |
| 69 | 5.5 |
| 70 | 22 |
| 71 | 100 |
| 72 | 180 |
| 73 | 219 |
| 74 | 285 |
| 75 | 345 |
| 76 | 400 |
| 77 | 640 |
| 78 | 755 |
| 79 | 50.3 |
| 80 | 90.5 |
| 81 | 22.2 |
| 82 | 102 |
| 83 | 98.1 |
| 84 | 65 |
| 85 | 600 |
| 86 | 28 |
| 87 | 25.5 |
| 88 | 489 |
| 89 | 230 |
| 90 | 97 |
| 91 | 284 |
| 92 | 85 |
| 93 | 630 |
| 94 | 150 |
| 95 | 800 |
| 96 | 1000 |
| 97 | 1300 |
| 98 | 1600 |
| 99 | 590 |
| 100 | 2.9 |
| 101 | 2.3 |
| 102 | 0.085 |
| 103 | 0.83 |
| 104 | 10 |
| 105 | 4.3 |
| 106 | 0.5 |
| 107 | 37 |
| 108 | 0.37 |
| 109 | 0.85 |
| 110 | 1.5 |
| 111 | 0.49 |
| 112 | 1.2 |
| 113 | 0.61 |
| 114 | 1.3 |
| 115 | 0.61 |
| 116 | 11 |
| 117 | 0.65 |
| 118 | 7.7 |
| 119 | 30 |
| 120 | 41 |
| 121 | 0.65 |
| 122 | 13 |
| 123 | 260 |
| 124 | 2.9 |
| 125 | 0.49 |
| 126 | 2.3 |
| 127 | 8.4 |
| 128 | 550 |
| 129 | 10 |
| 130 | 0.54 |
| 131 | 1.9 |
| 132 | 0.71 |
| 133 | 0.95 |
| 134 | 0.74 |
| 135 | 0.26 |
| 136 | 320 |
| 137 | 19 |
| 138 | 6.5 |
| 139 | 0.65 |
| 140 | 0.32 |
| 141 | 8.7 |
| 142 | 6.7 |
| 143 | 0.42 |
| 144 | 1.8 |
| 145 | 1.0 |
| 146 | 1.72 |
| 148 | 1.3 |
| 149 | 35 |
| 150 | 200 |
| 151 | 170 |
| 152 | 660 |

The data in Table 2a show that the test substances listed block the biological activity of the human ADRA2C receptor. Accordingly, the results in Table 1 confirm the mechanism of action of the compounds according to the invention as ADRA2C inhibitors.

B-3 Animal Model of Obstructive Sleep Apnoea in the Pig

Using negative pressure, it is possible to induce collapse and thus obstruction of the upper respiratory tract in anesthetized, spontaneously breathing pigs [Wirth et al., Sleep 36, 699-708 (2013)].

German Landrace pigs are used for the model. The pigs are anesthetized and tracheotomized. One cannula each is inserted into the rostral and the caudal part of the trachea. Using a T connector, the rostral cannula is connected on the one hand to a device generating negative pressures and on the other hand to the caudal cannula. Using a T connector, the caudal cannula is connected to the rostral cannula and to a tube which allows spontaneous breathing circumventing the upper respiratory tract. By appropriate closing and opening of the tubes it is thus possible for the pig to change from normal nasal breathing to breathing via the caudal cannula during the time when the upper respiratory tract is isolated and connected to the device for generating negative pressures. The muscle activity of the musculus genioglossus is recorded by electromyogram (EMG).

At certain points in time, the collapsibility of the upper respiratory tract is tested by having the pig breathe via the caudal cannula and applying negative pressures of −50, −100 and −150 cm water head (cmH₂O) to the upper respiratory tract. This causes the upper respiratory tract to collapse, which manifests itself in an interruption of the airflow and a pressure drop in the tube system. This test is conducted prior to the administration of the test substance and at certain intervals after the administration of the test substance. An appropriately effective test substance can prevent this collapse of the respiratory tract in the inspiratory phase.

Administration of the test substance can be intranasal, intravenous, subcutaneous, intraperitoneal, intraduodenal or intragastral.

C. Experimental Methods-Combination of an α2-Adrenoceptor Subtype C (Alpha-2C) Antagonists with a TASK⅓ Channel Blocker

Advantageous pharmacological properties of the combination of an α2-Adrenoceptor subtype C (alpha-2C) antagonists with a TASK⅓ channel blocker can be determined by the following methods.

The therapeutic potential of the the combination of an α2-Adrenoceptor subtype C (alpha-2C) antagonists with a TASK⅓ channel blocker according to the present invention in sleep apnea can be assessed preclinically in a pig model of obstructive sleep apnea (OSA).

Using negative pressure, it is possible to induce collapse and thus obstruction of the upper respiratory tract in anaesthetized, spontaneously breathing pigs (Wirth K. J. et al., Sleep 36(5) (2013) pp. 699-708).

German Landrace pigs are used for the model. The pigs are anaesthetized and tracheotomized. Two tracheal cannulas are inserted into the trachea, one into the rostral part and the other into the caudal part of the trachea. Using a connection piece, the rostral cannula is connected to a tube to the negative pressure device and to the distal tracheal cannula. The distal tracheal cannula is additionally connected to a tube with an open end to atmosphere via a connection piece that served for free tracheal breathing, circumventing the upper airway. By appropriate opening and clamping of those tubes breathing can be switched from nasal breathing to breathing through the caudal tracheal cannula, circumventing the upper airway, and the (isolated) upper airway can be connected to the negative pressure device, causing airflow in the inspiratory direction.

At certain points in time, the collapsibility of the upper respiratory tract is tested by having the pig breathe via the caudal cannula and applying negative pressures of −50, −100 and −150 cm water head (cm H₂O) to the upper respiratory tract. This causes the upper respiratory tract to collapse, which manifests itself in an interruption of the airflow and a pressure drop in the tube system. This test is conducted prior to the administration of the test substance and at certain intervals after the administration of the test substance. An appropriately effective test substance can prevent this collapse of the respiratory tract in the inspiratory phase.

In this OSA pig model, systemic application of the α2-Adrenoceptor subtype C (alpha-2C) antagonists of formula (I), such as N-[(3,5-difluoropyridin-2-yl)methyl]-2-[(3R)-3-methyl[1,4′-bipiperidin]-1′-yl]-1,3-thiazole-5-carboxamide with intraduodenal administration of 0.01 mg/kg inhibited upper airway collapsibility at all negative pressures of −50, −100 and −150 cm head (cm H₂O) in all pigs only at time point 150 and 180 min after intraduodenal application. At time point 230 min after intraduodenal administration, upper airway collapsibility was induced at all negative pressures of −50, −100 and −150 cm head (cm H₂O) in all pigs. The combination of this non effective dose of the α2-Adrenoceptor subtype C (alpha-2C) antagonists of formula (I) N-[(3,5-difluoropyridin-2-yl)methyl]-2-[(3R)-3-methyl[1,4′-bipiperidin]-1′-yl]-1,3-thiazole-5-carboxamide with the non effective dose of the TASK1/TASK3 channel blocker of 0.3 μg ((3-chloro-6-methoxypyridin-2-yl)(3-{[2-(4-isopropylphenyl) imidazo[1,2-a]pyrimidin-3-yl]methyl}J-3,8-diazabicyclo[3.2.1]oct-8-yl)methanone inhibits upper airway collapsibility at all negative pressures of −50, −100 and −150 cm head (cm H₂O) for more than three hours (see Table 1, 2 and 3 and FIG. 1).

FIG. 1: Effect of intraduodenal administration of 0.01 mg/kg of the α2-Adrenoceptor subtype C (alpha-2C) antagonists of formula (I) N-[(3,5-difluoropyridin-2-yl)methyl]-2-[(3R)-3-methyl[1,4′-bipiperidin]-1′-yl]-1,3-thiazole-5-carboxamide given at time point 0 min in combination with intranasal administration of 0.3 μg of the TASK1/TASK3 channel blocker ((3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone given at time point 230 min after beginning of the experiment on upper airway collapsibility at different levels of negative pressure. Percentages of pigs with no collapse are given. Mean values.

TABLE 1
Combination of non effective dose of N-[(3,5-difluoropyridin-2-yl)methyl]-2-[(3R)-3-
methyl[1,4′-bipiperidin]-1′-yl]-1,3-thiazole-5-carboxamide with the non effective dose
of 0.3 μg of ((3-chloro-6-methoxypyridin-2-yl)(3-{[2-(4-isopropylphenyl)imidazo[1,2-
a]pyrimidin-3-yl]methyl}-3,8-diazabicyclo[3.2.1]oct-8-yl)methanone inhibits upper
airway collapsibility at negative pressures of −50 cm head (cm H₂O)

| | Time, min | Percent pigs without collaps −50 cm H₂O, % |
|---|---|---|
| | 0 | 0 |
| | 30 | 0 |
| | 60 | 0 |
| | 90 | 33 |
| | 120 | 100 |
| | 150 | 100 |
| | 180 | 100 |
| | 210 | 67 |
| Nasal application TASK1/3 | 230 | 0 |
| channel blocker | | |
| | 240 | 100 |
| | 270 | 100 |
| | 300 | 100 |
| | 330 | 100 |
| | 360 | 100 |
| | 390 | 100 |
| | 420 | 100 |

TABLE 2
Combination of non effective dose of N-[(3,5-difluoropyridin-2-yl)methyl]-2-[(3R)-3-
methyl[1,4′-bipiperidin]-1′-yl]-1,3-thiazole-5-carboxamide with the non effective dose
of 0.3 μg of ((3-chloro-6-methoxypyridin-2-yl)(3-{[2-(4-isopropylphenyl)imidazo[1,2-
a]pyrimidin-3-yl]methyl}-3,8-diazabicyclo[3.2.1]oct-8-yl)methanone inhibits upper
airway collapsibility at negative pressures of −100 cm head (cm H₂O)

| | Time, min | Percent pigs without collaps −100 cm H₂O, % |
|---|---|---|
| | 0 | 0 |
| | 30 | 0 |
| | 60 | 0 |
| | 90 | 33 |
| | 120 | 100 |
| | 150 | 100 |
| | 180 | 100 |
| | 210 | 33 |
| Nasal application TASK1/3 | 230 | 0 |
| channel blocker | | |
| | 240 | 100 |
| | 270 | 100 |
| | 300 | 100 |
| | 330 | 100 |
| | 360 | 100 |
| | 390 | 100 |
| | 420 | 100 |

TABLE 3
Combination of non effective dose of N-[(3,5-difluoropyridin-2-yl)methyl]-2-[(3R)-3-
methyl[1,4′-bipiperidin]-1′-yl]-1,3-thiazole-5-carboxamide with the non effective dose
of 0.3 μg of ((3-chloro-6-methoxypyridin-2-yl)(3-{[2-(4-isopropylphenyl)imidazo[1,2-
a]pyrimidin-3-yl]methyl}-3,8-diazabicyclo[3.2.1]oct-8-yl)methanone inhibits upper
airway collapsibility at negative pressures of −150 cm head (cm H₂O)

| | Time, min | Percent pigs without collaps −150 cm H₂O, % |
|---|---|---|
| | 0 | 0 |
| | 30 | 0 |
| | 60 | 0 |
| | 90 | 33 |
| | 120 | 67 |
| | 150 | 100 |
| | 180 | 100 |
| | 210 | 0 |
| Nasal application TASK1/3 | 230 | 0 |
| channel blocker | | |
| | 240 | 100 |
| | 270 | 100 |
| | 300 | 100 |
| | 330 | 100 |
| | 360 | 100 |
| | 390 | 100 |
| | 420 | 100 |

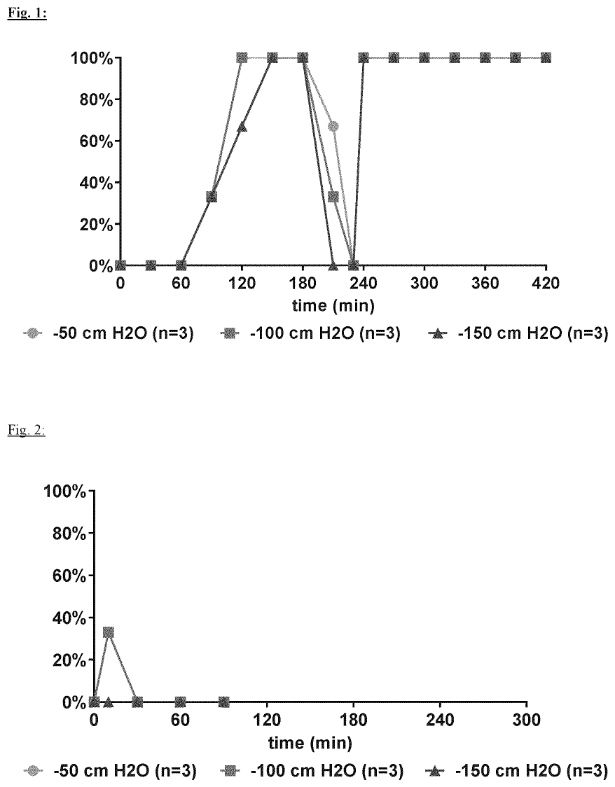

Table 4, 5 and 6 and FIG. 2: Effect of intranasal administration of 0.3 μg of the TASK1/TASK3 channel blocker ((3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone given at time point 0 min on upper airway collapsibility at different levels of negative pressure. Percentages of pigs with no collapse are given. Mean values.

TABLE 4
Intranasal administration of 0.3 μg of the TASK1/TASK3 channel blocker ((3-chloro-6-
methoxypyridin-2-yl)(3-{[2-(4-isopropylphenyl)imidazo[1,2-a]pyrimidin-3-yl]methyl}-
3,8-diazabicyclo[3.2.1]oct-8-yl)methanone at negative pressures of −50 cm head (cm H₂O)

| Time, min | Percent pigs without collaps −50 cm H₂O, % |
|---|---|
| 0 | 0 |
| 10 | 33 |
| 30 | 0 |
| 60 | 0 |
| 90 | 0 |

TABLE 5
Intranasal administration of 0.3 μg of the TASK1/TASK3 channel blocker ((3-chloro-6-
methoxypyridin-2-yl)(3-{[2-(4-isopropylphenyl)imidazo[1,2-a]pyrimidin-3-yl]methyl}-
3,8-diazabicyclo[3.2.1]oct-8-yl)methanone at negative pressures of −100 cm head (cm H₂O)

| Time, min | Percent pigs without collaps −100 cm H₂O, % |
|---|---|
| 0 | 0 |
| 10 | 33 |
| 30 | 0 |
| 60 | 0 |
| 90 | 0 |

TABLE 6
Intranasal administration of 0.3 μg of the TASK1/TASK3 channel blocker ((3-chloro-6-
methoxypyridin-2-yl)(3-{[2-(4-isopropylphenyl)imidazo[1,2-a]pyrimidin-3-yl]methyl}-
3,8-diazabicyclo[3.2.1]oct-8-yl)methanone at negative pressures of −150 cm head (cm H₂O)

| Time, min | Percent pigs without collaps −150 cm H₂O, % |
|---|---|
| 0 | 0 |
| 10 | 0 |
| 30 | 0 |
| 60 | 0 |
| 90 | 0 |

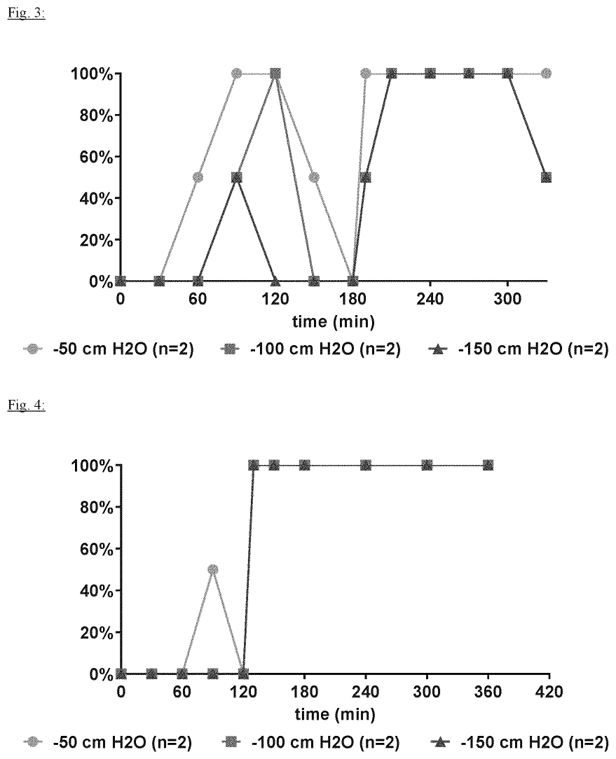

In a second set of experiments in this OSA pig model, systemic application of the α2-Adrenoceptor subtype C (alpha-2C) antagonists of formula (I), such as N-[(3,5-difluoropyridin-2-yl)methyl]-2-[(3R)-3-methyl[1,4′-bipiperidin]-1′-yl]-1,3-thiazole-5-carboxamide with intraduodenal administration of 0.01 mg/kg inhibited upper airway collapsibility at all negative pressures of −50, −100 and −150 cm head (cm H₂O) in all pigs at no time point after intraduodenal application. At time point 90 min after intraduodenal administration, upper airway collapsibility was induced at negative pressures of −100 and −150 cm head (cm H₂O), upper airway collapsibility was only inhibited at negative pressures of −50 cm head (cm H₂O). At time point 120 min after intraduodenal administration, upper airway collapsibility was induced at negative pressures of −150 cm head (cm H₂O), upper airway collapsibility was only inhibited at negative pressures of −50 and −100 cm head (cm H₂O). At time point 180 min after intraduodenal administration, upper airway collapsibility was induced at all negative pressures of −50, −100 and −150 cm head (cm H₂O) in all pigs. The combination of this non effective dose of the α2-Adrenoceptor subtype C (alpha-2C) antagonists of formula (I) N-[(3,5-difluoropyridin-2-yl)methyl]-2-[(3R)-3-methyl[1,4′-bipiperidin]-1′-yl]-1,3-thiazole-5-carboxamide with the non effective dose of the TASK1/TASK3 channel blocker of 0.3 μg (4-{[2-(4-chlorophenyl)imidazo[1,2-a]pyridin-3-yl]methyl}piperazin-1-yl)(6-methoxypyridin-2-yl)methanone inhibits upper airway collapsibility at all negative pressures of −50, −100 and −150 cm head (cm H₂O) for 90 minutes (see Table 7, 8 and 9 and FIG. 3).

FIG. 3: Effect of intraduodenal administration of 0.01 mg/kg of the α2-Adrenoceptor subtype C (alpha-2C) antagonists of formula (I) N-[(3,5-difluoropyridin-2-yl)methyl]-2-[(3R)-3-methyl[1,4′-bipiperidin]-1′-yl]-1,3-thiazole-5-carboxamide given at time point 0 min in combination with intranasal administration of 0.3 μg of the TASK1/TASK3 channel blocker (4-{[2-(4-chlorophenyl)imidazo[1,2-a]pyridin-3-yl]methyl}piperazin-1-yl)(6-methoxypyridin-2-yl)methanone given at time point 180 min after beginning of the experiment on upper airway collapsibility at different levels of negative pressure. Percentages of pigs with no collapse are given. Mean values.

TABLE 7
Combination of non effective dose of N-[(3,5-difluoropyridin-2-yl)methyl]-2-[(3R)-3-
methyl[1,4′-bipiperidin]-1′-yl]-1,3-thiazole-5-carboxamide with the non effective dose
of 0.3 μg of (4-{[2-(4-chlorophenyl)imidazo[1,2-a]pyridin-3-yl]methyl} piperazin-1-
yl)(6-methoxypyridin-2-yl)methanone inhibits upper airway collapsibility at negative
pressures of −50 cm head (cm H₂O)

| | Time, min | Percent pigs without collaps −50 cm H₂O, % |
|---|---|---|
| | 0 | 0 |
| | 30 | 0 |
| | 60 | 50 |
| | 90 | 100 |
| | 120 | 100 |
| | 150 | 50 |
| Nasal application TASK1/3 | 180 | 0 |
| channel blocker | | |
| | 190 | 100 |
| | 210 | 100 |
| | 240 | 100 |
| | 270 | 100 |
| | 300 | 100 |
| | 330 | 100 |

TABLE 8
Combination of non effective dose of N-[(3,5-difluoropyridin-2-yl)methyl]-2-[(3R)-3-
methyl[1,4′-bipiperidin]-1′-yl]-1,3-thiazole-5-carboxamide with the non effective dose
of 0.3 μg of (4-{[2-(4-chlorophenyl)imidazo[1,2-a]pyridin-3-yl]methyl} piperazin-1-
yl)(6-methoxypyridin-2-yl)methanone inhibits upper airway collapsibility at negative
pressures of −100 cm head (cm H₂O)

| | Time, min | Percent pigs without collaps −100 cm H₂O, % |
|---|---|---|
| | 0 | 0 |
| | 30 | 0 |
| | 60 | 0 |
| | 90 | 50 |
| | 120 | 100 |
| | 150 | 0 |
| Nasal application TASK1/3 | 180 | 0 |
| channel blocker | | |
| | 190 | 50 |
| | 210 | 100 |
| | 240 | 100 |
| | 270 | 100 |
| | 300 | 100 |
| | 330 | 50 |

TABLE 9
Combination of non effective dose of N-[(3,5-difluoropyridin-2-yl)methyl]-2-[(3R)-3-
methyl[1,4′-bipiperidin]-1′-yl]-1,3-thiazole-5-carboxamide with the non effective dose
of 0.3 μg of (4-{[2-(4-chlorophenyl)imidazo[1,2-a]pyridin-3-yl]methyl} piperazin-1-
yl)(6-methoxypyridin-2-yl)methanone inhibits upper airway collapsibility at negative
pressures of −150 cm head (cm H₂O)

| | Time, min | Percent pigs without collaps −150 cm H₂O, % |
|---|---|---|
| | 0 | 0 |
| | 30 | 0 |
| | 60 | 0 |
| | 90 | 50 |
| | 120 | 0 |
| | 150 | 0 |
| Nasal application TASK1/3 | 180 | 0 |
| channel blocker | | |
| | 190 | 50 |
| | 210 | 100 |
| | 240 | 100 |
| | 270 | 100 |
| | 300 | 100 |
| | 330 | 50 |

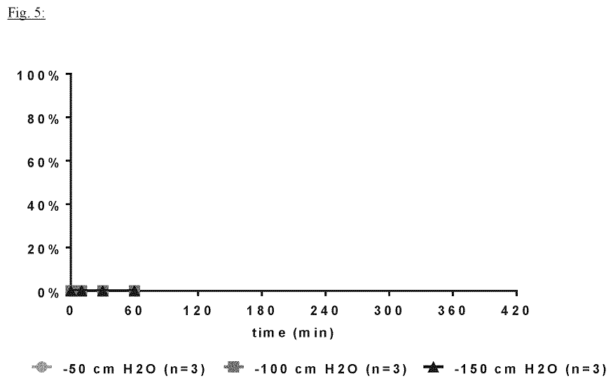

In a third set of experiments in this OSA pig model, systemic application of the α2-Adrenoceptor subtype C (alpha-2C) antagonists of formula (I), such as ent-N-[(3,5-Difluoropyridin-2-yl)methyl]-2-[3-(methoxymethyl)[1,4′-bipiperidin]-1′-yl]-1,3-thiazole-5-carboxamide with intravenous administration of 15 μg/kg as a bolus followed by i.v. infusion of 5 μg/kg/h for four hours inhibited upper airway collapsibility at all negative pressures of −50, −100 and −150 cm head (cm H₂O) in all pigs at no time point after intravenous application. At time point 120 min after intravenous administration, the non effective dose of the TASK1/TASK3 channel blocker of 0.3 μg (4-{[2-(4-chlorophenyl)imidazo[1,2-a]pyridin-3-yl]methyl}piperazin-1-yl)(6-methoxypyridin-2-yl)methanone was administered intranasally. The combination of this non effective dose of the α2-Adrenoceptor subtype C (alpha-2C) antagonists of formula (I), such as ent-N-[(3,5-Difluoropyridin-2-yl)methyl]-2-[3-(methoxymethyl)[1,4′-bipiperidin]-1′-yl]-1,3-thiazole-5-carboxamide with the non effective dose of the TASK1/TASK3 channel blocker of 0.3 μg (4-{[2-(4-chlorophenyl)imidazo[1,2-a]pyridin-3-yl]methyl}piperazin-1-yl)(6-methoxypyridin-2-yl)methanone inhibits upper airway collapsibility at all negative pressures of −50, −100 and −150 cm head (cm H₂O) for more than 4 hours (see Table 10, 11 and 12 and FIG. 4).

FIG. 4: Effect of intravenous administration of 15 μg/kg as a bolus followed by i.v. infusion of 5 μg/kg/h for four hours of the α2-Adrenoceptor subtype C (alpha-2C) antagonists of formula (I), such as ent-N-[(3,5-Difluoropyridin-2-yl)methyl]-2-[3-(methoxymethyl)[1,4′-bipiperidin]-1′-yl]-1,3-thiazole-5-carboxamide given at time point 0 min in combination with intranasal administration of 0.3 μg of the TASK1/TASK3 channel blocker (4-{[2-(4-chlorophenyl)imidazo[1,2-a]pyridin-3-yl]methyl}piperazin-1-yl)(6-methoxypyridin-2-yl)methanone given at time point 120 min after beginning of the experiment on upper airway collapsibility at different levels of negative pressure. Percentages of pigs with no collapse are given. Mean values.

TABLE 10
Combination of non effective dose of ent-N-[(3,5-Difluoropyridin-2-yl)methyl]-2-[3-
(methoxymethyl)[1,4′-bipiperidin]-1′-yl]-1,3-thiazole-5-carboxamide with the non
effective dose of 0.3 μg of (4-{[2-(4-chlorophenyl)imidazo[1,2-a]pyridin-3-
yl]methyl}piperazin-1-yl)(6-methoxypyridin-2-yl)methanone inhibits upper airway
collapsibility at negative pressures of −50 cm head (cm H₂O)

| | Time, min | Percent pigs without collaps −50 cm H₂O, % |
|---|---|---|
| | 0 | 0 |
| | 30 | 0 |
| | 60 | 0 |
| | 90 | 50 |
| Nasal application TASK1/3 | 120 | 0 |
| channel blocker | | |
| | 130 | 100 |
| | 150 | 100 |
| | 180 | 100 |
| | 240 | 100 |
| | 300 | 100 |
| | 360 | 100 |

TABLE 11
Combination of non effective dose of ent-N-[(3,5-Difluoropyridin-2-yl)methyl]-2-[3-
(methoxymethyl)[1,4′-bipiperidin]-1′-yl]-1,3-thiazole-5-carboxamide with the non
effective dose of 0.3 μg of (4-{[2-(4-chlorophenyl)imidazo[1,2-a]pyridin-3-
yl]methyl} piperazin-1-yl)(6-methoxypyridin-2-yl)methanone inhibits upper airway
collapsibility at negative pressures of −100 cm head (cm H₂O)

| | Time, min | Percent pigs without collaps −100 cm H₂O, % |
|---|---|---|
| | 0 | 0 |
| | 30 | 0 |
| | 60 | 0 |
| | 90 | 50 |
| Nasal application TASK1/3 | 120 | 0 |
| channel blocker | | |
| | 130 | 100 |
| | 150 | 100 |
| | 180 | 100 |
| | 240 | 100 |
| | 300 | 100 |
| | 360 | 100 |

TABLE 12
Combination of non effective dose of ent-N-[(3,5-Difluoropyridin-2-yl)methyl]-2-[3-
(methoxymethyl)[1,4′-bipiperidin]-1′-yl]-1,3-thiazole-5-carboxamide with the non
effective dose of 0.3 μg of (4-{[2-(4-chlorophenyl)imidazo[1,2-a]pyridin-3-
yl]methyl}piperazin-1-yl)(6-methoxypyridin-2-yl)methanone inhibits upper airway
collapsibility at negative pressures of −150 cm head (cm H₂O)

| | Time, min | Percent pigs without collaps −150 cm H₂O, % |
|---|---|---|
| | 0 | 0 |
| | 30 | 0 |
| | 60 | 0 |
| | 90 | 50 |
| Nasal application TASK1/3 | 120 | 0 |
| channel blocker | | |
| | 130 | 100 |
| | 150 | 100 |
| | 180 | 100 |
| | 240 | 100 |
| | 300 | 100 |
| | 360 | 100 |

Table 13, 14 and 15 and FIG. 5: Effect of intranasal administration of 0.3 μg of the TASK1/TASK3 channel blocker (4-{[2-(4-chlorophenyl)imidazo[1,2-a]pyridin-3-yl]methyl}piperazin-1-yl)(6-methoxypyridin-2-yl)methanone given at time point 0 min on upper airway collapsibility at different levels of negative pressure. Percentages of pigs with no collapse are given. Mean values.

TABLE 13
Intranasal administration of 0.3 μg of the TASK1/TASK3 channel blocker
(4-{[2-(4-chlorophenyl)imidazo[1,2-a]pyridin-3-yl]methyl}piperazin-
1-yl)(6-methoxypyridin-2-yl)methanone at negative pressures of −50 cm
head (cm H₂O)

| Time, min | Percent pigs without collaps −50 cm H₂O, % |
|---|---|
| 0 | 0 |
| 10 | 0 |
| 30 | 0 |
| 60 | 0 |

TABLE 14
Intranasal administration of 0.3 μg of the TASK1/TASK3 channel blocker
(4-{[2-(4-chlorophenyl)imidazo[1,2-a]pyridin-3-yl]methyl}piperazin-
1-yl)(6-methoxypyridin-2-yl)methanone at negative pressures of −100 cm
head (cm H₂O)

| Time, min | Percent pigs without collaps −100 cm H₂O, % |
|---|---|
| 0 | 0 |
| 10 | 0 |
| 30 | 0 |
| 60 | 0 |

TABLE 15
Intranasal administration of 0.3 μg of the TASK1/TASK3 channel blocker
(4-{[2-(4-chlorophenyl)imidazo[1,2-a]pyridin-3-yl]methyl}piperazin-
1-yl)(6-methoxypyridin-2-yl)methanone at negative pressures of −150 cm
head (cm H₂O)

| Time, min | Percent pigs without collaps −150 cm H₂O, % |
|---|---|
| 0 | 0 |
| 10 | 0 |
| 30 | 0 |
| 60 | 0 |

From the above mentioned data it can be deducted that the combination of an α2-Adrenoceptor subtype C (alpha-2C) antagonists of formula (I) with a TASK⅓ channel blocker inhibits upper airway collapsibility with improved efficacy compared to each treatment alone and is thus suitable to treat sleep-related breathing disorders, preferably obstructive and central sleep apneas and snoring.

1. A combination of compounds of formula (I) 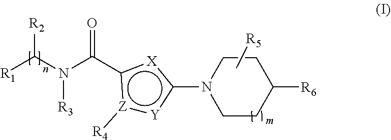
wherein
X is S, N, or O;
Y is N, S, or O,
wherein if X is S, then Y is N;
Z is C, O, or N,
wherein if X is N and Y is N, then Z is O;
R₁ is 5- or 6-membered heteroaryl or phenyl,
wherein 5- or 6-membered heteroaryl may be substituted by 1 or 2 substituents independently selected from the group consisting of (C₁-C₄)-alkyl, (C₁-C₄)-alkoxy, and halogen,
wherein (C₁-C₄)-alkyl may be substituted up to trisubstituted by halogen,
wherein (C₁-C₄)-alkoxy may be substituted up to trisubstituted by halogen,
wherein phenyl may be substituted by 1 or 2 substituents independently selected from the group consisting of (C₁-C₄)-alkyl, (C₃-C₅)-cycloalkyl, (C₁-C₄)-alkoxy, cyano, hydroxy, and halogen,
wherein (C₁-C₄)-alkyl may be substituted up to trisubstituted by halogen;
R₂ is hydrogen or (C₁-C₄)-alkyl,
wherein (C₁-C₄)-alkyl may be substituted up to trisubstituted by halogen, or
together with the carbon atom to which R₂ is attached, form a (C₃-C₄)-cycloalkyl ring;
R₃ represents hydrogen or (C₁-C₄)-alkyl,
wherein (C₁-C₄)-alkyl may be substituted up to trisubstituted by halogen;
R₄ is absent when Z represents N or O, or is hydrogen, (C₁-C₄)-alkyl, (C₃-C₄)-cycloalkyl, phenyl or halogen when Z represents C,
wherein (C₁-C₄)-alkyl may be substituted up to trisubstituted by halogen, and phenyl may be substituted by halogen;
R₅ is hydrogen, (C₁-C₄)-alkyl, (C₁-C₄)-alkoxy, or halogen;
R₆ is a group selected from the group consisting of the formula a), b), c), d), e), f), or g) 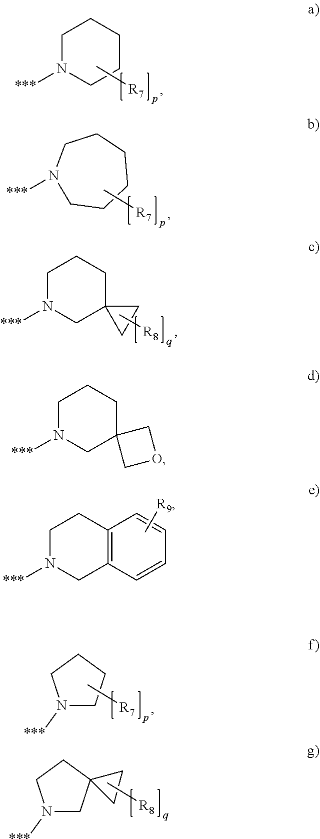
wherein *** marks the bond to the adjacent piperidine ring,
wherein R₇ is hydrogen, (C₁-C₄)-alkyl, (C₃-C₄)-cycloalkyl, (C₁-C₄)-alkoxy, (C₃-C₄)-cycloalkoxy, or phenyl,
wherein (C₁-C₄)-alkyl may be substituted by (C₃-C₄)-cycloalkyl, (C₁-C₄)-alkoxy, or (C₃-C₄)-cycloalkoxy and may be up to trisubstituted by halogen,
wherein (C₁-C₄)-alkoxy in turn may be substituted by (C₃-C₄)-cycloalkyl and may be up to trisubstituted by halogen,
wherein (C₃-C₄)-cycloalkyl may be substituted by monofluoromethyl, difluoromethyl, or trifluoromethyl and may be up to disubstituted by halogen,
wherein (C₁-C₄)-alkoxy may be substituted by (C₃-C₄)-cycloalkyl and may be up to trisubstituted by halogen,
in which (C₃-C₄)-cycloalkyl may be monosubstituted or disubstituted by halogen,
wherein (C₃-C₄)-cycloalkoxy may be up to disubstituted by halogen,
wherein R⁸ is hydrogen or fluoro,
wherein R₉ is hydrogen, (C₁-C₄)-alkyl, (C₁-C₄)-alkoxy, or halogen,
wherein (C₁-C₄)-alkyl in turn may be substituted by (C₁-C₄)-alkoxy;
n is 0 or 1;
m is 0, 1, or 2;
p is 0, 1, or 2; and
q is 0, 1, or 2,
with compounds of the formula (II) 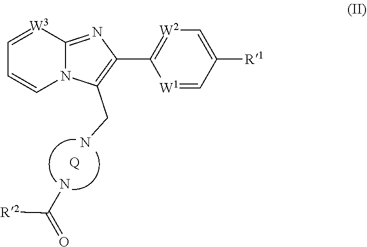
wherein
the ring Q is a piperazine or a diazaheterobicyclic system of the formula 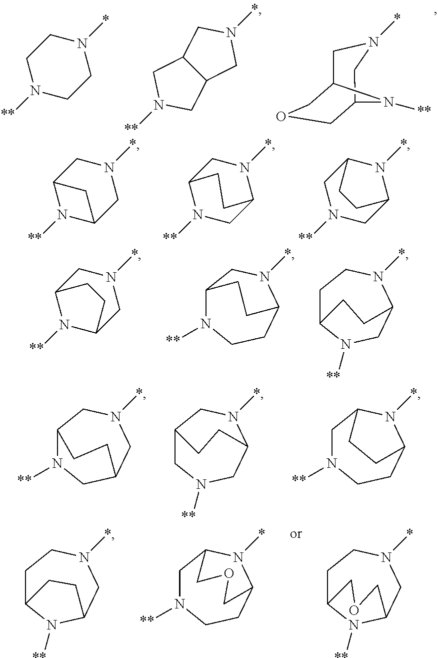
wherein * denotes the bond to the adjacent CHR′² group and ** denotes the bond to the carbonyl group;
W¹, W², and W³ are each independently CH or N;
R′¹ is halogen, cyano, (C₁-C₄)-alkyl, cyclopropyl, or cyclobutyl where (C₁-C₄)-alkyl may be up to trisubstituted by fluorine and cyclopropyl and cyclobutyl may be up to disubstituted by fluorine;
R′² is (C₄-C₆)-cycloalkyl in which a ring CH₂ group may be replaced by —O—,
or
R′² is a phenyl group of the formula (a), a pyridyl group of the formula (b) or (c), or an azole group of the formula (d), (e), (f), or (g), 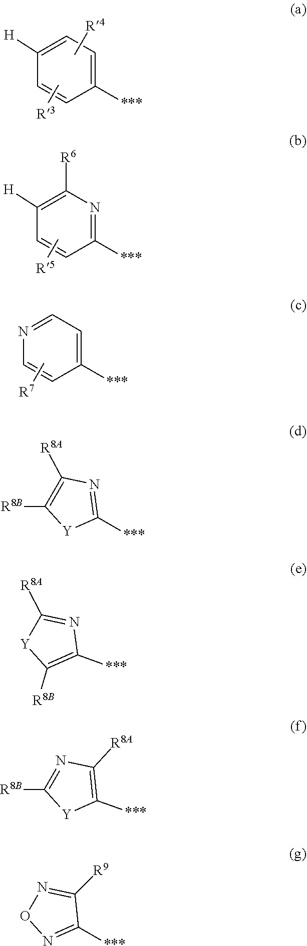
wherein *** marks the bond to the adjacent carbonyl group;
R′³ is hydrogen, fluorine, chlorine, bromine, or methyl,
R′⁴ is hydrogen, fluorine, chlorine, bromine, cyano, (C₁-C₃)-alkyl or (C₁-C₃)-alkoxy,
wherein (C₁-C₃)-alkyl and (C₁-C₃)-alkoxy may each be up to trisubstituted by fluorine;
R′⁵ is hydrogen, fluorine, chlorine, bromine, or methyl;
R′⁶ is hydrogen, (C₁-C₃)-alkoxy, cyclobutyloxy, oxetan-3-yloxy, tetrahydrofuran-3-yloxy, tetrahydro-2H-pyran-4-yloxy, mono-(C₁-C₃)-alkylamino, di-(C₁-C₃)-alkylamino or (C₁-C₃)-alkylsulfanyl,
wherein (C₁-C₃)-alkoxy may be up to trisubstituted by fluorine,
R⁷ is hydrogen, fluorine, chlorine, bromine, (C₁-C₃)-alkyl, or (C₁-C₃)-alkoxy;
R^{8A} and R^{8B} are each independently hydrogen, fluorine, chlorine, bromine, (C₁-C₃)-alkyl, cyclopropyl, or (C₁-C₃)-alkoxy,
wherein (C₁-C₃)-alkyl and (C₁-C₃)-alkoxy may each be up to trisubstituted by fluorine;
R⁹ is hydrogen, (C₁-C₃)-alkyl, or amino; and
in subformula (d), Y is O, S, or N(CH₃),
in subformula (e) and (f), Y is O or S,
or
R′² is —OR¹⁰ or —NR¹¹R¹², wherein
R¹⁰ is (C₁-C₆)-alkyl, (C₄-C₆)-cycloalkyl, or [(C₃-C₆)-cycloalkyl]methyl;
R¹¹ is hydrogen or (C₁-C₃)-alkyl
and
R¹² is (C₁-C₆)-alkyl, (C₃-C₆)-cycloalkyl, phenyl, benzyl, 1-phenylethyl, or 2-phenylethyl,
wherein (C₁-C₆)-alkyl may be up to trisubstituted by fluorine, and
wherein phenyl and the phenyl group in benzyl, 1-phenylethyl, and 2-phenylethyl may be up to trisubstituted by identical or different radicals selected from the group consisting of fluorine, chlorine, methyl, ethyl, trifluoromethyl, methoxy, ethoxy, trifluoromethoxy, and (trifluoromethyl)sulfanyl,
or
R¹¹ and R¹² are attached to one another and, together with the nitrogen atom to which they are bonded, form a pyrrolidine, piperidine, morpholine, or thiomorpholine ring, or
R¹¹ and R¹² are attached to one another and, together with the nitrogen atom to which they are bonded, form a tetrahydroquinoline ring of the formula (c) or a tetrahydroisoquinoline ring of the formula (d), 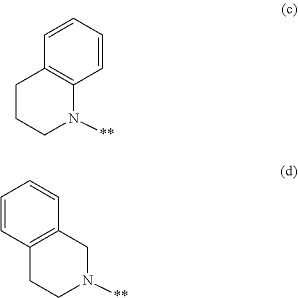
wherein ** marks the bond to the carbonyl group,
or a salt, solvate, or solvate of a salt thereof.

2. The combination of claim 1,
wherein
X, Y, and Z are selected from the group consisting of S, N, O, and C to form 1,3-thiazolyl, 1,3-oxazolyl, or 1,2,4-oxadiazolyl;
R₁ is pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, or 5-fluoro-2-thienyl;
R₂ is hydrogen or methyl;
R₃ is hydrogen or methyl;
R₄ is hydrogen, methyl, ethyl, or trifluormethyl;
R₅ is hydrogen or fluoro,
R₆ is a group of the formula a), c′), or c″), 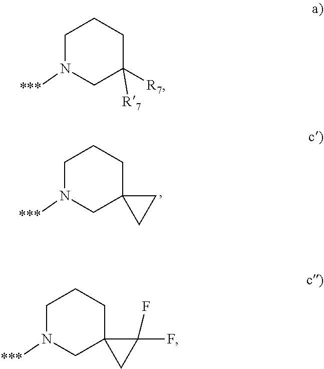
wherein *** marks the bond to the adjacent piperidine ring,
wherein R₇ and R′₇ are each independently hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy, or fluoro;
n is 0 or 1;
m is 1
the ring Q is a piperazine or a diazaheterobicyclic system of the formula 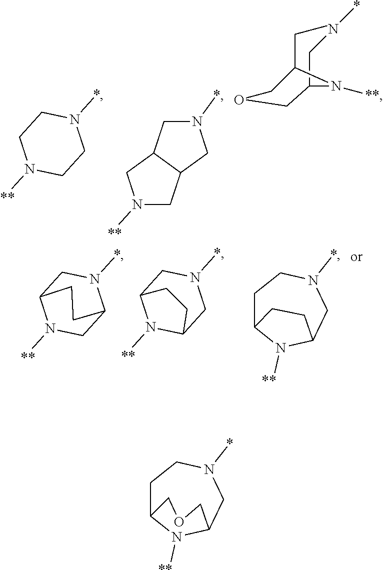
W² is CH,
W¹ and W³ are each independently CH or N,
R′¹ is fluorine, chlorine, bromine, methyl, tert-butyl, isopropyl, cyclopropyl, or cyclobutyl,
R′² is cyclobutyl, cyclopentyl, or cyclohexyl,
or
R′² is a phenyl group of the formula (a), a pyridyl group of the formula (b), or an azole group of the formula (d) or formula (g) 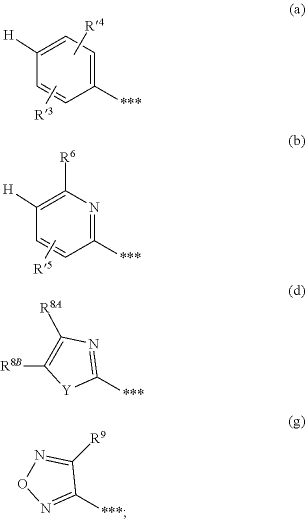
R′³ is hydrogen, fluorine, or chlorine;
R′⁴ is fluorine, chlorine, methyl, isopropyl, methoxy, or ethoxy;
R′⁵ is hydrogen, fluorine, chlorine, bromine, or methyl;
R⁶ is methoxy, difluoromethoxy, trifluoromethoxy, isopropoxy, cyclobutyloxy, or methylsulfanyl;
R^{8A} and R^{8B} are each independently hydrogen, methyl, trifluoromethyl, ethyl, isopropyl, or cyclopropyl;
R⁹ represents methyl or amino; and
Y is O, S, or N(CH₃),
or a salt, solvate, or solvate of a salt thereof.

3. The combination of claim 1, wherein
X, Y, and Z are selected from the group consisting of of S, N, O, and C to form 1,3-thiazolyl, 1,3-oxazolyl, or 1,2,4-oxadiazolyl;
R₁ is pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, or 5-fluoro-2-thienyl;
R₂ is hydrogen or methyl;
R₃ is hydrogen or methyl;
R₄ is hydrogen, methyl, ethyl, or trifluormethyl,
wherein phenyl may be substituted by chloro;
R₅ is hydrogen or fluoro;
R₆ is a group of the formula a), c′), or c″), 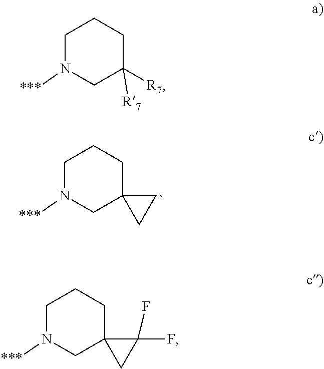
wherein R₇ and R′₇ are each independently hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy, or fluoro;
n is 0 or 1;
m is 1;
the ring Q is a diazaheterobicyclic system of the formula 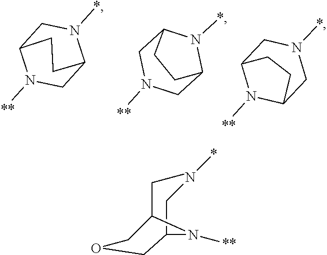
W¹ is CH;
W² is CH;
W³ is N;
R′¹ is chlorine, bromine, isopropyl, or cyclobutyl;
R′² is cyclopentyl or cyclohexyl,
or
R′² is a phenyl group of the formula (a), a pyridyl group of the formula (b), or an azole group of the formula (d), (e), or (f) 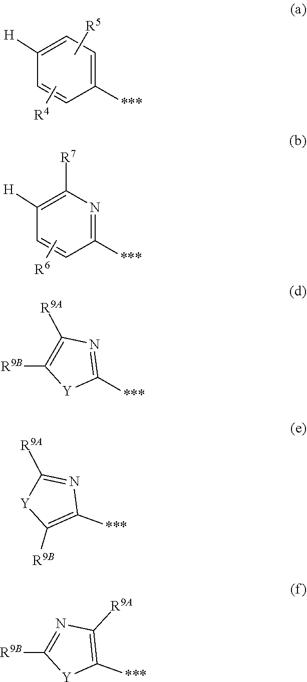
R⁴ is hydrogen, fluorine or chlorine;
R⁵ is fluorine, chlorine, methyl, isopropyl, methoxy, or ethoxy;
R⁶ is hydrogen, fluorine, chlorine, bromine, or methyl;
R⁷ is methoxy, difluoromethoxy, trifluoromethoxy, isopropoxy, cyclobutyloxy, or methylsulfanyl;
R^{9A} and R^{9B} are and each independently hydrogen, methyl, trifluoromethyl, ethyl, isopropyl, or cyclopropyl; and
Y is O or S,
or a salt, solvate, or solvate of a salt thereof.

4. The combination of claim 1, wherein the compound of formula (I) is selected from the group consisting of:
N-[(3,5-difluoropyridin-2-yl)methyl]-2-[(3R)-3-methyl[1,4′-bipiperidin]-1′-yl]-1,3-thiazole-5-carboxamide,
2-[4-(5-azaspiro[2.5]octan-5-yl)piperidin-1-yl]-N-[(3,5-difluoropyridin-2-yl)methyl]-1,3-thiazole-5-carboxamide,
N-[(3,5-difluoropyridin-2-yl)methyl]-2-[(3R*)-3-(methoxymethyl)[1,4′-bipiperidin]-1′-yl]-1,3-thiazole-5-carboxamide,
4-chloro-N-[(3,5-difluoropyridin-2-yl)methyl]-2-[(3R)-3-methyl[1,4′-bipiperidin]-1′-yl]-1,3-thiazole-5-carboxamide, and
N-[1-(3,5-difluoropyridin-2-yl)cyclopropyl]-2-[(3R)-3-methyl[1,4′-bipiperidin]-1′-yl]-1,3-thiazole-5-carboxamide,
and the compound of formula (II) is selected from the group consisting of:
(4-{[2-(4-Chlorophenyl)imidazo[1,2-a]pyridin-3-yl]methyl}piperazin-1-yl)(6-methoxypyridin-2-yl)methanone,
(5-{[2-(4-Chlorophenyl)imidazo[1,2-a]pyrimidin-3-yl]methyl}-2,5-diazabicyclo[2.2.2]oct-2-yl)(3-fluoro-6-methoxypyridin-2-yl)methanone,
(3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, and
(3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

5. The combination of claim 1, wherein the compound of formula (I) is:
N-[(3,5-difluoropyridin-2-yl)methyl]-2-[(3R)-3-methyl[1,4′-bipiperidin]-1′-yl]-1,3-thiazole-5-carboxamide,
and the compound of formula (II) is selected from the group consisting of:
(4-{[2-(4-Bromophenyl)imidazo[1,2-a]pyridin-3-yl]methyl}piperazin-1-yl)(cyclopentyl)methanone,
(4-{[2-(4-Chlorophenyl)imidazo[1,2-a]pyridin-3-yl]methyl}piperazin-1-yl)(cyclopentyl)methanone,
(4-{[2-(4-Chlorophenyl)imidazo[1,2-a]pyridin-3-yl]methyl}piperazin-1-yl)(6-methoxypyridin-2-yl)methanone,
(4-{[2-(4-Bromophenyl)imidazo[1,2-a]pyridin-3-yl]methyl}piperazin-1-yl)(2-fluorophenyl)methanone,
(4-{[2-(4-chlorophenyl)imidazo[1,2-a]pyridin-3-yl]methyl}piperazin-1-yl)(6-isopropoxypyridin-2-yl)methanone,
(4-{[2-(4-bromophenyl)imidazo[1,2-a]pyridin-3-yl]methyl}piperazin-1-yl)(6-methoxypyridin-2-yl)methanone,
(4-{[2-(4-Chlorophenyl)imidazo[1,2-a]pyridin-3-yl]methyl}piperazin-1-yl)[6-(trifluoromethoxy)pyridin-2-yl]methanone,
(4-{[2-(4-Chlorophenyl)imidazo[1,2-a]pyridin-3-yl]methyl}piperazin-1-yl)(3-fluoro-6-methoxypyridin-2-yl)methanone,
[5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone,
[5-{[2-(4-Isopropylphenyl)imidazo[1,2-a]pyridin-3-yl]methyl}hexahydropyrrolo[3,4-c]pyrrol-2(1H)-yl](6-methoxypyridin-2-yl)methanone,
(3-Fluoro-6-methoxypyridin-2-yl)[5-{[2-(4-isopropylphenyl)imidazo[1,2-a]pyridin-3-yl]methyl}hexahydropyrrolo[3,4-c]pyrrol-2(1H)-yl]methanone,
[5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone,
(−)-[(1S,4S)-5-{[2-(4-Chlorophenyl)imidazo[1,2-a]pyridin-3-yl]methyl}-2,5-diazabicyclo[2.2.2]oct-2-yl](6-methoxypyridin-2-yl)methanone,
(−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone,
(−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone,
(5-{[2-(5-Chloropyridin-2-yl)imidazo[1,2-a]pyridin-3-yl]methyl}-2,5-diazabicyclo[2.2.2]oct-2-yl)(3-fluoro-6-methoxypyridin-2-yl)methanone,
(3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone,
(−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone,
(5-{[2-(5-Chloropyridin-2-yl)imidazo[1,2-a]pyridin-3-yl]methyl}-2,5-diazabicyclo[2.2.2]oct-2-yl)[6-(difluoromethoxy)pyridin-2-yl]methanone,
(3-{[2-(4-Chlorophenyl)imidazo[1,2-a]pyrimidin-3-yl]methyl}-3,8-diazabicyclo[3.2.1]oct-8-yl)(6-methoxypyridin-2-yl)methanone,
(3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone,
(3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone,
(3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone,
(3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone,
(3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone,
(3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone,
(3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone,
3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone,
3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone,
[3-{[2-(5-Chloropyridin-2-yl)imidazo[1,2-a]pyridin-3-yl]methyl}-3,9-diazabicyclo[4.2.1]non-9-yl](3-fluoro-6-methoxypyridin-2-yl)methanone,
(3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

6. The combination of claim 1, wherein the compound of formula (I) is:
N-[(3,5-difluoropyridin-2-yl)methyl]-2-[(3R)-3-methyl[1,4′-bipiperidin]-1′-yl]-1,3-thiazole-5-carboxamide,
and the compound of formula (II) is-(4-{[2-(4-Chlorophenyl)imidazo[1,2-a]pyridin-3-yl]methyl}piperazin-1-yl)(6-methoxypyridin-2-yl)methanone or (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

7-8. (canceled)

9. A pharmaceutical composition comprising the combination of claim 1 and one or more inert, nontoxic, pharmaceutically suitable excipients.

10. A pharmaceutical composition comprising the combination of claim 1 and one or more further active ingredients selected from the group consisting of muscarinic receptor antagonists, mineralocorticoid receptor antagonists, diuretics, and corticosteroids.

11. (canceled)

12. A method of treatment and/or prevention of respiratory disorders, sleep-related respiratory disorders, obstructive sleep apnoea, central sleep apnoea, snoring, cardiac arrhythmias, neurodegenerative disorders, neuroinflammatory disorders, or neuroimmunological disorders in humans and animals comprising administering an effective amount of at least one combination of claim 1.

13. The method of claim 12, wherein the method treats and/or prevents obstructive sleep apnoea, central sleep apnoea, or snoring.

14. A method of treatment and/or prevention of respiratory disorders, sleep-related respiratory disorders, obstructive sleep apnoea, central sleep apnoea, snoring, cardiac arrhythmias, neurodegenerative disorders, neuroinflammatory disorders, or neuroimmunological disorders comprising administering the medicament of claim 9 to a subject in need thereof.

15. The method of claim 14, wherein the method treats and/or prevents obstructive sleep apnoea, central sleep apnoea, or snoring.

16. A method of treatment and/or prevention of respiratory disorders, sleep-related respiratory disorders, obstructive sleep apnoea, central sleep apnoea, snoring, cardiac arrhythmias, neurodegenerative disorders, neuroinflammatory disorders, or neuroimmunological disorders comprising administering the medicament of claim 10 to a subject in need thereof.

17. The method of claim 16, wherein the method treats and/or prevents obstructive sleep apnoea, central sleep apnoea, or snoring.
